(19) United States
(12) Patent Application Publication
(10) Pub. No.: US 2006/0150283 A1
(43) Pub. Date: Jul. 6, 2006

(54) Sequence-determined DNA fragments and corresponding polypeptides encoded thereby

(76) Inventors: Nickolai Alexandrov, Thousand Oaks, CA (US); Vyacheslav Brover, Simi Valley, CA (US)

Correspondence Address: BIRCH STEWART KOLASCH & BIRCH, PO BOX 747, FALLS CHURCH, VA, 22040-0747, US

(21) Appl. No.: 11/056,355

(22) Filed: Feb. 14, 2005

Related U.S. Application Data
(60) Provisional application No. 60/544,190, filed on Feb. 13, 2004.

(51) Int. Cl. A01H 1/00; C12Q 1/68; C07H 21/04; C12N 15/82; C12N 5/04

(52) U.S. Cl. 800/288; 435/6; 435/419; 435/468; 536/23.6

(57) ABSTRACT

The present invention provides DNA molecules that constitute fragments of the genome of a plant, and polypeptides encoded thereby. The DNA molecules are useful for specifying a gene product in cells, either as a promoter or as a protein coding sequence or as an UTR or as a 3′ termination sequence, and are also useful in controlling the behavior of a gene in the chromosome, in controlling the expression of a gene or as tools for genetic mapping, recognizing or isolating identical or related DNA fragments, or identification of a particular individual organism, or for clustering of a group of organisms with a common trait. One of ordinary skill in the art, having this data, can obtain cloned DNA fragments, synthetic DNA fragments or polypeptides constituting desired sequences by recombinant methodology known in the art or described herein.

This Non-provisional application claims priority under 35 U.S.C. §119(e) on U.S. Provisional Application No. 60/544,190 filed on Feb. 13, 2004, the entire contents of which are hereby incorporated by reference.

This application contains a CDR, the entire contents of which are hereby incorporated by reference. The CDR contains the following files:

| Creation Date | File Size | File Name |
|---|---|---|
| 2005-02-14 | 54998 KB | 2005-02-14 Sequence Listing-Misc |
| | | Features (1) |
| 2005-02-14 | 56333 KB | 2005-02-14 Sequence Listing-Misc |
| | | Features (2) |
| 2005-02-14 | 129064 KB | 2005-02-14 Sequence Listing-Misc |
| | | Features (3) |
| 2005-02-14 | 21127 KB | 2005-02-14 Sequence Listing-Misc |
| | | Features (4) |
| 2005-02-14 | 185063 KB | 2005-02-14 Sequence Listing |
| | | (Batch 3 plus) |
| 2005-02-14 | 45865 KB | 2005-02-14 Sequence Listing (Batch 1a) |
| 2005-02-14 | 119718 KB | 2005-02-14 Sequence Listing (Batch 1b) |
| 2005-02-14 | 48031 KB | 2005-02-14 Sequence Listing (Batch 2) |

### FIELD OF THE INVENTION

The present invention relates to over 100,000 isolated polynucleotides from plants that include a complete coding sequence, or a fragment thereof, that is expressed. In addition, the present invention relates to the polypeptide or protein corresponding to the coding sequence of these polynucleotides. The present invention also relates to isolated polynucleotides that represent regulatory regions of genes. The present invention also relates to isolated polynucleotides that represent untranslated regions of genes. The present invention further relates to the use of these isolated polynucleotides and polypeptides and proteins.

### BACKGROUND OF THE INVENTION

There are more than 300,000 species of plants. They show a wide diversity of forms, ranging from delicate liverworts, adapted for life in a damp habitat, to cacti, capable of surviving in the desert. The plant kingdom includes herbaceous plants, such as corn, whose life cycle is measured in months, to the giant redwood tree, which can live for thousands of years. This diversity reflects the adaptations of plants to survive in a wide range of habitats. This is seen most clearly in the flowering plants (phylum Angiospermophyta), which are the most numerous, with over 250,000 species. They are also the most widespread, being found from the tropics to the arctic.

The process of plant breeding involving man's intervention in natural breeding and selection is some 20,000 years old. It has produced remarkable advances in adapting existing species to serve new purposes. The world's economics was largely based on the successes of agriculture for most of these 20,000 years.

Plant breeding involves choosing parents, making crosses to allow recombination of gene (alleles) and searching for and selecting improved forms. Success depends on the genes/alleles available, the combinations required and the ability to create and find the correct combinations necessary to give the desired properties to the plant. Molecular genetics technologies are now capable of providing new genes, new alleles and the means of creating and selecting plants with the new, desired characteristics.

When the molecular and genetic basis for different plant characteristics are understood, a wide variety of polynucleotides, both endogenous polynucleotides and created variants, polypeptides, cells, and whole organisms, can be exploited to engineer old and new plant traits in a vast range of organisms including plants. These traits can range from the observable morphological characteristics, through adaptation to specific environments to biochemical composition and to molecules that the plants (organisms) exude. Such engineering can involve tailoring existing traits, such as increasing the production of taxol in yew trees, to combining traits from two different plants into a single organism, such as inserting the drought tolerance of a cactus into a corn plant. Molecular and genetic knowledge also allows the creation of new traits. For example, the production of chemicals and pharmaceuticals that are not native to particular species or the plant kingdom as a whole.

The achievements described in this application were possible because of the results from a cluster of technologies, a genomic engine, depicted below in Schematic 1, that allows information on each gene to be integrated to provide a more comprehensive understanding of gene structure and function and the deployment of genes and gene components to make new products.

### I. The Discoveries of the Instant Application

Applicants have isolated and identified over one hundred thousand genes, gene components and their products and thousands of promoters. Specific genes were isolated and/or characterized from arabidopsis, soybean, maize, wheat and rice. These species were selected because of their economic value and scientific importance and were deliberately chosen to include representatives of the evolutionary divergent dicotyledonous and monocotyledonous groups of the plant kingdom.

The techniques used initially to isolate and characterize most of the genes, namely sequencing of full-length cDNAs, were deliberately chosen to provide information on complete coding sequences and on the complete sequences of their protein products.

Gene components and products the Applicants have identified include exons, introns, promoters, coding sequences, antisense sequences, terminators and other regulatory sequences. The exons are characterized by the proteins they encode and Arabidopsis promoters are characterized by their position in the genomic DNA relative to where mRNA synthesis begins and in what cells and to what extent they promote mRNA synthesis.

Further exploitation of molecular genetics technologies has helped the Applicants to understand the functions and characteristics of each gene and their role in a plant. Three powerful molecular genetics approaches were used to this end:
(a) Analyses of the phenotypic changes when the particular gene sequence is interrupted or activated differentially; (arabidopsis)
(b) Analyses of in what plant organs, to what extent, and in response to what environmental signals mRNA is synthesized from the gene; (arabidopsis and maize) and
(c) Analysis of the gene sequence and its relatives. (all species)

These were conducted using the genomics engine depicted in FIG. 1 that allows information on each gene to be integrated to provide a more comprehensive understanding of gene structure and function and linkage to potential products.

The species Arabidopsis was used extensively in these studies for several reasons: (1) the complete genomic sequence, though poorly annotated in terms of gene recognition, was being produced and published by others and (2) genetic experiments to determine the role of the genes in planta are much quicker to complete.

The phenotypic data, MA data, and reference data and sequence data indicate the results of these analyses and thus the specific functions and characteristics that are ascribed to the genes and gene components and products.

[image]

### II. Integration of Discoveries to Provide Scientific Understanding

From the discoveries made, Applicants have deduced the biochemical activities, pathways, cellular roles, and developmental and physiological processes that can be modulated using these components. These are discussed and summarized in sections based on the gene functions characteristics from the analyses and role in determining phenotypes. These sections illustrate and emphasize that each gene, gene component or product influences biochemical activities, cells or organisms in complex ways, from which there can be many phenotypic consequences.

An illustration of how the discoveries on gene structure, function, expression and phenotypic observation can be integrated together to understand complex phenotypes is provided in schematic 2. This sort of understanding enables conclusions to be made as to how the genes, gene components and product are useful for changing the properties of plants and other organisms. This example also illustrates how single gene changes in, for example, a metabolic pathway can cause gross phenotypic changes.

[image]

Furthermore, the development and properties of one part of plant can be interconnected with other parts. The dependence of shoot and leaf development on root cells is a classic example. Here, shoot growth and development require nutrients supplied from roots, so the protein complement of root cells can affect plant development, including flowers and seed production. Similarly, root development is dependent on the products of photosynthesis from leaves. Therefore, proteins in leaves can influence root developmental physiology and biochemistry.

Thus, the following sections describe both the functions and characteristics of the genes, gene components and products and also the multiplicity of biochemical activities, cellular functions, and the developmental and physiological processes influenced by them.

A. Analyses to Reveal Function and In Vivo Roles of Single Genes in One Plant Species

The genomics engine has focused on individual genes to reveal the multiple functions or characteristics that are associated to each gene, gene components and products of the instant invention in the living plant. For example, the biochemical activity of a protein is deduced based on its similarity to a protein of known function. In this case, the protein may be ascribed with, for example, an oxidase activity. Where and when this same protein is active can be uncovered from differential expression experiments, which show that the mRNA encoding the protein is differentially expressed in response to drought and in seeds but not roots. The gene disruption experiments reveal that absence of the same protein causes embryo lethality.

Thus, this protein is characterized as a seed protein and drought-responsive oxidase that is critical for embryo viability.

B. Analyses to Reveal Function and Roles of Single Genes in Different Species

The genomics engine has also been used to extrapolate knowledge from one species to many plant species. For example, proteins from different species, capable of performing identical or similar functions, preserve many features of amino acid sequence and structure during evolution. Complete protein sequences have been compared and contrasted within and between species to determine the functionally vital domains and signatures characteristic of each of the proteins that is the subject of this application. Thus, functions and characteristics of arabidopsis proteins have been extrapolated to proteins containing similar domains and signatures of corn, soybean, rice and wheat and by implication to all other (plant) species.

[image]

Schematic 3 provides an example. Two proteins with related structures, one from corn, a monocot, and one from arabidopsis, a dicot, have been concluded to be orthologs. The known characteristics of the arabidopsis protein (seed protein, drought responsive oxidase) can then be attributed to the corn protein.

C. Analyses Over Multiple Experiments to Reveal Gene Networks and Links Across Species

The genomics engine can identify networks or pathways of genes concerned with the same process and hence linked to the same phenotype(s). Genes specifying functions of the same pathway or developmental environmental responses are frequently co-regulated i.e. they are regulated by mechanisms that result in coincident increases or decreases for all gene members in the group. The Applicants have divided the genes of arabidopsis and maize into such co-regulated groups on the basis of their expression patterns and the function of each group has been deduced. This process has provided considerable insight into the function and role of thousands of the plant genes in diverse species included in this application.

D. Applications of Applicant's Discoveries

It will be appreciated while reading the sections that the different experimental molecular genetic approaches focused on different aspects of the pathway from gene and gene product through to the properties of tissues, organs and whole organisms growing in specific environments. For each endogenous gene, these pathways are delineated within the existing biology of the species. However, Applicants' inventions allow gene components or products to be mixed and matched to create new genes and placed in other cellular contexts and species, to exhibit new combinations of functions and characteristics not found in nature, or to enhance and modify existing ones. For instance, gene components can be used to achieve expression of a specific protein in a new cell type to introduce new biochemical activities, cellular attributes or developmental and physiological processes. Such cell-specific targeting can be achieved by combining polynucleotides encoding proteins with any one of a large array of promoters to facilitate synthesis of proteins in a selective set of plant cells. This emphasizes that each gene, component and protein can be used to cause multiple and different phenotypic effects depending on the biological context. The utilities are therefore not limited to the existing in vivo roles of the genes, gene components, and gene products.

While the genes, gene components and products disclosed herein can act alone, combinations are useful to modify or modulate different traits. Useful combinations include different polynucleotides and/or gene components or products that have (1) an effect in the same or similar developmental or biochemical pathways; (2) similar biological activities; (3) similar transcription profiles; or (4) similar physiological consequences.

Of particular interest are the transcription factors and key factors in regulatory transduction pathways, which are able to control entire pathways, segments of pathways or large groups of functionally related genes. Therefore, manipulation of such proteins, alone or in combination is especially useful for altering phenotypes or biochemical activities in plants. Because interactions exist between hormone, nutrition, and developmental pathways, combinations of genes and/or gene products from these pathways also are useful to produce more complex changes. In addition to using polynucleotides having similar transcription profiles and/or biological activities, useful combinations include polynucleotides that may exhibit different transcription profiles but which participate in common or overlapping pathways. Also, polynucleotides encoding selected enzymes can be combined in novel ways in a plant to create new metabolic pathways and hence new metabolic products.

The utilities of the various genes, gene components and products of the Application are described below in the sections entitled as follows:
I. Organ Affecting Genes, Gene Components, Products (Including Differentiation Function)
   I.A. Root Genes, Gene Components And Products
      I.A.1. Root Genes, Gene Components And Products
      I.A.2. Root Hair Genes, Gene Components And Products
   I.B. Leaf Genes, Gene Components And Products
      I.B.1. Leaf Genes, Gene Components And Products
      I.B.2. Trichome Genes And Gene Components
      I.B.3. Chloroplast Genes And Gene Components
   I.C. Reproduction Genes, Gene Components And Products
      I.C.1. Reproduction Genes, Gene Components And Products
      I.C.2. Ovule Genes, Gene Components And Products
      I.C.3. Seed And Fruit Development Genes, Gene Components And Products
   I.D. Development Genes, Gene Components And Products
      I.D.1. Imbibition And Germination Responsive Genes, Gene Components And Products
      I.D.2. Early Seedling Phase Genes, Gene Components And Products
      I.D.3. Size And Stature Genes, Gene Components And Products
      I.D.4. Shoot-Apical Meristem Genes, Gene Components And Products
      I.D.5. Vegetative-Phase Specific Responsive Genes, Gene Components And Products
II. Hormones Responsive Genes, Gene Components And Products
   II.A. Abscissic Acid Responsive Genes, Gene Components And Products
   II.B. Auxin Responsive Genes, Gene Components And Products
   II.C. Brassinosteroid Responsive Genes, Gene Components And Products
   II.D. Cytokinin Responsive Genes, Gene Components And Products
   II.E. Gibberellic Acid Responsive Genes, Gene Components And Products
III. Metabolism Affecting Genes, Gene Components And Products
   III.A. Nitrogen Responsive Genes, Gene Components And Products
   III.B. Circadian Rhythm Responsive Genes, Gene Components And Products
   III.C. Blue Light (Phototropism) Responsive Genes, Gene Components And Products
   III.D. Co2 Responsive Genes, Gene Components And Products
   III.E. Mitochondria Electron Transport Genes, Gene Components And Products
   III.F. Protein Degradation Genes, Gene Components And Products
   III.G. Carotenogenesis Responsive Genes, Gene Components And Products
IV. Viability Genes, Gene Components And Products
   IV.A. Viability Genes, Gene Components And Products
   IV.B. Histone Deacetylase (Axel) Responsive Genes, Gene Components And Products
V. Stress Responsive Genes, Gene Components And Products
   V.A. Cold Responsive Genes, Gene Components And Products
   V.B. Heat Responsive Genes, Gene Components And Products
   V.C. Drought Responsive Genes, Gene Components And Products
   V.D. Wounding Responsive Genes, Gene Components And Products
   V.E. Methyl Jasmonate Responsive Genes, Gene Components And Products
   V.F. Reactive Oxygen Responsive Genes, Gene Components And H2O2 Products
   V.G. Salicylic Acid Responsive Genes, Gene Components And Products
   V.H. Nitric Oxide Responsive Genes, Gene Components And Products
   V.I. Osmotic Stress Responsive Genes, Gene Components And Products
   V.J. Aluminum Responsive Genes, Gene Components And Products
   V.K. Cadmium Responsive Genes, Gene Components And Products
   V.L. Disease Responsive Genes, Gene Components And Products
   V.M. Defense Responsive Genes, Gene Components And Products
   V.N. Iron Responsive Genes, Gene Components And Products
   V.O. Shade Responsive Genes, Gene Components And Products
   V.P. Sulfur Responsive Genes, Gene Components And Products
   V.Q. Zinc Responsive Genes, Gene Components And Products
VI. Enhanced Food
VII. Promoters As Sentinels

### SUMMARY OF THE INVENTION

The present invention comprises polynucleotides, such as complete cDNA sequences and/or sequences of genomic DNA encompassing complete genes, fragments of genes, and/or regulatory elements of genes and/or regions with other functions and/or intergenic regions, hereinafter collectively referred to as Sequence-Determined DNA Fragments (SDFs) or sometimes collectively referred to as “genes or gene components”, or sometimes as “genes, gene components or products”, from different plant species, particularly corn, wheat, soybean, rice and Arabidopsis thaliana, and other plants and or mutants, variants, fragments or fusions of said SDFs and polypeptides or proteins derived therefrom. In some instances, the SDFs span the entirety of a protein-coding segment. In some instances, the entirety of an MRNA is represented. Other objects of the invention that are also represented by SDFs of the invention are control sequences, such as, but not limited to, promoters. Complements of any sequence of the invention are also considered part of the invention.

Other objects of the invention are polynucleotides comprising exon sequences, polynucleotides comprising intron sequences, polynucleotides comprising introns together with exons, intron/exon junction sequences, 5′ untranslated sequences, and 3′ untranslated sequences of the SDFs of the present invention. Polynucleotides representing the joinder of any exons described herein, in any arrangement, for example, to produce a sequence encoding any desirable amino acid sequence are within the scope of the invention.

The present invention also resides in probes useful for isolating and identifying nucleic acids that hybridize to an SDF of the invention. The probes can be of any length, but more typically are 12-2000 nucleotides in length; more typically, 15 to 200 nucleotides long; even more typically, 18 to 100 nucleotides long.

Yet another object of the invention is a method of isolating and/or identifying nucleic acids using the following steps:

(a) contacting a probe of the instant invention with a polynucleotide sample under conditions that permit hybridization and formation of a polynucleotide duplex; and

(b) detecting and/or isolating the duplex of step (a).

The conditions for hybridization can be from low to moderate to high stringency conditions. The sample can include a polynucleotide having a sequence unique in a plant genome. Probes and methods of the invention are useful, for example, without limitation, for mapping of genetic traits and/or for positional cloning of a desired fragment of genomic DNA.

Probes and methods of the invention can also be used for detecting alternatively spliced messages within a species. Probes and methods of the invention can further be used to detect or isolate related genes in other plant species using genomic DNA (gDNA) and/or cDNA libraries. In some instances, especially when longer probes and low to moderate stringency hybridization conditions are used, the probe will hybridize to a plurality of cDNA and/or gDNA sequences of a plant. This approach is useful for isolating representatives of gene families which are identifiable by possession of a common functional domain in the gene product or which have common cis-acting regulatory sequences. This approach is also useful for identifying orthologous genes from other organisms.

The present invention also resides in constructs for modulating the expression of the genes comprised of all or a fragment of an SDF. The constructs comprise all or a fragment of the expressed SDF, or of a complementary sequence. Examples of constructs include ribozymes comprising RNA encoded by an SDF or by a sequence complementary thereto, antisense constructs, constructs comprising coding regions or parts thereof and constructs comprising promoters. Such constructs can be constructed using viral, plasmid, bacterial artificial chromosomes (BACs), plasmid artificial chromosomes (PACs), autonomous plant plasmids, plant artificial chromosomes or other types of vectors and exist in the plant as autonomous replicating sequences or as DNA integrated into the genome. When inserted into a host cell the construct is, preferably, functionally integrated with, or operatively linked to, a heterologous polynucleotide. For instance, a coding region from an SDF might be operably linked to a promoter that is functional in a plant.

The present invention also resides in host cells, including bacterial or yeast cells or plant cells, and plants that harbor constructs such as described above. Another aspect of the invention relates to methods for modulating expression of specific genes in plants by expression of the coding sequence of the constructs, by regulation of expression of one or more endogenous genes in a plant or by suppression of expression of the polynucleotides of the invention in a plant. Methods of modulation of gene expression include without limitation (1) inserting into a host cell additional copies of a polynucleotide comprising a coding sequence; (2) modulating an endogenous promoter in a host cell; (3) inserting antisense or ribozyine constructs into a host cell and (4) inserting into a host cell a polynucleotide comprising a sequence encoding a variant, fragment, or fusion of the native polypeptides of the instant invention.

### DETAILED DESCRIPTION OF THE INVENTION

### I. DESCRIPTION OF THE DATA

As noted above, the Applicants have obtained and analyzed an extensive amount of information on a large number of genes by use of the Ceres Genomic Engine to determine. This information can be categorized into three basic types:

A. Sequence Information for the Inventions

B. Transcriptional Information for the Inventions

C. Phenotypic Information for the Inventions

### I.A. Sequence Information

To harness the potential of the plant genome, Applicants began by elucidating a large number gene sequences, including the sequences of gene components and products, and analyzing the data. The list of sequences and associated data are presented in the Sequence Listing and Sequence Listing-Miscellaneous Features documents of the present application (sometimes referred to as the “REF” and “SEQ” Tables). The REF and SEQ tables include:
cDNA sequence;
coding sequence;
5′ & 3′ UTR;
transcription start sites;
exon and intron boundaries in genomic sequence; and
protein sequence.

The REF and SEQ tables also include computer-based, comparative analyses between the protein sequences of the invention and sequences with known function. Proteins with similar sequences typically exhibit similar biochemical activities. The REF table notes:
sequences of known function that are similar to the Applicants' proteins; and
biochemical activity that is associated with Applicants' proteins.

To identify gene components and products, Applicants took a cDNA/coding sequence approach. That is, Applicants initiated their studies either by isolating cDNAs and determining their sequences experimentally, or by identifying the coding sequence from genomic sequence with the aid of predictive algorithms. The cDNA sequences and coding sequences also are referred to as “Maximum Length Sequences” in the REF tables. The cDNA and coding sequences were given this designation to indicate these were the maximum length of coding sequences identified by Applicants.

Due to this cDNA/coding sequence focus of the present application, the REF and SEQ Tables were organized around cDNA and coding sequences. Each of these Maximum Length Sequences was assigned a unique identifier: Ceres Sequence ID NO, which is reported in the Tables.

All data that relate to these Maximum Length Sequences are grouped together, including 5′ & 3′ UTRs; transcription start sites; exon and intron boundaries in genomic sequence; protein sequence, etc.

Below, a more detailed explanation of the organization of the REF and SEQ Tables and how the data in the tables were generated is provided.

a. cDNA

Applicants have ascertained the sequences of mRNAs from different organisms by reverse transcription of mRNA to DNA, which was cloned and then sequenced. These complementary DNA or cDNA sequences also are referred to as Maximum Length Sequences in the REF Tables, which contain details on each of the sequences in the SEQ Tables.

Each sequence was assigned a Pat. Appln. Sequence ID NO: and an internal Ceres Sequence ID NO: as reported in the REF Table, the section labeled “(Ac) cDNA Sequence.” An example is shown below:
Max Len. Seq.:
(Ac) cDNA Sequence
   Pat. Appln. Sequence ID NO: 174538
   Ceres Sequence ID NO: 5673127

Both numbers are included in the Sequence Table to aid in tracking of information, as shown below:

<210> 174538 (Pat. Appln. Sequence ID NO:)
<211> 1846
<212> DNA (genomic)
<213> Arabidopsis thaliana
<220>
<221> misc_feature
<222> (1) . . . (1846)
<223> Ceres Seq. ID no. 5673127
<220>
<221> misc_feature
<222> () . . . ()
<223> n is a, c, t, g, unknown, or other
<400> 174538
acaagaacaa caaaacagag gaagaagaag aagaagatga agcttctggc tctgtttcca 60
tttctagcga tcgtgatcca actcagctgt . . . etc.

The Sequence and REF Tables are divided into sections by organism: Arabidopsis thaliana, Brassica napus, Glycine max, Zea mays, Triticum aestivum; and Oryza sativa.

b. Coding Sequence

The coding sequence portion of the cDNA was identified by using computer-based algorithms and comparative biology. The sequence of each coding sequence of the cDNA is reported in the “PolyP Sequence” section of the REF Tables, which are also divided into sections by organism. An example shown below for the peptides that relate to the cDNA sequence above

PolyP Sequence
Pat. Appln. Sequence ID NO 174539
Ceres Sequence ID NO 5673128
Loc. Sequence ID NO 174538: @ 1 nt.
Loc. Sig. P. Sequence ID NO 174539: @ 37 aa.

The polypeptide sequence can be found in the SEQ Tables by either the Pat. Appln. Sequence ID NO or by the Ceres Sequence ID NO: as shown below:

<210> 174539 (Pat. Appln. Sequence ID NO)
<211> 443
<212> PRT
<213> Arabidopsis thaliana
<220>
<221> peptide
<222> (1) . . . (443)
<223> Ceres Seq. ID no. 5673128
<220>
<221> misc_feature
<222> () . . . ()
<223> xaa is any aa, unknown or other
<400> 174539
Thr Arg Thr Thr Lys Gln Arg Lys Lys Lys Lys Lys Met Lys Leu Leu
1 5 10 15
Ala Leu Phe Pro Phe Leu Ala Ile . . . etc.
25

The PolyP section also indicates where the coding region begins in the Maximum Length Sequence. More than one coding region may be indicated for a single polypeptide due to multiple potential translation start codons. Coding sequences were identified also by analyzing genomic sequence by predictive algorithms, without the actual cloning of a cDNA molecule from a mRNA. By default, the cDNA sequence was considered the same as the coding sequence, when Maximum Length Sequence was spliced together from a genomic annotation.

c. 5′ and 3′ UTR

The 5′ UTR can be identified as any sequence 5′ of the initiating codon of the coding sequence in the cDNA sequence. Similarly, the 3′ UTR is any sequence 3′ of the terminating codon of the coding sequence.

d. Transcription Start Sites

Applicants cloned a number of cDNAs that encompassed the same coding sequence but comprised 5′ UTRs of different lengths. These different lengths revealed the multiple transcription start sites of the gene that corresponded to the cDNA. These multiple transcription start sites are reported in the “Sequence # w. TSS” section” of the REF Tables.

e. Exons & Introns

Alignment of the cDNA sequences and coding portions to genomic sequence permitted Applicants to pinpoint the exon/intron boundaries. These boundaries are identified in the REF Table under the “Pub gDNA” section. That section reports the gi number of the public BAC sequence that contains the introns and exons of interest. An example is shown below:

Max Len. Seq.:

Pub gDNA:
gi No: 1000000005
Gen. seq. in cDNA:
   115777 . . . 115448 by Method #1
   115105 . . . 114911 by Method #1
   114822 . . . 114700 by Method #1
   114588 . . . 114386 by Method #1
   114295 . . . 113851 by Method #1
   115777 . . . 115448 by Method #2
   115105 . . . 114911 by Method #2
   114822 . . . 114700 by Method #2
   114588 . . . 114386 by Method #2
   114295 . . . 113851 by Method #2
   115813 . . . 115448 by Method #3
   115105 . . . 114911 by Method #3
   114822 . . . 114700 by Method #3
   114588 . . . 114386 by Method #3
   114295 . . . 113337 by Method #3

(Ac) cDNA Sequence

All the gi numbers were assigned by Genbank to track the public genomic sequences except:

gi 1000000001

gi 1000000002

gi 1000000003

gi 1000000004; and

gi 1000000005.

These gi numbers were assigned by Applicants to the five Arabidopsis chromosome sequences that were published by the Institute of Genome Research (TIGR). Gi 1000000001 corresponds to chromosome 1, Gi 1000000002 to chromosome 2, etc.

The method of annotation is indicated as well as any similar public annotations.

f. Promoters & Terminators

Promoter sequences are 5′ of the translational start site in a gene; more typically, 5′ of the transcriptional start site or sites. Terminator sequences are 3′ of the translational terminator codon; more typically, 3′ of the end of the 3′ UTR.

For even more specifics of the REF and SEQ Tables, see the section below titled “Brief Description of the Tables.”

### I.B. Transcriptional (Differental Expression) Information-Introduction to Differential Expression Data & Analyses

A major way that a cell controls its response to internal or external stimuli is by regulating the rate of transcription of specific genes. For example, the differentiation of cells during organogenensis into forms characteristic of the organ is associated with the selective activation and repression of large numbers of genes. Thus, specific organs, tissues and cells are functionally distinct due to the different populations of mRNAs and protein products they possess. Internal signals program the selective activation and repression programs. For example, internally synthesized hormones produce such signals. The level of hormone can be raised by increasing the level of transcription of genes encoding proteins concerned with hormone synthesis.

To measure how a cell reacts to internal and/or external stimuli, individual mRNA levels can be measured and used as an indicator for the extent of transcription of the gene. Cells can be exposed to a stimulus, and mRNA can be isolated and assayed at different time points after stimulation. The mRNA from the stimulated cells can be compared to control cells that were not stimulated. The mRNA levels of particular Maxiumum Length Sequences that are higher in the stimulated cell versus the control indicate a stimulus-specific response of the cell. The same is true of mRNA levels that are lower in stimulated cells versus the control condition.

Similar studies can be performed with cells taken from an organism with a defined mutation in their genome as compared with cells without the mutation. Altered mRNA levels in the mutated cells indicate how the mutation causes transcriptional changes. These transcriptional changes are associated with the phenotype that the mutated cells exhibit that is different from the phenotype exhibited by the control cells.

Applicants have utilized microarray techniques to measure the levels of mRNAs in cells from mutant plants, stimulated plants, and/or selected from specific organs. The differential expression of various genes in the samples versus controls are listed in the MA_diff Tables. Applicants have analyzed the differential data to identify genes whose mRNA transcription levels are positively correlated. From these analyses, Applicants were able to group different genes together whose transcription patterns are correlated. The results of the analyses are reported in the MA_clust Tables.

a. Experimental Detail

A microarray is a small solid support, usually the size of a microscope slide, onto which a number of polynucleotides have been spotted onto or synthesized in distinct positions on the slide (also referred to as a chip). Typically, the polynucleotides are spotted in a grid formation. The polynucleotides can either be Maximum Length Sequences or shorter synthetic oligonucleotides, whose sequence is complementary to specific Maximum Length Sequence entities. A typical chip format is as follows:

| | | |
|---|---|---|
| Oligo #1 | Oligo #2 | Oligo #3 |
| Oligo #4 | Oligo #5 | Oligo #6 |
| Oligo #7 | Oligo #8 | Oligo #9 |

For Applicants' experiments, samples were hybridized to the chips using the “two-color” microarray procedure. A fluorescent dye was used to label cDNA reverse-transcribed from mRNA isolated from cells that had been stimulated, mutated, or collected from a specific organ or developmental stage. A second fluorescent dye of another color was used to label cDNA prepared from control cells.

The two differentially-labeled cDNAs were mixed together. Microarray chips were incubated with this mixture. For Applicants' experiments the two dyes that are used are Cy3, which fluoresces in the red color range, and Cy5, which fluoresces in the green/blue color range. Thus, if:

cDNA#1 binds to Oligo #1;

cDNA#1 from the sample is labeled red;

cDNA#1 from the control is labeled green, and

cDNA#1 is in both the sample and control,

then cDNA#1 from both the sample and control will bind to Oligo#1 on the chip. If the sample has 10 times more cDNA#1 than the control, then 10 times more of the cDNA#1 would be hybridized to Oligo#1. Thus, the spot on the chip with Oligo#1 spot would look red.

| | | |
|---|---|---|
| | Oligo #2 | Oligo #3 |
| Oligo #4 | Oligo #5 | Oligo #6 |
| Oligo #7 | Oligo #8 | Oligo #9 |

If the situation were reversed, the spot would appear green. If the sample has approximately the same amount of cDNA#1 as the control, then the Oligo#1 spot on the chip would look yellow. These color differentials are measured quantitatively and used to deduce the relative concentration of mRNAs from individual genes in particular samples.

b. MA Diff Data

To generate data, Applicants labeled and hybridized the sample and control mRNA in duplicate experiments. One chip was exposed to a mixture of cDNAs from both a sample and control, where the sample cDNA was labeled with Cy3, and the control was labeled with Cy5 dye. For the second labeling and chip hybridization experiments, the fluorescent labels were reversed; that is, the Cy5 dye for the sample, and the Cy3 dye for the control.

Whether Cy5 or Cy3 was used to label the sample, the fluorescence produced by the sample was divided by the fluorescence of the control. A cDNA was determined to be differentially expressed in response to the stimulus in question if a statistically-significantly ration difference in the sample versus the control was measured by both chip hybridization experiments.

The MA_diff data show which cDNA were significantly up-regulated as designated by a “+” and which were significantly down-regulated as designated by a “−” for each pair of chips using the same sample and control.

### I.C. Phenotypic Information

One means of determining the phenotypic effect of a gene is either to insert extra active copies of the gene or coding sequence, or to disrupt an existing copy of the gene in a cell or organism and measure the effects of the genetic change on one or more phenotypic characters or traits. “Knock-in” is used herein to refer to insertion of additional active copies of a gene or coding sequence. “Knock-out” refers to a plant where an endogenous gene(s) is disrupted. Applicants have used both methods of addition or disruption to determine the phenotypic effects of gene or gene components or products, and have thereby discovered the function of the genes and their utilities.

1. Knock-In Results

The coding sequence of a desired protein can be functionally linked to a heterologous promoter to facilitate expression. Here, Applicants have operably linked a number of coding sequences to either one of the promoters listed below:

| | Specific Promoter | Plant Line |
|---|---|---|
| GFP Pattern | activity | Descriptor |
| Root epidermis/mostly toward the lower | Specific to the root basal | Root basal |
| region of root (more intense than CS9094) | region. | |
| Root-endodermis/cortex (initials sharp); | Specific to the root | Root/Petiole/Flowers |
| shoot-mesophyll of one leaf, sharp guard cell | endodermis-cortex | |
| marking. New leaf petioles near tip of | region, leaf petiole, and | |
| primary inflorescence; floral stems; in | flowers. | |
| flowers at base of sepal, anther stems, and | | |
| pistil | | |
| Broad root exp. (some dermal, some cortical, | Specific to root and stem. | Root/Stem1 |
| some vascular); shoot apex. Faintly in | | |
| petiole; stem | | |
| High expression in stem, excluded from 1st | Specific to stem and root. | Root/Stem2 |
| true leaves/High in root. Faint expression in | | |
| stem | | |
| Shoot meristem/whole root region; little bit | Specific to roots, shoot | Root/Stem/Leaves/Flowers |
| on cotyledons. Base of leaves(axillary | meristem, base of leaves | |
| meristem?); base of sepals; inflorescence | and flowers. | |
| meristem; small amount in unfertilized pistil. | | |
| root tip vascular initials; vascular system | Specific to vascular | Vascular/Ovule/Young |
| throughout plant; Bud petal vasculature and | systems. | Seed/Embryo |
| pistil septum; Flower petal vascualture; | | |
| Flower pistil septum; Pre fertilization ovules; | | |
| Post fertilization ovule at chalazal end; | | |
| Developing seed (young, maturing siliques); | | |
| Seed coat and young embryos. GFP not | | |
| observed in mature embryos. | | |
| Flower, sepal/vascular tissue of root, stem, | Specific to flowers, seed | Flowers/Seed/Vasculature/ |
| and cotyledons. Stems of new flowers; | and vasculature. | Embryo |
| vasculature or petals, anthers, sepals, and | | |
| pistil/silique; Vasculature throughtout | | |
| seedling: root, hypocotyl, petioles, stem, | | |
| cotyledons, first true leaves; Rosette | | |
| vasculature; Cauline leaf vasculature; Bud | | |
| pedicel vasculature; Flower vasculature: | | |
| (sepals, petals, filaments, pistil); Bud | | |
| vasculature (sepal, petal, filament, pistil); | | |
| Funiculus in both flower and bud; Some | | |
| possible seed coat expression; Silique | | |
| funiculus; Very faint fluorescence in mature | | |
| embryo (auto fluorescence perhaps); | | |
| Root expression - primarily in cortex (upper | Specific to root. | Roots2 |
| refion of the root). No shoot expression | | |
| Root expression - less intense in whole root | Specific to root and shoot | Root/SAM |
| of young seedling. Shoot apical meristem; | apical meristem. | |
| organ primordia in SAM region. | | |
| Root epidermis/tip; shoot epidermis/vascular; | Specific to seed and to | Seed/Epidermis/Ovary/ |
| leaf epidermis; expression in developing | epidermal layers of roots, | Fruit |
| seed/ovule - mature embryo; Primary and | shoots and leaves. | |
| lateral root cortex; Very strong in root cap; | | |
| Base of flower bud and epidermis of carpels; | | |
| Base of flower, epidermis of filaments, | | |
| epidermis of carpels; Trichomes; Weak | | |
| (hardly detectable) gfp expression in | | |
| vasculature throughout seedling; Strong | | |
| expression in trichomes; POST- fertilization | | |
| SEED only; GFP strength increases as | | |
| silique matures; Weak at suspensor end of | | |
| the embryo; GFP observed in seed coat; Root | | |
| and post fertilization seed specific gfp | | |
| expression; Expression in seed coat. | | |
| Young root dermis; dermal/cortical?/vascular | Specific to roots, shoots, | Roots/Shoots/Ovule |
| in older root; general (epidermal?) shoot | and ovules. | |
| expression; ovules. some in sepals; | | |
| vasculature of stem | | |
| Vascular tissue of root; Meristem tissues: | Specific to root structural | Vasculature/Meristem |
| axillary meristems, floral meristems, base of | leaf vascular region and | |
| flowers/sepals; Weak expression in | to floral buds and axillary | |
| hypocotyl, petiole and cotyledon | meristem | |
| vasculature.. | | |

The chimeric constructs were transformed into Arabidopsis thaliana. The resulting transformed lines were screened to determine what phenotypes were changed due to introduced transgene. The phenotype changes, relative to the control, are reported in the Knock-in tables.

### I.D. Brief Description of the Individual Tables

1. Reference and Sequence Tables

The sequences of exemplary SDFs and polypeptides corresponding to the coding sequences of the instant invention are described in the Sequence Listing and Sequence Listing-Miscellaneous Feature documents (sometimes referred to as the REF and SEQ Tables. The REF Table refers to a number of “Maximum Length Sequences” or “MLS.” Each MLS corresponds to the longest cDNA obtained, either by cloning or by the prediction from genomic sequence. The sequence of the MLS is the cDNA sequence as described in the Av subsection of the REF Table.

The REF Table includes the following information relating to each MLS:

I. cDNA Sequence
A. 5′ UTR
B. Coding Sequence
C. 3′ UTR

II. Genomic Sequence
A. Exons
B. Introns
C. Promoters

III. Link of cDNA Sequences to Clone IDs

IV. Multiple Transcription Start Sites

V. Polypeptide Sequences
A. Signal Peptide
B. Domains
C. Related Polypeptides

VI. Related Polynucleotide Sequences

I. cDNA Sequence

The REF Table indicates which sequence in the SEQ Table represents the sequence of each MLS. The MLS sequence can comprise 5′ and 3′ UTR as well as coding sequences. In addition, specific cDNA clone numbers also are included in the REF Table when the MLS sequence relates to a specific cDNA clone.

A. 5′ UTR

The location of the 5′ UTR can be determined by comparing the most 5′ MLS sequence with the corresponding genomic sequence as indicated in the Reference Table. The sequence that matches, beginning at any of the transcriptional start sites and ending at the last nucleotide before any of the translational start sites corresponds to the 5′ UTR.

B. Coding Region

The coding region is the sequence in any open reading frame found in the MLS. Coding regions of interest are indicated in the PolyP SEQ subsection of the REF Table.

C. 3′ UTR

The location of the 3′ UTR can be determined by comparing the most 3′ MLS sequence with the corresponding genomic sequence as indicated in the REF Table. The sequence that matches, beginning at the translational stop site and ending at the last nucleotide of the MLS corresponds to the 3′ UTR.

II. Genomic Sequence

Further, the REF Table indicates the specific “gi” number of the genomic sequence if the sequence resides in a public databank. For each genomic sequence, REF tables indicate which regions are included in the MLS. These regions can include the 5′ and 3′ UTRs as well as the coding sequence of the MLS. See, for example, the scheme below:

```
                              Region 1 Region 2 Region 3
---------| 5′ UTR | Exon |---------| Exon |--------| Exon | 3′ UTR |-------
        \hat{ } _{—————————————————} \hat{ } _{——————————} \hat{ } _{—————————————————} \hat{ }
        | \hat{ } | | \hat{ } |
Promoter | Intron Intron |
                        Translational Stop Codon
                          Start Site
```

The REF Table reports the first and last base of each region that are included in an MLS sequence. An example is shown below:

gi No. 47000:

37102 . . . 37497

37593 . . . 37925

The numbers indicate that the MLS contains the following sequences from two regions of gi No. 47000; a first region including bases 37102-37497, and a second region including bases 37593-37925.

A. Exon Sequences

The location of the exons can be determined by comparing the sequence of the regions from the genomic sequences with the corresponding MLS sequence as indicated by the REF Table.

i. Initial Exon

To determine the location of the initial exon, information from the

(1) polypeptide sequence section;

(2) cDNA polynucleotide section; and

(3) the genomic sequence section

of the REF Table is used. First, the polypeptide section will indicate where the translational start site is located in the MLS sequence. The MLS sequence can be matched to the genomic sequence that corresponds to the MLS. Based on the match between the MLS and corresponding genomic sequences, the location of the translational start site can be determined in one of the regions of the genomic sequence. The location of this translational start site is the start of the first exon.

Generally, the last base of the exon of the corresponding genomic region, in which the translational start site was located, will represent the end of the initial exon. In some cases, the initial exon will end with a stop codon, when the initial exon is the only exon.

In the case when sequences representing the MLS are in the positive strand of the corresponding genomic sequence, the last base will be a larger number than the first base. When the sequences representing the MLS are in the negative strand of the corresponding genomic sequence, then the last base will be a smaller number than the first base.

ii. Internal Exons

Except for the regions that comprise the 5′ and 3′ UTRs, initial exon, and terminal exon, the remaining genomic regions that match the MLS sequence are the internal exons. Specifically, the bases defining the boundaries of the remaining regions also define the intron/exon junctions of the internal exons.

iii. Terminal Exon

As with the initial exon, the location of the terminal exon is determined with information from the

(1) polypeptide sequence section;

(2) cDNA polynucleotide section; and

(3) the genomic sequence section

of the REF Table. The polypeptide section will indicate where the stop codon is located in the MLS sequence. The MLS sequence can be matched to the corresponding genomic sequence. Based on the match between MLS and corresponding genomic sequences, the location of the stop codon can be determined in one of the regions of the genomic sequence. The location of this stop codon is the end of the terminal exon. Generally, the first base of the exon of the corresponding genomic region that matches the cDNA sequence, in which the stop codon was located, will represent the beginning of the terminal exon. In some cases, the translational start site will represent the start of the terminal exon, which will be the only exon.

In the case when the MLS sequences are in the positive strand of the corresponding genomic sequence, the last base will be a larger number than the first base. When the MLS sequences are in the negative strand of the corresponding genomic sequence, then the last base will be a smaller number than the first base.

B. Intron Sequences

In addition, the introns corresponding to the MLS are defined by identifying the genomic sequence located between the regions where the genomic sequence comprises exons. Thus, introns are defined as starting one base downstream of a genomic region comprising an exon, and end one base upstream from a genomic region comprising an exon.

C. Promoter Sequences

As indicated below, promoter sequences corresponding to the MLS are defined as sequences upstream of the first exon; more usually, as sequences upstream of the first of multiple transcription start sites; even more usually as sequences about 2,000 nucleotides upstream of the first of multiple transcription start sites.

III. Link of cDNA Sequences to Clone IDs

As noted above, the REF Table identifies the cDNA clone(s) that relate to each MLS. The MLS sequence can be longer than the sequences included in the cDNA clones. In such a case, the REF Table indicates the region of the MLS that is included in the clone. If either the 5′ or 3′ termini of the cDNA clone sequence is the same as the MLS sequence, no mention will be made.

IV. Multiple Transcription Start Sites

Initiation of transcription can occur at a number of sites of the gene. The REF Table indicates the possible multiple transcription sites for each gene. In the REF Table, the location of the transcription start sites can be either a positive or negative number.

The positions indicated by positive numbers refer to the transcription start sites as located in the MLS sequence. The negative numbers indicate the transcription start site within the genomic sequence that corresponds to the MLS.

To determine the location of the transcription start sites with the negative numbers, the MLS sequence is aligned with the corresponding genomic sequence. In the instances when a public genomic sequence is referenced, the relevant corresponding genomic sequence can be found by direct reference to the nucleotide sequence indicated by the “gi” number shown in the public genomic DNA section of the Reference Table. When the position is a negative number, the transcription start site is located in the corresponding genomic sequence upstream of the base that matches the beginning of the MLS sequence in the alignment. The negative number is relative to the first base of the MLS sequence which matches the genomic sequence corresponding to the relevant “gi” number.

In the instances when no public genomic DNA is referenced, the relevant nucleotide sequence for alignment is the nucleotide sequence associated with the amino acid sequence designated by “gi” number of the later PolyP SEQ subsection.

V. Polypeptide Sequences

The PolyP SEQ subsection lists SEQ ID NOs and Ceres SEQ ID NO for polypeptide sequences corresponding to the coding sequence of the MLS sequence and the location of the translational start site with the coding sequence of the MLS sequence.

The MLS sequence can have multiple translational start sites and can be capable of producing more than one polypeptide sequence.

A. Signal Peptide

The REF tables also indicate in subsection (B) the cleavage site of the putative signal peptide of the polypeptide corresponding to the coding sequence of the MLS sequence. Typically, signal peptide coding sequences comprise a sequence encoding the first residue of the polypeptide to the cleavage site residue.

B. Domains

Subsection (C) provides information regarding identified domains (where present) within the polypeptide and (where present) a name for the polypeptide domain.

C. Related Polypeptides

Subsection (Dp) provides (where present) information concerning amino acid sequences that are found to be related and have some percentage of sequence identity to the polypeptide sequences of the Reference and Sequence Tables. These related sequences are identified by a “gi” number.

VI. Related Polynucleotide Sequences

Subsection (Dn) provides polynucleotide sequences (where present) that are related to and have some percentage of sequence identity to the MLS or corresponding genomic sequence.

| Abbreviation | Description |
|---|---|
| Max Len. Seq. | Maximum Length Sequence |
| rel to | Related to |
| Clone Ids | Clone ID numbers |
| Pub gDNA | Public Genomic DNA |
| gi No. | gi number |
| Gen. Seq. in Cdna | Genomic Sequence in cDNA |
| | (Each region for a single gene prediction is |
| | listed on a separate line. |
| | In the case of multiple gene predictions, the |
| | group of regions relating to a single prediction |
| | are separated by a blank line) |
| (Ac) cDNA SEQ | cDNA sequence |
| Pat. Appln. SEQ ID NO | Patent Application SEQ ID NO: |
| Ceres SEQ ID NO: | Ceres SEQ ID NO: |
| 1673877 | |
| SEQ # w. TSS | Location within the cDNA sequence, SEQ ID |
| | NO:, of Transcription Start Sites which are |
| | listed below |
| Clone ID #: # -> # | Clone ID comprises bases # to # of the cDNA |
| | Sequence |
| PolyP SEQ | Polypeptide Sequence |
| Pat. Appln. SEQ ID NO: | Patent Application SEQ ID NO: |
| Ceres SEQ ID NO | Ceres SEQ ID NO: |
| Loc. SEQ ID NO: @ nt. | Location of translational start site in cDNA of |
| | SEQ ID NO: at nucleotide number |
| (C) Pred. PP Nom. & | Nomination and Annotation of Domains within |
| Annot. | Predicted Polypeptide(s) |
| (Title) | Name of Domain |
| Loc. SEQ ID NO #: | Location of the domain within the polypeptide |
| # -> # aa. | of SEQ ID NO: from # to # amino acid |
| | residues. |
| (Dp) Rel. AA SEQ | Related Amino Acid Sequences |
| Align. NO | Alignment number |
| gi No | Gi number |
| Desp. | Description |
| % Idnt. | Percent identity |
| Align. Len. | Alignment Length |
| Loc. SEQ ID NO: | Location within SEQ ID NO: from # to # |
| # -> # aa | amino acid residue. |

2. MA DATA

The MA DATA presents the results of the differential expression experiments for the mRNAs, as reported by their corresponding cDNA ID number, that were differentially transcribed under a particular set of conditions as compared to a control sample. The cDNA ID numbers correspond to those utilized in the Reference and Sequence Tables. Increases in mRNA abundance levels in experimental plants versus the controls are denoted with the plus sign (+). Likewise, reductions in mRNA abundance levels in the experimental plants are denoted with the minus (−) sign.

The “cDNA_ID provides the identifier number for the cDNA tracked in the experiment. The column headed “SHORT_NAME” (e.g. At_{—}0.001%_MeJa_cDNA_P) provides a short description of the experimental conditions used. The column headed “EXPT_REP_ID” provides an identifier number for the particular experiment conducted. The values in the column heades “Differential” indicate whether expression of the cDNA was increased (+) or decreased (−) compared to the control.

In some cases, data relating to how the experiment was conducted follows the results of the experiment. Here, the data following the expression results provides the experimental parameters used in conducting the microarray experiment. Again, the “SHORT_NAME” identifies the experiment t(e.g. At_{—}0.001%_MeJa_cDNA_P). The firest column, “EXPT_REP_ID,” indicates the individual experiment (e.g. 108569). The comnd column, “PARAM_NAME,” identifies the parameter used (e.g. Timepoint (hr)), while the third column, “Value” provides the descriptor for the particular parameter (e.g. “6”). As an example, when read together one understands that the “methyl jasmonate” section of the Specification provides information pertinent to the 0.001% MeJA (methyl jasmonate experiment 108569, which contains data taken from a 6 hr timepoint.

3. MA Parameters Data

This data provides the experimental parameters used in conducting the microarray experiments. The first column indicates the pertinent section of the Specification. The second column provides the “Short Name” for the experiment (e.g. At_{—}0.001%_MeJA_cDNA_P). The third column gives the “Experiment ID” number. The fourth column is the particular parameter being described (e.g. Timepoint (hr)). The last column provides the descriptor for the particular parameter (e.g. “6”). As an example, when read together one understands that the “Methyl Jasmonate” section of the Specification provides information pertinent to the 0.001% MeJA (methyl jasmonate) experiment 108569, which contains data taken from a 6 hr Timepoint.

4. Ortholog Pair Data

This table lists pairs of orthologs that were identified using the T Blast X program. Each column contains a cDNA_id number corresponding to a sequence from Arabidopsis, wheat, corn, soybean or canola. The sequence corresponding to all cDNA_id numbers can be found in either the SEQ Table or the REF Table.

5. Phenotype Data

This table provides information regarding the phenotype associated with expression of particular cDNAs. The first column identifies the cDNA_id or clone_id number of the sequence associated with the experiment.

The “Promoter” column identifies the promoter used to drive expression of the cDNA. “35S” refers to the Cauliflower Mosaic Virus (CMV) 35S promoter while the remaining entries signify a particular cDNA_id number. The endogenous promoter for the identified cDNA sequence, located immediately upstream from the cDNA start site, was used. These sequences appear in the “Promoter Table”

The“line_id” column gives the identifier number associated with the plant transformed with the CDNA or clone listed in the first column. For example, “ME0589-01” is a plant that resulted from a unique independent insertion of cDNA 3265003 into the plant genome. Likewise, “ME0589-02” was also the result of a unique independent insertion event. Consequently, the portion of the identifier located after the dash (e.g. “-03,” “-04,” etc.) indicates which of the series of “T1” plants generated was scored for a phenotype. That is, if ten T1 plants were grown from the transformation involving cDNA 3265003, these would be identified as ME0589-01 to ME0589-10.

The “Phenotype Present?” column identifies whether a gross visual phenotype was present. “Yes” indicates that a phenotype different from wildtype was observed while “No” indicates no visible change from wildtype. “Questionable” indicates that the transformant showed a phenotype, but that it was uncertain whether the phenotype was due to the gene inserted or to other factors (e.g. environmental).

The “Developmental Stage” column provides information as to the developmental stage of the plant when it was scored for phenotype. The following chart correlates a numerical value with a short description of a particular developmental stage.

| DEV_STAGE | DESCRIPTION |
|---|---|
| 0 | N/A |
| 0.10 | Seed imbibition |
| 0.50 | Radical emergence |
| 0.70 | Hypocotyl and cotyledon emergence |
| 1.00 | Cotyledons fully opened |
| 1.02 | 2 rosette leaves 1 mm in length |
| 1.03 | 3 rosette leaves 1 mm in length |
| 1.04 | 4 rosette leaves 1 mm in length |
| 1.05 | 5 rosette leaves 1 mm in length |
| 1.06 | 6 rosette leaves 1 mm in length |
| 1.07 | 7 rosette leaves 1 mm in length |
| 1.08 | 8 rosette leaves 1 mm in length |
| 1.09 | 9 rosette leaves 1 mm in length |
| 1.10 | 10 rosette leaves 1 mm in length |
| 1.11 | 11 rosette leaves 1 mm in length |
| 1.12 | 12 rosette leaves 1 mm in length |
| 1.13 | 13 rosette leaves 1 mm in length |
| 1.14 | 14 rosette leaves 1 mm in length |
| 3.50 | Rosette is 50% of final size |
| 5.10 | First flower buds visible |
| 6.00 | First flower open |
| 6.10 | 10% of flowers to be produced have opened |
| 6.30 | 30% of flowers to be produced have opened |
| 6.50 | 50% of flowers to be produced have opened |
| 6.90 | Flowering complete |
| 8.00 | First silique shattered |
| 8.50 | 50% of the siliques have shattered |
| 9.70 | Senescence complete |
| 10.0 | All Stages |
| [NULL] | No mutant phenotype or plant died |

The “Phenotype Observed” column describes the phenotype associated with the plant transformed with the particular cDNA_id or clone_id. The term “[NULL]” appears when no gross visual phenotype was present or where the plant died. Note that a single plant line may have more than one phenotype associated with it. For example, ME05809-01 has a short petiole phenotype associated with its rosette leaves as well as an oval leaf shape.

22. Promoter Table

This data identifies nucleic acid promoter sequences using the heading “PROMOTER ID NO.” The “PROMOTER ID NO” is a number that identifies the sequence of the promoter used in the experiments

### II. HOW THE INVENTIONS REVEAL HOW GENES, GENE COMPONENTS AND PRODUCTS FUNCTION

The different experimental molecular genetic approaches focused on different aspects of genes, gene components, and gene products of the inventions. The variety of the data demonstrates the multiple functions and characteristics of single genes, gene components, and products. The data also explain the pathways and networks in which individual genes and products participate and interact. As a result, the circumstances or conditions are now known when these genes and networks are active. These new understandings of biology are relevant for many plant species. The following section describes the process by which Applicants analyzed the experimental result relavent to the present invention.

### II.A. Experimental Results Reveal Many Facets of a Single Gene

The experimental results are used to dissect the function of individual components and products of the genes. For example, the biochemical activity of the encoded protein could be surmised from sequence analyses, and promoter specificity could be identified through transcriptional analyses. Generally, the data presented herein can be used to functionally annotate either the protein sequence and/or the regulatory sequence that control transcription and translation.

### II.A.1. Functions of Coding Sequences Revealed by the Ceres Genomic Engine

II.A.1.a. Sequence Similarity to Proteins of Known Function Can be Used to Associate Biochemical Activities and Molecular Interaction to the Proteins of the Invention

The protein sequences of the invention were analyzed to determine if they shared any sequence characteristics with proteins of known activity. Proteins can be grouped together based on sequence similarity, either localized or throughout the length of the proteins. Typically, such groups of proteins exhibit common biochemical activities or interact with similar molecules.

II.A.1.a.1 Presence of Amino Acid Motifs Indicates Biological Function

Localized protein sequence similarity, also referred to as amino acid motifs, have been attributed to enzyme or protein functions. A library of motifs, important for function, have been documented in PROSITE, a public database available at http://www.expasy.ch/prosite/. This library includes descriptions of the motifs and their functions. The zinc finger motif is one such entry in PROSITE, which reports that the zinc finger domain of DNA-binding proteins is typically defined by a 25-30 amino acid motif containing specific cysteine or histidine residues that are involved in the tetrahedral coordination of a zinc ion. Any protein comprising a sequence similar to the zinc finger amino acid motif will have similar functional activity (specific binding of DNA).

Protein sequences of the invention have been compared to a library of amino acid motifs in the pFAM database, which is linked to the PROSITE database. If any of Applicants' protein sequences exhibit similarity to these amino acid motifs or domains, the Reference Table notes the name and location of the motif in the “Pred. PP Nom. & Annot” section of the Reference tables. A description of any biochemical activities that are associated to these domains, and therefore associated with Applicants' proteins, is included in the Protein Domain table.

For example, polypeptide, CERES Sequence ID NO: 1545823 is associated with zinc finger motif as follows in the Reference Table:

(C) Pred. PP Nom. & Annot.
Zinc finger, C3HC4 type (RING finger)
Loc. Sequence ID NO 133059: 58→106 aa.
   II.A.1.a.2 Related Amino Acid Sequences Share Similar Biological Functions

When studying protein sequence families, it is apparent that some regions have been better conserved than others during evolution. These regions are generally important for the function of a protein and/or for the maintenance of its three-dimensional structure.

The Reference Table reports in section “(Dp) Rel. AA Sequence” when a protein shares amino acid similarity with a protein of known activity. The section reports the gi number of the protein of known activity, a brief description of the activity, and the location where it shares sequence similarity to Applicants' polypeptide sequence.

Using this analysis, biochemical activity of the known protein is associated with Applicants' proteins. An example for the polypeptide described above is as follows:

(Dp) Rel. AA Sequence
Align. NO 524716
gi No 2502079
Desp.: (AF022391) immediate early protein; ICPO [Feline herpesvirus 1]
% Idnt.: 33.7
Align. Len.: 87
Loc. Sequence ID NO 133059: 52→137 aa.
   II.A.1.b. Differential Expression Results Explain in Which Cellular Responses the Proteins of the Invention are Involved

Differential expression results show when the coding sequence is transcribed, and therefore when the activity of the protein is deployed by the cell. Similar coding sequences can have very different physiological consequences because the sequences are expressed at different times or places, rather than because of any differences in protein activity. Therefore, modified levels (increased or decreased) of expression as compared to a control provide an indication of the function of a corresponding gene, gene components, and gene products.

These experiments can determine which are genes “over-expressed” under a given stimulus. Such over-expressed genes give rise to higher transcript levels in a plant or cell that is stimulated as compared to the transcript levels of the same genes in a control organism or cell. Similarly, differential expression experiments can reveal “under-expressed” genes.

To increase the cellular response to a stimulus, additional copies of the coding sequences of a gene that is over-expressed are inserted into a cell. Increasing transcript levels of an over-expressed gene can either heighten or prolong the particular cellular response. A similar enhancement can occur when transcription of an under-expressed gene is inhibited. In contrast, the cellular response will be shortened or less severe when the over-expressed genes are inhibited or when expression of the under-expressed genes are increased.

In addition to analyzing the levels of transcription, the data were also analyzed to gain insight into the changes in transcription over time. That is, while the plants in the experiments were reacting to either an external or internal stimulus, a differential experiment takes a snapshot of the transcription levels in the cells at one specific time. However, a number of snap-shots can be taken at different time points during an external stimulus regime or at different stages of development during an internal stimulus. These results show how the plant changes transcription levels over time, and therefore protein levels in response to specific stimuli to produce phenotypic changes. These results show that a protein can be implicated in a single, but more likely, in a number of cellular responses.

II.A.1.b.1. The Transcript Levels of a Protein Over Time in Response to a Stimuli are Revealed by Transcriptional Analyses Over Many Experiments

Applicants produced data from plants at different times after a specific stimulus. These results show whether the expression level of a gene spikes at a key moment during the cellular response, or whether the transcript level remains constant. Thus, coding sequences not only can be determined to be over- or under-expressed, but also can be classified by the initial timing and duration of differential expression. This understanding of timing can be used to increase or decrease any desired cellular response.

Generally, Applicants have assayed plants at 2 to 4 different time points after exposing the plants to the desired stimuli. From these experiments, “early” and “late” responders were identified. These labels are applied to either the regulatory sequences driving transcription of the gene as well as to the protein encoded by the gene.

The following example illustrates how the genes, gene components and products were classified as either early or late responders following a specific. The mRNAs from plants exposed to drought conditions were isolated 1 hour and 6 hours after exposure to drought conditions. These mRNAs were tested utilizing microarray techniques. The graph below illuminates possible transcription profiles over the time course, plotting all the (+) data points as +1 and all the (−) data points as −1:

[image]

(The value for each time point was determined using a pair of microarray chips as described above.)

Data acquired from this type of time course experiment are useful to understand how one may increase or decrease the speed of the cellular response. Inserting into a cell extra copies of the coding sequence of early responders in order to over-express the specific gene can trigger a faster cellular response. Alternatively, coding sequences of late responders that are over-expressed can be placed under the control of promoters of early responders as another means to increase the cellular response.

Inserting anti-sense or sense mRNA suppression constructs of the early responders that are over-expressed can retard action of the late responders, thereby delaying the desired cellular response. In another embodiment, extra copies of the promoters of both early and late responders can be added to inhibit expression of both types of over-expressed genes.

The experiments described herein are grouped together to determine the time course of the transcript levels of different coding sequences in response to different stimuli.

II.A.1.b.2. The Transcript Levels of a Protein Over Different Developmental Stages Can be Identified by Transcriptional Analyses Over Many Experiments

Differential expression data were produced for different development stages of various organs and tissues. Measurement of transcript levels can divulge whether specific genes give rise to spikes of transcription at specific times during development, or whether transcription levels remain constant. This understanding can be used to increase speed of development, or to arrest development at a specific stage.

Like the time-course experiments, the developmental stage data can classify genes as being transcribed at early or late stages of development. Generally, Applicants assayed different organs or tissues at 2-4 different stages.

Inhibiting under-expressed genes at either early or late stages can trigger faster development times. The overall development time also can be increased by this means to allow organs and tissue to grow to a larger size or to allow more organs or tissues to be produced. Alternatively, coding sequences of late stage genes that are under-expressed can be placed under the control of promoters of early stage genes to increase heighten development.

Inserting extra copies of the coding sequence early stage genes that are under-expressed can retard action of the late-stage genes and delay the desired development.

Fruit development of Arabidopsis is one example that can be studied. Siliques of varying sizes, which are representative of different stages, were assayed by microarray techniques. Specifically, mRNA was isolated from siliques between 0-5 mm, between 5-10 mm and >10 mm in length. The graph below shows expression pattern of a cell wall synthesis gene, cDNA ID 1595707, during fruit development:

[image]

The developmental course shows that the gene encoding a cell wall synthesis protein is up-regulated when the fruit is 0-5 mm but returns to normal levels at 5-10 mm and >10 mm. Increase of cell wall synthesis can lead to larger cells and/or greater number of cells. This type of increase can boost fruit yield. The coding sequence of the cell wall synthesis protein under the control of a strong early stage promoter would increase fruit size or number.

A pectinesterase gene was also differentially expressed during fruit development, cDNA ID 1396123. Pectinesterase catalyzes the hydrolysis of pectin into pectate and methanol. This biochemical activity plays an important role in cell wall metabolism during fruit ripening. To shorten the time for fruit ripening, extra copies of this gene with its endogenous promoter can be inserted into a desired plant. With its native promoter, the extra copies of the gene will be expressed at the normal time, to promote extra pectinesterase at the optimal stage of fruit development thereby shortening ripening time.

II.A.1.b.3. Proteins that are Common in a Number of Similar Responses Can be Identified by Transcriptional Analyses Over a Number of Experiments

The differential expression experiments also reveal the genes, and therefore the coding sequence, that are common to a number of cellular responses. By identifying the genes that are differentially expressed in a number of similar responses, the genes at the nexus of a range of responses are discovered. For example, genes that are differentially expressed in all the stress responses are at the hub of many of the stress response pathways.

These types of nexus genes, proteins, and pathways are differentially expressed in many or majority of the responses or developmental conditions of interest. Typically, a nexus gene, protein, or pathway is differentially expressed in generally the same direction in many or majority of all the desired experiments. By doing so, the nexus gene can be responsible for triggering the same or similar set of pathways or networks for various cellular responses. This type of gene is useful in modulating pleiotropic effects or triggering or inhibiting a general class of responses.

When nexus genes are differentially expressed in a set of responses, but in different directions, these data indicate that a nexus gene is responsible for creating the specificity in a response by triggering the same pathway but to a different degree. Placing such nexus genes under a constitutive promoter to express the proteins at a more constant level can remove the fluctuations. For example, a plant that is better drought adapted, but not cold adapted can be modified to be tolerant to both conditions by placing under the control of a constitutive promoter a nexus gene that is up-regulated in drought but down regulated in cold.

Applicants' experiments can be grouped together to identify such nexus genes. Examples of these groups are as follows:
Herbicide Response
   Trimec, Finale, Glean, Round-up
Stress Response
   Drought, Cold, Heat, Osmotic, PEG, Trimec, Finale, Glean, Round-up
   Wounding, SA, MeJA, Reactive Oxygen, NO
Hormone Responses
   NAA, BA, BR, GA,
   NAA, Trimec
      II.A.1.b.4. Proteins that are Common to Disparate Responses Can be Identified by Transcriptional Analyses Over a Number of Experiments

Phenotypes and traits result from complex interactions between cellular pathways and networks. Which pathways are linked by expression of common genes to specify particular traits can be discerned by identifying the genes that show differential expression of seemingly disparate responses or developmental stages. For example, hormone fluxes in a plant can direct cell patterning and organ development. Genes that are differentially expressed both in the hormone experiments and organ development experiments would be of particular interest to control plant development.

Examples of Such Pathway Interactions Include:
(i) The Interaction Between Stress Tolerance Pathways And Metabolism Pathways;
(ii) Interaction Between Hormone Responses And Developmental Changes In The Plant;
(iii) Interactions Between Nutrient Uptake And Developmental Changes;
(iv) Mediation of Stress Response by Hormone Responses; And
(v) Interactions Between Stress Response And Development.
   Applicant's experiments can be grouped together to identify proteins that participate in interacting pathways or networks. Specific groups of experiments include, for example:
(i) Stress & Metabolism
   Germinating Seeds
(ii) Hormones & Development
   NAA, BA & Root Tips, Roots And/Or Root Tips, Leaf, ABA & Siliques (Of Any Size), GA, Imbibed & Germinating Seeds, Tissue Specific Expression
(iii) Nutrient Uptake and Development
   Any or All Nitrogen Experiments With Siliques (Of Any Size), Roots Or Root Tips
(iv) Stress & Hormones
   ABA, Drought, Cold, Heat, & Wounding, Tissue Specific Expression
(v) Stress & Hormones Stress & Hormones
   Nitrogen High transition to Low, Tissue Specific Expression
      II.A.1.c. Observations of Phenotypic Changes Show what Physiological Consequences Applicants' Proteins Can Produce

Another direct means of determining the physiological consequences of a protein is to make aberrant decreases or increases of its expression level in a cell. To this end, Applicants have produced plants where specific genes have been disrupted, or produced plants that include an extra expressed copy of the gene. The plants were then planted under various conditions to determine if any visible physiological changes are caused. These changes then are attributed to the changes in protein levels.

### II.A.2. Differential Expression Results Explain which External or Internal Stimuli Trigger the Regulatory Sequences

Transcriptional studies can reveal the time and place that genes are expressed. Typically, regulatory sequences, such as promoters, introns, UTRs, etc., control when and in which cells transcription occurs. Differential studies can explain the temporal- and location-specific regulatory sequences that control transcription.

Using the experiments that are provided herein, one skilled in the art can choose a promoter or any other regulatory sequence that is capable of facilitating the desired pattern of transcription. For example, if a promoter is needed to give rise to increased levels of transcription in response to Auxin, but little expression in response to cytokinin, then the promoters of cDNAs that were up-regulated in the Auxin experiments, but down-regulated the cytokinin experiments would be of interest.

Time Course Experiments—Time Sensitive

Evaluation of time-course data as described above is also useful to identify time-specific promoters. Promoters or regulatory sequences, like the coding sequences, can be classified as early or late responding according to the microarray data. Promoters that facilitate expression of early or late genes are useful to direct expression of heterologous coding sequences to modulate the cellular response. In the drought data, promoters from “early” responding genes can be selected to activate expression of any desired coding sequence. Thus, a coding sequence for a salt-tolerance protein that is not typically expressed early in response to drought could be linked to an “early” responding promoter to increase salt tolerance within one hour after exposure to drought conditions.

Developmental Experiments—Time Sensitive

Another class of time-sensitive promoters and other regulatory sequence can be identified from the experiments examining different developmental stages. These regulatory sequences can drive transcription of heterologous sequence at particular times during development. For example, expression of stress-responsive genes during fruit development can protect any gain in fruit yield.

Common to Many Pathways—Cause General Effects

Promoters and other regulatory sequence associated with cDNAs that are differentially expressed in a number of similar responses can be used to cause general effects. These types of regulatory sequences can be used to inhibit or increase expression of a desired coding sequence in a number circumstances. For example, protein that is capable of acting as an insecticide can be placed under the control a general “stress” promoter to increase expression, not only when the plant is wounded, but under other stress attack.

### II.B. Experimental Results Also Reveal the Functions of Genes

### II.B.1. Linking Signature Sequences to Conservation of Biochemical Activities and Molecular Interactions

Proteins that possess the same defined domains or motifs are likely to carry out the same biochemical activity or interact with a similar class of target molecule, e.g., DNA, RNA, proteins, etc. Thus, the pFAM domains listed in the Reference Tables are routinely used as predictors of these properties. Substrates and products for the specific reactions can vary from protein to protein. Where the substrates, ligands, or other molecules bound are identical the affinities may differ between the proteins. Typically, the affinities exhibited by different functional equivalents varies no more than 50%; more typically, no more than 25%; even more typically, no more than 10%; or even less.

Proteins with very similar biochemical activities or molecular interactions will share similar structural properties, such as substrate grooves, as well as sequence similarity in more than one motif. Usually, the proteins will share at least two motifs of the signature sequence; more usually, three motifs; even more usually four motifs or greater. Typically, the proteins exhibit 70% sequence identity in the shared motifs; more typically, 80% sequence identity; even more typically, 90% sequence identity or greater. These proteins also often share sequence similarity in the variable regions between the constant motif regions. Further, the shared motifs will be in the same order from amino- to carboxyl-termini. The length of the variable regions between the motifs in these proteins, generally, is similar. Specifically, the number of residues between the shared motifs in these proteins varies by less than 25%; more usually, does not vary by less than 20%; even more usually, less than 15%; even more usually less than 10% or even less.

### II.B.2. Linking Signature Sequences to Conservation of Cellular Responses or Activities

Proteins that exhibit similar cellular response or activities will possess the structural and conserved domain/motifs as described in the Biochemical Activities and Molecular Interactions above.

Proteins can play a larger role in cellular response than just their biochemical activities or molecular interactions suggest. A protein can initiate gene transcription, which is specific to the drought response of a cell. Other cellular responses and activities include: stress responses, hormonal responses, growth and differential of a cell, cell to cell interactions, etc.

The cellular role or activities of protein can be deduced by transcriptional analyses or phenotypic analyses as well as by determining the biochemical activities and molecular interactions of the protein. For example, transcriptional analyses can indicate that transcription of gene A is greatly increased during flower development. Such data would implicate protein A encoded by gene A, in the process of flower development. Proteins that shared sequence similarity in more than one motif would also act as functional equivalents for protein A during flower development.

### III. DESCRIPTION OF THE GENES, GENE COMPONENTS AND PRODUCTS, TOGETHER WITH THEIR USE AND APPLICATION

As described herein, the results of Applicant's experiments provide an understanding of the function and phenotypic implications of the genes, gene components and products of the present invention. Bioinformatic analysis provides such information. The sections of the present application containing the bioinformatic analysis, together with the Sequence and Reference Tables, teach those skilled in the art how to use the genes, gene components and products of the present invention to provide plants with novel characteristics. Similarly, differential expression analysis provides additional such information and the sections of the present application on that analysis; together with the MA_Diff Tables and MA_Cluster Tables, describe the functions of the genes, gene components and products of the present invention which are understood from the results of the differential expression experiments. The same is true with respect to the phenotype data, wherein the results of the Knock-in and Knock-out experiments and the sections of the present application on those experiments provide the skilled artisan with further description of the functions of the genes, gene components and products of the present invention.

As a result, one reading each of these sections of the present application as an independent report will understand the function of the genes, gene components and products of the present invention. But those sections and descriptions can also be read in combination, in an integrated manner, to gain further insight into the functions and uses for the genes, gene components and products of the present invention. Such an integrated analysis does not require extending beyond the teachings of the present application, but rather combining and integrating the teachings depending upon the particular purpose of the reader.

Some sections of the present application describe the function of genes, gene components and products of the present invention with reference to the type of plant tissue (e.g. root genes, leaf genes, etc.), while other sections describe the function of the genes, gene components and products with respect to responses under certain conditions (e.g. Auxin-responsive genes, heat-responsive genes, etc.). Thus, if one desires to utilize a gene understood from the application to be a particular tissue-type of gene, then the condition-specific responsiveness of that gene can be understood from the differential expression tables, and very specific characteristics of actions of that gene in a transformed plant will be understood by recognizing the overlap or intersection of the gene functions as understood from the two different types of information. Thus, for example, if one desires to transform a plant with a root gene for enhancing root growth and performance, one can know the useful root genes from the results reported in the knock-in and knock-out tables. A review of the differential expression data may then show that a specific root gene is also over-expressed in response to heat and osmotic stress. The function of that gene is then described in (1) the section of the present application that discusses root genes, (2) the section of the present application that discusses heat-responsive genes, and (3) the section of the application that discusses osmotic stress-responsive genes. The function(s) which are commonly described in those three sections will then be particularly characteristic of a plant transformed with that gene. This type of integrated analysis of data can be viewed from the following schematic that summarizes, for one particular gene, the function of that gene as understood from the phenotype and differential expression experiments.

| Gene function known | Gene function known | Gene function known |
|---|---|---|
| from phenotype | from first differential | from second differential |
| experiments | expression experiment | expression experiment |
| Function A | Function A | Function A |
| Function B | | |
| | Function C | Function C |
| | Function D | |
| | | Function E |
| Function F | Function F | Function F |
| Function G | Function G | |
| | | Function H |
| Function I | | Function I |
| | Function J | |

In the above example, one skilled in the art will understand that a plant transformed with this particular gene will particularly exhibit functions A and F because those are the functions which are understood in common from the three different experiments.

Similar analyses can be conducted on various genes of the present invention, by which one skilled in the art can effectively modulate plant functions depending upon the particular use or conditions envisioned for the plant.

### III.A. Organ-Affecting Genes, Gene Components, Products (Including Diferentation and Function)

### III.A.1. Root Genes, Gene Components and Products

The economic values of roots arise not only from harvested adventitious roots or tubers, but also from the ability of roots to funnel nutrients to support growth of all plants and increase their vegetative material, seeds, fruits, etc. Roots have four main functions. First, they anchor the plant in the soil. Second, they facilitate and regulate the molecular signals and molecular traffic between the plant, soil, and soil fauna. Third, the root provides a plant with nutrients gained from the soil or growth medium. Fourth, they condition local soil chemical and physical properties.

### Use of Promoters of Root Genes

Promoters of root genes, as described in the Reference tables, for example, can be used to modulate transcription that is induced by root development or any of the root biological processes or activities above. For example, when a selected polynucleotide sequence is operably linked to a promoter of a root gene, then the selected sequence is transcribed in the same or similar temporal, development or environmentally-specific patterns as the root gene from which the promoter was taken. The root promoters can also be used to activate antisense copies of any coding sequence to achieve down regulation of its protein product in roots. They can also be used to activate sense copies of mRNAs by RNA interference or sense suppression in roots.

### III.A.2. Root Hair Genes, Gene Components and Products

Root hairs are specialized outgrowths of single epidermal cells termed trichoblasts. In many and perhaps all species of plants, the trichoblasts are regularly arranged around the perimeter of the root. In Arabidopsis, for example, trichoblasts tend to alternate with non-hair cells or atrichoblasts. This spatial patterning of the root epidermis is under genetic control, and a variety of mutants have been isolated in which this spacing is altered or in which root hairs are completely absent.

### Use of Promotors of Root Hair Genes

Promoters of root hair development genes, as described in the Reference tables, for example, are useful to modulate transcription that is induced by root hair development or any of the following phenotypes or biological activities above. For example, any desired sequence can be transcribed in similar temporal, tissue, or environmentally-specific patterns as the root hair genes when the desired sequence is operably linked to a promoter of a root hair responsive gene.

### III.A.3. Leaf Genes, Gene Components and Products

Leaves are responsible for producing most of the fixed carbon in a plant and are critical to plant productivity and survival. Great variability in leaf shapes and sizes is observed in nature. Leaves also exhibit varying degrees of complexity, ranging from simple to multi-compound. Leaf genes as defined here, not only modulate morphology, but also influence the shoot apical meristem, thereby affecting leaf arrangement on the shoot, intemodes, nodes, axillary buds, photosynthetic capacity, carbon fixation, photorespiration and starch synthesis. Leaf genes elucidated here can be used to modify a number of traits of economic interest from leaf shape to plant yield, including stress tolerance, and to modify the efficiency of synthesis and accumulation of specific metabolites and macromolecules.

### Use of Leaf Gene Promoters

Promoters of leaf genes are useful for transcription of desired polynucleotides, both plant and non-plant. If the leaf gene is expressed only in leaves, or specifically in certain kinds of leaf cells, the promoter is used to drive the synthesis of proteins specifically in those cells. For example, extra copies of carbohydrate transporter cDNAs operably linked to a leaf gene promoter and inserted into a plant increase the “sink” strength of leaves. Similarly, leaf promoters are used to drive transcription of metabolic enzymes that alter the oil, starch, protein, or fiber contents of a leaf. Alternatively, leaf promoters direct expression of non-plant genes that can, for instance, confer insect resistance specifically to a leaf. Additionally the promoters are used to synthesize an antisense mRNA copy of a gene to inactivate the normal gene expression into protein. The promoters are used to drive synthesis of sense RNAs to inactivate protein production via RNA interference.

### III.A.4. Trichome Genes and Gene Components

Trichomes, defined as hair-like structures that extend from the epidermis of aerial tissues, are present on the surface of most terrestrial plants. Plant trichomes display a diverse set of structures, and many plants contain several types of trichomes on a single leaf. The presence of trichomes can increase the boundary layer thickness between the epidermal tissue and the environment, and can reduce heat and water loss. In many species, trichomes are thought to protect the plant against insect or pathogen attack, either by secreting chemical components or by physically limiting insect access to or mobility on vegetative tissues. The stellate trichomes of Arabidopsis do not have a secretory anatomy, but at a functional level, they might limit herbivore access to the leaf in the field. In addition, trichomes are known to secrete economically valuable substances, such as menthol in mint plants.

### Use of Promoters of Trichome Genes

Promoters of trichome genes are useful for facilitating transcription of desired polynucleotides, both plant and non-plant in trichomes. For example, extra copies of existing terpenoid synthesis coding sequences can be operably linked to a trichome gene promoter and inserted into a plant to increase the terpenoids in the trichome. Alternatively, trichome promoters can direct expression of non-plant genes or genes from another plant species that can, for instance, lead to new terpenoids being made. The promoters can also be operably linked to antisense copies of coding sequences to achieve down regulation of these gene products in cells.

### III.A5. Chloroplasts Genes, Gene Components and Products

The chloroplast is a complex and specialized organelle in plant cells. Its complexity comes from the fact that it has at least six suborganellar compartments subdivided by double-membrane envelope and internal thylakoid membranes. It is specialized to carry out different biologically important processes including photosynthesis and amino acid and fatty acid biosynthesis. The biogenesis and development of chloroplast from its progenitor (the proplasptid) and the conversion of one form of plastid to another (e.g., from chloroplast to amyloplast) depends on several factors that include the developmental and physiological states of the cells.

One of the contributing problems that complicate the biogenesis of chloroplast is the fact that some, if not most, of its components must come from the outside of the organelle itself. The import mechanisms must take into account to what part within the different sub-compartments the proteins are being targeted; hence the proteins being imported from the cytoplasm must be able to cross the different internal membrane barriers before they can reach their destinations. The import mechanism must also take into account how to tightly coordinate the interaction between the plastid and the nucleus such that both nuclear and plastidic components are expressed in a synchronous and orchestrated manner. Changes in the developmental and physiological conditions within or surrounding plant cells can consequently change this tight coordination and therefore change how import mechanisms are regulated as well. Manipulation of these conditions and modulation of expression of the import components and their function can have critical and global consequences to the development of the plant and to several biochemical pathways occurring outside the chloroplast.

### Use of Promoters of Chloroplast Genes

Promoters of Chloroplast genes are useful for transcription of any desired polynucleotide or plant or non-plant origin. Further, any desired sequence can be transcribed in a similar temporal, tissue, or environmentally specific patterns as the Chloroplast genes where the desired sequence is operably linked to a promoter of a Chloroplast gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression

### III.A.6. Reproduction Genes, Gene Components and Products

Reproduction genes are defined as genes or components of genes capable of modulating any aspect of sexual reproduction from flowering time and inflorescence development to fertilization and finally seed and fruit development. These genes are of great economic interest as well as biological importance. The fruit and vegeTable industry grosses over $1 billion USD a year. The seed market, valued at approximately $15 billion USD annually, is even more lucrative.

### Use of Promoters and Reproduction Genes

Promoter of reproduction genes are useful for transcription of desired polynucleotides, both plant and non-plant. For example, extra copies of carbohydrate transporter genes can be operably linked to a reproduction gene promoter and inserted into a plant to increase the “sink” strength of flowers or siliques. Similarly, reproduction gene promoters can be used to drive transcription of metabolic enzymes capable of altering the oil, starch, protein or fiber of a flower or silique. Alternatively, reproduction gene promoters can direct expression of non-plant genes that can, for instance confer insect resistance specifically to a flower.

### III.A.7. Ovule Genes, Gene Components and Products

The ovule is the primary female sexual reproductive organ of flowering plants. It contains the egg cell and, after fertilization occurs, contains the developing seed. Consequently, the ovule is at times comprised of haploid, diploid and triploid tissue. As such, ovule development requires the orchestrated transcription of numerous polynucleotides, some of which are ubiquitous, others that are ovule-specific and still others that are expressed only in the haploid, diploid or triploid cells of the ovule.

Although the morphology of the ovule is well known, little is known of these polynucleotides and polynucleotide products. Mutants allow identification of genes that participate in ovule development. As an example, the pistillata (PI) mutant replaces stamens with carpels, thereby increasing the number of ovules present in the flower.

### Use of Promoters of Ovule Genes

Promoters of Ovule genes are useful for transcription of any desired polynucleotide or plant or non-plant origin. Further, any desired sequence can be transcribed in a similar temporal, tissue, or environmentally specific patterns as the Ovule genes where the desired sequence is operably linked to a promoter of a Ovule gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.A.8. Seed and Fruit Development Genes, Gene Components and Products

The ovule is the primary female sexual reproductive organ of flowering plants. At maturity it contains the egg cell and one large central cell containing two polar nuclei encased by two integuments that, after fertilization, develops into the embryo, endosperm, and seed coat of the mature seed, respectively. As the ovule develops into the seed, the ovary matures into the fruit or silique. As such, seed and fruit development requires the orchestrated transcription of numerous polynucleotides, some of which are ubiquitous, others that are embryo-specific and still others that are expressed only in the endosperm, seed coat, or fruit. Such genes are termed fruit development responsive genes.

### Use of Promoters of Seed and Fruit Development Genes

Promoters of seed and fruit development genes are useful for transcription of any desired polynucleotide or plant or non-plant origin. Further, any desired sequence can be transcribed in a similar temporal, tissue, or environmentally specific patterns as the seed and fruit development genes where the desired sequence is operably linked to a promoter of a seed and fruit development gene. The protein product of such a polynucleotide is usually synthesized in the same cells, in response to the same stimuli as the protein product of the gene from which the promoter was derived. Such a promoter is also useful to produce antisense mRNAs to down-regulate the product of proteins, or to produce sense mRNAs to down-regulate mRNAs via sense suppression.

### III.B. Development Genes, Gene Components and Products

### III.B.1. Imbibition and Germination Responsive Genes, Gene Components and Products

Seeds are a vital component of the world's diet. Cereal grains alone, which comprise ˜90% of all cultivated seeds, contribute up to half of the global per capita energy intake. The primary organ system for seed production in flowering plants is the ovule. At maturity, the ovule consists of a haploid female gametophyte or embryo sac surrounded by several layers of maternal tissue including the nucleus and the integuments. The embryo sac typically contains seven cells including the egg cell, two synergids, a large central cell containing two polar nuclei, and three antipodal cells. That pollination results in the fertilization of both egg and central cell. The fertilized egg develops into the embryo. The fertilized central cell develops into the endosperm. And the integuments mature into the seed coat. As the ovule develops into the seed, the ovary matures into the fruit or silique. Late in development, the developing seed ends a period of extensive biosynthetic and cellular activity and begins to desiccate to complete its development and enter a dormant, metabolically quiescent state. Seed dormancy is generally an undesirable characteristic in agricultural crops, where rapid germination and growth are required. However, some degree of dormancy is advantageous, at least during seed development. This is particularly true for cereal crops because it prevents germination of grains while still on the ear of the parent plant (preharvest sprouting), a phenomenon that results in major losses to the agricultural industry. Extensive domestication and breeding of crop species have ostensibly reduced the level of dormancy mechanisms present in the seeds of their wild ancestors, although under some adverse environmental conditions, dormancy may reappear. By contrast, weed seeds frequently mature with inherent dormancy mechanisms that allow some seeds to persist in the soil for many years before completing germination.

Germination commences with imbibition, the uptake of water by the dry seed, and the activation of the quiescent embryo and endosperm. The result is a burst of intense metabolic activity. At the cellular level, the genome is transformed from an inactive state to one of intense transcriptional activity. Stored lipids, carbohydrates and proteins are catabolized fueling seedling growth and development. DNA and organelles are repaired, replicated and begin functioning. Cell expansion and cell division are triggered. The shoot and root apical meristem are activated and begin growth and organogenesis. Schematic 4 summarizes some of the metabolic and cellular processes that occur during imbibition. Germination is complete when a part of the embryo, the radicle, extends to penetrate the structures that surround it. In Arabidopsis, seed germination takes place within twenty-four (24) hours after imbibition. As such, germination requires the rapid and orchestrated transcription of numerous polynucleotides. Germination is followed by expansion of the hypocotyl and opening of the cotyledons. Meristem development continues to promote root growth and shoot growth, which is followed by early leaf formation.

### Use of Promoters of Imbibition and Germination Genes

These promoters can be used to control expression of any polynucleotide, plant or non-plant, in a plant host. Selected promoters when operably linked to a coding sequence can direct synthesis of the protein in specific cell types or to loss of a protein product, for example when the coding sequence is in the antisense configuration. They are thus useful in controlling changes in imbibition and germination phenotypes or enabling novel proteins to be made in germinating seeds.

### III.B.2. Early Seedling-Phase Specific Responsive Genes, Gene Components and Products

One of the more active stages of the plant life cycle is a few days after germination is complete, also referred to as the early seedling phase. During this period the plant begins development and growth of the first leaves, roots, and other organs not found in the embryo. Generally this stage begins when germination ends. The first sign that germination has been completed is usually that there is an increase in length and fresh weight of the radicle.

### Use of Promoters of Early Seedling-Phase Genes

Promoters of early seedling phase genes are useful for transcription of desired polynucleotides, both plant and non-plant. If the gene is expressed only in the post-germination seedling, or in certain kinds of leaf cells, the promoter is used to drive the synthesis of proteins specifically in those cells. For example, extra copies of carbohydrate transporter cDNAs operably linked to a early seedling phase gene promoter and inserted into a plant increase the “sink” strength of leaves. Similarly, early seedling phase promoters are used to drive transcription of metabolic enzymes that alter the oil, starch, protein, or fiber contents of the seedling. Alternatively, the promoters direct expression of non-plant genes that can, for instance, confer resistance to specific pathogen. Additionally the promoters are used to synthesize an antisense mRNA copy of a gene to inactivate the normal gene expression into protein. The promoters are used to drive synthesis of sense RNAs to inactivate protein production via RNA interference.

### III.B.3. Size and Stature Genes, Gene Components and Products

Great agronomic value can result from modulating the size of a plant as a whole or of any of its organs. For example, the green revolution came about as a result of creating dwarf wheat plants, which produced a higher seed yield than taller plants because they could withstand higher levels and inputs of fertilizer and water. Size and stature genes elucidated here are capable of modifying the growth of either an organism as a whole or of localized organs or cells. Manipulation of such genes, gene components and products can enhance many traits of economic interest from increased seed and fruit size to increased lodging resistance. Many kinds of genes control the height attained by a plant and the size of the organs. For genes additional to the ones in this section other sections of the Application should be consulted.

### Use of Promoters of “Size and Stature” Genes

Promoters of “size and stature” genes are useful for controlling the transcription of any desired polynucleotides, both plant and non-plant. They can be discovered from the “size and stature” genes in the Reference Tables, and their patterns of activity from the MA Tables. When operably linked to any polynucleotide encoding a protein, and inserted into a plant, the protein will be synthesized in those cells in which the promoter is active. Many “size and stature” genes will function in meristems, so the promoters will be useful for expressing proteins in meristems. The promoters can be used to cause loss of, as well as synthesis of, specific proteins via antisense and sense suppression approaches.

### III.B.4. Shoot-Apical Meristem Genes, Gene Components and Products

New organs, stems, leaves, branches and inflorescences develop from the stem apical meristem (SAM). The growth structure and architecture of the plant therefore depends on the behavior of SAMs. Shoot apical meristems (SAMs) are comprised of a number of morphologically undifferentiated, dividing cells located at the tips of shoots. SAM genes elucidated here are capable of modifying the activity of SAMs and thereby many traits of economic interest from ornamental leaf shape to organ number to responses to plant density.

### Use of SAM Gene Promoters to Modify SAMS

Promoters of SAM genes, as described in the Reference tables, for example, can be used to modulate transcription of coding sequences in SAM cells to influence growth, differentiation or patterning of development or any of the phenotypes or biological activities above. For example, any desired sequence can be transcribed in similar temporal, tissue, or environmentally specific patterns as a SAM gene when the desired sequence is operably linked to the promoter of the SAM gene.

A specific instance is linking of a SAM gene promoter normally active in floral meristem primordia, to a phytotoxic protein coding sequence to inhibit apical meristem switching into an inflorescence and/or floral meristem, thereby preventing flowering.

SAM gene promoters can also be used to induce transcription of antisense RNA copies of a gene or an RNA variant to achieve reduced synthesis of a specific protein in specific SAM cells. This provides an alternative way to the example above, to prevent flowering.

### III.B.5. Vegetative-Phase Specific Responsive Genes, Gene Components and Products

Often growth and yield are limited by the ability of a plant to tolerate stress conditions, including water loss. To combat such conditions, plant cells deploy a battery of responses that are controlled by a phase shift, from so called juvenile to adult. These changes at distinct times involve, for example, cotyledons and leaves, guard cells in stomata, and biochemical activities involved with sugar and nitrogen metabolism. These responses depend on the functioning of an internal clock, that becomes entrained to plant development, and a series of downstream signaling events leading to transcription-independent and transcription-dependent stress responses. These responses involve changes in gene expression.

### Use of Promoters of Phase Responsive Genes

Promoters of phase responsive genes are useful for transcription of any desired polynucleotide or plant or non-plant origin. Further, any desired sequence can be transcribed in a similar temporal, tissue, or environmentally specific patterns as the phase responsive genes where the desired sequence is operably linked to a promoter of a phase responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.C. Horomone Responsive Genes, Gene Components and Products

### III.C.1. Abiscissic Acid Responsive Genes, Gene Components and Products

Plant hormones are naturally occurring substances, effective in very small amounts, which act as signals to stimulate or inhibit growth or regulate developmental processes in plants. Abscisic acid (ABA) is a ubiquitous hormone in vascular plants that has been detected in every major organ or living tissue from the root to the apical bud. The major physiological responses affected by ABA are dormancy, stress stomatal closure, water uptake, abscission and senescence. In contrast to Auxins, cytokinins and gibberellins, which are principally growth promoters, ABA primarily acts as an inhibitor of growth and metabolic processes.

### Use of Promoters of ABA Responsive Genes

Promoters of ABA responsive genes are useful for transcription of any desired polynucleotide or plant or non-plant origin. Further, any desired sequence can be transcribed in a similar temporal, tissue, or environmentally specific patterns as the ABA responsive genes where the desired sequence is operably linked to a promoter of a ABA responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.C.2. Auxin Responsive Genes, Gene Components and Products

Plant hormones are naturally occurring substances, effective in very small amounts that stimulate or inhibit growth or regulate developmental processes in plants. One of the plant hormones is indole-3-acetic acid (IAA), often referred to as Auxin.

### Use of Promoters of NAA Responsive Genes

Promoters of NAA responsive genes are useful for transcription of any desired polynucleotide or plant or non-plant origin. Further, any desired sequence can be transcribed in a similar temporal, tissue, or environmentally specific patterns as the NAA responsive genes where the desired sequence is operably linked to a promoter of a NAA responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.C.3. Brassinosteroid Responsive Genes, Gene Components and Products

Plant hormones are naturally occuring substances, effective in very small amounts, which act as signals to stimulate or inhibit growth or regulate developmental processes in plants. Brassinosteroids (BRs) are the most recently discovered, and least studied, class of plant hormones. The major physiological response affected by BRs is the longitudinal growth of young tissue via cell elongation and possibly cell division. Consequently, disruptions in BR metabolism, perception and activity frequently result in a dwarf phenotype. In addition, because BRs are derived from the sterol metabolic pathway, any perturbations to the sterol pathway can affect the BR pathway. In the same way, perturbations in the BR pathway can have effects on the later part of the sterol pathway and thus the sterol composition of membranes.

### Use of Promoters of BR Responsive Genes

Promoters of BR responsive genes are useful for transcription of any desired polynucleotide or plant or non-plant origin. Further, any desired sequence can be transcribed in a similar temporal, tissue, or environmentally specific patterns as the BR responsive genes where the desired sequence is operably linked to a promoter of a BR responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.C.4. Cytokinin Responsive Genes, Gene Components and Products

Plant hormones are naturally occurring substances, effective in very small amounts, which act as signals to stimulate or inhibit growth or regulate developmental processes in plants. Cytokinins (BA) are a group of hormones that are best known for their stimulatory effect on cell division, although they also participate in many other processes and pathways. All naturally occurring BAs are aminopurine derivatives, while nearly all synthetic compounds with BA activity are 6-substituted aminopurine derivatives. One of the most common synthetic BAs used in agriculture is benzylaminopurine (BAP).

### Use of Promoters of BA Responsive Genes

Promoters of Ba responsive genes are useful for transcription of any desired polynucleotide or plant or non-plant origin. Further, any desired sequence can be transcribed in a similar temporal, tissue, or environmentally specific patterns as the BA responsive genes where the desired sequence is operably linked to a promoter of a BA responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.C.5. Gibberellic Acid Responsive Genes, Gene Components and Products

Plant hormones are naturally occuring substances, effective in very small amounts, which act as signals to stimulate or inhibit growth or regulate developmental processes in plants. Gibberellic acid (GA) is a hormone in vascular plants that is synthesized in proplastids (giving rise to chloroplasts or leucoplasts) and vascular tissues. The major physiological responses affected by GA are seed germination, stem elongation, flower induction, anther development and seed and pericarp growth. GA is similar to Auxins, cytokinins and gibberellins, in that they are principally growth promoters.

### Use of Promoters of GA Responsive Genes

Promoters of GA responsive genes are useful for transcription of any desired polynucleotide or plant or non-plant origin. Further, any desired sequence can be transcribed in a similar temporal, tissue, or environmentally specific patterns as the GA responsive genes where the desired sequence is operably linked to a promoter of a GA responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D. Metabolism Affecting Genes, Gene Components and Products

### III.D.1. Nitrogen Responsive Genes, Gene Components and Products

Nitrogen is often the rate-limiting element in plant growth, and all field crops have a fundamental dependence on exogenous nitrogen sources. Nitrogenous fertilizer which is usually supplied as ammonium nitrate, potassium nitrate, or urea, typically accounts for 40% of the costs associated with crops, such as corn and wheat in intensive agriculture. Increased efficiency of nitrogen use by plants should enable the production of higher yields with existing fertilizer inputs and/or enable existing yields of crops to be obtained with lower fertilizer input, or better yields on soils of poorer quality. Also, higher amounts of proteins in the crops could also be produced more cost-effectively.

### Use of Promoters of GA Responsive Genes

Promoters of nitrogen responsive genes are useful for transcription of any desired polynucleotide or plant or non-plant origin. Further, any desired sequence can be transcribed in a similar temporal, tissue, or environmentally specific patterns as the nitrogen responsive genes where the desired sequence is operably linked to a promoter of a nitrogen responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D.2. Circidian Rhythm (Clock) Responsive Genes, Gene Components and Products

Often growth and yield are limited by the ability of a plant to tolerate stress conditions, including water loss. To combat such conditions, plant cells deploy a battery of responses that are controlled by an internal circadian clock, including the timed movement of cotyledons and leaves, timed movements in guard cells in stomata, and timed biochemical activities involved with sugar and nitrogen metabolism. These responses depend on the functioning of an internal circadian clock, that becomes entrained to the ambient light/dark cycle, and a series of downstream signaling events leading to transcription independent and transcription dependent stress responses.

A functioning circadian clock can anticipate dark/light transitions and prepare the physiology and biochemistry of a plant accordingly. For example, expression of a chlorophyll a/b binding protein (CAB) is elevated before daybreak, so that photosynthesis can operate maximally as soon as there is light to drive it. Similar considerations apply to light/dark transitions and to many areas of plant physiology such as sugar metabolism, nitrogen metabolism, water uptake and water loss, flowering and flower opening, epinasty, germination, perception of season, and senescence.

### Use of Promoters of Clock Responsive Genes

Promoters of Clock responsive genes are useful for transcription of any desired polynucleotide or plant or non-plant origin. Further, any desired sequence can be transcribed in a similar temporal, tissue, or environmentally specific patterns as the Clock responsive genes where the desired sequence is operably linked to a promoter of a Clock responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D.3. Blue Light (Phototropism) Responsive Genes, Gene Components and Products

Phototropism is the orientation or growth of a cell, an organism or part of an organism in relation to a source of light. Plants can sense red (R), far-red (FR) and blue light in their environment and respond differently to particular ratios of these. For example, a low R:FR ratio enhances cell elongation and favors flowering over leaf production, but blue light regulated cryptochromes also appear to be involved in determining hypocotyl growth and flowering time. Phototropism of Arabidopsis thaliana seedlings in response to a blue light source is initiated by nonphototropic hypocotyl 1 (NPH1), a blue light-activated serine-threonine protein kinase, but the downstream signaling events are not entirely known. Blue light treatment leads to changes in gene expression. These genes have been identified by comparing the levels of mRNAs of individual genes in dark-grown seedlings, compared with in dark grown seedlings treated with 1 hour of blue light. Auxin also affects blue light phototropism. The effect of Auxin on gene expression stimulated by blue light has been explored by studying mRNA levels in a mutant of Arabidopsis thaliana nph4-2, grown in the dark and, treated with blue light for 1 hour compared with wild type seedlings treated similarly. This mutant is disrupted for Auxin-related growth and Auxin-induced gene transcription.

### Use of Promoters of Blue Light Responsive Genes

Promoters of Blue Light responsive genes are useful for transcription of any desired polynucleotide or plant or non-plant origin. Further, any desired sequence can be transcribed in a similar temporal, tissue, or environmentally specific patterns as the Blue Light responsive genes where the desired sequence is operably linked to a promoter of a Blue Light responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D.4 Responsive Genes, Gene Components and Products

There has been a recent and significant increase in the level of atmospheric carbon dioxide. This rise in level is projected to continue over the next 50 years. The effects of the increased level of carbon dioxide on vegetation are just now being examined, generally in large scale, whole plant (often trees) experiments. Some researchers have initiated physiological experiments in attempts to define the biochemical pathways that are either affected by and/or are activated to allow the plant to avert damage from the elevated carbon dioxide levels. A genomics approach to this issue, using a model plant system, allows identification of those pathways affected by and/or as having a role in averting damage due to the elevated carbon dioxide levels and affecting growth. Higher agronomic yields can be obtained for some crops grown in elevated CO₂.

### Use of Promoters of CO2 Responsive Genes

Promoters of CO2 responsive genes are useful for transcription of any desired polynucleotide or plant or non-plant origin. Further, any desired sequence can be transcribed in a similar temporal, tissue, or environmentally specific patterns as the CO2 responsive genes where the desired sequence is operably linked to a promoter of a CO2 responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D.5. Mitochrondria Electron Transport (Respiration) Genes, Gene Components and Products

One means to alter flux through metabolic pathways is to alter the levels of proteins in the pathways. Plant mitochondria contain many proteins involved in various metabolic processes, including the TCA cycle, respiration, and photorespiration and particularly the electron transport chain (mtETC). Most mtETC complexes consist of nuclearly-encoded mitochondrial proteins (NEMPs) and mitochondrially-encoded mitochondrial proteins (MEMPs). NEMPs are produced in coordination with MEMPs of the same complex and pathway and with other proteins in multi-organelle pathways. Enzymes involved in photorespiration, for example, are located in chloroplasts, mitochondria, and peroxisomes and many of the proteins are nuclearly-encoded. Manipulation of the coordination of protein levels within and between organelles can have critical and global consequences to the growth and yield of a plant.

### Use of Promoters of Resperation Genes

Promoters of Respiration genes are useful for transcription of any desired polynucleotide or plant or non-plant origin. Further, any desired sequence can be transcribed in a similar temporal, tissue, or environmentally specific patterns as the Respiration genes where the desired sequence is operably linked to a promoter of a Respiration gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D.6. Protien Degradation Genes, Gene Components and Products

One of the components of molecular mechanisms that operate to support plant development is the “removal” of a gene product from a particular developmental circuit once the substrate protein is not functionally relevant anymore in temporal and/or spatial contexts. The “removal” mechanisms can be accomplished either by protein inactivation (e.g., phosphorylation or protein-protein interaction) or protein degradation most notably via ubiquitination-proteasome pathway. The ubiquitination-proteasome pathway is responsible for the degradation of a plethora of proteins involved in cell cycle, cell division, transcription, and signal transduction, all of which are required for normal cellular functions. Ubiquitination occurs through the activity of ubiquitin-activating enzymes (E1), ubiquitin-conjugating enzymes (E2), and ubiquitin-protein ligases (E3), which act sequentially to catalyze the attachment of ubiquitin (or other modifying molecules that are related to ubiquitin) to substrate proteins (Hochstrasser 2000, Science 289: 563). Ubiquitinated proteins are then routed to proteasomes for degradation processing [2000, Biochemistry and Molecular Biology of Plants, Buchanan, Gruissem, and Russel (eds), Amer. Soc. of Plant Physiologists, Rockville, Md.]. The degradation mechanism can be selective and specific to the concerned target protein (Joazeiro and Hunter 2001, Science 289: 2061; Sakamoto et al., 2001, PNAS Online 141230798). This selectivity and specificity may be one of the ways that the activity of gene products is modulated.

### Use of Promoters and “Protien Degradation Genes, Gene Components and Products”

Promoters of “protein degradation” genes, as described in the Reference tables, for example, can be used to modulate transcription of any polynucleotide, plant or non plant to achieve synthesis of a protein in association with production of the ubiquitination-proteasome pathway or the various cellular systems associated with it. Additionally such promoters can be used to synthesize antisense RNA copies of any gene to reduce the amount of protein product produced, or to synthesize RNA copies that reduce protein formation by RNA interference. Such modifications can make phenotypic changes and produce altered plants as described above.

### III.D.7. Cartenogenesis Responsive Genes, Gene Components and Products

Carotenoids serve important biochemical functions in both plants and animals. In plants, carotenoids function as accessory light harvesting pigments for photosynthesis and to protect chloroplasts and photosystem II from heat and oxidative damage by dissipating energy and scavenging oxygen radicals produced by high light intensities and other oxidative stresses. Decreases in yield frequently occur as a result of light stress and oxidative stress in the normal growth ranges of crop species. In addition light stress limits the geographic range of many crop species. Modest increases in oxidative stress tolerance would greatly improve the performance and growth range of many crop species. The development of genotypes with increased tolerance to light and oxidative stress would provide a more reliable means to minimize crop losses and diminish the use of energy-costly practices to modify the soil environment.

In animals carotenoids such as beta-carotene are essential provitamins required for proper visual development and function. In addition, their antioxidative properties are also thought to provide valuable protection from diseases such as cancer. Modest increases in carotenoid levels in crop species could produce a dramatic effect on plant nutritional quality. The development of genotypes with increased carotenoid content would provide a more reliable and effective nutritional source of Vitamin A and other carotenoid derived antioxidants than through the use of costly nutritional supplements.

### Use of Promoters of Carotenogenesis Responsive Genes

Promoters of Carotenogenesis responsive genes are useful for transcription of any desired polynucleotide or plant or non-plant origin. Further, any desired sequence can be transcribed in a similar temporal, tissue, or environmentally specific patterns as the Carotenogenesis responsive genes where the desired sequence is operably linked to a promoter of a Carotenogenesis responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.D.8. Viability Genes, Gene Components and Products

Plants contain many proteins and pathways that when blocked or induced lead to cell, organ or whole plant death. Gene variants that influence these pathways can have profound effects on plant survival, vigor and performance. The critical pathways include those concerned with metabolism and development or protection against stresses, diseases and pests. They also include those involved in apoptosis and necrosis. The applicants have elucidated many such genes and pathways by discovering genes that when inactivated lead to cell or plant death.

Herbicides are, by definition, chemicals that cause death of tissues, organs and whole plants. The genes and pathways that are activated or inactivated by herbicides include those that cause cell death as well as those that function to provide protection. The applicants have elucidated these genes.

The genes defined in this section have many uses including manipulating which cells, tissues and organs are selectively killed, which are protected, making plants resistant to herbicides, discovering new herbicides and making plants resistant to various stresses.

### Use of Promoters of Viability Genes, Gene Components and Products

Promoters of viability genes can include those that are induced by (1) destructive chemicals, e.g. herbicides, (2) stress, or (3) death. These promoters can be linked operably to achieve expression of any polynucleotide from any organism. Specific promoters from viability genes can be selected to ensure transcription in the desired tissue or organ. Proteins expressed under the control of such promoters can include those that can induce or accelerate death or those that can protect plant cells organ death. For example, stress tolerance can be increased by using promoters of viability genes to drive transcription of cold tolerance proteins, for example. Alternatively, promoters induced by apoptosis can be utilized to drive transcription of antisense constructs that inhibit cell death.

### III.D.9. Histone Deactylase (Axel) Responsive Genes, Gene Components and Products

The deacetylation of histones is known to play an important role in regulating gene expression at the chromatin level in eukaryotic cells. Histone deacetylation is catalyzed by proteins known as histone deacetylases (HDAcs). HDAcs are found in multisubunit complexes that are recruited to specific sites on nuclear DNA thereby affecting chromatin architecture and target gene transcription. Mutations in plant HDAc genes cause alterations in vegetative and reproductive growth that result from changes in the expression and activities of HDAc target genes or genes whose expression is governed by HDAc target genes. For example, transcription factor proteins control whole pathways or segments of pathways and proteins also control the activity of signal transduction pathways. Therefore, manipulation of these types of protein levels is especially useful for altering phenotypes and biochemical activities.

### Use of Promoters of Histone Deacetylase Responsive Genes

Promoters of Histone Deacetylase responsive genes are useful for transcription of any desired polynucleotide or plant or non-plant origin. Further, any desired sequence can be transcribed in a similar temporal, tissue, or environmentally specific patterns as the Histone Deacetylase responsive genes where the desired sequence is operably linked to a promoter of a Histone Deacetylase responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E. Stress Responsive Genes, Gene Components and Products

### III.E.1. Cold Responsive Genes, Gene Components and Products

The ability to endure low temperatures and freezing is a major determinant of the geographical distribution and productivity of agricultural crops. Even in areas considered suitable for the cultivation of a given species or cultivar, can give rise to yield decreases and crop failures as a result of aberrant, freezing temperatures. Even modest increases (1-2° C.) in the freezing tolerance of certain crop species would have a dramatic impact on agricultural productivity in some areas. The development of genotypes with increased freezing tolerance would provide a more reliable means to minimize crop losses and diminish the use of energy-costly practices to modify the microclimate.

### Use of Promoters of Cold Responsive Genes

Promoters of cold responsive genes are useful for transcription of any desired polynucleotide or plant or non-plant origin. Further, any desired sequence can be transcribed in a similar temporal, tissue, or environmentally specific patterns as the cold responsive genes where the desired sequence is operably linked to a promoter of a cold responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.2. Heat Responsive Genes, Gene Components and Products

The ability to endure high temperatures is a major determinant of the geographical distribution and productivity of agricultural crops. Decreases in yield and crop failure frequently occur as a result of aberrant, hot conditions even in areas considered suiTable for the cultivation of a given species or cultivar. Only modest increases in the heat tolerance of crop species would have a dramatic impact on agricultural productivity. The development of genotypes with increased heat tolerance would provide a more reliable means to minimize crop losses and diminish the use of energy-costly practices to modify the microclimate.

### Use of Promoters of Heat Responsive Genes

Promoters of heat responsive genes are useful for transcription of any desired polynucleotide or plant or non-plant origin. Further, any desired sequence can be transcribed in a similar temporal, tissue, or environmentally specific patterns as the heat responsive genes where the desired sequence is operably linked to a promoter of a heat responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.3. Drought Responsive Genes, Gene Components and Products

The ability to endure drought conditions is a major determinant of the geographical distribution and productivity of agricultural crops. Decreases in yield and crop failure frequently occur as a result of aberrant, drought conditions even in areas considered suiTable for the cultivation of a given species or cultivar. Only modest increases in the drought tolerance of crop species would have a dramatic impact on agricultural productivity. The development of genotypes with increased drought tolerance would provide a more reliable means to minimize crop losses and diminish the use of energy-costly practices to modify the microclimate.

### Use of Promoters Drought Responsive Genes

Promoters of Drought responsive genes are useful for transcription of any desired polynucleotide or plant or non-plant origin. Further, any desired sequence can be transcribed in a similar temporal, tissue, or environmentally specific patterns as the Drought responsive genes where the desired sequence is operably linked to a promoter of a Drought responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.4. Wounding Responsive Genes, Gene Components and Products

Plants are continuously subjected to various forms of wounding from physical attacks including the damage created by pathogens and pests, wind, and contact with other objects. Therefore, survival and agricultural yields depend on constraining the damage created by the wounding process and inducing defense mechanisms against future damage.

Plants have evolved complex systems to minimize and/or repair local damage and to minimize subsequent attacks by pathogens or pests or their effects. These involve stimulation of cell division and cell elongation to repair tissues, induction of programmed cell death to isolate the damage caused mechanically and by invading pests and pathogens, and induction of long-range signaling systems to induce protecting molecules, in case of future attack. The genetic and biochemical systems associated with responses to wounding are connected with those associated with other stresses such as pathogen attack and drought.

### Use of Promoters of Wounding Responsive Genes

Promoters of Wounding responsive genes are useful for transcription of any desired polynucleotide or plant or non-plant origin. Further, any desired sequence can be transcribed in a similar temporal, tissue, or environmentally specific patterns as the Wounding responsive genes where the desired sequence is operably linked to a promoter of a Wounding responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.5. Methyl Jasmonate (Jasmonate) Responsive Genes, Gene Components and Products

Jasmonic acid and its derivatives, collectively referred to as jasmonates, are naturally occurring derivatives of plant lipids. These substances are synthesized from linolenic acid in a lipoxygenase-dependent biosynthetic pathway. Jasmonates are signalling molecules which have been shown to be growth regulators as well as regulators of defense and stress responses. As such, jasmonates represent a separate class of plant hormones.

### Use of Promoters Jasmonate Responsive Genes

Promoters of Jasmonate responsive genes are useful for transcription of any desired polynucleotide or plant or non-plant origin. Further, any desired sequence can be transcribed in a similar temporal, tissue, or environmentally specific patterns as the Jasmonate responsive genes where the desired sequence is operably linked to a promoter of a Jasmonate responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.6. Reactive Oxygen Responsive Genes, Gene Components and H2O2 Products

Often growth and yield are limited by the ability of a plant to tolerate stress conditions, including pathogen attack, wounding, extreme temperatures, and various other factors. To combat such conditions, plant cells deploy a battery of inducible defense responses, including triggering an oxidative burst. The burst of reactive oxygen intermediates occurs in time, place and strength to suggest it plays a key role in either pathogen elimination and/or subsequent signaling of downstream defense functions. For example, H₂O₂ can play a key role in the pathogen resistance response, including initiating the hypersensitive response (HR). HR is correlated with the onset of systemic acquired resistance (SAR) to secondary infection in distal tissues and organs.

### Use of Promoters of Reactive Oxygen Responsive Genes

Promoters of Reactive Oxygen responsive genes are useful for transcription of any desired polynucleotide or plant or non-plant origin. Further, any desired sequence can be transcribed in a similar temporal, tissue, or environmentally specific patterns as the Reactive Oxygen responsive genes where the desired sequence is operably linked to a promoter of a Reactive Oxygen responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.7. Salicycle Acid Responsive Genes, Gene Components and Products

Plant defense responses can be divided into two groups: constitutive and induced. Salicylic acid (SA) is a signaling molecule necessary for activation of the plant induced defense system known as systemic acquired resistance or SAR. This response, which is triggered by prior exposure to avirulent pathogens, is long lasting and provides protection against a broad spectrum of pathogens. Another induced defense system is the hypersensitive response (HR). HR is far more rapid, occurs at the sites of pathogen (avirulent pathogens) entry and precedes SAR. SA is also the key signaling molecule for this defense pathway.

### Use of Promoters of Salicycle Acid Responsive Genes

Promoters of Salicylic Acid responsive genes are useful for transcription of any desired polynucleotide or plant or non-plant origin. Further, any desired sequence can be transcribed in a similar temporal, tissue, or environmentally specific patterns as the Salicylic Acid responsive genes where the desired sequence is operably linked to a promoter of a Salicylic Acid responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.8. Nitric Oxide Responsive Genes, Gene Components and Products

The rate-limiting element in plant growth and yield is often its ability to tolerate suboptimal or stress conditions, including pathogen attack conditions, wounding and the presence of various other factors. To combat such conditions, plant cells deploy a battery of inducible defense responses, including synergistic interactions between nitric oxide (NO), reactive oxygen intermediates (ROS), and salicylic acid (SA). NO has been shown to play a critical role in the activation of innate immune and inflammatory responses in animals. At least part of this mammalian signaling pathway is present in plants, where NO is known to potentiate the hypersensitive response (HR). In addition, NO is a stimulator molecule in plant photomorphogenesis.

### Use of Promoters of No Responsive Genes

Promoters of NO responsive genes are useful for transcription of any desired polynucleotide or plant or non-plant origin. Further, any desired sequence can be transcribed in a similar temporal, tissue, or environmentally specific patterns as the NO responsive genes where the desired sequence is operably linked to a promoter of a NO responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

111.9. Osmotic Stress Responsive Genes, Gene Components and Products

The ability to endure and recover from osmotic and salt related stress is a major determinant of the geographical distribution and productivity of agricultural crops. Osmotic stress is a major component of stress imposed by saline soil and water deficit. Decreases in yield and crop failure frequently occur as a result of aberrant or transient environmental stress conditions even in areas considered suitable for the cultivation of a given species or cultivar. Only modest increases in the osmotic and salt tolerance of a crop species would have a dramatic impact on agricultural productivity. The development of genotypes with increased osmotic tolerance would provide a more reliable means to minimize crop losses and diminish the use of energy-costly practices to modify the soil environment.

### Use of Promoters of Osmotic Stress Responsive Genes

Promoters of Osmotic Stress responsive genes are useful for transcription of any desired polynucleotide or plant or non-plant origin. Further, any desired sequence can be transcribed in a similar temporal, tissue, or environmentally specific patterns as the Osmotic Stress responsive genes where the desired sequence is operably linked to a promoter of a Osmotic Stress responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.10. Aluminum Responsive Genes, Gene Components and Products

Aluminum is toxic to plants in soluble form (Al³⁺). Plants grown under aluminum stress have inhibited root growth and function due to reduced cell elongation, inhibited cell division and metabolic interference. As an example, protein inactivation frequently results from displacement of the Mg2+ cofactor with aluminum. These types of consequences result in poor nutrient and water uptake. In addition, because stress perception and response occur in the root apex, aluminum exposure leads to the release of organic acids, such as citrate, from the root as the plant attempts to prevent aluminum uptake.

The ability to endure soluble aluminum is a major determinant of the geographical distribution and productivity of agricultural crops. Decreases in yield and crop failure frequently occur as a result of aberrant, hot conditions even in areas considered suiTable for the cultivation of a given species or cultivar. Only modest increases in the aluminum tolerance of crop species would have a dramatic impact on agricultural productivity. The development of genotypes with increased aluminum tolerance would provide a more reliable means to minimize crop losses and diminish the use of costly practices to modify the environment.

### Use of Promoters of Aluminum Responsive Genes

Promoters of Aluminum responsive genes are useful for transcription of any desired polynucleotide or plant or non-plant origin. Further, any desired sequence can be transcribed in a similar temporal, tissue, or environmentally specific patterns as the Aluminum responsive genes where the desired sequence is operably linked to a promoter of a Aluminum responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.11. Cadium Responsive Genes, Gene Components and Products

Cadmium (Cd) has both toxic and non-toxic effects on plants. Plants exposed to non-toxic concentrations of cadmium are blocked for viral disease due to the inhibition of systemic movement of the virus. Surprisingly, higher, toxic levels of Cd do not inhibit viral systemic movement, suggesting that cellular factors that interfere with the viral movement are triggered by non-toxic Cd concentrations but repressed in high Cd concentrations. Furthermore, exposure to non-toxic Cd levels appears to reverse posttranslational gene silencing, an inherent plant defense mechanism. Consequently, exploring the effects of Cd exposure has potential for advances in plant disease control in addition to soil bio-remediation and the improvement of plant performance in agriculture.

### Use of Promoters of Cadium Responsive Genes

Promoters of Cadmium responsive genes are useful for transcription of any desired polynucleotide or plant or non-plant origin. Further, any desired sequence can be transcribed in a similar temporal, tissue, or environmentally specific patterns as the Cadmium responsive genes where the desired sequence is operably linked to a promoter of a Cadmium responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.12. Disease Responsive Genes, Gene Components and Products

Often growth and yield are limited by the ability of a plant to tolerate stress conditions, including pathogen attack. To combat such conditions, plant cells deploy a battery of inducible defense responses, including the triggering of an oxidative burst and the transcription of pathogenesis-related protein (PR protein) genes. These responses depend on the recognition of a microbial avirulence gene product (avr) by a plant resistance gene product (R), and a series of downstream signaling events leading to transcription- independent and transcription-dependent disease resistance responses. Reactive oxygen species (ROS) such as H₂O₂ and NO from the oxidative burst plays a signaling role, including initiation of the hypersensitive response (HR) and induction of systemic acquired resistance (SAR) to secondary infection by unrelated pathogens. PR proteins are able to degrade the cell walls of invading microorganisms, and phytoalexins are directly microbicidal.

### Use of Promoters of Disease Responsive Genes

Promoters of Disease responsive genes are useful for transcription of any desired polynucleotide or plant or non-plant origin. Further, any desired sequence can be transcribed in a similar temporal, tissue, or environmentally specific patterns as the Disease responsive genes where the desired sequence is operably linked to a promoter of a Disease responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### II.E.13. Defense (LOL2) Responsive Genes, Gene Components and Products

Often growth and yield are limited by the ability of a plant to tolerate stress conditions, including pathogen attack. To combat such conditions, plant cells deploy a battery of inducible defense responses, including the triggering of an oxidative burst and the transcription of pathogenesis-related protein (PR protein) genes. Reactive oxygen species (ROS) such as H₂O₂ and NO from the oxidative burst play a signaling role, including initiation of the hypersensitive response (HR) and induction of systemic acquired resistance (SAR) to secondary infection by unrelated pathogens. Some PR proteins are able to degrade the cell walls of invading microorganisms, and phytoalexins are directly microbicidal. Other defense related pathways are regulated by salicylic acid (SA) or methyl jasmonate (MeJ).

These responses depend on the recognition of a microbial avirulence gene product (avr) by a plant resistance gene product (R), and a series of downstream signaling events leading to transcription-independent and transcription-dependent disease resistance responses. Current models suggest that R-gene-encoded receptors specifically interact with pathogen-encoded ligands to trigger a signal transduction cascade. Several components include ndr1 and eds1 loci. NDR1, EDS1, PR1, as well as PDF1.2, a MeJ regulated gene and Nim1, a SA regulated gene, are differentially regulated in plants with mutations in the LOL2 gene.

LOL2 shares a novel zinc finger motif with LSD1, a negative regulator of cell death and defense response. Due to an alternative splice site the LOL2 gene encodes two different proteins, one of which contains an additional, putative DNA binding motif. Northern analysis demonstrated that LOL2 transcripts containing the additional DNA binding motif are predominantly upregulated after treatment with both virulent and avirulent Pseudomonas syringae pv maculicola strains. Modulation in this gene can also confer enhanced resistance to virulent and avirulent Peronospora parasitica isolates.

### Use of Promoters of Defense Responsive Genes

Promoters of Defense responsive genes are useful for transcription of any desired polynucleotide or plant or non-plant origin. Further, any desired sequence can be transcribed in a similar temporal, tissue, or environmentally specific patterns as the Defense responsive genes where the desired sequence is operably linked to a promoter of a Defense responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.14. Iron Responsive Genes, Gene Components and Products

Iron (Fe) deficiency in humans is the most prevalent nutritional problem worldwide today. Increasing iron availability via diet is a sustainable malnutrition solution for many of the world's nations. One-third of the world's soils, however, are iron deficient. Consequently, to form a food-based solution to iron malnutrition, we need a better understanding of iron uptake, storage and utilization by plants. Furthermore, exposure to non-toxic Fe levels appears to affect inherent plant defense mechanisms. Consequently, exploring the effects of Fe exposure has potential for advances in plant disease resistance in addition to human nutrition.

### Use of Promoters of Iron Responsive Genes

Promoters of Iron responsive genes are useful for transcription of any desired polynucleotide or plant or non-plant origin. Further, any desired sequence can be transcribed in a similar temporal, tissue, or environmentally specific patterns as the Iron responsive genes where the desired sequence is operably linked to a promoter of a Iron responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.15. Shade Responsive Genes, Gene Components and Products

Plants sense the ratio of Red (R): Far Red (FR) light in their environment and respond differently to particular ratios. A low R:FR ratio, for example, enhances cell elongation and favors flowering over leaf production. The changes in R:FR ratios mimic and cause the shading response effects in plants. The response of a plant to shade in the canopy structures of agricultural crop fields influences crop yields significantly. Therefore manipulation of genes regulating the shade avoidance responses can improve crop yields. While phytochromes mediate the shade avoidance response, the down-stream factors participating in this pathway are largely unknown. One potential downstream participant, ATHB-2, is a member of the HD-Zip class of transcription factors and shows a strong and rapid response to changes in the R:FR ratio. ATHB-2 overexpressors have a thinner root mass, smaller and fewer leaves and longer hypocotyls and petioles. This elongation arises from longer epidermal and cortical cells, and a decrease in secondary vascular tissues, paralleling the changes observed in wild-type seedlings grown under conditions simulating canopy shade. On the other hand, plants with reduced ATHB-2 expression have a thick root mass and many larger leaves and shorter hypocotyls and petioles. Here, the changes in the hypocotyl result from shorter epidermal and cortical cells and increased proliferation of vascular tissue. Interestingly, application of Auxin is able to reverse the root phenotypic consequences of high ATHB-2 levels, restoring the wild-type phenotype. Consequently, given that ATHB-2 is tightly regulated by phytochrome, these data suggest that ATHB-2 may link the Auxin and phytochrome pathways in the shade avoidance response pathway.

### Use of Promoters of Shade Avoidance Genes

Promoters of Shade Avoidance genes are useful for transcription of any desired polynucleotide or plant or non-plant origin. Further, any desired sequence can be transcribed in a similar temporal, tissue, or environmentally specific patterns as the Shade Avoidance genes where the desired sequence is operably linked to a promoter of a Shade Avoidance gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.16. Sulfur Responsive Genes, Gene Components and Products

Sulfur is one of the important macronutrients required by plants. It is taken up from the soil solution by roots as in the form of sulfate anion which higher plants are dependent on to fulfill their nutritional sulfur requirement. After uptake from the soil, sulfate is either accumulated and stored in vacuole or it is assimilated into various organic compounds, e.g. cysteine, glutathione, methionine, etc. Thus, plants also serve as nutritional sulfur sources for animals. Sulfur can be assimilated in one of two ways: it is either incorporated as sulfate in a reaction called sulfation, or it is first reduced to sulfide, the substrate for cysteine synthesis. In plants, majority of sulfur is assimilated in reduced form.

Sulfur comprises a small by vital fraction of the atoms in many protein molecules. As disulfide bridges, the sulfur atoms aid in stabilizing the folded proteins, such cysteine residues. Cys is the first sulfur-containing amino acids, which in proteins form disulfide bonds that may affect the tertiary structures and enzyme activities. This redox balance is mediated by the disulfide/thiol interchange of thioredoxin or glutaredoxin using NADPH as an electron donor. Sulfur can also become sulfhydryl (SH) groups participating in the active sites of some enzymes and some enzymes require the aid of small molecules that contain sulfur. In addition, the machinery of photosynthesis includes some sulfur-containing compounds, such as ferrodoxin. Thus, sulfate assimilation plays important roles not only in the sulfur nutrition but also in the ubiquitous process that may regulate the biochemical reactions of various metabolic pathways.

Deficiency of sulfur leads to a marked chlorosis in younger leaves, which may become white in color. Other symptoms of sulfur deficiency also include weak stems and reduced growth. Adding sulfur fertilizer to plants can increase root development and a deeper green color of the leaves in sulfur-deficient plants. However, Sulfur is generally sufficient in soils for two reasons: it is a contaminant in potassium and other fertilizers and a product of industrial combustion. Sulfur limitation in plants is thus likely due to the limitation of the uptake and distribution of sulfate in plants. Seven cell type specific sulfate transporter genes have been isolated from Arabidopsis. In sulfate-starved plants, expression of the high-affinity transporter, AtST1-1, is induced in root epidermis and cortex for acquisition of sulfur. The low affinity transporter, AtST2-1 (AST68), accumulates in the root vascular tissue by sulfate starvation for root-to-shoot transport of sulfate. These studies have shown that the whole-plant process of sulfate transport is coordinately regulated by the expression of these 2 sulfate transporter genes under sulfur limited conditions. Recent studies have proposed that feeding of O-acetylserine, GSH and selenate may regulate the expression of AtSTl-I and AtST2-1 (AST68) in roots either positively or negatively. However, regulatory proteins that may directly control the expression of these genes have not been identified yet.

It has been established that there are regulatory interactions between assimilatory sulfate and nitrate reduction in plants. The two assimilatory pathways are very similar and well coordinated; deficiency for one element was shown to repress the other pathway. The coordination between them should be taken into consideration when one tries to alter one of pathways.

### Use of Promoters of Sulfur Responsive Genes

Promoters of Sulfur responsive genes are useful for transcription of any desired polynucleotide or plant or non-plant origin. Further, any desired sequence can be transcribed in a similar temporal, tissue, or environmentally specific patterns as the Sulfur responsive genes where the desired sequence is operably linked to a promoter of a Sulfur responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### III.E.17. Zinc Responsive Genes, Gene Components and Products

Phytoremediation of soils contaminated with toxic levels of heavy metals requires the understanding of plant metal transport and tolerance. The numerous Arabidopsis thaliana studies have given scientists the potential for dissection and elucidation of plant micronutrient/heavy metal uptake and accumulation pathways. It has been shown altered regulation of ZNT1, a Zn/Cd transporter, contributes to high Zn uptake. Isolation and characterization of Zn/Cd hyperaccumulation genes may allow expression in higher biomass plant species for efficient contaminated soil clean up. Identification of additional Zn transport, tolerance and nutrition-related genes involved in heavy metal accumulation will enable manipulation of increased uptake (for phytoremediation) as well as limitation of uptake or leak pathways that contribute to toxicity in crop plants. Additionally, Zn-binding ligands involved in Zn homeostasis or tolerance may be identified, as well as factors affecting the activity or expression of Zn binding transcription factors. Gene products acting in concert to effect Zn uptake, which would not have been identified in complementation experiments, including multimeric transporter proteins, could also be identified.

### Use of Promoters of Zinc Responsive Genes

Promoters of Zinc responsive genes are useful for transcription of any desired polynucleotide or plant or non-plant origin. Further, any desired sequence can be transcribed in a similar temporal, tissue, or environmentally specific patterns as the Zinc responsive genes where the desired sequence is operably linked to a promoter of a Zinc responsive gene. The protein product of such a polynucleotide is usually synthesized in the same cells, in response to the same stimuli as the protein product of the gene from which the promoter was derived. Such promoter are also useful to produce antisense mRNAs to down-regulate the product of proteins, or to produce sense mRNAs to down-regulate mRNAs via sense suppression.

### IV. UTILITIES OF PARTICULAR INTEREST

Genes capable of modulating the phenotypes in the following table are useful produce the associated utilities in the table. Such genes can be identified by their cDNA ID number in the Knock-in and Knock-out Tables. That is, those genes noted in those Tables to have a phenotype as listed in the following column entitled “Phenotype Modulated by a Gene” are useful for the purpose identified in the corresponding position in the column entitled “Utilities”.

| Phenotype | | |
|---|---|---|
| Modulated by a | | |
| Gene | | Utilities |
| Leaf shape | Cordate | decrease wind opacity, |
| | Cup-shaped | decrease lodging (plant fall over), |
| | Curled | increase biomass by making larger or |
| | | different shaped leaves, |
| | Laceolate | improve the efficiency of mechanical |
| | | harvesting, |
| | Lobed | decrease transpiration for better drought |
| | | tolerance, |
| | Oval | changing leaf shape to collect and absorb |
| | | water, |
| | Ovate | modulation of canopy structure and shading |
| | | for altered irradiance close to the ground, |
| | Serrate | enhanced uptake of pesticides (herbicides, |
| | | fungicides, etc), |
| | Trident | creation of ornamental leaf shapes, |
| | Undulate | increase resistance to pathogens by |
| | | decreasing amount of water that collects on |
| | | leaves, |
| | Vertically Oblong | change proporation of cell types in the |
| | | leaves for enhanced photosynthesis, |
| | | decreased transpiration, and enhanced |
| | Other Shapes | accumulation of desirable compounds |
| | | including secondary metabolites in |
| | | specialized cells, |
| | | decrease insect feeding, |
| | Long petioles | decrease wind opacity, |
| | Short petioles | decrease lodging (plant fall over), |
| | | increase biomass by better positioning of |
| | | the leaf blade, |
| | | decrease insect feeding, |
| | | decrease transpiration for better drought |
| | | tolerance, |
| | | position leaves most effectively for |
| | | photosynthetic efficiency |
| | Fused | ornamental applications to make distinctive |
| | | plants, |
| Reduced fertility | Short siliques | increase or decrease the number of seeds in |
| | | a fruit, |
| | | increasing fruit size, |
| | | modulating fruit shape to better fit |
| | | harvesting or packaging requirements, |
| | | useful for controlling dehisence and seed |
| | | scatter |
| | Reduced fertility | useful in hybrid breeding programs, |
| | Sterility | increasing fruit size, |
| | | production of seedless fruit, |
| | | useful as targets for gametocides, |
| | | modulating fruit shape to better fit |
| | | harvesting or packaging requirements, |
| | | useful for controlling dehisence and seed |
| | | scatter |
| | Flower size | useful for edible flowers |
| | | useful for flower derived products such as |
| | | fragrances |
| | | useful for modulating seed size and number |
| | | in combination with seed-specific genes |
| | | value in the ornamental industry |
| Stature | Large | increasing or decreasing plant biomass, |
| | Small | optimizing plant stature to increase yield |
| | | under various diverse environmental |
| | | conditions, e.g., when water or nutrients |
| | | are limiting, |
| | Dwarfs | decreasing lodging, |
| | | increasing fruit number and size, |
| | | controlling shading and canopy effects |
| Meristems | | Change plant architecture, |
| | | increase or decrease number of leaves as |
| | | well as change the types of leaves to |
| | | increase biomass, |
| | | improve photosynthetic efficiency, |
| | | create new varieties of ornamental plants |
| | | with enhanced leaf design, |
| | | preventing flowering to opimize vegetative |
| | | growth, |
| | | control of apical dominace, |
| | | increase or decrease flowering time to fit |
| | | season, water or fertilizer schedules, |
| | | change arrangement of leaves on the stem |
| | | (phyllotaxy) to optimize plant density, |
| | | decrease insect feeding, |
| | | or decrease pathogen infection, |
| | | increase number of trichome/glandular |
| | | trichome producing leaves |
| | | targets for herbicides, |
| | | generate ectopic meristems and ectopic |
| | | growth of vegetative and floral tissues and |
| | | seeds and fruits |
| Stem | Strong | modify lignin content/composition for |
| | | creation of harder woods or reduce |
| | | difficulty/costs in pulping for |
| | Weak | paper production or increase |
| | | digestibility of forage crops, |
| | | decrease lodging, |
| | | modify cell wall polysaccharides in stems |
| | | and fruits for improved texture and |
| | | nutrition. |
| | | increase biomass |
| | Late/Early Bolting | Break the need for long vernalization of |
| | | vernalization-dependent crops, e.g., winter |
| | | wheat, thereby increasing yield |
| | | decrease or increase generaton time |
| | | increase biomass |
| Lethals | Embryo-lethal | produce seedless fruit, |
| | | use as herbicide targets |
| | Embryo-defective | produce seedless fruit, |
| | | use as herbicide targets |
| | Seedling | use as herbicide targets, |
| | | useful for metabolic engineering, |
| | Pigment-lethals | use as herbicide targets, |
| | | increase photosynthetic efficiency |
| Pigment | Dark Green | Increase nutritional value, |
| | | enhanced photosynthesis and carbon |
| | | dioxide combustion and therefore increase |
| | | plant vigor and biomass, |
| | | enhanced photosynthetic efficiency and |
| | | therefore increase plant vigor and biomass, |
| | | prolong vegetative development, |
| | | enhanced protection against pathogens, |
| | YGV1 | Useful as targets for herbicides, |
| | | increase photosynthetic efficiency and |
| | | therefore increase plant vigor and biomass, |
| | YGV2 | Useful as targets for herbicides, |
| | | control of change from embryonic to adult |
| | | organs, |
| | | increase metabolic efficiency, |
| | | increase photosynthetic efficiency and |
| | | therefore increased plant vigor and biomass, |
| | YGV3 | Useful as targets for herbicides, |
| | | nitrogen sensing/uptake/usage, |
| | | increase metabolic efficiency and therefore |
| | | increased plant vigor and biomass, |
| | Interveinal chlorosis | to increase photosynthetic efficiency and |
| | | therefore increase plant vigor and biomass |
| | | to increase or decrease nitrogen transport |
| | | and therefore increase plant vigor and |
| | | biomass |
| | | use as herbicide targets |
| | | increase metabolic efficiency, |
| Roots | Short (primary root) | to access water from rainfall, |
| | | to access rhizobia spray application, for |
| | | anaerobic soils, |
| | | useful to facilitate harvest of root crops, |
| | Thick (primary root) | useful for increasing biomass of root crops, |
| | | for preventing plants dislodging during |
| | | picking and harvesting, |
| | | as root grafts, for animal feeds |
| | Branching (primary root) | modulation allows betters access to water, |
| | | minerals, fertilizers, rhizobia prevent soil |
| | | erosion, s |
| | | increasing root biomass |
| | | decrease root lodging, |
| | Long (lateral roots) | modulation allows improved access to |
| | | water, nutrients, fertilizer, rhizobia, prevent |
| | | soil erosion |
| | | increase root biomass |
| | | decrease root lodging |
| | | modulation allows control on the depth of |
| | | root growth in soil to access water and |
| | | nutriennts |
| | | modulation allows hormonal control of root |
| | | growth and development (size) |
| | Agravitropic | modulation allows control on the depth of |
| | | root growth in soil |
| | Curling (primary root) | modulation allows hormonal control of root |
| | | growth and development (size) |
| | | useful in anaerobic soils in allowing roots |
| | | to stay close to surface |
| | | harvesting of root crops |
| | Poor germination | |
| Trichome | Reduced Number | Genes useful for decreasing transpiration, |
| | Glabrous | increased production of glandular trichomes |
| | | for oil or other secreted chemicals of value, |
| | Increased Number | use as deterrent for insect herbivory and |
| | | ovipostion |
| | | modulation will increase resistance to UV |
| | | light, |
| Wax mutants | | decrease insect herbivory and oviposition, |
| | | compostion changes for the cosmetics |
| | | industry, |
| | | decrease transpiration, |
| | | provide pathogen resistance, |
| | | UV protection, |
| | | modulation of leaf runoff properties and |
| | | improved access for herbicides and |
| | | fertilizers |
| Cotyledons | | modulation of seeds structure in legumes, |
| | | increase nutritional value, |
| | | improve seedling competion under field |
| | | conditions, |
| Seeds | Transparent testa | genes useful for metabolic engineering |
| | Light | anthocyanin and flavonoid pathways |
| | Dark | mproved nutritional content |
| | | decrease petal abscission |
| Flowers | Other | decrease pod shattering |
| Hypocotysl | Long | to improve germination rates |
| | | to improve plant survivability |
| | Short | to improve germination rates |
| | | to improve plant survivability |

### V. ENHANCED FOODS

Animals require external supplies of amino acids that they cannot synthesize themselves. Also, some amino acids are required in larger quantities. The nutritional values of plants for animals and humans can thus be modified by regulating the amounts of the constituent amino acids that occur as free amino acids or in proteins. For instance, higher levels of lysine and/or methionine would enhance the nutritional value of corn seed. Applicants herein provide several methods for modulating the amino acid content:
(1) expressing a naturally occurring protein that has a high percentage of the desired amino acid(s);
(2) expressing a modified or synthetic coding sequence that has an enhanced percentage of the desired amino acids; or
(3) expressing the protein(s) that are capable of synthesizing more of the desired amino acids.
   A specific example is expressing proteins with enhanced, for example, methionine content, preferentially in a corn or cereal seed used for animal nutrition or in the parts of plants used for nutritional purposes.

A protein is considered to have a high percentage of an amino acid if the amount of the desired amino acid is at least 1% of the total number of residues in a protein; more preferably 2% or greater. Amino acids of particular interest are tryptophan, lysine, methionine, phenylalanine, threonine leucine, valine, and isoleucine. Examples of naturally occurring proteins with a high percentage of any one of the amino acid of particular interest are listed in the Enhanced Amino Acid Table.

The sequence(s) encoding the selected protein(s) are operably linked to a promoter and other regulatory sequences and transformed into a plant as described below. The promoter is chosen optimally for promoting the desired level of expression of the protein in the selected organ e.g. a promoter highly functional in seeds. Modifications may be made to the sequence encoding the protein to ensure protein transport into, for example, organelles or storage bodies or its accumulation in the organ. Such modifications may include addition of signal sequences at or near the N terminus and amino acid residues to modify protein stability or appropriate glycosylation. Other modifications may be made to the transcribed nucleic acid sequence to enhance the stability or translatability of the mRNA, in order to ensure accumulation of more of the desired protein. Suitable versions of the gene construct and transgenic plants are selected on the basis of, for example, the improved amino acid content and nutritional value measured by standard biochemical tests and animal feeding trials.

### VI. USE OF NOVEL GENES TO FACILITATE EXPLOITATION OF PLANTS AS FACTORIES FOR THE SYNTHESIS OF VALUABLE MOLECULES

Plants and their constituent cells, tissues, and organs are factories that manufacture small organic molecules such as sugars, amino acids, fatty acids, vitamins, etc., as well as macromolecules such as proteins, nucleic acids, oils/fats and carbohydrates. Plants have long been a source of pharmaceutically beneficial chemicals; particularly, the secondary metabolites and hormone-related molecules synthesized by plants. Plants can also be used as factories to produce carbohydrates or lipids that comprises a carbon backbone useful as precursors of plastics, fiber, fuel, paper, pulp, rubber, solvents, lubricants, construction materials, detergents, and other cleaning materials. Plants can also generate other compounds that are of economic value, such as dyes, flavors, and fragrances. Both the intermediates as well as the end-products of plant bio-synthetic pathways have been found useful.

With the polynucleotides and polypeptides of the instant invention, modification of both in-vitro and in-vivo synthesis of such products is possible. One method of increasing the amount of either the intermediates or the end-products synthesized in a cell is to increase the expression of one or more proteins in the synthesis pathway as discussed below. Another method of increasing production of an intermediate is to inhibit expression of protein(s) that synthesize the end-product from the intermediate. Levels of end-products and intermediates can also be modified by changing the levels of enzymes that specifically change or degrade them. The kinds of molecules made can be also be modified by changing the genes encoding specific enzymes performing reactions at specific steps of the biosynthetic pathway. These genes can be from the same or a different organism. The molecular structures in the biosynthetic pathways can thus be modified or diverted into different branches of a pathway to make novel end-products.

The modifications are made by designing one or more novel genes per application comprising promoters, to ensure production of the enzyme(s) in the relevant cells, in the right amount, and polynucleotides encoding the relevant enzyme. The promoters and polynucleotides are the subject of this application. The novel genes are transformed into the relevant species using standard procedures. Their effects are measured by standard assays for the specific chemical/biochemical products.

These polynucleotides and proteins of the invention that participate in the relevant pathways and are useful for changing production of the above chemicals and biochemicals are identified in the Reference tables by their enzyme function. More specifically, proteins of the invention that have the enzymatic activity of one of the entries in the following table entitled “Emzymes Effecting Modulation of Biological Pathways” are of interest to modulate the corresponding pathways to produce precursors or final products noted above that are of industrial use. Biological activities of particular interest are listed below.

Other polynucleotides and proteins that regulate where, when and to what extent a pathway is active in a plant are extremely useful for modulating the synthesis and accumulation of valuable chemicals. These elements including transcription factors, proteins involved in signal transduction and other proteins in the control of gene expression are described elsewhere in this application.

| Pathway | | |
|---|---|---|
| Name | Enzyme Description | Comments |
| Alkaloid | Morphine 6- | Also acts on other alkaloids, including codeine, |
| biosynthesis I | dehydrogenase | normorphine and ethylmorphine, but only very |
| | | slowly on 7,8-saturated derivatives such as |
| | | dihydromorphine and dihydrocodeine In the |
| | | reverse direction, also reduces naloxone to the |
| | | 6-alpha-hydroxy analog Activated by 2- |
| | | mercaptoethanol |
| | Codeinone reductase | Stereospecifically catalyses the reversible |
| | (NADPH) | reduction of codeinone to codeine, which is a |
| | | direct precursor of morphine in the opium |
| | | poppy plant, Papaver somniferum |
| | Salutaridine reductase | Stereospecifically catalyses the reversible |
| | (NADPH) | reduction of salutaridine to salutaridinol, which |
| | | is a direct precursor of morphinan alkaloids in |
| | | the poppy plant, Papaver somniferum |
| | (S)-stylopine synthase | Catalyses an oxidative reaction that does not |
| | | incorporate oxygen into the product Forms the |
| | | second methylenedioxy bridge of the |
| | | protoberberine alkaloid stylopine from |
| | | oxidative ring closure of adjacent phenolic and |
| | | methoxy groups of cheilanthifoline |
| | (S)-cheilanthifoline | Catalyses an oxidative reaction that does not |
| | synthase | incorporate oxygen into the product Forms the |
| | | methylenedioxy bridge of the protoberberine |
| | | alkaloid cheilanthifoline from oxidative ring |
| | | closure of adjacent phenolic and methoxy |
| | | groups of scoulerine |
| | Salutaridine synthase | Forms the morphinan alkaloid salutaridine by |
| | | intramolecular phenol oxidation of reticuline |
| | | without the incorporation of oxygen into the |
| | | product |
| | (S)-canadine synthase | Catalyses an oxidative reaction that does not |
| | | incorporate oxygen into the product Oxidation |
| | | of the methoxyphenol group of the alkaloid |
| | | tetrahydrocolumbamine results in the |
| | | formation of the methylenedioxy bridge of |
| | | canadine |
| | Protopine 6- | Involved in benzophenanthridine alkaloid |
| | monooxygenase | synthesis in higher plants |
| | Dihydrosanguinarine | Involved in benzophenanthridine alkaloid |
| | 10-monooxygenase | synthesis in higher plants |
| | Monophenol | A group of copper proteins that also catalyse |
| | monooxygenase | the reaction of BC 1.10.3.1, if only 1,2- |
| | | benzenediols are available as substrate |
| | L-amino acid oxidase | |
| | 1,2-dehydroreticulinium | Stereospecifically reduces the 1,2- |
| | reductase (NADPH) | dehydroreticulinium ion to (R)-reticuline, |
| | | which is a direct precursor of morphinan |
| | | alkaloids in the poppy plant, papaver |
| | | somniferum The enzyme does not catalyse the |
| | | reverse reaction to any significant extent under |
| | | physiological conditions |
| | Dihydrobenzophenanthridine | Also catalyzes: dihydrochelirubine + O(2) = chelirubine + H(2)O(2) |
| | oxidase | Also catalyzes: |
| | | dihydromacarpine + O(2) = macarpine + H(2)O(2) |
| | | Found in higher plants Produces |
| | | oxidized forms of the benzophenanthridine |
| | | alkaloids |
| | Reticuline oxidase | The product of the reaction, (S)-scoulerine, is a |
| | | precursor of protopine, protoberberine and |
| | | benzophenanthridine alkaloid biosynthesis in |
| | | plants Acts on (S)-reticuline and related |
| | | compounds, converting the N-methyl group |
| | | into the methylene bridge (‘berberine |
| | | bridge[PRIME]) of (S)- |
| | | tetrahydroprotoberberines |
| | 3[PRIME]-hydroxy-N- | Involved in isoquinoline alkaloid metabolism |
| | methyl-(S)-coclaurine | in plants Has also been shown to catalyse the |
| | 4[PRIME]-O- | methylation of (R,S)-laudanosoline, (S)- |
| | methyltransferase | 3[PRIME]-hydroxycoclaurine and (R,S)-7-O- |
| | | methylnoraudanosoline |
| | (S)-scoulerine 9-O- | The product of this reaction is a precursor for |
| | methyltransferase | protoberberine alkaloids in plants |
| | Columbamine O- | The product of this reaction is a protoberberine |
| | methyltransferase | alkaloid that is widely distributed in the plant |
| | | kingdom Distinct in specificity from EC |
| | | 2.1.1.88 |
| | 10-hydroxydihydro- | Part of the pathway for synthesis of |
| | sanguinarine 10-O- | benzophenanthridine alkaloids in plants |
| | methyltransferase | |
| | 12-hydroxydi- | Part of the pathway for synthesis of |
| | hydrochelirubine 12-O- | benzophenanthridine alkaloid macarpine in |
| | methyltransferase | plants |
| | (R,S)-norcoclaurine 6- | Norcoclaurine is 6,7-dihydroxy-1-[(4- |
| | O-methyltransferase | hydroxyphenyl)methyl]-1,2,3,4- |
| | | tetrahydroisoquinoline The enzyme will also |
| | | catalyse the 6-O-methylation of (R,S)- |
| | | norlaudanosoline to form 6-O-methyl- |
| | | norlaudanosoline, but this alkaloid has not |
| | | been found to occur in plants |
| | Salutaridinol 7-O- | At higher pH values the product, 7-O- |
| | acetyltransferase | acetylsalutaridinol, spontaneously closes the 4- |
| | | >5 oxide bridge by allylic elimination to form |
| | | the morphine precursor thebaine From the |
| | | opium poppy plant, Papaver somniferum |
| | Aspartate | Also acts on L-tyrosine, L-phenylalanine and |
| | aminotransferase | L-tryptophan. This activity can be formed from |
| | | EC 2.6.1.57 by controlled proteolysis |
| | Tyrosine | L-phenylalanine can act instead of L-tyrosine |
| | aminotransferase | The mitochondrial enzyme may be identical |
| | | with EC 2.6.1.1 The three isoenzymic forms |
| | | are interconverted by EC 3.4.22.4 |
| | Aromatic amino acid | L-methionine can also act as donor, more |
| | transferase | slowly Oxaloacetate can act as acceptor |
| | | Controlled proteolysis converts the enzyme to |
| | | EC 2.6.1.1 |
| | Tyrosine decarboxylase | The bacterial enzyme also acts on 3- |
| | | hydroxytyrosine and, more slowly, on 3- |
| | | hydroxyphenylalanine |
| | Aromatic-L-amino-acid | Also acts on L-tryptophan, 5-hydroxy-L- |
| | decarboxylase | tryptophan and dihydroxy-L-phenylalanine |
| | | (DOPA) |
| Alkaloid | Tropine dehydrogenase | Oxidizes other tropan-3-alpha-ols, but not the |
| biosynthesis | | corresponding beta-derivatives |
| II | | |
| | Tropinone reductase | |
| | Hyoscyamine (6S)- | |
| | dioxygenase | |
| | 6-beta- | |
| | hydroxyhyoscyamine | |
| | epoxidase | |
| | Amine oxidase (copper- | A group of enzymes including those oxidizing |
| | containing) | primary amines, diamines and histamine One |
| | | form of EC 1.3.1.15 from rat kidney also |
| | | catalyses this reaction |
| | Putrescine N- | |
| | methyltransferase | |
| | Ornithine | |
| | decarboxylase | |
| | Oxalyl-CoA | |
| | decarboxylase | |
| | Phenylalanine | May also act on L-tyrosine |
| | ammonia-lyase | |
| Androgen and | 3-beta-hydroxy- | Acts on 3-beta-hydroxyandrost-5-en-17-one to |
| estrogen | delta(5)-steroid | form androst-4-ene-3,17-dione and on 3-beta- |
| metabolism | dehydrogenase | hydroxypregn-5-en-20-one to form |
| | | progesterone |
| | 11-beta-hydroxysteroid | |
| | dehydrogenase | |
| | Estradiol 17-alpha- | |
| | dehydrogenase | |
| | 3-alpha-hydroxy-5- | |
| | beta-androstane-17-one | |
| | 3-alpha-dehydrogenase | |
| | 3-alpha (17-beta)- | Also acts on other 17-beta-hydroxysteroids, on |
| | hydroxysteroid | the 3-alpha-hydroxy group of pregnanes and |
| | dehydrogenase (NAD+) | bile acids, and on benzene dihydrodiol |
| | | Different from EC 1.1.1.50 or EC 1.1.1.213 |
| | 3-alpha-hydroxysteroid | Acts on other 3-alpha-hydroxysteroids and on |
| | dehydrogenase (B- | 9-, 11- and 15-hydroxyprostaglandin B- |
| | specific) | specific with respect to NAD(+) or NADP(+) |
| | | (cf. EC 1.1.1.213) |
| | 3(or 17)beta- | Also acts on other 3-beta- or 17-beta- |
| | hydroxysteroid | hydroxysteroids (cf EC 1.1.1.209) |
| | dehydrogenase | |
| | Estradiol 17 beta- | Also acts on (S)-20-hydroxypregn-4-en-3-one |
| | dehydrogenase | and related compounds, oxidizing the (S)-20- |
| | | group B-specific with respect to NAD(P)(+) |
| | Testosterone 17-beta- | |
| | dehydrogenase | |
| | Testosterone 17-beta- | Also oxidizes 3-hydroxyhexobarbital to 3- |
| | dehydrogenase | oxohexobarbital |
| | (NADP+) | |
| | Steroid 11-beta- | Also hydroxylates steroids at the 18-position, |
| | monooxygenase | and converts 18-hydroxycorticosterone into |
| | | aldosterone |
| | Estradiol 6-beta- | |
| | monooxygenase | |
| | Androst-4-ene-3,17- | Has a wide specificity A single enzyme from |
| | dione monooxygenase | Cylindrocarpon radicicola (EC 1.14.13.54) |
| | | catalyses both this reaction and that catalysed |
| | | by EC 1.14.99.4 |
| | 3-oxo-5-alpha-steroid | |
| | 4-dehydrogenase | |
| | 3-oxo-5-beta-steroid 4- | |
| | dehydrogenase | |
| | UDP- | Family of enzymes accepting a wide range of |
| | glucuronosyltransferase | substrates, including phenols, alcohols, amines |
| | | and fatty acids Some of the activities catalysed |
| | | were previously listed separately as EC |
| | | 2.4.1.42, EC 2.4.1.59, EC 2.4.1.61, EC |
| | | 2.4.1.76, EC 2.4.1.77, EC 2.4.1.84, EC |
| | | 2.4.1.107 and EC 2.4.1.108 A temporary |
| | | nomenclature for the various forms whose |
| | | delineation is in a state of flux |
| | Steroid sulfotransferase | Broad specificity resembling EC 2.8.2.2, but |
| | | also acts on estrone |
| | Alcohol | Primary and secondary alcohols, including |
| | sulfotransferase | aliphatic alcohols, ascorbate, chloramphenicol, |
| | | ephedrine and hydroxysteroids, but not |
| | | phenolic steroids, can act as acceptors (cf. EC |
| | | 2.8.2.15) |
| | Estrone sulfotransferase | |
| | Arylsulfatase | A group of enzymes with rather similar |
| | | specificities |
| | Steryl-sulfatase | Also acts on some related steryl sulfates |
| | 17-alpha- | |
| | hydroxyprogesterone | |
| | aldolase | |
| | Steroid delta-isomerase | |
| C21-Steroid | 3-beta-hydroxy- | Acts on 3-beta-hydroxyandrost-5-en-17-one to |
| hormone | delta(5)-steroid | form androst-4-ene-3,17-dione and on 3-beta- |
| metabolism | dehydrogenase | hydroxypregn-5-en-20-one to form |
| | | progesterone |
| | 11-beta-hydroxysteroid | |
| | dehydrogenase | |
| | 20-alpha- | A-specific with respect to NAD(P)(+) |
| | hydroxysteroid | |
| | dehydrogenase | |
| | 3-alpha-hydroxysteroid | Acts on other 3-alpha-hydroxysteroids and on |
| | dehydrogenase (B- | 9-, 11- and 15-hydroxyprostaglandin B- |
| | specific) | specific with respect to NAD(+) or NADP(+) |
| | | (cf. EC 1.1.1.213) |
| | 3-alpha(or 20-beta)- | The 3-alpha-hydroxyl group or 20-beta- |
| | hydroxysteroid | hydroxyl group of pregnane and androstane |
| | dehydrogenase | steroids can act as donors |
| | Steroid 11-beta- | Also hydroxylates steroids at the 18-position, |
| | monooxygenase | and converts 18-hydroxycorticosterone into |
| | | aldosterone |
| | Corticosterone 18- | |
| | monooxygenase | |
| | Cholesterol | The reaction proceeds in three stages, with |
| | monooxygenase (side- | hydroxylation at C-20 and C-22 preceding |
| | chain cleaving) | scission of the side-chain at C-20 |
| | Steroid 21- | |
| | monooxygenase | |
| | Progesterone 11-alpha- | |
| | monooxygenase | |
| | Steroid 17-alpha- | |
| | monooxygenase | |
| | Cholestenone 5-beta- | |
| | reductase | |
| | Cortisone beta- | |
| | reductase | |
| | Progesterone 5-alpha- | Testosterone and 20-alpha-hydroxy-4-pregnen- |
| | reductase | 3-one can act in place of progesterone |
| | 3-oxo-5-beta-steroid 4- | |
| | dehydrogenase | |
| | Steroid delta-isomerase | |
| Flavonoids, | Coniferyl-alcohol | Specific for coniferyl alcohol; does not act on |
| stilbene and | dehydrogenase | cinnamyl alcohol, 4-coumaryl alcohol or |
| lignin | | sinapyl alcohol |
| biosynthesis | | |
| | Cinnamyl-alcohol | Acts on coniferyl alcohol, sinapyl alcohol, 4- |
| | dehydrogenase | coumaryl alcohol and cinnamyl alcohol (cf. EC |
| | | 1.1.1.194) |
| | Dihydrokaempferol 4- | Also acts, in the reverse direction, on (+)- |
| | reductase | dihydroquercetin and (+)-dihydromyricetin |
| | | Each dihydroflavonol is reduced to the |
| | | corresponding cis-flavon-3,4-diol NAD(+) can |
| | | act instead of NADP(+), more slowly Involved |
| | | in the biosynthesis of anthocyanidins in plants |
| | Flavonone 4-reductase | Involved in the biosynthesis of 3- |
| | | deoxyanthocyanidins from flavonones such as |
| | | naringenin or eriodictyol |
| | Peroxidase | |
| | Caffeate 3,4- | |
| | dioxygenase | |
| | Naringenin 3- | |
| | dioxygenase | |
| | Trans-cinnamate 4- | Also acts on NADH, more slowly |
| | monooxygenase | |
| | Trans-cinnamate 2- | |
| | monooxygenase | |
| | Flavonoid 3[PRIME]- | Acts on a number of flavonoids, including |
| | monooxygenase | naringenin and dihydrokaempferol Does not |
| | | act on 4-coumarate or 4-coumaroyl-CoA |
| | Monophenol | A group of copper proteins that also catalyse |
| | monooxygenase | the reaction of EC 1.10.3.1, if only 1,2- |
| | | benzenediols are available as substrate |
| | Cinnamoyl-CoA | Also acts on a number of substituted |
| | reductase | cinnamoyl esters of coenzyme A |
| | Caffeoyl-CoA O- | |
| | methyltransferase | |
| | Luteolin O- | Also acts on luteolin-7-O-beta-D-glucoside |
| | methyltransferase | |
| | Caffeate O- | 3,4-dihydroxybenzaldehyde and catechol can |
| | methyltransferase | act as acceptor, more slowly |
| | Apigenin 4[PRIME]-O- | Converts apigenin into acacetin Naringenin |
| | methyltransferase | (5,7,4[PRIME]-trihydroxyflavonone) can also |
| | | act as acceptor, more slowly |
| | Quercetin 3-O- | Specific for quercetin. Related enzymes bring |
| | methyltransferase | about the 3-O-methylation of other flavonols, |
| | | such as galangin and kaempferol |
| | Isoflavone-7-O-beta- | The 6-position of the glucose residue of |
| | glucoside | formononetin can also act as acceptor Some |
| | 6[PRIME][PRIME]-O- | other 7-O-glucosides of isoflavones, flavones |
| | malonyltransferase | and flavonols can also act, more slowly |
| | Pinosylvin synthase | Not identical with EC 2.3.1.74 or EC 2.3.1.95 |
| | Naringenin-chalcone | In the presence of NADH and a reductase, |
| | synthase | 6[PRIME]-deoxychalcone is produced |
| | Trihydroxystilbene | Not identical with EC 2.3.1.74 or EC 2.3.1.146 |
| | synthase | |
| | Quinate O- | Caffeoyl-CoA and 4-coumaroyl-CoA can also |
| | hydroxycinnamoyltransferase | act as donors, more slowly Involved in the |
| | | biosynthesis of chlorogenic acid in sweet |
| | | potato and, with EC 2.3.1.98 in the formation |
| | | of caffeoyl-CoA in tomato |
| | Coniferyl-alcohol | Sinapyl alcohol can also act as acceptor |
| | glucosyltransferase | |
| | 2-coumarate O-beta- | Coumarinate (cis-2-hydroxycinnamate) does |
| | glucosyltransferase | not act as acceptor |
| | Scopoletin | |
| | glucosyltransferase | |
| | Flavonol-3-O-glucoside | Converts flavonol 3-O-glucosides to 3-O- |
| | L-rhamnosyltransferase | rutinosides Also acts, more slowly, on rutin, |
| | | quercetin 3-O-galactoside and flavonol O3- |
| | | rhamnosides |
| | Flavone 7-O-beta- | A number of flavones, flavonones and |
| | glucosyltransferase | flavonols can function as acceptors Different |
| | | from EC 2.4.1.91 |
| | Flavonol 3-O- | Acts on a variety of flavonols, including |
| | glucosyltransferase | quercetin and quercetin 7-O-glucoside |
| | | Different from EC 2.4.1.81 |
| | Flavone | 7-O-beta-D-glucosides of a number of |
| | apiosyltransferase | flavonoids and of 4-substituted phenols can act |
| | | as acceptors |
| | Coniferin beta- | Also hydrolyzes syringin, 4-cinnamyl alcohol |
| | glucosidase | beta-glucoside, and, more slowly, some other |
| | | aryl beta-glycosides A plant cell-wall enzyme |
| | | involved in the biosynthesis of lignin |
| | Beta-glucosidase | Wide specificity for beta-D-glucosides. Some |
| | | examples also hydrolyse one or more of the |
| | | following: beta-D-galactosides, alpha-L- |
| | | arabinosides, beta-D-xylosides, and beta-D- |
| | | fucosides |
| | Chalcone isomerase | |
| | 4-coumarate-CoA | |
| | ligase | |
| TABLE Continued | | |
| Pathway Name | Enzyme Description | Enzyme Comments |
| Ascorbate and aldarate | D-threo-aldose 1- | Acts on L-fucose, D-arabinose and L- |
| metabolism | dehydrogenase | xylose The animal enzyme was also |
| | | shown to act on L-arabinose, and the |
| | | enzyme from Pseudomonas caryophylli |
| | | on L-glucose |
| | L-threonate 3- | |
| | dehydrogenase | |
| | Glucuronate reductase | Also reduces D-galacturonate May be |
| | | identical with EC 1.1.1.2 |
| | Glucuronolactone | |
| | reductase | |
| | L-arabinose 1- | |
| | dehydrogenase | |
| | L-galactonolactone | Acts on the 1,4-lactones of L-galactonic, |
| | oxidase | D-altronic, L-fuconic, D-arabinic and D- |
| | | threonic acids Not identical with EC |
| | | 1.1.3.8 (cf. EC 1.3.2.3) |
| | L-gulonolactone | The product spontaneously isomerizes to |
| | oxidase | L-ascorbate |
| | L-ascorbate oxidase | |
| | L-ascorbate peroxidase | |
| | Ascorbate 2,3- | |
| | dioxygenase | |
| | 2,5-dioxovalerate | |
| | dehydrogenase | |
| | Aldehyde | Wide specificity, including oxidation of |
| | dehydrogenase (NAD+) | D-glucuronolactone to D-glucarate |
| | Galactonolactone | Cf. EC 1.1.3.24 |
| | dehydrogenase | |
| | Monodehydroascorbate | |
| | reductase (NADH) | |
| | Glutathione | |
| | dehydrogenase | |
| | (ascorbate) | |
| | L-arabinonolactonase | |
| | Gluconolactonase | Acts on a wide range of hexono-1,5- |
| | | lactones |
| | Uronolactonase | |
| | 1,4-lactonase | Specific for 1,4-lactones with 4-8 carbon |
| | | atoms Does not hydrolyse simple |
| | | aliphatic esters, acetylcholine, sugar |
| | | lactones or substituted aliphatic lactones, |
| | | e.g. 3-hydroxy-4-butyrolactone |
| | 2-dehydro-3- | |
| | deoxyglucarate aldolase | |
| | L-arabinonate | |
| | dehydratase | |
| | Glucarate dehydratase | |
| | 5-dehydro-4- | |
| | deoxyglucarate | |
| | dehydratase | |
| | Galactarate dehydratase | |
| | 2-dehydro-3-deoxy-L- | |
| | arabinonate | |
| | dehydratase | |
| Carbon fixation | Malate dehydrogenase | Also oxidizes some other 2- |
| | | hydroxydicarboxylic acids |
| | Malate dehydrogenase | Does not decarboxylates added |
| | (decarboxylating) | oxaloacetate |
| | Malate dehydrogenase | Also decarboxylates added oxaloacetate |
| | (oxaloacetate | |
| | decarboxylating) | |
| | (NADP+) | |
| | Malate dehydrogenase | Activated by light |
| | (NADP+) | |
| | Glyceraldehyde-3- | |
| | phosphate | |
| | dehydrogenase | |
| | (NADP+) | |
| | (phosphorylating) | |
| | Transketolase | Wide specificity for both reactants, e.g. |
| | | converts hydroxypyruvate and R—CHO |
| | | into CO(2) and R—CHOH—CO—CH(2)OH |
| | | Transketolase from Alcaligenes faecalis |
| | | shows high activity with D-erythrose as |
| | | acceptor |
| | Aspartate | Also acts on L-tyrosine, L-phenylalanine |
| | aminotransferase | and L-tryptophan. This activity can be |
| | | formed from EC 2.6.1.57 by controlled |
| | | proteolysis |
| | Alanine | 2-aminobutanoate acts slowly instead of |
| | aminotransferase | alanine |
| | Sedoheptulokinase | |
| | Phosphoribulokinase | |
| | Pyruvate kinase | UTP, GTP, CTP, ITP and dATP can also |
| | | act as donors Also phosphorylates |
| | | hydroxylamine and fluoride in the |
| | | presence of CO(2) |
| | Phosphoglycerate | |
| | kinase | |
| | Pyruvate, phosphate | |
| | dikinase | |
| | Fructose- | The animal enzyme also acts on |
| | bisphosphatase | sedoheptulose 1,7-bisphosphate |
| | Sedoheptulose- | |
| | bisphosphatase | |
| | Phosphoenolpyruvate | |
| | carboxylase | |
| | Ribulose-bisphosphate | Will utilize O(2) instead of CO(2), |
| | carboxylase | forming 3-phospho-D-glycerate and 2- |
| | | phosphoglycolate |
| | Phosphoenolpyruvate | |
| | carboxykinase (ATP) | |
| | Fructose-bisphosphate | Also acts on (3S,4R)-ketose 1-phosphates |
| | aldolase | The yeast and bacterial enzymes are zinc |
| | | proteins The enzymes increase electron- |
| | | attraction by the carbonyl group, some |
| | | (Class I) forming a protonated imine with |
| | | it, others (Class II), mainly of microbial |
| | | origin, polarizing it with a metal ion, e.g |
| | | zinc |
| | Phosphoketolase | |
| | Ribulose-phosphate 3- | Also converts D-erythrose 4-phosphate |
| | epimerase | into D-erythrulose 4-phosphate and D- |
| | | threose 4-phosphate |
| | Triosephosphate | |
| | isomerase | |
| | Ribose 5-phosphate | Also acts on D-ribose 5-diphosphate and |
| | epimerase | D-ribose 5-triphosphate |
| Phenylalanine | (R)-4- | Also acts, more slowly, on (R)-3- |
| metabolism | hydroxyphenyllactate | phenyllactate, (R)-3-(indole-3-yl)lactate |
| | dehydrogenase | and (R)-lactate |
| | Hydroxyphenyl- | Also acts on 3-(3,4- |
| | pyruvate reductase | dihydroxyphenyl)lactate Involved with |
| | | EC 2.3.1.140 in the biosynthesis of |
| | | rosmarinic acid |
| | Aryl-alcohol | A group of enzymes with broad |
| | dehydrogenase | specificity towards primary alcohols with |
| | | an aromatic or cyclohex-1-ene ring, but |
| | | with low or no activity towards short- |
| | | chain aliphatic alcohols |
| | Peroxidase | |
| | Catechol 1,2- | Involved in the metabolism of nitro- |
| | dioxygenase | aromatic compounds by a strain of |
| | | Pseudomonas putida |
| | 2,3-dihydroxybenzoate | |
| | 3,4-dioxygenase | |
| | 3-carboxyethylcatechol | |
| | 2,3-dioxygenase | |
| | Catechol 2,3- | The enzyme from Alcaligines sp. strain |
| | dioxygenase | O-1 has also been shown to catalyse the |
| | | reaction: 3-Sulfocatechol + O(2) + H(2)O = 2- |
| | | hydroxymuconate + bisulfite. It has |
| | | been referred to as 3-sulfocatechol 2,3- |
| | | dioxygenase. Further work will be |
| | | necessary to show whether or not this is a |
| | | distinct enzyme |
| | 4- | |
| | hydroxyphenylpyruvate | |
| | dioxygenase | |
| | Protocatechuate 3,4- | |
| | dioxygenase | |
| | Hydroxyquinol 1,2- | The product isomerizes to 2- |
| | dioxygenase | maleylacetate (cis-hex-2-enedioate) |
| | | Highly specific; catechol and pyrogallol |
| | | are acted on at less than 1% of the rate at |
| | | which benzene-1,2,4-triol is oxidized |
| | Protocatechuate 4,5- | |
| | dioxygenase | |
| | Phenylalanine 2- | Also catalyses a reaction similar to that |
| | monooxygenase | of EC 1.4.3.2, forming 3-phenylpyruvate, |
| | | NH(3) and H(2)O(2), but more slowly |
| | Anthranilate 1,2- | |
| | dioxygenase | |
| | (deaminating, | |
| | decarboxylating) | |
| | Benzoate 1,2- | A system, containing a reductase which |
| | dioxygenase | is an iron-sulfur flavoprotein (FAD), and |
| | | an iron-sulfur oxygenase |
| | Toluene dioxygenase | A system, containing a reductase which |
| | | is an iron-sulfur flavoprotein (FAD), an |
| | | iron-sulfur oxygenase, and a ferredoxin |
| | | Some other aromatic compounds, |
| | | including ethylbenzene, 4-xylene and |
| | | some halogenated toluenes, are converted |
| | | into the corresponding cis-dihydrodiols |
| | Naphthalene 1,2- | A system, containing a reductase which |
| | dioxygenase | is an iron-sulfur flavoprotein (FAD), an |
| | | iron-sulfur oxygenase, and ferredoxin |
| | Benzene 1,2- | A system, containing a reductase which |
| | dioxygenase | is an iron-sulfur flavoprotein, an iron- |
| | | sulfur oxygenase and ferredoxin |
| | Salicylate 1- | |
| | monooxygenase | |
| | Trans-cinnamate 4- | Also acts on NADH, more slowly |
| | monooxygenase | |
| | Benzoate 4- | |
| | monooxygenase | |
| | 4-hydroxybenzoate 3- | Most enzymes from Pseudomonas are |
| | monooxygenase | highly specific for NAD(P)H (cf EC |
| | | 1.14.13.33) |
| | 3-hydroxybenzoate 4- | Also acts on a number of analogs of 3- |
| | monooxygenase | hydroxybenzoate substituted in the 2, 4, 5 |
| | | and 6 positions |
| | 3-hydroxybenzoate 6- | Also acts on a number of analogs of 3- |
| | monooxygenase | hydroxybenzoate substituted in the 2, 4, 5 |
| | | and 6 positions NADPH can act instead |
| | | of NADH, more slowly |
| | 4-hydroxybenzoate 3- | The enzyme from Corynebacterium |
| | monooxygenase | cyclohexanicum is highly specific for 4- |
| | (NAD(P)H) | hydroxybenzoate, but uses NADH and |
| | | NADPH at approximately equal rates (cf. |
| | | EC 1.14.13.2). It is less specific for |
| | | NADPH than EC 1.14.13.2 |
| | Anthranilate 3- | The enzyme from Aspergillus niger is an |
| | monooxygenase | iron protein; that from the yeast |
| | (deaminating) | Trichosporon cutaneum is a flavoprotein |
| | | (FAD) |
| | Melilotate 3- | |
| | monooxygenase | |
| | Phenol 2- | Also active with resorcinol and O-cresol |
| | monooxygenase | |
| | Mandelate 4- | |
| | monooxygenase | |
| | 3-hydroxybenzoate 2- | |
| | monooxygenase | |
| | 4-cresol dehydrogenase | Phenazine methosulfate can act as |
| | (hydroxylating) | acceptor A quinone methide is probably |
| | | formed as intermediate The product is |
| | | oxidized further to 4-hydroxybenzoate |
| | Benzaldehyde | |
| | dehydrogenase (NAD+) | |
| | Aminomuconate- | Also acts on 2-hydroxymuconate |
| | semialdehyde | semialdehyde |
| | dehydrogenase | |
| | Phenylacetaldehyde | |
| | dehydrogenase | |
| | 4-carboxy-2- | Does not act on unsubstituted aliphatic or |
| | hydroxymuconate-6- | aromatic aldehydes or glucose NAD(+) |
| | semialdehyde | can replace NADP(+), but with lower |
| | dehydrogenase | affinity |
| | Aldehyde | |
| | dehydrogenase | |
| | (NAD(P)+) | |
| | Benzaldehyde | |
| | dehydrogenase | |
| | (NADP+) | |
| | Coumarate reductase | |
| | Cis-1,2- | |
| | dihydrobenzene-1,2- | |
| | diol dehydrogenase | |
| | Cis-1,2-dihydro-1,2- | Also acts, at half the rate, on cis- |
| | dihydroxynaphthalene | anthracene dihydrodiol and cis- |
| | dehydrogenase | phenanthrene dihydrodiol |
| | 2-enoate reductase | Acts, in the reverse direction, on a wide |
| | | range of alkyl and aryl alpha, beta- |
| | | unsaturated carboxylate ions 2-butenoate |
| | | was the best substrate tested |
| | Maleylacetate reductase | |
| | Phenylalanine | The enzyme from Bacillus badius and |
| | dehydrogenase | Sporosarcina ureae are highly specific for |
| | | L-phenylalanine, that from Bacillus |
| | | sphaericus also acts on L-tyrosine |
| | L-amino acid oxidase | |
| | Amine oxidase (flavin- | Acts on primary amines, and usually also |
| | containing) | on secondary and tertiary amines |
| | Amine oxidase (copper- | A group of enzymes including those |
| | containing) | oxidizing primary amines, diamines and |
| | | histamine One form of EC 1.3.1.15 from |
| | | rat kidney also catalyses this reaction |
| | D-amino-acid | Acts to some extent on all D-amino acids |
| | dehydrogenase | except D-aspartate and D-glutamate |
| | Aralkylamine | Phenazine methosulfate can act as |
| | dehydrogenase | acceptor Acts on aromatic amines and, |
| | | more slowly, on some long-chain |
| | | aliphatic amines, but not on methylamine |
| | | or ethylamine (cf EC 1.4.99.3) |
| | Glutamine N- | |
| | phenylacetyltransferase | |
| | Acetyl-CoA C- | |
| | acyltransferase | |
| | D-amino-acid N- | |
| | acetyltransferase | |
| | Phenylalanine N- | Also acts, more slowly, on L-histidine |
| | acetyltransferase | and L-alanine |
| | Glycine N- | Not identical with EC 2.3.1.13 or EC |
| | benzoyltransferase | 2.3.1.68 |
| | Aspartate | Also acts on L-tyrosine, L-phenylalanine |
| | aminotransferase | and L-tryptophan. This activity can be |
| | | formed from EC 2.6.1.57 by controlled |
| | | proteolysis |
| | D-alanine | Acts on the D-isomers of leucine, |
| | aminotransferase | aspartate, glutamate, aminobutyrate, |
| | | norvaline and asparagine |
| | Tyrosine | L-phenylalanine can act instead of L- |
| | aminotransferase | tyrosine The mitochondrial enzyme may |
| | | be identical with EC 2.6.1.1 The three |
| | | isoenzymic forms are interconverted by |
| | | EC 3.4.22.4 |
| | Aromatic amino acid | L-methionine can also act as donor, more |
| | transferase | slowly Oxaloacetate can act as acceptor |
| | | Controlled proteolysis converts the |
| | | enzyme to EC 2.6.1.1 |
| | Histidinol-phosphate | |
| | aminotransferase | |
| | 3-oxoadipate CoA- | |
| | transferase | |
| | 3-oxoadipate enol- | Acts on the product of EC 4.1.1.44 |
| | lactonase | |
| | Carboxymethylene- | |
| | butenolidase | |
| | 2-pyrone-4,6- | The product isomerizes to 4- |
| | dicarboxylate lactonase | oxalmesaconate |
| | Hippurate hydrolase | Acts on various N-benzoylamino acids |
| | Amidase | |
| | Acylphosphatase | |
| | 2-hydroxymuconate- | |
| | semialdehyde hydrolase | |
| | Aromatic-L-amino-acid | Also acts on L-tryptophan, 5-hydroxy-L- |
| | decarboxylase | tryptophan and dihydroxy-L- |
| | | phenylalanine (DOPA) |
| | Phenylpyruvate | Also acts on indole-3-pyruvate |
| | decarboxylase | |
| | 4-carboxymucono- | |
| | lactone decarboxylase | |
| | O-pyrocatechuate | |
| | decarboxylase | |
| | Phenylalanine | Also acts on tyrosine and other aromatic |
| | decarboxylase | amino acids |
| | 4-hydroxybenzoate | |
| | decarboxylase | |
| | Protocatechuate | |
| | decarboxylase | |
| | Benzoylformate | |
| | decarboxylase | |
| | 4-oxalocrotonate | Involved in the meta-cleavage pathway |
| | decarboxylase | for the degradation of phenols, cresols |
| | | and catechols |
| | 4-hydroxy-4-methyl-2- | Also acts on 4-hydroxy-4-methyl-2- |
| | oxoglutarate aldolase | oxoadipate and 4-carboxy-4-hydroxy-2- |
| | | oxohexadioate |
| | 2-oxopent-4-enoate | Also acts, more slowly, on cis-2-oxohex- |
| | hydratase | 4-enoate, but not on the trans-isomer |
| | Phenylalanine | May also act on L-tyrosine |
| | ammonia-lyase | |
| | Phenylalanine racemase | |
| | (ATP-hydrolysing) | |
| | Mandelate racemase | |
| | Phenylpyruvate | Also acts on other arylpyruvates |
| | tautomerase | |
| | 5-carboxymethyl-2- | |
| | hydroxymuconate | |
| | delta-isomerase | |
| | Muconolactone delta- | |
| | isomerase | |
| | Muconate | Also acts, in the reverse reaction, on 3- |
| | cycloisomerase | methyl-cis-cis-hexa-dienedioate and, |
| | | very slowly, on cis-trans-hexadienedioate |
| | | Not identical with EC 5.5.1.7 or EC |
| | | 5.5.1.11 |
| | 3-carboxy-cis,cis- | |
| | muconate | |
| | cycloisomerase | |
| | Carboxy-cis,cis- | |
| | muconate cyclase | |
| | Chloromuconate | Spontaneous elimination of HCl produces |
| | cycloisomerase | cis-4-carboxymethylenebut-2-en-4-olide |
| | | Also acts in reverse direction on 2- |
| | | chloro-cis,cis-muconate Not identical |
| | | with EC 5.5.1.1 or EC 5.5.1.11 |
| | Phenylacetate-CoA | Phenoxyacetate can replace phenylacetate |
| | ligase | |
| | Benzoate-CoA ligase | Also acts on 2-, 3- and 4-fluorobenzoate, |
| | | but only very slowly on the |
| | | corresponding chlorobenzoates |
| | 4-hydroxybenzoate-CoA | |
| | ligase | |
| | Phenylacetate-CoA | Also acts, more slowly, on acetate, |
| | ligase | propanoate and butanoate, but not on |
| | | hydroxy derivatives of phenylacetate and |
| | | related compounds |
| Phenylalanine, tyrosine | Quinate 5- | |
| and tryptophan biosynthesis | dehydrogenase | |
| | Shikimate 5- | |
| | dehydrogenase | |
| | Quinate dehydrogenase | |
| | (pyrroloquinoline- | |
| | quinone) | |
| | Phenylalanine 4- | |
| | monooxygenase | |
| | Prephenate | This enzyme in the enteric bacteria also |
| | dehydrogenase | possesses chorismate mutase activity (EC |
| | | 5.4.99.5) and converts chorismate into |
| | | prephenate |
| | Prephenate | |
| | dehydrogenase | |
| | (NADP+) | |
| | Cyclohexadienyl | Also acts on prephenate and D- |
| | dehydrogenase | prephenyllactate (cf. EC 1.3.1.12) |
| | 2-methyl-branched- | From Ascaris suum The reaction |
| | chain-enoyl-CoA | proceeds only in the presence of another |
| | reductase | flavoprotein (ETF = [PRIME]Electron- |
| | | Transferring Flavoprotein[PRIME]) |
| | Phenylalanine | The enzyme from Bacillus badius and |
| | dehydrogenase | Sporosarcina ureae are highly specific for |
| | | L-phenylalanine, that from Bacillus |
| | | sphaericus also acts on L-tyrosine |
| | L-amino acid oxidase | |
| | Anthranilate | In some organisms, this enzyme is part of |
| | phosphoribosyl- | a multifunctional protein together with |
| | transferase | one or more components of the system |
| | | for biosynthesis of tryptophan (EC |
| | | 4.1.1.48, EC 4.1.3.27, EC 4.2.1.20, and |
| | | EC 5.3.1.24) |
| | 3-phosphoshikimate 1- | |
| | carboxyvinyl- | |
| | transferase | |
| | Aspartate | Also acts on L-tyrosine, L-phenylalanine |
| | aminotransferase | and L-tryptophan. This activity can be |
| | | formed from EC 2.6.1.57 by controlled |
| | | proteolysis |
| | Tyrosine | L-phenylalanine can act instead of L- |
| | aminotransferase | tyrosine The mitochondrial enzyme may |
| | | be identical with EC 2.6.1.1 The three |
| | | isoenzymic forms are interconverted by |
| | | EC 3.4.22.4 |
| | Aromatic amino acid | L-methionine can also act as donor, more |
| | transferase | slowly Oxaloacetate can act as acceptor |
| | | Controlled proteolysis converts the |
| | | enzyme to EC 2.6.1.1 |
| | Histidinol-phosphate | |
| | aminotransferase | |
| | Shikimate kinase | |
| | Indole-3-glycerol- | In some organisms, this enzyme is part of |
| | phosphate synthase | a multifunctional protein together with |
| | | one or more components of the system |
| | | for biosynthesis of tryptophan (EC |
| | | 2.4.2.18, EC 4.1.3.27, EC 4.2.1.20, and |
| | | EC 5.3.1.24) |
| | 2-dehydro-3- | |
| | deoxyphosphoheptonate | |
| | aldolase | |
| | Anthranilate synthase | In some organisms, this enzyme is part of |
| | | a multifunctional protein together with |
| | | one or more components of the system |
| | | for biosynthesis of tryptophan (EC |
| | | 2.4.2.18, EC 4.1.1.48, EC 4.2.1.20, and |
| | | EC 5.3.1.24) The native enzyme in the |
| | | complex with uses either glutamine or |
| | | (less efficiently) NH(3). The enzyme |
| | | separated from the complex uses NH(3) |
| | | only |
| | 3-dehydroquinate | |
| | dehydratase | |
| | Phosphopyruvate | Also acts on 3-phospho-D-erythronate |
| | hydratase | |
| | Tryptophan synthase | Also catalyses the conversion of serine |
| | | and indole into tryptophan and water and |
| | | of indoleglycerol phosphate into indole |
| | | and glyceraldehyde phosphate In some |
| | | organisms, this enzyme is part of a |
| | | multifunctional protein together with one |
| | | or more components of the system for |
| | | biosynthesis of tryptophan (EC 2.4.2.18, |
| | | EC 4.1.1.48, EC 4.1.3.27, and EC |
| | | 5.3.1.24) |
| | Prephenate dehydratase | This enzyme in the enteric bacteria also |
| | | possesses chorismate mutase activity and |
| | | converts chorismate into prephenate |
| | Carboxycyclohexadienyl | Also acts on prephenate and D- |
| | dehydratase | prephenyllactate Cf. EC 4.2.1.51 |
| | 3-dehydroquinate | The hydrogen atoms on C-7 of the |
| | synthase | substrate are retained on C-2 of the |
| | | products |
| | Chorismate synthase | Shikimate is numbered so that the |
| | | double-bond is between C-1 and C-2, but |
| | | some earlier papers numbered in the |
| | | reverse direction |
| | Phosphoribosylanthranilate | In some organisms, this enzyme is part of |
| | isomerase | a multifunctional protein together with |
| | | one or more components of the system |
| | | for biosynthesis of tryptophan (EC |
| | | 2.4.2.18, EC 4.1.1.48, EC 4.1.3.27, and |
| | | EC 4.2.1.20) |
| | Chorismate mutase | |
| | Tyrosine-tRNA ligase | |
| | Phenylalanine-tRNA | |
| | ligase | |
| Starch and sucrose | UDP-glucose 6- | Also acts on UDP-2-deoxyglucose |
| metabolism | dehydrogenase | |
| | Glucoside 3- | The enzyme acts on D-glucose, D- |
| | dehydrogenase | galactose, D-glucosides and D- |
| | | galactosides, but D-glucosides react more |
| | | rapidly than D-galactosides |
| | CDP-4-dehydro-6- | Two proteins are involved but no partial |
| | deoxyglucose reductase | reaction has been observed in the |
| | | presence of either alone |
| | Phosphorylase | The recommended name should be |
| | | qualified in each instance by adding the |
| | | name of the natural substance, e.g. |
| | | maltodextrin phosphorylase, starch |
| | | phosphorylase, glycogen phosphorylase |
| | Levansucrase | Some other sugars can act as D-fructosyl |
| | | acceptors |
| | Glycogen (starch) | The recommended name varies according |
| | synthase | to the source of the enzyme and the |
| | | nature of its synthetic product Glycogen |
| | | synthase from animal tissues is a |
| | | complex of a catalytic subunit and the |
| | | protein glycogenin The enzyme requires |
| | | glucosylated glycogenin as a primer; this |
| | | is the reaction product of EC 2.4.1.186 A |
| | | similar enzyme utilizes ADP-glucose (Cf. |
| | | EC 2.4.1.21) |
| | Cellulose synthase | Involved in the synthesis of cellulose A |
| | (UDP-forming) | similar enzyme utilizes GDP-glucose (Cf. |
| | | EC 2.4.1.29) |
| | Sucrose synthase | |
| | Sucrose-phosphate | |
| | synthase | |
| | Alpha,alpha-trehalose- | See also EC 2.4.1.36 |
| | phosphate synthase | |
| | (UDP-forming) | |
| | UDP- | Family of enzymes accepting a wide |
| | glucuronosyltransferase | range of substrates, including phenols, |
| | | alcohols, amines and fatty acids Some of |
| | | the activities catalysed were previously |
| | | listed separately as EC 2.4.1.42, EC |
| | | 2.4.1.59, EC 2.4.1.61, EC 2.4.1.76, EC |
| | | 2.4.1.77, EC 2.4.1.84, EC 2.4.1.107 and |
| | | EC 2.4.1.108 A temporary nomenclature |
| | | for the various forms whose delineation |
| | | is in a state of flux |
| | 1,4-alpha-glucan | Converts amylose into amylopectin The |
| | branching enzyme | recommended name requires a |
| | | qualification depending on the product, |
| | | glycogen or amylopectin, e.g. glycogen |
| | | branching enzyme, amylopectin |
| | | branching enzyme. The latter has |
| | | frequently been termed Q-enzyme |
| | Cellobiose | |
| | phosphorylase | |
| | Starch (bacterial | The recommended name various |
| | glycogen) synthase | according to the source of the enzyme |
| | | and the nature of its synthetic product, |
| | | e.g. starch synthase, bacterial glycogen |
| | | synthase A similar enzyme utilizes UDP- |
| | | glucose (Cf. EC 2.4.1.11) |
| | 4-alpha- | An enzymic activity of this nature forms |
| | glucanotransferase | part of the mammalian and Yeast |
| | | glycogen branching system (see EC |
| | | 3.2.1.33) |
| | Cellulose synthase | Involved in the synthesis of cellulose A |
| | (GDP-forming) | similar enzyme utilizes UDP-glucose (Cf. |
| | | EC 2.4.1.12) |
| | 1,3-beta-glucan | |
| | synthase | |
| | Phenol beta- | Acts on a wide range of phenols |
| | glucosyltransferase | |
| | Amylosucrase | |
| | Polygalacturonate 4- | |
| | alpha- | |
| | galacturonosyltransferase | |
| | Dextransucrase | |
| | Alpha,alpha-trehalose | |
| | phosphorylase | |
| | Sucrose phosphorylase | In the forward reaction, arsenate may |
| | | replace phosphate In the reverse reaction |
| | | various ketoses and L-arabinose may |
| | | replace D-fructose |
| | Maltose phosphorylase | |
| | 1,4-beta-D-xylan | |
| | synthase | |
| | Hexokinase | D-glucose, D-mannose, D-fructose, |
| | | sorbitol and D-glucosamine can act as |
| | | acceptors ITP and dATP can act as |
| | | donors The liver isoenzyme has |
| | | sometimes been called glucokinase |
| | Phosphoglucokinase | |
| | Glucose-1,6- | D-glucose 6-phosphate can act as |
| | bisphosphate synthase | acceptor, forming D-glucose 1,6- |
| | | bisphosphate |
| | Glucokinase | A group of enzymes found in |
| | | invertebrates and microorganisms highly |
| | | specific for glucose |
| | Fructokinase | |
| | Glucose-1-phosphate | |
| | phosphodismutase | |
| | Protein-N(PI)- | Comprises a group of related enzymes |
| | phosphohistidine-sugar | The protein substrate is a phosphocarrier |
| | phosphotransferase | protein of low molecular mass (9.5 Kd) A |
| | | phosphoenzyme intermediate is formed |
| | | The enzyme translocates the sugar it |
| | | phosphorylates into bacteria Aldohexoses |
| | | and their glycosides and alditols are |
| | | phosphorylated on O-6; fructose and |
| | | sorbose on O-1 Glycerol and |
| | | disaccharides are also substrates |
| | Glucose-1-phosphate | |
| | adenylyltransferase | |
| | Glucose-1-phosphate | |
| | cytidylyltransferase | |
| | Glucose-1-phosphate | Also acts, more slowly, on D-mannose 1- |
| | guanylyltransferase | phosphate |
| | UTP-glucose-1- | |
| | phosphate | |
| | uridylyltransferase | |
| | Pectinesterase | |
| | Trehalose-phosphatase | |
| | Sucrose-phosphatase | |
| | Glucose-6-phosphatase | Wide distribution in animal tissues Also |
| | | catalyses potent transphosphorylations |
| | | from carbamoyl phosphate, hexose |
| | | phosphates, pyrophosphate, |
| | | phosphoenolpyruvate and nucleoside di- |
| | | and triphosphates, to D-glucose, D- |
| | | mannose, 3-methyl-D-glucose, or 2- |
| | | deoxy-D-glucose (cf. EC 2.7.1.62, EC |
| | | 2.7.1.79, and EC 3.9.1.1) |
| | Alpha-amylase | Acts on starch, glycogen and related |
| | | polysaccharides and oligosaccharides in a |
| | | random manner; reducing groups are |
| | | liberated in the alpha-configuration |
| | Oligo-1,6-glucosidase | Also hydrolyses palatinose The enzyme |
| | | from intestinal mucosa is a single |
| | | polypeptide chain also catalysing the |
| | | reaction of EC 3.2.1.48 |
| | Maltose-6[PRIME]- | Hydrolyses a variety of 6-phospho-D- |
| | phosphate glucosidase | glucosides, including maltose 6- |
| | | phosphate, alpha[PRIME]alpha-trehalose |
| | | 6-phosphate, sucrose 6-phosphate and p- |
| | | nitrophenyl-alpha-D-glucopyranoside 6- |
| | | phosphate (as a chromogenic substrate) |
| | | The enzyme is activated by Fe(II), |
| | | Mn(II), Co(II) and Ni(II). It is rapidly |
| | | inactivated in air |
| | Polygalacturonase | |
| | Beta-amylase | Acts on starch, glycogen and related |
| | | polysaccharides and oligosaccharides |
| | | producing beta-maltose by an inversion |
| | Alpha-glucosidase | Group of enzymes whose specificity is |
| | | directed mainly towards the |
| | | exohydrolysis of 1,4-alpha-glucosidic |
| | | linkages, and that hydrolyse |
| | | oligosaccharides rapidly, relative to |
| | | polysaccharides, which are hydrolysed |
| | | relatively slowly, or not at all The |
| | | intestinal enzyme also hydrolyses |
| | | polysaccharides, catalysing the reactions |
| | | of EC 3.2.1.3, and, more slowly, |
| | | hydrolyses 1,6-alpha-D-glucose links |
| | Beta-glucosidase | Wide specificity for beta-D-glucosides. |
| | | Some examples also hydrolyse one or |
| | | more of the following: beta-D- |
| | | galactosides, alpha-L-arabinosides, beta- |
| | | D-xylosides, and beta-D-fucosides |
| | Beta-fructofuranosidase | Substrates include sucrose Also catalyses |
| | | fructotransferase reactions |
| | Alpha,alpha-trehalase | |
| | Glucan 1,4-alpha- | Most forms of the enzyme can rapidly |
| | glucosidase | hydrolyse 1,6-alpha-D-glucosidic bonds |
| | | when the next bond in sequence is 1,4, |
| | | and some preparations of this enzyme |
| | | hydrolyse 1,6- and 1,3-alpha-D- |
| | | glucosidic bonds in other polysaccharides |
| | | This entry covers all such enzymes acting |
| | | on polysaccharides more rapidly than on |
| | | oligosaccharides EC 3.2.1.20 from |
| | | mammalian intestine can catalyse similar |
| | | reactions |
| | Beta-glucuronidase | |
| | Amylo-1,6-glucosidase | In mammals and yeast this enzyme is |
| | | linked to a glycosyltransferase similar to |
| | | EC 2.4.1.25; together these two activities |
| | | constitute the glycogen debranching |
| | | system |
| | Xylan 1,4-beta- | Also hydrolyses xylobiose Some other |
| | xylosidase | exoglycosidase activities have been |
| | | found associated with this enzyme in |
| | | sheep liver |
| | Glucan endo-1,3-beta- | Very limited action on mixed-link (1,3- |
| | D-glucosidase | 1,4-)-beta-D-glucans Hydrolyses |
| | | laminarin, paramylon and pachyman |
| | | Different from EC 3.2.1.6 |
| | Cellulase | Will also hydrolyse 1,4-linkages in beta- |
| | | D-glucans also containing 1,3-linkages |
| | Sucrose alpha- | This enzyme is isolated from intestinal |
| | glucosidase | mucosa as a single polypeptide chain also |
| | | displaying activity towards isomaltose |
| | | (oligo-1,6-glucosidase, cf. EC 3.2.1.10) |
| | Cyclomaltodextrinase | Also hydrolyses linear maltodextrin |
| | Glucan 1,3-beta- | Acts on oligosaccharides but very slowly |
| | glucosidase | on laminaribiose |
| | Levanase | |
| | Galacturan 1,4-alpha- | |
| | galacturonidase | |
| | Glucan 1,4-beta- | Acts on 1,4-beta-D-glucans and related |
| | glucosidase | oligosaccharides Cellobiose is |
| | | hydrolysed, very slowly |
| | Cellulose 1,4-beta- | |
| | cellobiosidase | |
| | Alpha,alpha- | |
| | phosphotrehalase | |
| | ADP-sugar | Has a distinct specificity from the UDP- |
| | diphosphatase | sugar pyrophosphatase (EC 3.6.1.45) |
| | Nucleotide | Substrates include NAD(+), NADP(+), |
| | pyrophosphatase | FAD, CoA and also ATP and ADP |
| | UDP-glucuronate | |
| | decarboxylase | |
| | CDP-glucose 4,6- | |
| | dehydratase | |
| | CDP-abequose | |
| | epimerase | |
| | UDP-glucuronate 4- | |
| | epimerase | |
| | Glucose-6-phosphate | Also catalyses the anomerization of D- |
| | isomerase | glucose 6-phosphate |
| | Phosphoglucomutase | Maximum activity is only obtained in the |
| | | presence of alpha-D-glucose 1,6- |
| | | bisphosphate. This bisphosphate is an |
| | | intermediate in the reaction, being |
| | | formed by transfer of a phosphate residue |
| | | from the enzyme to the substrate, but the |
| | | dissociation of bisphosphate from the |
| | | enzyme complex is much slower than the |
| | | overall isomerization Also, more slowly, |
| | | catalyses the interconversion of 1- |
| | | phosphate and 6-phosphate isomers of |
| | | many other alpha-D-hexoses, and the |
| | | interconversion of alpha-D-ribose 1- |
| | | phosphate and 5-phosphate |
| | Beta- | |
| | phosphoglucomutase | |
| | Maltose alpha-D- | |
| | glucosyltransferase | |
| Tryptophan metabolism | Indole-3-lactate | |
| | dehydrogenase | |
| | Indole-3-acetaldehyde | |
| | reductase (NADH) | |
| | Indole-3-acetaldehyde | |
| | reductase (NADPH) | |
| | 3-hydroxyacyl-CoA | Also oxidizes S-3-hydroxyacyl-N- |
| | dehydrogenase | acylthioethanolamine and S-3- |
| | | hydroxyacylhydrolipoate Some enzymes |
| | | act, more slowly, with NADP(+) Broad |
| | | specificity to acyl chain-length (cf. EC |
| | | 1.1.1.211) |
| | O-aminophenol oxidase | Isophenoxazine may be formed by a |
| | | secondary condensation from the initial |
| | | oxidation product |
| | Catalase | This enzyme can also act as a peroxidase |
| | | (EC 1.11.1.7) for which several organic |
| | | substances, especially ethanol, can act as |
| | | a hydrogen donor A manganese protein |
| | | containing Mn(III) in the resting state, |
| | | which also belongs here, is often called |
| | | pseudocatalase Enzymes from some |
| | | microorganisms, such as Penicillium |
| | | simplicissimum, which exhibit both |
| | | catalase and peroxidase activity, have |
| | | sometimes been referred to as catalase- |
| | | peroxidase |
| | 7,8- | |
| | dihydroxykynurenate | |
| | 8,8A-dioxygenase | |
| | Tryptophan 2,3- | Broad specificity towards tryptamine and |
| | dioxygenase | derivatives including D- and L- |
| | | tryptophan, 5-hydroxytryptophan and |
| | | serotonin |
| | Indole 2,3-dioxygenase | The enzyme from jasminum is a |
| | | flavoprotein containing copper, and |
| | | forms anthranilate as the final product |
| | | One enzyme from Tecoma stans is also a |
| | | flavoprotein containing copper and uses |
| | | three atoms of oxygen per molecule of |
| | | indole, to form anthranil (3,4- |
| | | benzisoxazole) A second enzyme from |
| | | Tecoma stans, which is not a |
| | | flavoprotein, uses four atoms of oxygen |
| | | and forms anthranilate as the final |
| | | product |
| | 2,3-dihydroxyindole | |
| | 2,3-dioxygenase | |
| | Indoleamine-pyrrole | Acts on many substituted and |
| | 2,3-dioxygenase | unsubstituted indoleamines, including |
| | | melatonin Involved in the degradation of |
| | | melatonin |
| | 3-hydroxyanthranilate | The product of the reaction |
| | 3,4-dioxygenase | spontaneously rearrange to quinolinic |
| | | acid (quin) |
| | Tryptophan 2- | |
| | monooxygenase | |
| | Tryptophan 2[PRIME]- | Acts on a number of indolyl-3-alkane |
| | dioxygenase | derivatives, oxidizing the 3-side-chain in |
| | | the 2[PRIME]-position. Best substrates |
| | | are L-tryptophan and 5-hydroxy-L- |
| | | tryptophan |
| | Kynurenine 3- | |
| | monooxygenase | |
| | Unspecific | Acts on a wide range of substrates |
| | monooxygenase | including many xenobiotics, steroids, |
| | | fatty acids, vitamins and prostaglandins |
| | | Reactions catalysed include |
| | | hydroxylation, epoxidation, N-oxidation, |
| | | sulfooxidation, N-, S- and O- |
| | | dealkylations, desulfation, deamination, |
| | | and reduction of azo, nitro, and N-oxide |
| | | groups |
| | Anthranilate 3- | |
| | monooxygenase | |
| | Tryptophan 5- | Activated by phosphorylation, catalysed |
| | monooxygenase | by a CA(2+)-activated protein kinase |
| | Kynurenine 7,8- | |
| | hydroxylase | |
| | Aldehyde | Wide specificity, including oxidation of |
| | dehydrogenase (NAD+) | D-glucuronolactone to D-glucarate |
| | Aminomuconate- | Also acts on 2-hydroxymuconate |
| | semialdehyde | semialdehyde |
| | dehydrogenase | |
| | Aldehyde oxidase | Also oxidizes quinoline and pyridine |
| | | derivatives May be identical with EC |
| | | 1.1.3.22 |
| | Indole-3-acetaldehyde | Also oxidizes indole-3-aldehyde and |
| | oxidase | acetaldehyde, more slowly |
| | Oxoglutarate | Component of the multienzyme 2- |
| | dehydrogenase | oxoglutarate dehydrogenase complex |
| | (lipoamide) | |
| | Kynurenate-7,8- | |
| | dihydrodiol | |
| | dehydrogenase | |
| | Glutaryl-CoA | |
| | dehydrogenase | |
| | L-amino acid oxidase | |
| | Amine oxidase (flavin- | Acts on primary amines, and usually also |
| | containing) | on secondary and tertiary amines |
| | Amine oxidase (copper- | A group of enzymes including those |
| | containing) | oxidizing primary amines, diamines and |
| | | histamine One form of EC 1.3.1.15 from |
| | | rat kidney also catalyses this reaction |
| | Acetylindoxyl oxidase | |
| | Acetylserotonin O- | Some other hydroxyindoles also act as |
| | methyltransferase | acceptor, more slowly |
| | Indole-3-pyruvate C- | |
| | methyltransferase | |
| | Amine N- | A wide range of primary, secondary, and |
| | methyltransferase | tertiary amines can act as acceptors, |
| | | including tryptamine, aniline, nicotine |
| | | and a variety of drugs and other |
| | | xenobiotics |
| | Aralkylamine N- | Narrow specificity towards |
| | acetyltransferase | aralkylamines, including serotonin Not |
| | | identical with EC 2.3.1.5 |
| | Acetyl-CoA C- | |
| | acetyltransferase | |
| | Tryptophan | Also acts on 5-hydroxytryptophan and, to |
| | aminotransferase | a lesser extent on the phenyl amino acids |
| | Kynurenine- | Also acts on 3-hydroxykynurenine |
| | oxoglutarate | |
| | aminotransferase | |
| | Thioglucosidase | Has a wide specificity for thioglycosides |
| | Amidase | |
| | Formamidase | Also acts, more slowly, on acetamide, |
| | | propanamide and butanamide |
| | Arylformamidase | Also acts on other aromatic |
| | | formylamines |
| | Nitrilase | Acts on a wide range of aromatic nitriles |
| | | including (indole-3-yl)-acetonitrile and |
| | | also on some aliphatic nitriles, and on the |
| | | corresponding acid amides (cf. EC |
| | | 4.2.1.84) |
| | Kynureninase | Also acts on 3[PRIME]- |
| | | hydroxykynurenine and some other (3- |
| | | arylcarbonyl)-alanines |
| | Aromatic-L-amino-acid | Also acts on L-tryptophan, 5-hydroxy-L- |
| | decarboxylase | tryptophan and dihydroxy-L- |
| | | phenylalanine (DOPA) |
| | Phenylpyruvate | Also acts on indole-3-pyruvate |
| | decarboxylase | |
| | Aminocarboxymuconate- | The product rearranges non-enzymically |
| | semialdehyde | to picolinate |
| | decarboxylase | |
| | Tryptophanase | Also catalyses the synthesis of |
| | | tryptophan from indole and serine Also |
| | | catalyses 2,3-elimination and beta- |
| | | replacement reactions of some indole- |
| | | substituted tryptophan analogs of L- |
| | | cysteine, L-serine and other 3-substituted |
| | | amino acids |
| | Enoyl-CoA hydratase | Acts in the reverse direction With cis- |
| | | compounds, yields (3R)-3-hydroxyacyl- |
| | | CoA (cf. EC 4.2.1.74) |
| | Nitrile hydratase | Acts on short-chain aliphatic nitriles, |
| | | converting them into the corresponding |
| | | acid amides Does not act on these amides |
| | | or on aromatic nitriles (cf EC 3.5.5.1) |
| | Tryptophan-tRNA | |
| | ligase | |
| Tyrosine metabolism | Alcohol dehydrogenase | Acts on primary or secondary alcohols or |
| | | hemiacetals The animal, but not the |
| | | yeast, enzyme acts also on cyclic |
| | | secondary alcohols |
| | (R)-4- | Also acts, more slowly, on (R)-3- |
| | hydroxyphenyllactate | phenyllactate, (R)-3-(indole-3-yl)lactate |
| | dehydrogenase | and (R)-lactate |
| | Hydroxyphenylpyruvate | Also acts on 3-(3,4- |
| | reductase | dihydroxyphenyl)lactate Involved with |
| | | EC 2.3.1.140 in the biosynthesis of |
| | | rosmarinic acid |
| | Aryl-alcohol | A group of enzymes with broad |
| | dehydrogenase | specificity towards primary alcohols with |
| | | an aromatic or cyclohex-1-ene ring, but |
| | | with low or no activity towards short- |
| | | chain aliphatic alcohols |
| | Catechol oxidase | Also acts on a variety of substituted |
| | | catechols Many of these enzymes also |
| | | catalyse the reaction listed under EC |
| | | 1.14.18.1; this is especially true for the |
| | | classical tyrosinase |
| | Iodide peroxidase | |
| | 3,4- | |
| | dihydroxyphenylacetate | |
| | 2,3-dioxygenase | |
| | 4- | |
| | hydroxyphenylpyruvate | |
| | dioxygenase | |
| | Stizolobate synthase | The intermediate product undergoes ring |
| | | closure and oxidation, with NAD(P)(+) |
| | | as acceptor, to stizolobic acid |
| | Stizolobinate synthase | The intermediate product undergoes ring |
| | | closure and oxidation, with NAD(P)(+) |
| | | as acceptor, to stizolobinic acid |
| | Gentisate 1,2- | |
| | dioxygenase | |
| | Homogentisate 1,2- | |
| | dioxygenase | |
| | 4-hydroxyphenylacetate | Also acts on 4-hydroxyhydratropate |
| | 1-monooxygenase | forming 2-methylhomogentisate and on |
| | | 4-hydroxyphenoxyacetate forming |
| | | hydroquinone and glycolate |
| | 4-hydroxyphenylacetate | |
| | 3-monooxygenase | |
| | Tyrosine N- | |
| | monooxygenase | |
| | Hydroxyphenylacetonitrile | |
| | 2-monooxygenase | |
| | Tyrosine 3- | Activated by phosphorylation, catalysed |
| | monooxygenase | by EC 2.7.1.128 |
| | Dopamine-beta- | Stimulated by fumarate |
| | monooxygenase | |
| | Monophenol | A group of copper proteins that also |
| | monooxygenase | catalyse the reaction of EC 1.10.3.1, if |
| | | only 1,2-benzenediols are available as |
| | | substrate |
| | Succinate- | |
| | semialdehyde | |
| | dehydrogenase | |
| | (NAD(P)+) | |
| | Aryl-aldehyde | Oxidizes a number of aromatic |
| | dehydrogenase | aldehydes, but not aliphatic aldehydes |
| | Aldehyde | Wide specificity, including oxidation of |
| | dehydrogenase (NAD+) | D-glucuronolactone to D-glucarate |
| | 4-carboxy-2- | Does not act on unsubstituted aliphatic or |
| | hydroxymuconate-6- | aromatic aldehydes or glucose NAD(+) |
| | semialdehyde | can replace NADP(+), but with lower |
| | dehydrogenase | affinity |
| | Aldehyde | |
| | dehydrogenase | |
| | (NAD(P)+) | |
| | 4- | With EC 4.2.1.87, brings about the |
| | hydroxyphenylacetaldehyde | metabolism of octopamine in |
| | dehydrogenase | Pseudomonas |
| | Aldehyde oxidase | Also oxidizes quinoline and pyridine |
| | | derivatives May be identical with EC |
| | | 1.1.3.22 |
| | L-amino acid oxidase | |
| | Amine oxidase (flavin- | Acts on primary amines, and usually also |
| | containing) | on secondary and tertiary amines |
| | Amine oxidase (copper- | A group of enzymes including those |
| | containing) | oxidizing primary amines, diamines and |
| | | histamine One form of EC 1.3.1.15 from |
| | | rat kidney also catalyses this reaction |
| | Aralkylamine | Phenazine methosulfate can act as |
| | dehydrogenase | acceptor Acts on aromatic amines and, |
| | | more slowly, on some long-chain |
| | | aliphatic amines, but not on methylamine |
| | | or ethylamine (cf EC 1.4.99.3) |
| | Phenol O- | Acts on a wide variety of simple alkyl-, |
| | methyltransferase | methoxy- and halo-phenols |
| | Tyramine N- | Has some activity on phenylethylamine |
| | methyltransferase | analogs |
| | Phenylethanolamine N- | Acts on various phenylethanolamines; |
| | methyltransferase | converts noradrenalin into adrenalin |
| | Catechol O- | The mammalian enzymes act more |
| | methyltransferase | rapidly on catecholamines such as |
| | | adrenaline or noradrenaline than on |
| | | catechols |
| | Glutamine N- | |
| | phenylacetyltransferase | |
| | Rosmarinate synthase | Involved with EC 1.1.1.237 in the |
| | | biosynthesis of rosmarinic acid |
| | Hydroxymandelonitrile | 3,4-dihydroxymandelonitrile can also act |
| | glucosyltransferase | as acceptor |
| | Aspartate | Also acts on L-tyrosine, L-phenylalanine |
| | aminotransferase | and L-tryptophan. This activity can be |
| | | formed from EC 2.6.1.57 by controlled |
| | | proteolysis |
| | Dihydroxyphenylalanine | |
| | aminotransferase | |
| | Tyrosine | L-phenylalanine can act instead of L- |
| | aminotransferase | tyrosine The mitochondrial enzyme may |
| | | be identical with EC 2.6.1.1 The three |
| | | isoenzymic forms are interconverted by |
| | | EC 3.4.22.4 |
| | Aromatic amino acid | L-methionine can also act as donor, more |
| | transferase | slowly Oxaloacetate can act as acceptor |
| | | Controlled proteolysis converts the |
| | | enzyme to EC 2.6.1.1 |
| | Histidinol-phosphate | |
| | aminotransferase | |
| | Fumarylacetoacetase | Also acts on other 3,5- and 2,4-dioxo |
| | | acids |
| | Acylpyruvate hydrolase | Acts on formylpyruvate, 2,4- |
| | | dioxopentanoate, 2,4-dioxohexanoate and |
| | | 2,4-dioxoheptanoate |
| | Tyrosine decarboxylase | The bacterial enzyme also acts on 3- |
| | | hydroxytyrosine and, more slowly, on 3- |
| | | hydroxyphenylalanine |
| | Aromatic-L-amino-acid | Also acts on L-tryptophan, 5-hydroxy-L- |
| | decarboxylase | tryptophan and dihydroxy-L- |
| | | phenylalanine (DOPA) |
| | Gentisate | |
| | decarboxylase | |
| | 5-oxopent-3-ene-1,2,5- | |
| | tricarboxylate | |
| | decarboxylase | |
| | Tyrosine phenol-lyase | Also slowly catalyses pyruvate formation |
| | | from D-tyrosine, S-methyl-L-cysteine, |
| | | L-cysteine, L-serine and D-serine |
| | (S)-norcoclaurine | The reaction makes a 6-membered ring |
| | synthase | by forming a bond between C-6 of the |
| | | 3,4-dihydroxyphenyl group of the |
| | | dopamine and C-1 of the aldehyde in the |
| | | imine formed between the substrates The |
| | | product is the precursor of the |
| | | benzylisoquinoline alkaloids in plants |
| | | Will also catalyse the reaction of 4-(2- |
| | | aminoethyl)benzene-1,2-diol + (3,4- |
| | | dihydroxyphenyl)acetaldehyde to form |
| | | (S)-norlaudanosoline, but this alkaloid |
| | | has not been found to occur in plants |
| | Dihydroxyphenylalanine | |
| | ammonia-lyase | |
| | Phenylalanine | May also act on L-tyrosine |
| | ammonia-lyase | |
| | Maleylacetoacetate | Also acts on maleylpyruvate |
| | isomerase | |
| | Maleylpyruvate | |
| | isomerase | |
| | Phenylpyruvate | Also acts on other arylpyruvates |
| | tautomerase | |
| | 5-carboxymethyl-2- | |
| | hydroxymuconate | |
| | delta-isomerase | |
| | Tyrosine 2,3- | |
| | aminomutase | |
| | Phenylacetate-CoA | Also acts, more slowly, on acetate, |
| | ligase | propanoate and butanoate, but not on |
| | | hydroxy derivatives of phenylacetate and |
| | | related compounds |

### VII. PROMOTERS AS SENTINELS

Useful promoters include those that are capable of facilitating preferential transcription, i.e. tissue-specific or developmentally regulated gene expression and being a component of facile systems to evaluate the metabolic/physiological state of a plant cell, tissue or organ. Many such promoters are included in this application. Operably linking a sequence to these promoters that can act as a reporter and inserting the construct into a plant allows detection of the preferential in plantar transcription. For example, the quantitative state of responses to environmental conditions can be detected by using a plant having a construct that contains a stress-inducible promoter linked to and controlling expression of a sequence encoding GFP. The greater the stress promoter is induced, the greater the levels of fluorescence from GFP will be produced and this provides a measure of the level of stress being expressed by the plant and/or the ability of the plant to respond internally to the stress.

More specifically, using this system the activities of any metabolic pathway (catabolic and anabolic), stress-related pathways as on any plant gene repeated activity can be monitored. In addition, assays can be developed using this sentinel system to select for superior genotypes with greater yield characteristics or to select for plants with altered responses to chemical, herbicide, or plant growth regulators or to identify chemical, herbicides or plant growth regulators by their response on such sentinels.

Specifically, a promoter that is regulated in plants in the desired way, is operably linked to a reporter such as GFP, RFP, etc., and the constructs are introduced into the plant of interest. The behavior of the reporter is monitored using technologies typically specific for that reporter. With GFP, RFP, etc., it could typically be by microscopy of whole plants, organs, tissues or cells under excitation by an appropriate wavelength of UV light.

### VIII. HOW TO MAKE DIFFERENT EMBODIMENTS OF THE INVENTION

The invention relates to (I) polynucleotides and methods of use thereof, such as

IA. Probes, Primers and Substrates;

IB. Methods of Detection and Isolation;
B.1. Hybridization;
B.2. Methods of Mapping;
B.3. Southern Blotting;
B.4. Isolating cDNA from Related Organisms;
B.5. Isolating and/or Identifying Orthologous Genes

IC. Methods of Inhibiting Gene Expression
C.1. Antisense
C.2. Ribozyme Constructs;
C.3. Chimeraplasts;
C.4. Co-Suppression;
C.5. Transcriptional Silencing
C.6. Other Methods to Inhibit Gene Expression

ID. Methods of Functional Analysis;

IE. Promoter Sequences and Their Use;

IF. UTRs and/or Intron Sequences and Their Use; and

IG. Coding Sequences and Their Use.

The invention also relates to (II) polypeptides and proteins and methods of use thereof, such as

IIA. Native Polypeptides and Proteins
A.1 Antibodies
A.2 In Vitro Applications

IIB. Polypeptide Variants, Fragments and Fusions
B.1 Variants
B.2 Fragments
B.3 Fusions

The invention also includes (III) methods of modulating polypeptide production, such as

IIIA. Suppression
A.1 Antisense
A.2 Ribozymes
A.3 Co-suppression
A.4 Insertion of Sequences into the Gene to be Modulated
A.5 Promoter Modulation
A.6 Expression of Genes containing Dominant-Negative Mutations

IIIB. Enhanced Expression
B.1 Insertion of an Exogenous Gene
B.2 Promoter Modulation

The invention further concerns (IV) gene constructs and vector construction, such as

IVA. Coding Sequences

IVB. Promoters

IVC. Signal Peptides

The invention still further relates to

V. Transformation Techniques

I. Polynucleotides

Exemplified SDFs of the invention represent fragments of the genome of corn, wheat, rice, soybean or Arabidopsis and/or represent mRNA expressed from that genome. The isolated nucleic acid of the invention also encompasses corresponding fragments of the genome and/or cDNA complement of other organisms as described in detail below.

Polynucleotides of the invention are isolated from polynucleotide libraries using primers comprising sequences similar to those described, in the attached Reference and Sequences Tables or complements thereof. See, for example, the methods described in Sambrook et al., supra.

Alternatively, the polynucleotides of the invention can be produced by chemical synthesis. Such synthesis methods are described below.

It is contemplated that the nucleotide sequences presented herein contain some small percentage of errors. These errors arise in the normal course of determination of nucleotide sequences. Sequence errors can be corrected by obtaining seeds deposited under the accession numbers cited above, propagating them, isolating genomic DNA or appropriate mRNA from the resulting plants or seeds thereof, amplifying the relevant fragment of the genomic DNA or mRNA using primers having a sequence that flanks the erroneous sequence and sequencing the amplification product.

I.A. Probes, Primers and Substrates

SDFs of the invention can be applied to substrates for use in array applications such as, but not limited to, assays of global gene expression, under varying conditions of development, and growth conditions. The arrays are also used in diagnostic or forensic methods (WO95/35505, U.S. Pat. No. 5,445,943 and U.S. Pat. No. 5,410,270).

Probes and primers of the instant invention hybridize to a polynucleotide comprising a sequence in or encoded by those in the Reference and Sequence Tables or fragments or complements thereof. Though many different nucleotide sequences can encode an amino acid sequence, the sequences of the Reference and Sequence Table are generally preferred for encoding polypeptides of the invention. However, the sequence of the probes and/or primers of the instant invention need not be identical to those in the Reference and Sequence Tables or the complements thereof. For example, some variation in probe or primer sequence and/or length allows detection of additional family members as well as orthologous genes and more taxonomically distant related sequences. Similarly, probes and/or primers of the invention include additional nucleotides that serve as a label for detecting the formed duplex or for subsequent cloning purposes.

Probe length varys depending on the application. For use as primers, probes are 12-40 nucleotides, preferably 18-30 nucleotides long. For use in mapping, probes are preferably 50 to 500 nucleotides, preferably 100-250 nucleotides long. For Southern hybridizations, probes as long as several kilobases are used as explained below.

The probes and/or primers are produced by synthetic procedures such as the triester method of Matteucci et al. J. Am. Chem. Soc. 103:3185 (1981) or according to Urdea et al. Proc. Natl. Acad. 80:7461 (1981) or using commercially available automated oligonucleotide synthesizers.

I.B. Methods of Detection and Isolation

The polynucleotides of the invention can be utilized in a number of methods known to those skilled in the art as probes and/or primers to isolate and detect polynucleotides including, without limitation: Southerns, Northerns, Branched DNA hybridization assays, polymerase chain reaction microarray assays and variations thereof. Specific methods given by way of examples, and discussed below include:

Hybridization

Methods of Mapping

Southern Blotting

Isolating cDNA from Related Organisms

Isolating and/or Identifying Orthologous Genes.

Also, the nucleic acid molecules of the invention can used in other methods, such as high density oligonucleotide hybridizing assays, described, for example, in U.S. Pat. Nos. 6,004,753; 5,945,306; 5,945,287; 5,945,308; 5,919,686; 5,919,661; 5,919,627; 5,874,248; 5,871,973; 5,871,971; 5,871,930; and PCT Pub. Nos. WO 9946380; WO 9933981; WO 9933870; WO 9931252; WO 9915658; WO 9906572; WO 9858052; WO 9958672; and WO 9810858.

B.1. Hybridization

The isolated SDFs of the Reference and Sequence tables or fragments thereof of the present invention can be used as probes and/or primers for detection and/or isolation of related polynucleotide sequences through hybridization. Hybridization of one nucleic acid to another constitutes a physical property that defines the subject SDF of the invention and the identified related sequences. Also, such hybridization imposes structural limitations on the pair. A good general discussion of the factors for determining hybridization conditions is provided by Sambrook et al. (“Molecular Cloning, a Laboratory Manual, 2and ed., c. 1989 by Cold Spring Harbor Laboratory Press, Cold Spring Harbor, N.Y.; see esp., chapters 11 and 12). Additional considerations and details of the physical chemistry of hybridization are provided by G. H. Keller and M. M. Manak “DNA Probes”, 2^{and} Ed. pp. 1-25, c. 1993 by Stockton Press, New York, N.Y.

Depending on the stringency of the conditions under which these probes and/or primers are used, polynucleotides exhibiting a wide range of similarity to those in the Reference and Sequence or fragments thereof are detected or isolated. When the practitioner wishes to examine the result of membrane hybridizations under a variety of stringencies, an efficient way to do so is to perform the hybridization under a low stringency condition, then to wash the hybridization membrane under increasingly stringent conditions.

When using SDFs to identify orthologous genes in other species, the practitioner will preferably adjust the amount of target DNA of each species so that, as nearly as is practical, the same number of genome equivalents are present for each species examined. This prevents faint signals from species having large genomes, and thus small numbers of genome equivalents per mass of DNA, from erroneously being interpreted as absence of the corresponding gene in the genome.

The probes and/or primers of the instant invention can also be used to detect or isolate nucleotides that are “identical” to the probes or primers. Two nucleic acid sequences or polypeptides are said to be “identical” if the sequence of nucleotides or amino acid residues, respectively, in the two sequences is the same when aligned for maximum correspondence as described below.

Isolated polynucleotides within the scope of the invention also include allelic variants of the specific sequences presented in the Reference and Sequence tables. The probes and/or primers of the invention are also used to detect and/or isolate polynucleotides exhibiting at least 80% sequence identity with the sequences of the Reference and Sequence tables or fragments thereof.

With respect to nucleotide sequences, degeneracy of the genetic code provides the possibility to substitute at least one nucleotide of the nucleotide sequence of a gene with a different nucleotide without changing the amino acid sequence of the polypeptide. Hence, the DNA of the present invention also has any base sequence that has been changed from a sequence in the Reference and Sequence tables by substitution in accordance with degeneracy of genetic code. References describing codon usage include: Carels et al., J. Mol. Evol. 46: 45 (1998) and Fennoy et al., Nucl. Acids Res. 21(23): 5294 (1993).

B.2. Mapping

The isolated SDF DNA of the invention is used to create various types of genetic and physical maps of the genome of corn, Arabidopsis, soybean, rice, wheat, or other plants. Some SDFs are absolutely associated with particular phenotypic traits, allowing construction of gross genetic maps. While not all SDFs are immediately associated with a phenotype, all SDFs can be used as probes for identifying polymorphisms associated with phenotypes of interest. Briefly, one method of mapping involves total DNA isolation from individuals. The DNA is subsequently cleaved with one or more restriction enzymes, separated according to mass, transferred to a solid support, hybridized with SDF DNA and the pattern of fragments compared. Polymorphisms associated with a particular SDF are visualized as differences in the size of fragments produced between individual DNA samples after digestion with a particular restriction enzyme and hybridization with the SDF. After identification of polymorphic SDF sequences, linkage studies are conducted. By using the polymeric individuals as parents in crossing programs, F2 progeny recombinants or recombinant inbreds, for example, are then analyzed. The order of DNA polymorphisms along the chromosomes is determined based on the frequency with which they are inherited together versus independently. The closer the location of two polymorphisms on a chromosome, the higher the probability that they are inherited together. Integration of the relative positions of all the polymorphisms and associated marker SDFs produce a genetic map of the species where the distances between markers reflect the recombination frequencies in that chromosome segment.

The use of recombinant inbred lines for such genetic mapping is described for Arabidopsis by Alonso-Blanco et al. (Methods in Molecular Biology, vol.82, “Arabidopsis Protocols”, pp. 137-146, J. M. Martinez-Zapater and J. Salinas, eds., c. 1998 by Humana Press, Totowa, N.J.) and for corn by Burr (“Mapping Genes with Recombinant Inbreds”, pp. 249-254. In Freeling, M. and V. Walbot (Ed.), The Maize Handbook, c. 1994 by Springer-Verlag New York, Inc.: New York, N.Y., USA; Berlin Germany; Burr et al. Genetics (1998) 118: 519; Gardiner, J. et al., (1993) Genetics 134: 917). This procedure, however, is not limited to plants and is used for other organisms (such as yeast) or for individual cells.

The SDFs of the present invention are also used for simple sequence repeat (SSR) mapping. Rice SSR mapping is described by Morgante et al. (The Plant Journal (1993) 3: 165), Panaud et al. (Genome (1995) 38: 1170); Senior et al. (Crop Science (1996) 36: 1676), Taramino et al. (Genome (1996) 39: 277) and Ahn et al. (Molecular and General Genetics (1993) 241: 483-90). SSR mapping is achieved using various methods. In one instance, polymorphisms are identified when sequence specific probes contained within an SDF flanking an SSR are made and used in polymerase chain reaction (PCR) assays with template DNA from two or more individuals of interest. Here, a change in the number of tandem repeats between the SSR-flanking sequences produces differently sized fragments (U.S. Pat. No. 5,766,847). Alternatively, polymorphisms are identified by using the PCR fragment produced from the SSR-flanking sequence specific primer reaction as a probe against Southern blots representing different individuals (U. H. Refseth et al., (1997) Electrophoresis 18: 1519).

Genetic and physical maps of crop species have many uses. For example, these maps are used to devise positional cloning strategies for isolating novel genes from the mapped crop species. In addition, because the genomes of closely related species are largely syntenic (i.e. they display the same ordering of genes within the genome), these maps are used to isolate novel alleles from relatives of crop species by positional cloning strategies.

The various types of maps discussed above are used with the SDFs of the invention to identify Quantitative Trait Loci (QTLs). Many important crop traits, such as the solids content of tomatoes, are quantitative traits and result from the combined interactions of several genes. These genes reside at different loci in the genome, often times on different chromosomes, and generally exhibit multiple alleles at each locus. The SDFs of the invention are used to identify QTLs and isolate specific alleles as described by de Vicente and Tanksley (Genetics 134:585 (1993)). In addition to isolating QTL alleles in present crop species, the SDFs of the invention are also used to isolate alleles from the corresponding QTL of wild relatives. Transgenic plants having various combinations of QTL alleles are then created and the effects of the combinations measured. Once a desired allele combination is identified, crop improvement is accomplished either through biotechnological means or by directed conventional breeding programs (for review see Tanksley and McCouch, Science 277:1063 (1997)).

In another embodiment, the SDFs are used to help create physical maps of the genome of corn, Arabidopsis and related species. Where SDFs are ordered on a genetic map, as described above, they are used as probes to discover which clones in large libraries of plant DNA fragments in YACs, BACs, etc. contain the same SDF or similar sequences, thereby facilitating the assignment of the large DNA fragments to chromosomal positions. Subsequently, the large BACs, YACs, etc. are ordered unambiguously by more detailed studies of their sequence composition (e.g. Marra et al. (1997) Genomic Research 7:1072-1084) and by using their end or other sequences to find the identical sequences in other cloned DNA fragments. The overlapping of DNA sequences in this way allows building large contigs of plant sequences to be built that, when sufficiently extended, provide a complete physical map of a chromosome. Sometimes the SDFs themselves provide the means of joining cloned sequences into a contig. All scientific and patent publications cited in this paragraph are hereby incorporated by reference.

The patent publication WO95/35505 and U.S. Pat. Nos. 5,445,943 and 5,410,270, both hereby incorporated by reference, describe scanning multiple alleles of a plurality of loci using hybridization to arrays of oligonucleotides. These techniques are useful for each of the types of mapping discussed above.

Following the procedures described above and using a plurality of the SDFs of the present invention, any individual is genotyped. These individual genotypes are used for the identification of particular cultivars, varieties, lines, ecotypes and genetically modified plants or can serve as tools for subsequent genetic studies involving multiple phenotypic traits.

B.3 Southern Blot Hybridization

The sequences from Reference and Sequence tables or fragments thereof can be used as probes for various hybridization techniques. These techniques are useful for detecting target polynucleotides in a sample or for determining whether transgenic plants, seeds or host cells harbor a gene or sequence of interest and thus are expected to exhibit a particular trait or phenotype.

In addition, the SDFs from the invention are used to isolate additional members of gene families from the same or different species and/or orthologous genes from the same or different species. This is accomplished by hybridizing an SDF to, for example, a Southern blot containing the appropriate genomic DNA or cDNA. Given the resulting hybridization data, one of ordinary skill in the art distinguishes and isolates the correct DNA fragments by size, restriction sites, sequence and stated hybridization conditions from a gel or from a library.

Identification and isolation of orthologous genes from closely related species and alleles within a species is particularly desirable because of their potential for crop improvement. Many important crop traits, such as the solid content of tomatoes, result from the combined interactions of the products of several genes residing at different loci in the genome. Generally, alleles at each of these loci make quantitative differences to the trait. By identifying and isolating numerous alleles for each locus from within or different species, transgenic plants with various combinations of alleles are created and the effects of the combinations measured. Once a more favorable allele combination is identified, crop improvement is accomplished either through biotechnological means or by directed conventional breeding programs (Tanksley et al. Science 277:1063(1997)). All scientific and patent publications cited in this paragraph are hereby incorporated by reference.

The results from hybridizations of the SDFs of the invention to, for example, Southern blots containing DNA from another species are also used to generate restriction fragment maps for the corresponding genomic regions. These maps provide additional information about the relative positions of restriction sites within fragments, further distinguishing mapped DNA from the remainder of the genome.

Physical maps are made by digesting genomic DNA with different combinations of restriction enzymes.

Probes for Southern blotting to distinguish individual restriction fragments can range in size from 15 to 20 nucleotides to several thousand nucleotides. More preferably, the probe is 100 to 1,000 nucleotides long for identifying members of a gene family when it is found that repetitive sequences would complicate the hybridization. For identifying an entire corresponding gene in another species, the probe is more preferably the length of the gene, typically 2,000 to 10,000 nucleotides, but probes 50-1,000 nucleotides long are also used. Some genes, however, require probes up to 1,500 nucleotides long or overlapping probes constituting the full-length sequence to span their lengths.

Also, while it is preferred that the probe be homogeneous with respect to its sequence, it is not necessary. For example, as described below, a probe representing members of a gene family having diverse sequences is generated using PCR to amplify genomic DNA or RNA templates using primers derived from SDFs that include sequences that define the gene family.

For identifying corresponding genes in another species, the next most preferable probe is a cDNA spanning the entire coding sequence, which allows all of the mRNA-coding fragment of the gene to be identified. Probes for Southern blotting are easily generated from SDFs by making primers having the sequence at the ends of the SDF and using corn or Arabidopsis genomic DNA as a template. In instances where the SDF includes sequence conserved among species, primers including the conserved sequence are used for PCR with genomic DNA from a species of interest to obtain a probe.

Similarly, if the SDF includes a domain of interest, that fragment of the SDF is used to make primers and, with appropriate template DNA, used to make a probe to identify genes containing the domain. Alternatively, the PCR products are resolved, for example by gel electrophoresis and cloned and/or sequenced. Using Southern hybridization, the variants of the domain among members of a gene family, both within and across species, are examined.

B.4.1 Isolating DNA from Related Organisms

The SDFs of the invention are used to isolate the corresponding DNA from other organisms. Either cDNA or genomic DNA is isolated. For isolating genomic DNA, a lambda, cosmid, BAC, YAC, or other large insert genomic library from the plant of interest is constructed using standard molecular biology techniques as described in detail by Sambrook et al. 1989 (Molecular Cloning: A Laboratory Manual, 2^{nd} ed. Cold Spring Harbor Laboratory Press, New York) and by Ausubel et al. 1992 (Current Protocols in Molecular Biology, Greene Publishing, New York).

To screen a phage library, for example, recombinant lambda clones are plated out on appropriate bacterial medium using an appropriate E. coli host strain. The resulting plaques are lifted from the plates using nylon or nitrocellulose filters. The plaque lifts are processed through denaturation, neutralization, and washing treatments following the standard protocols outlined by Ausubel et al. (1992). The plaque lifts are hybridized to either radioactively labeled or non-radioactively labeled SDF DNA at room temperature for about 16 hours, usually in the presence of 50% formamide and 5×SSC (sodium chloride and sodium citrate) buffer and blocking reagents. The plaque lifts are then washed at 42° C. with 1% Sodium Dodecyl Sulfate (SDS) and at a particular concentration of SSC. The SSC concentration used is dependent upon the stringency at which hybridization occurred in the initial Southern blot analysis performed. For example, if a fragment hybridized under medium stringency (e.g., Tm −20° C.), then this condition is maintained or preferably adjusted to a less stringent condition (e.g., Tm −30° C.) to wash the plaque lifts. Positive clones show detectable hybridization, e.g. by exposure to X-ray films or chromogen formation. The positive clones are then subsequently isolated for purification using the same general protocol outlined above. Once the clone is purified, restriction analysis is conducted to narrow the region corresponding to the gene of interest. The restriction analysis and succeeding subcloning steps are done using procedures described by, for example Sambrook et al. (1989) cited above.

The procedures outlined for the lambda library are essentially similar to those used for YAC library screening, except that the YAC clones are harbored in bacterial colonies. The YAC clones are plated out at reasonable density on nitrocellulose or nylon filters supported by appropriate bacterial medium in petri plates. Following the growth of the bacterial clones, the filters are processed through the denaturation, neutralization, and washing steps following the procedures of Ausubel et al. 1992. The same hybridization procedures for lambda library screening are followed.

To isolate cDNA, similar procedures using appropriately modified vectors are employed. For instance, the library is constructed in a lambda vector appropriate for cloning cDNA such as λgt11. Alternatively, the cDNA library is made in a plasmid vector. cDNA for cloning is prepared by any of the methods known in the art, but is preferably prepared as described above. Preferably, a cDNA library includes a high proportion of full-length clones.

B.5. Isolating and/or Identifying Orthologous Genes

Probes and primers of the invention are used to identify and/or isolate polynucleotides related to those in the Reference and Sequence tables. Related polynucleotides are those that are native to other plant organisms and exhibit either similar sequence or encode polypeptides with similar biological activity. One specific example is an orthologous gene. Orthologous genes have the same functional activity. As such, orthologous genes are distinguished from homologous genes. The percentage of identity is a function of evolutionary separation and, in closely related species, the percentage of identity can be 98% to 100%. The amino acid sequence of a protein encoded by an orthologous gene can be less than 75% identical, but tends to be at least75% or at least 80% identical, more preferably at least 90%, most preferably at least 95% identical to the amino acid sequence of the reference protein.

To find orthologous genes, the probes are hybridized to nucleic acids from a species of interest under low stringency conditions, preferably one where sequences containing as much as 40-45% mismatches are able to hybridize. This condition is established by Tₘ−40° C. to Tm −48° C. (see below). Blots are then washed under conditions of increasing stringency. It is preferable that the wash stringency be such that sequences that are 85 to 100% identical will hybridize. More preferably, sequences 90 to 100% identical hybridize and most preferably only sequences greater than 95% identical hybridize. One of ordinary skill in the art will recognize that, due to degeneracy in the genetic code, amino acid sequences that are identical can be encoded by DNA sequences as little as 67% identity or less. Thus, it is preferable, for example, to make an overlapping series of shorter probes, on the order of 24 to 45 nucleotides, and individually hybridize them to the same arrayed library to avoid the problem of degeneracy introducing large numbers of mismatches.

As evolutionary divergence increases, genome sequences also tend to diverge. Thus, one of skill will recognize that searches for orthologous genes between more divergent species require the use of lower stringency conditions compared to searches between closely related species. Also, degeneracy of the genetic code is more of a problem for searches in the genome of a species more evolutionarily distant from the species that is the source of the SDF probe sequence(s).

Therefore the method described in Bouckaert et al., U.S. Ser. No. 60/121,700 Atty. Dkt. No. 2750-117P, Client Dkt. No. 00010.001, filed Feb. 25, 1999, hereby incorporated in its entirety by reference, is applied to the SDFs of the present invention to isolate related genes from plant species which do not hybridize to the corn, Arabidopsis, soybean, rice, wheat, and other plant sequences of the Reference and Sequence tables.

The SDFs of the invention are also used as probes to search for genes that are related to the SDF within a species. Such related genes are typically considered to be members of a gene family. In such a case, the sequence similarity is often concentrated into one or a few fragments of the sequence. The fragments of similar sequence that define the gene family typically encode a fragment of a protein or RNA that has an enzymatic or structural function. The percentage of identity in the amino acid sequence of the domain that defines the gene family is preferably at least 70%, more preferably at least 80 to 95%, most preferably at least 85 to 99%. To search for members of a gene family within a species, a low stringency hybridization is usually performed, but this will depend upon the size, distribution and degree of sequence divergence of domains that define the gene family. SDFs encompassing regulatory regions are used to identify coordinately expressed genes by using the regulatory region sequence of the SDF as a probe.

In the instances where the SDFs are identified as being expressed from genes that confer a particular phenotype, then the SDFs are also used as probes to assay plants of different species for those phenotypes.

I.C. Methods to Inhibit Gene Expression

The nucleic acid molecules of the present invention are used to inhibit gene transcription and/or translation. Example of such methods include, without limitation:

Antisense Constructs;

Ribozyme Constructs;

Chimeraplast Constructs;

Co-Suppression;

Transcriptional Silencing; and

Other Methods of Gene Expression.

C.1 Antisense

In some instances it is desirable to suppress expression of an endogenous or exogenous gene. A well-known instance is the FLAVOR-SAVOR™ tomato, in which the gene encoding ACC synthase is inactivated by an antisense approach, thus delaying softening of the fruit after ripening. See for example, U.S. Pat. No. 5,859,330; U.S. Pat. No. 5,723,766; Oeller, et al, Science, 254:437-439(1991); and Hamilton et al, Nature, 346:284-287 (1990). As another example, timing of flowering is controlled by suppression of the FLOWERING LOCUS C (FLC). High levels of this transcript are associated with late flowering, while absence of FLC is associated with early flowering (S. D. Michaels et al., Plant Cell 11:949 (1999). Other examples include the transition of apical meristem from leaf and shoot production to flowering which is regulated by TERMINAL FLOWER1, APETALA1 and LEAFY. Suppressing TFL1 expression induce a transition from shoot production to flowering (S. J. Liljegren, Plant Cell 11: 1007 (1999)). In yet another example, arrested ovule development and female sterility result from suppression of the ethylene forming enzyme, but can be reversed by application of ethylene (D. De Martinis et al., Plant Cell 11:1061 (1999)). The ability to manipulate female fertility of plants is useful in increasing fruit production and creating hybrids.

Some polynucleotide SDFs in the Reference and Sequence tables represent sequences that are expressed in corn, wheat, rice, soybean Arabidopsis and/or other plants. Thus the invention includes using these sequences to generate antisense constructs to inhibit translation and/or degradation of transcripts of said SDFs, typically in a plant cell.

To accomplish this, a polynucleotide segment from the desired gene that hybridizes to the mRNA expressed from the desired gene (the “antisense segment”) is operably linked to a promoter such that the antisense strand of RNA is transcribed when the construct is present in a host cell. A regulated promoter is used in the construct to control transcription of the antisense segment so that transcription occurs only under desired circumstances.

The antisense segment introduced is typically substantially identical to at least a fragment of the endogenous gene or genes to be repressed. The sequence, however, need not be perfectly identical to inhibit expression. Further, the antisense product may hybridize to the untranslated region instead of or in addition to the coding sequence of the gene. The vectors of the present invention designed such that the inhibitory effect applies to other proteins within a family of genes exhibiting homology or substantial homology to the target gene.

For antisense suppression, the introduced antisense segment sequence also need not be full length relative to either the primary transcription product or the fully processed mRNA. Generally, a higher percentage of sequence identity is used to compensate for the use of a shorter sequence. Furthermore, the introduced sequence need not have the same intron or exon pattern, and homology of non-coding segments are equally effective. Normally, a sequence of between about 30 or 40 nucleotides and the full length of the transcript can be used, although a sequence of at least about 100 nucleotides is preferred, a sequence of at least about 200 nucleotides is more preferred, and a sequence of at least about 500 nucleotides is especially preferred.

C.2. Ribozymes

It is also contemplated that gene constructs representing ribozymes and based on the SDFs in the Reference and Sequence tables tables and fragment thereof are an object of the invention. Ribozymes are also used to inhibit expression of genes by suppressing the translation of the mRNA into a polypeptide. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs.

A number of classes of ribozymes are known. One class of ribozymes is derived from a number of small circular RNAs, which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Haseloff et al. Nature, 334:585 (1988).

Like the antisense constructs above, the ribozyme sequence fragment necessary for pairing need not be identical to the target nucleotides to be cleaved, nor identical to the sequences in the Reference and Sequence tables or fragments thereof. Ribozymes are constructed by combining the ribozyme sequence and some fragment of the target gene which allows recognition of the target gene mRNA by the resulting ribozyme molecule. Generally, the sequence in the ribozyme capable of binding to the target sequence exhibits a percentage of sequence identity with at least 80%, preferably with at least 85%, more preferably with at least 90% and most preferably with at least 95%, even more preferably, with at least 96%, 97%, 98% or 99% sequence identity to some fragment of a sequence in the Reference and Sequence tables or the complements thereof. The ribozyme is equally effective in inhibiting mRNA translation by cleaving either in the untranslated or coding regions. Generally, a higher percentage of sequence identity is used to compensate for the use of a shorter sequence. Furthermore, the introduced sequence need not have the same intron or exon pattern, and homology of non-coding segments are equally effective.

C.3. Chimeraplasts

The SDFs of the invention, such as those described by Reference and Sequence tables are also used to construct chimeraplasts that introduced into a cell to produce at least one specific nucleotide change in a sequence corresponding to the SDF of the invention. A chimeraplast is an oligonucleotide comprising DNA and/or RNA that specifically hybridizes to a target region in a manner which creates a mismatched base-pair. This mismatched base-pair signals the cell's repair enzyme machinery which acts on the mismatched region and results in the replacement, insertion or deletion of designated nucleotide(s). The altered sequence is then expressed by the cell's normal cellular mechanisms. Chimeraplasts are designed to repair mutant genes, modify genes, introduce site-specific mutations, and/or act to interrupt or alter normal gene function (U.S. Pat. Nos. 6,010,907 and 6,004,804; and PCT Pub. No. WO99/58723 and WO99/07865).

C.4. Sense Suppression

The SDFs of the Reference and Sequence tables of the present invention are also useful to modulate gene expression by sense suppression. Sense suppression represents another method of gene suppression that introduces at least one exogenous copy or fragment of the endogenous sequence to be suppressed.

Introduction of expression cassettes in which a nucleic acid is configured in the sense orientation with respect to the promoter into the chromosome of a plant or by a self-replicating virus is an effective means by which to induce degradation of mRNAs of target genes. For an example of the use of this method to modulate expression of endogenous genes see, Napoli et al., The Plant Cell 2:279 (1990), and U.S. Pat. Nos. 5,034,323, 5,231,020, and 5,283,184. Inhibition of expression requires some transcription of the introduced sequence.

For sense suppression, the introduced sequence generally is substantially identical to the endogenous sequence intended to be inactivated. The minimal percentage of sequence identity is typically greater than about 65%, but a higher percentage of sequence identity might exert a more effective reduction in the level of normal gene products. Sequence identity of more than about 80% is preferred, though about 95% to absolute identity is most preferred. As with antisense regulation, the effect applys to any other proteins within a similar family of genes exhibiting homology or substantial homology to the suppressing sequence.

C.5. Transcriptional Silencing

The nucleic acid sequences of the invention, including the SDFs of the reference, Sequence, Protein Group, and Protein Group Matrix tables, and fragments thereof, contain sequences that can be inserted into the genome of an organism resulting in transcriptional silencing. Such regulatory sequences need not be operatively linked to coding sequences to modulate transcription of a gene. Specifically, a promoter sequence without any other element of a gene can be introduced into a genome to transcriptionally silence an endogenous gene (see, for example, Vaucheret, H et al. (1998) The Plant Journal 16: 651-659). As another example, triple helices can be formed using oligonucleotides based on sequences from Reference, Sequence, Protein Group, and Protein Group Matrix tables, fragments thereof, and substantially similar sequence thereto. The oligonucleotide can be delivered to the host cell and can bind to the promoter in the genome to form a triple helix and prevent transcription. An oligonucleotide of interest is one that can bind to the promoter and block binding of a transcription factor to the promoter. In such a case, the oligonucleotide can be complementary to the sequences of the promoter that interact with transcription binding factors.

C.6. Other Methods to Inhibit Gene Expression

Yet another means of suppressing gene expression is to insert a polynucleotide into the gene of interest to disrupt transcription or translation of the gene.

Low frequency homologous recombination is used to target a polynucleotide insert to a gene by flanking the polynucleotide insert with sequences that are substantially similar to the gene to be disrupted. Sequences from the Reference and Sequence tables, fragments thereof and substantially similar sequences thereto are used for homologous recombination.

In addition, random insertion of polynucleotides into a host cell genome is used to disrupt the gene of interest (Azpiroz-Leehan et al., Trends in Genetics 13:152 (1997). In this method, screening for clones from a library containing random insertions is preferred to identifying those that have polynucleotides inserted into the gene of interest. Such screening is performed using probes and/or primers described above based on sequences from Reference and Sequence tables, fragments thereof, and substantially similar sequence thereto. The screening is also performed by selecting clones or R₁ plants having a desired phenotype.

I.D. Methods of Functional Analysis

The constructs described in the methods under I.C. above are used to determine the function of the polypeptide encoded by the gene that is targeted by the constructs.

Down-regulating the transcription and translation of the targeted gene in the host cell or organisms, such as a plant, produces phenotypic changes as compared to a wild-type cell or organism. In addition, in vitro assays are used to determine if any biological activity, such as calcium flux, DNA transcription, nucleotide incorporation, etc., are being modulated by the down-regulation of the targeted gene.

Coordinated regulation of sets of genes, e.g. those contributing to a desired polygenic trait, is sometimes necessary to obtain a desired phenotype. SDFs of the invention representing transcription activation and DNA binding domains are assembled into hybrid transcriptional activators. These hybrid transcriptional activators are used with their corresponding DNA elements (i.e. those bound by the DNA-binding SDFs) to effect coordinated expression of desired genes (J. J. Schwarz et al., Mol. Cell. Biol. 12:266 (1992), A. Martinez et al., Mol. Gen. Genet. 261:546 (1999)).

The SDFs of the invention are also used in the two-hybrid genetic systems to identify networks of protein-protein interactions (L. McAlister-Henn et al., Methods 19:330 (1999), J. C. Hu et al., Methods 20:80 (2000), M. Golovkin et al., J. Biol. Chem. 274:36428 (1999), K. Ichimura et al., Biochem. Biophys. Res. Comm. 253:532 (1998)). The SDFs of the invention also are used in various expression display methods to identify important protein-DNA interactions (e.g. B. Luo et al., J. Mol. Biol. 266:479 (1997)).

I.E. Promoters

The SDFs of the invention are also useful as structural or regulatory sequences in a construct for modulating the expression of the corresponding gene in a plant or other organism, e.g. a symbiotic bacterium. For example, promoter sequences associated to SDFs of the reference, Sequence, Protein Group, and Protein Group Matrix tables of the present invention can be useful in directing expression of coding sequences either as constitutive promoters or to direct expression in particular cell types, tissues, or organs or in response to environmental stimuli.

With respect to the SDFs of the present invention a promoter is likely to be a relatively small portion of a genomic DNA (gDNA) sequence located in the first 2000 nucleotides upstream from an initial exon identified in a gDNA sequence or initial “ATG” or methionine codon or translational start site in a corresponding cDNA sequence. Such promoters are more likely to be found in the first 1000 nucleotides upstream of an initial ATG or methionine codon or translational start site of a cDNA sequence corresponding to a gDNA sequence. In particular, the promoter is usually located upstream of the transcription start site. The fragments of a particular gDNA sequence that function as elements of a promoter in a plant cell will preferably be found to hybridize to gDNA sequences presented and described in the Reference table at medium or high stringency, relevant to the length of the probe and its base composition.

Promoters are generally modular in nature. Promoters can consist of a basal promoter that functions as a site for assembly of a transcription complex comprising an RNA polymerase, for example RNA polymerase II. A typical transcription complex will include additional factors such as TF_{II}B, TF_{II}D, and TF_{II}E. Of these, TF_{II}D appears to be the only one to bind DNA directly. The promoter might also contain one or more enhancers and/or suppressors that function as binding sites for additional transcription factors that have the function of modulating the level of transcription with respect to tissue specificity and of transcriptional responses to particular environmental or nutritional factors, and the like.

Short DNA sequences representing binding sites for proteins can be separated from each other by intervening sequences of varying length. For example, within a particular functional module, protein binding sites may be constituted by regions of 5 to 60, preferably 10 to 30, more preferably 10 to 20 nucleotides. Within such binding sites, there are typically 2 to 6 nucleotides that specifically contact amino acids of the nucleic acid binding protein. The protein binding sites are usually separated from each other by 10 to several hundred nucleotides, typically by 15 to 150 nucleotides, often by 20 to 50 nucleotides. DNA binding sites in promoter elements often display dyad symmetry in their sequence. Often elements binding several different proteins, and/or a plurality of sites that bind the same protein, will be combined in a region of 50 to 1,000 basepairs.

Elements that have transcription regulatory function can be isolated from their corresponding endogenous gene, or the desired sequence can be synthesized, and recombined in constructs to direct expression of a coding region of a gene in a desired tissue-specific, temporal-specific or other desired manner of inducibility or suppression. When hybridizations are performed to identify or isolate elements of a promoter by hybridization to the long sequences presented in the Reference tables, conditions are adjusted to account for the above-described nature of promoters. For example short probes, constituting the element sought, are preferably used under low temperature and/or high salt conditions. When long probes, which might include several promoter elements are used, low to medium stringency conditions are preferred when hybridizing to promoters across species.

If a nucleotide sequence of an SDF, or part of the SDF, functions as a promoter or fragment of a promoter, then nucleotide substitutions, insertions or deletions that do not substantially affect the binding of relevant DNA binding proteins would be considered equivalent to the exemplified nucleotide sequence. It is envisioned that there are instances where it is desirable to decrease the binding of relevant DNA binding proteins to silence or down-regulate a promoter, or conversely to increase the binding of relevant DNA binding proteins to enhance or up-regulate a promoter and vice versa. In such instances, polynucleotides representing changes to the nucleotide sequence of the DNA-protein contact region by insertion of additional nucleotides, changes to identity of relevant nucleotides, including use of chemically-modified bases, or deletion of one or more nucleotides are considered encompassed by the present invention. In addition, fragments of the promoter sequences described by Reference tables and variants thereof can be fused with other promoters or fragments to facilitate transcription and/or transcription in specific type of cells or under specific conditions.

Promoter function can be assayed by methods known in the art, preferably by measuring activity of a reporter gene operatively linked to the sequence being tested for promoter function. Examples of reporter genes include those encoding luciferase, green fluorescent protein, GUS, neo, cat and bar.

I.F. UTRs and Junctions

Polynucleotides comprising untranslated (UTR) sequences and intron/exon junctions are also within the scope of the invention. UTR sequences include introns and 5′ or 3′ untranslated regions (5′ UTRs or 3′ UTRs). Fragments of the sequences shown in the Reference and Sequence tables comprise UTRs and intron/exon junctions.

Some of these fragments of SDFs, especially UTRs, have regulatory functions related to, for example, translation rate and mRNA stability. Thus, these fragments of SDFs are isolated for use as elements of gene constructs for regulated production of polynucleotides encoding desired polypeptides.

Some introns of genomic DNA segments also have regulatory functions. Sometimes regulatory elements, especially transcription enhancer or suppressor elements, are found within introns. Also, elements related to stability of heteronuclear RNA and efficiency of splicing and of transport to the cytoplasm for translation are found in intron elements. Thus, these segments also find use as elements of expression vectors intended for use to transform plants.

Just as with promoters UTR sequences and intron/exon junctions vary from those shown in the Reference and Sequence tables. Such changes from those sequences preferably do not affect the regulatory activity of the UTRs or intron/exon junction sequences on expression, transcription, or translation unless selected to do so. However, in some instances, down- or up-regulation of such activity may be desired to modulate traits or phenotypic or in vitro activity.

I.G. Coding Sequences

Isolated polynucleotides of the invention include coding sequences that encode polypeptides comprising an amino acid sequence encoded by sequences described in the Reference and Sequence tables.

A nucleotide sequence encodes a polypeptide if a cell (or a cell free in vitro system) expressing that nucleotide sequence produces a polypeptide having the recited amino acid sequence when the nucleotide sequence is transcribed and the primary transcript is subsequently processed and translated by a host cell (or a cell free in vitro system) harboring the nucleic acid. Thus, an isolated nucleic acid that encodes a particular amino acid sequence is a genomic sequence comprising exons and introns or a cDNA sequence that represents the product of splicing thereof. An isolated nucleic acid encoding an amino acid sequence also encompasses heteronuclear RNA, which contains sequences that are spliced out during expression, and mRNA, which lacks those sequences.

Coding sequences are constructed using chemical synthesis techniques or by isolating coding sequences or by modifying such synthesized or isolated coding sequences as described above.

In addition to coding sequences encoding the polypeptide sequences of the Reference and Sequence tables, which are native to corn, Arabidopsis, soybean, rice, wheat, and other plants, the isolated polynucleotides are polynucleotides that encode variants, fragments, and fusions of those native proteins. Such polypeptides are described below in part II.

In variant polynucleotides generally, the number of substitutions, deletions or insertions is preferably less than 20%, more preferably less than 15%; even more preferably less than 10%, 5%, 3% or 1% of the number of nucleotides comprising a particularly exemplified sequence. It is generally expected that non-degenerate nucleotide sequence changes that result in I to 10, more preferably 1 to 5 and most preferably 1 to 3 amino acid insertions, deletions or substitutions do not greatly affect the function of an encoded polypeptide. The most preferred embodiments are those wherein 1 to 20, preferably 1 to 10, most preferably 1 to 5 nucleotides are added to, or deleted from and/or substituted in the sequences specifically disclosed in the Reference and Sequence tables or fragments thereof.

Insertions or deletions in polynucleotides intended to be used for encoding a polypeptide preferably preserve the reading frame. This consideration is not so important in instances when the polynucleotide is intended to be used as a hybridization probe.

II. Polypeptides and Proteins

IIA. Native Polypeptides and Proteins

Polypeptides within the scope of the invention include both native proteins as well as variants, fragments, and fusions thereof. Polypeptides of the invention are those encoded by any of the six reading frames of sequences shown in the Reference and Sequence tables, preferably encoded by the three frames reading in the 5′ to 3′ direction of the sequences as shown.

Native polypeptides include the proteins encoded by the sequences shown in the Reference and Sequence tables. Such native polypeptides include those encoded by allelic variants.

Polypeptide and protein variants will exhibit at least 75% sequence identity to those native polypeptides of the Reference and Sequence tables. More preferably, the polypeptide variants will exhibit at least 85% sequence identity; even more preferably, at least 90% sequence identity; more preferably at least 95%, 96%, 97%, 98%, or 99% sequence identity. Fragments of polypeptide or fragments of polypeptides exhibit similar percentages of sequence identity to the relevant fragments of the native polypeptide. Fusions exhibit a similar percentage of sequence identity in that fragment of the fusion represented by the variant of the native peptide.

Furthermore, polypeptide variants exhibit at least one of the functional properties of the native protein. Such properties include, without limitation, protein interaction, DNA interaction, biological activity, immunological activity, receptor binding, signal transduction, transcription activity, growth factor activity, secondary structure, three-dimensional structure, etc. As to properties related to in vitro or in vivo activities, the variants preferably exhibit at least 60% of the activity of the native protein; more preferably at least 70%, even more preferably at least 80%, 85%, 90% or 95% of at least one activity of the native protein.

One type of variant of native polypeptides comprises amino acid substitutions, deletions and/or insertions. Conservative substitutions are preferred to maintain the function or activity of the polypeptide.

Within the scope of percentage of sequence identity described above, a polypeptide of the invention may have additional individual amino acids or amino acid sequences inserted into the polypeptide in the middle thereof and/or at the N-terminal and/or C-terminal ends thereof. Likewise, some of the amino acids or amino acid sequences may be deleted from the polypeptide.

A.1 Antibodies

Isolated polypeptides are utilized to produce antibodies. Polypeptides of the invention are generally used, for example, as antigens for raising antibodies by known techniques. The resulting antibodies are useful as reagents for determining the distribution of the antigen protein within the tissues of a plant or within a cell of a plant. The antibodies are also useful for examining the production level of proteins in various tissues, for example in a wild-type plant or following genetic manipulation of a plant, by methods such as Western blotting.

Antibodies of the present invention, both polyclonal and monoclonal, are prepared by conventional methods. In general, the polypeptides of the invention are first used to immunize a suitable animal, such as a mouse, rat, rabbit, or goat. Rabbits and goats are preferred for the preparation of polyclonal sera due to the volume of serum obtainable and the availability of labeled anti-rabbit and anti-goat antibodies as detection reagents. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization.

Polyclonal antisera is obtained by bleeding the immunized animal into a glass or plastic container, incubating the blood at 25° C. for one hour, followed by incubating the blood at 4° C. for 2-18 hours. The serum is recovered by centrifugation (e.g., 1,000×g for 10 minutes). About 20-50 ml per bleed may be obtained from rabbits.

Monoclonal antibodies are prepared using the method of Kohler and Milstein, Nature 256: 495 (1975), or modification thereof. Typically, a mouse or rat is immunized as described above. However, rather than bleeding the animal to extract serum, the spleen (and optionally several large lymph nodes) is removed and dissociated into single cells. If desired, the spleen cells can be screened (after removal of nonspecifically adherent cells) by applying a cell suspension to a plate or well coated with the protein antigen. B-cells producing membrane-bound immunoglobulin specific for the antigen bind to the plate and are not rinsed away with the rest of the suspension. Resulting B-cells, or all dissociated spleen cells, are then induced to fuse with myeloma cells to form hybridomas and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected Mab-secreting hybridomas are then cultured either in vitro (e.g., in tissue culture bottles or hollow fiber reactors) or in vivo (as ascites in mice).

Other methods for sustaining antibody-producing B-cell clones, such as by EBV transformation, are known.

If desired, the antibodies (whether polyclonal or monoclonal) may be labeled using conventional techniques. Suitable labels include fluorophores, chromophores, radioactive atoms (particularly ³²p and ¹²⁵I, electron-dense reagents, enzymes and ligands having specific binding partners. Enzymes are typically detected by their activity. For example, horseradish peroxidase is usually detected by its ability to convert 3,3′,5,5′-tetramethylbenzidine (TNB) to a blue pigment, quantifiable with a spectrophotometer.

A.2 In Vitro Applications of Polypeptides

Some polypeptides of the invention will have enzymatic activities that are useful in vitro. For example, the soybean trypsin inhibitor (Kunitz) family is one of the numerous families of proteinase inhibitors. It comprises plant proteins which have inhibitory activity against serine proteinases from the trypsin and subtilisin families, thiol proteinases and aspartic proteinases. Thus, these peptides find in vitro use in protein purification protocols and in therapeutic settings requiring topical application of protease inhibitors.

Delta-aminolevulinic acid dehydratase (EC 4.2.1.24) (ALAD) catalyzes the second step in the biosynthesis of heme, the condensation of two molecules of 5-aminolevulinate to form porphobilinogen and is also involved in chlorophyll biosynthesis (Kaczor et al. (1994) Plant Physiol. 1-4: 1411-7; Smith (1988) Biochem. J. 249: 423-8; Schneider (1976) Z. naturforsch. [C] 31: 55-63). Thus, ALAD proteins can be used as catalysts in synthesis of heme derivatives. Enzymes of biosynthetic pathways generally can be used as catalysts for in vitro synthesis of the compounds representing products of the pathway.

Polypeptides encoded by SDFs of the invention are engineered to provide purification reagents to identify and purify additional polypeptides that bind to them. This allows one to identify proteins that function as multimers or elucidate signal transduction or metabolic pathways. In the case of DNA binding proteins, the polypeptide are used in a similar manner to identify the DNA determinants of specific binding (S. Pierrou et al., Anal. Biochem. 229:99 (1995), S. Chusacultanachai et al., J. Biol. Chem. 274:23591 (1999), Q. Lin et al., J. Biol. Chem. 272:27274 (1997)).

II.B. Polypeptide Variants, Fragments, and Fusions

Generally, variants , fragments, or fusions of the polypeptides encoded by the maximum length sequence(MLS) can exhibit at least one of the activities of the identified domains and/or related polypeptides described in Sections (C) and (D) of The Reference tables corresponding to the MLS of interest.

II.B1 Variants

A type of variant of the native polypeptides comprises amino acid substitutions. Conservative substitutions, described above (see II.), are preferred to maintain the function or activity of the polypeptide. Such substitutions include conservation of charge, polarity, hydrophobicity, size, etc. For example, one or more amino acid residues within the sequence is substituted with another amino acid of similar polarity that acts as a functional equivalent, for example providing a hydrogen bond in an enzymatic catalysis. Substitutes for an amino acid within an exemplified sequence are preferably made among the members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Within the scope of percentage of sequence identity described above, a polypeptide of the invention may have additional individual amino acids or amino acid sequences inserted into the polypeptide in the middle thereof and/or at the N-terminal and/or C-terminal ends thereof. Likewise, some of the amino acids or amino acid sequences may be deleted from the polypeptide. Amino acid substitutions may also be made in the sequences; conservative substitutions being preferred.

One preferred class of variants are those that comprise (1) the domain of an encoded polypeptide and/or (2) residues conserved between the encoded polypeptide and related polypeptides. For this class of variants, the encoded polypeptide sequence is changed by insertion, deletion, or substitution at positions flanking the domain and/or conserved residues.

Another class of variants includes those that comprise an encoded polypeptide sequence that is changed in the domain or conserved residues by a conservative substitution.

Yet another class of variants includes those that lack one of the in vitro activities, or structural features of the encoded polypeptides. One example is polypeptides or proteins produced from genes comprising dominant negative mutations. Such a variant may comprise an encoded polypeptide sequence with non-conservative changes in a particular domain or group of conserved residues.

II.A.2 Fragments

Fragments of particular interest are those that comprise a domain identified for a polypeptide encoded by an MLS of the instant invention and variants thereof. Also, fragments that comprise at least one region of residues conserved between an MLS encoded polypeptide and its related polypeptides are of great interest. Fragments are sometimes useful as polypeptides corresponding to genes comprising dominant negative mutations.

II.A.3 Fusions

Of interest are chimeras comprising (1) a fragment of the MLS encoded polypeptide or variants thereof of interest and (2) a fragment of a polypeptide comprising the same domain. For example, an AP2 helix encoded by a MLS of the invention fused to second AP2 helix from ANT protein, which comprises two AP2 helices. The present invention also encompasses fusions of MLS encoded polypeptides, variants, or fragments thereof fused with related proteins or fragments thereof.

Definition of Domains

The polypeptides of the invention possess identifying domains as shown in The Reference tables, which indicate specific domains within the MLS encoded polypeptides. In addition, the domains within the MLS encoded polypeptide are defined by the region that exhibits at least 70% sequence identity with the consensus sequences listed in the detailed description below of each of the domains.

The majority of the protein domain descriptions given in the protein domain table are obtained from the Prosite and the Pfam websites available on the internet.

A. Activities of Polypeptides Comprising Signal Peptides

Polypeptides comprising signal peptides are a family of proteins that are typically targeted to (1) a particular organelle or intracellular compartment, (2) interact with a particular molecule or (3) for secretion outside of a host cell. Example of polypeptides comprising signal peptides include, without limitation, secreted proteins, soluble proteins, receptors, proteins retained in the ER, etc.

These proteins comprising signal peptides are useful to modulate ligand-receptor interactions, cell-to-cell communication, signal transduction, intracellular communication, and activities and/or chemical cascades that take part in an organism outside or within of any particular cell.

One class of such proteins are soluble proteins which are transported out of the cell. These proteins act as ligands that bind to receptor to trigger signal transduction or to permit communication between cells.

Another class is receptor proteins which also comprise a retention domain that lodges the receptor protein in the membrane when the cell transports the receptor to the surface of the cell. Like the soluble ligands, receptors also modulate signal transduction and communication between cells.

In addition the signal peptide itself can serve as a ligand for some receptors. An example is the interaction of the ER targeting signal peptide with the signal recognition particle (SRP). Here, the SRP binds to the signal peptide, halting translation, and the resulting SRP complex then binds to docking proteins located on the surface of the ER, prompting transfer of the protein into the ER.

A description of signal peptide residue composition is described below in Subsection IV.C. 1.

III. Methods of Modulating Polypeptide Production

It is contemplated that polynucleotides of the invention are incorporated into a host cell or in-vitro system to modulate polypeptide production. For instance, the SDFs prepared as described herein are used to prepare expression cassettes useful in a number of techniques for suppressing or enhancing expression.

An example are polynucleotides comprising sequences to be transcribed, such as coding sequences, of the present invention are inserted into nucleic acid constructs to modulate polypeptide production. Typically, such sequences to be transcribed are heterologous to at least one element of the nucleic acid construct to generate a chimeric gene or construct.

Another example of useful polynucleotides are nucleic acid molecules comprising regulatory sequences of the present invention. Chimeric genes or constructs are generated when the regulatory sequences of the invention linked to heterologous sequences in a vector construct. Within the scope of the invention are such chimeric gene and/or constructs.

Also within the scope of the invention are nucleic acid molecules, whereof at least a part or fragment of these DNA molecules are presented in the Reference and Sequence tables of the present application, and wherein the coding sequence is under the control of its own promoter and/or its own regulatory elements. Such molecules are useful for transforming the genome of a host cell or an organism regenerated from said host cell for modulating polypeptide production.

Additionally, a vector capable of producing the oligonucleotide can be inserted into the host cell to deliver the oligonucleotide.

More detailed description of components to be included in vector constructs are described both above and below.

Whether the chimeric vectors or native nucleic acids are utilized, such polynucleotides are incorporated into a host cell to modulate polypeptide production. Native genes and/or nucleic acid molecules are effective when exogenous to the host cell.

Methods of modulating polypeptide expression includes, without limitation:

Suppression methods, such as
Antisense
Ribozymes
Co-suppression
Insertion of Sequences into the Gene to be Modulated
Regulatory Sequence Modulation.

as well as Methods for Enhancing Production, such as
Insertion of Exogenous Sequences; and

Regulatory Sequence Modulation.

III.A. Suppression

Expression cassettes of the invention are used to suppress expression of endogenous genes which comprise the SDF sequence. Inhibiting expression is useful, for instance, to tailor the ripening characteristics of a fruit (Oeller et al., Science 254:437 (1991)) or to influence seed size (WO98/07842) or to provoke cell ablation (Mariani et al., Nature 357: 384-387 (1992).

As described above, a number of methods are used to inhibit gene expression in plants, such as antisense, ribozyme, introduction of exogenous genes into a host cell, insertion of a polynucleotide sequence into the coding sequence and/or the promoter of the endogenous gene of interest and the like.

III.A.1. Antisense

An expression cassette as described above transformed into host cell or plant to produce an antisense strand of RNA. For plant cells, antisense RNA inhibits gene expression by preventing the accumulation of mRNA which encodes the enzyme of interest, see, e.g., Sheehy et al., Proc. Nat. Acad. Sci. USA, 85:8805 (1988), and Hiatt et al., U.S. Pat. No. 4,801,340.

III.A.2. Ribozymes

Similarly, ribozyme constructs are transformed into a plant to cleave mRNA and down-regulate translation.

III.A.3. Co-Suppression

Another method of suppression occurs by introducing an exogenous copy of the gene to be suppressed. Introduction of expression cassettes in which a nucleic acid is configured in the sense orientation with respect to the promoter prevents the accumulation of mRNA. A detailed description of this method is described above.

III.A.4. Insertion of Sequences into the Gene to be Modulated

Yet another means of suppressing gene expression is to insert a polynucleotide into the gene of interest to disrupt transcription or translation of the gene.

Homologous recombination could be used to target a polynucleotide insert to a gene using the Cre-Lox system (A. C. Vergunst et al., Nucleic Acids Res. 26:2729 (1998), A. C. Vergunst et al., Plant Mol. Biol. 38:393 (1998), H. Albert et al., Plant J 7:649 (1995)).

In addition, random insertion of polynucleotides into a host cell genome are also used to disrupt the gene of interest (Azpiroz-Leehan et al., Trends in Genetics 13:152 (1997)). In this method, screening for clones from a library containing random insertions is preferred for identifying those that have polynucleotides inserted into the gene of interest. Such screening is performed using probes and/or primers described above based on sequences from the Reference and Sequence tables, fragments thereof, and substantially similar sequence thereto. The screening is also performed by selecting clones or any transgenic plants having a desired phenotype.

III.A.5. Regulatory Sequence Modulation

The SDFs described in the Reference and Sequence tables or polynucleotides encoding polypeptides of the Protein Group or Protein Group Matrix tables, and fragments thereof are examples of nucleotides of the invention that contain regulatory sequences that can be used to suppress or inactivate transcription and/or translation from a gene of interest as discussed in I.C.5.

III.A.6. Genes Comprising Dominant-Negative Mutations

When suppression of production of the endogenous, native protein is desired it is often helpful to express a gene comprising a dominant negative mutation. Genes comprising dominant negative mutations produce a variant polypeptide that is capable of competing with the native polypeptide, but which does not produce the native result. Consequently, over-expression of genes comprising these mutations titrate out an undesired activity of the native protein. For example, the product from a gene comprising a dominant negative mutation of a receptor is used to constitutively activate or suppress a signal transduction cascade, allowing examination of the phenotype and thus the trait(s) controlled by that receptor and pathway. Alternatively, the protein arising from the gene comprising a dominant-negative mutation is an inactive enzyme still capable of binding to the same substrate as the native protein and therefore competes with such native proteins.

Products from genes comprising dominant-negative mutations also act upon the native protein itself to prevent activity. For example, the native protein may be active only as a homo-multimer or as one subunit of a hetero-multimer. Incorporation of an inactive subunit into the multimer with native subunit(s) inhibits activity.

Thus, gene function is modulated in host cells of interest by insertion into these cells vector constructs comprising a gene comprising a dominant-negative mutation.

III.B. Enhanced Expression

Enhanced expression of a gene of interest in a host cell is accomplished by either (1) insertion of an exogenous gene; or (2) promoter modulation.

III.B.1. Insertion of an Exogenous Gene

Insertion of an expression construct encoding an exogenous gene boosts the number of gene copies expressed in a host cell.

Such expression constructs comprise genes that either encode the native protein that is of interest or that encode a variant that exhibits enhanced activity as compared to the native protein. Such genes encoding proteins of interest are constructed from the sequences from the Reference and Sequence tables, fragments thereof, and substantially similar sequence thereto.

Such an exogenous gene includes a constitutive promoter permitting expression in any cell in a host organism or a promoter that directs transcription only in particular cells or times during a host cell life cycle or in response to environmental stimuli.

III.B.2. Regulatory Sequence Modulation

The SDFs of the Reference and Sequence tables, and fragments thereof, contain regulatory sequences that are used to enhance expression of a gene of interest. For example, some of these sequences contain useful enhancer elements. In some cases, duplication of enhancer elements or insertion of exogenous enhancer elements increases expression of a desired gene from a particular promoter. As other examples, all 11 promoters require binding of a regulatory protein to be activated, while some promoters may need a protein that signals a promoter binding protein to expose a polymerase binding site. In either case, over-production of such proteins are used to enhance expression of a gene of interest by increasing the activation time of the promoter.

Such regulatory proteins are encoded by some of the sequences in the Reference and Sequence tables, fragments thereof, and substantially similar sequences thereto.

Coding sequences for these proteins are constructed as described above.

IV. Gene Constructs and Vector Construction

To use isolated SDFs of the present invention or a combination of them or parts and/or mutants and/or fusions of said SDFs in the above techniques, recombinant DNA vectors that comprise said SDFs and are suitable for transformation of cells, such as plant cells, are usually prepared. The SDF construct are made using standard recombinant DNA techniques (Sambrook et al. 1989) and is introduced to the species of interest by Agrobacterium mediated transformation or by other means of transformation (e.g. particle gun bombardment) as referenced below.

The vector backbone can be any of those typical in the art such as plasmids, viruses, artificial chromosomes, BACs, YACs, PACs and vectors of the sort described by

(a) BAC: Shizuya et al., Proc. Natl. Acad. Sci. USA 89: 8794-8797 (1992); Hamilton et al., Proc. Natl. Acad. Sci. USA 93: 9975-9979 (1996);

(b) YAC: Burke et al., Science 236:806-812 (1987);.

(c) PAC: Sternberg N. et al., Proc Natl Acad Sci U S A. January;87(1):103-7 (1990);

(d) Bacteria-Yeast Shuttle Vectors: Bradshaw et al., Nucl Acids Res 23: 4850-4856 (1995);

(e) Lambda Phage Vectors: Replacement Vector, e.g., Frischauf et al., J. Mol Biol 170: 827-842 (1983); or Insertion vector, e.g., Huynh et al., In: Glover N M (ed) DNA Cloning: A practical Approach, Vol.1 Oxford: IRL Press (1985);

(f) T-DNA gene fusion vectors Walden et al., Mol Cell Biol 1: 175-194 (1990); and

(g) Plasmid vectors: Sambrook et al., infra.

Typically, a vector comprises the exogenous gene, which in its turn comprises an SDF of the present invention to be introduced into the genome of a host cell, and which gene may be an antisense construct, a ribozyme construct chimeraplast, or a coding sequence with any desired transcriptional and/or translational regulatory sequences, such as promoters, UTRs, and 3′ end termination sequences. Vectors of the invention also include origins of replication, scaffold attachment regions (SARs), markers, homologous sequences, introns, etc.

A DNA sequence coding for the desired polypeptide, for example a cDNA sequence encoding a full length protein, are preferably combined with transcriptional and translational initiation regulatory sequences which direct the transcription of the sequence from the gene in the intended tissues of the transformed plant. For example, for over-expression, a plant promoter fragment is employed that direct transcription of the gene in all tissues of a regenerated plant. Alternatively, the plant promoter directs transcription of an SDF of the invention in a specific tissue (tissue-specific promoters) or is otherwise under more precise environmental control (inducible promoters).

If proper polypeptide production is desired, a polyadenylation region at the 3′-end of the coding region is typically included. The polyadenylation region is derived from the natural gene, from a variety of other plant genes, or from T-DNA.

The vector comprising the sequences from genes or SDF or the invention comprises a marker gene that confers a selectable phenotype on plant cells. The vector includes promoter and coding sequence, for instance. For example, the marker may encode biocide resistance, particularly antibiotic resistance, such as resistance to kanamycin, G418, bleomycin, hygromycin, or herbicide resistance, (e.g. resistance to chlorosulfuron or phosphinotricin).

IV.A. Coding Sequences

Generally, the sequence in the transformation vector and to be introduced into the genome of the host cell does not need to be absolutely identical to an SDF of the present invention. Also, it is not necessary for it to be full length, relative to either the primary transcription product or fully processed mRNA. Furthermore, the introduced sequence need not have the same intron or exon pattern as a native gene. Also, heterologous non-coding segments can be incorporated into the coding sequence without changing the desired amino acid sequence of the polypeptide to be produced.

IV.B. Promoters

As explained above, introducing an exogenous SDF from the same species or an orthologous SDF from another species is useful to modulate the expression of a native gene corresponding to that SDF of interest. Such an SDF construct is under the control of either a constitutive promoter or a highly regulated inducible promoter (e.g., a copper inducible promoter). The promoter of interest is initially either endogenous or heterologous to the species in question. When re-introduced into the genome of said species, such promoter becomes exogenous to said species. Over-expression of an SDF transgene leads to co-suppression of the homologous endogeneous sequence thereby creating some alterations in the phenotypes of the transformed species as demonstrated by similar analysis of the chalcone synthase gene (Napoli et al., Plant Cell 2:279 (1990) and van der Krol et al., Plant Cell 2:291 (1990)). If an SDF is found to encode a protein with desirable characteristics, its over-production is controlled so that its accumulation is manipulated in an organ- or tissue-specific manner utilizing a promoter having such specificity.

Likewise, if the promoter of an SDF (or an SDF that includes a promoter) is found to be tissue-specific or developmentally regulated, such a promoter is utilized to drive or facilitate the transcription of a specific gene of interest (e.g., seed storage protein or root-specific protein). Thus, the level of accumulation of a particular protein is manipulated or its spatial localization in an organ- or tissue- specific manner is altered.

IV.C Signal Peptides

SDFs of the present invention containing signal peptides are indicated in the Reference and Sequence tables. In some cases it may be desirable for the protein encoded by an introduced exogenous or orthologous SDF to be targeted (1) to a particular organelle intracellular compartment, (2) to interact with a particular molecule such as a membrane molecule or (3) for secretion outside of the cell harboring the introduced SDF. This is accomplished using a signal peptide.

Signal peptides direct protein targeting, are involved in ligand-receptor interactions and act in cell to cell communication. Many proteins, especially soluble proteins, contain a signal peptide that targets the protein to one of several different intracellular compartments. In plants, these compartments include, but are not limited to, the endoplasmic reticulum (ER), mitochondria, plastids (such as chloroplasts), the vacuole, the Golgi apparatus, protein storage vessicles (PSV) and, in general, membranes. Some signal peptide sequences are conserved, such as the Asn-Pro-Ile-Arg amino acid motif found in the N-terminal propeptide signal that targets proteins to the vacuole (Marty (1999) The Plant Cell 11: 587-599). Other signal peptides do not have a consensus sequence per se, but are largely composed of hydrophobic amino acids, such as those signal peptides targeting proteins to the ER (Vitale and Denecke (1999) The Plant Cell 11: 615-628). Still others do not appear to contain either a consensus sequence or an identified common secondary sequence, for instance the chloroplast stromal targeting signal peptides (Keegstra and Cline (1999) The Plant Cell 11: 557-570). Furthermore, some targeting peptides are bipartite, directing proteins first to an organelle and then to a membrane within the organelle (e.g. within the thylakoid lumen of the chloroplast; see Keegstra and Cline (1999) The Plant Cell 11: 557-570). In addition to the diversity in sequence and secondary structure, placement of the signal peptide is also varied. Proteins destined for the vacuole, for example, have targeting signal peptides found at the N-terminus, at the C-terminus and at a surface location in mature, folded proteins. Signal peptides also serve as ligands for some receptors.

These characteristics of signal proteins are used to more tightly control the phenotypic expression of introduced SDFs. In particular, associating the appropriate signal sequence with a specific SDF allows sequestering of the protein in specific organelles (plastids, as an example), secretion outside of the cell, targeting interaction with particular receptors, etc. Hence, the inclusion of signal proteins in constructs involving the SDFs of the invention increases the range of manipulation of SDF phenotypic expression. The nucleotide sequence of the signal peptide is isolated from characterized genes using common molecular biological techniques or is synthesized in vitro.

In addition, the native signal peptide sequences, both amino acid and nucleotide, described in the Reference and Sequence tables is used to modulate polypeptide transport. Further variants of the native signal peptides described in the Reference and Sequence tables are contemplated. Insertions, deletions, or substitutions can be made. Such variants retain at least one of the functions of the native signal peptide as well as exhibiting some degree of sequence identity to the native sequence.

Also, fragments of the signal peptides of the invention are useful and are fused with other signal peptides of interest to modulate transport of a polypeptide.

V. Transformation Techniques

A wide range of techniques for inserting exogenous polynucleotides are known for a number of host cells, including, without limitation, bacterial, yeast, mammalian, insect and plant cells. Techniques for transforming a wide variety of higher plant species are well known and described in the technical and scientific literature. See, e.g. Weising et al., Ann. Rev. Genet. 22:421 (1988); and Christou, Euphytica, v. 85, n.1-3:13-27, (1995).

DNA constructs of the invention are introduced into the genome of the desired plant host by a variety of conventional techniques. For example, the DNA construct is introduced directly into the genomic DNA of the plant cell using techniques such as electroporation and microinjection of plant cell protoplasts, or the DNA constructs are introduced directly to plant tissue using ballistic methods, such as DNA particle bombardment. Alternatively, the DNA constructs are combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria (McCormac et al., Mol. Biotechnol. 8:199 (1997); Hamilton, Gene 200:107 (1997); Salomon et al. EMBO J. 3:141 (1984); Herrera-Estrella et al. EMBO J. 2:987 (1983)).

Microinjection techniques are known in the art and are described in the scientific and patent literature. The introduction of DNA constructs using polyethylene glycol precipitation is described in Paszkowski et al. EMBO J. 3:2717 (1984). Electroporation techniques are described in Fromm et al. Proc. Natl Acad. Sci. USA 82:5824 (1985). Ballistic transformation techniques are described in Klein et al. Nature 327:773 (1987). Agrobacterium tumefaciens-mediated transformation techniques, including disarming and use of binary or co-integrate vectors, are well described in the scientific literature. See, for example Hamilton, C M., Gene 200:107 (1997); Müller et al. Mol. Gen. Genet. 207:171 (1987); Komari et al. Plant J. 10: 165 (1996); Venkateswarlu et al. Biotechnology 9:1103 (1991) and Gleave, A P., Plant Mol. Biol. 20:1203 (1992); Graves and Goldman, Plant Mol. Biol. 7:34 (1986) and Gould et al., Plant Physiology 95:426 (1991).

Transformed plant cells which are derived by any of the above transformation techniques are cultured to regenerate a whole plant that possesses the transformed genotype and thus the desired phenotype, for example seedlessness. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture in “Handbook of Plant Cell Culture,” pp. 124-176, MacMillan Publishing Company, New York, 1983; and Binding, Regeneration of plants, Plant Protoplasts, pp. 21-73, CRC Press, Boca Raton, 1988. Regeneration is also obtained from plant callus, explants, organs, or parts thereof. Such regeneration techniques are described generally in Klee et al. Ann. Rev. of plant Phys. 38:467 (1987). Regeneration of monocots (rice) is described by Hosoyama et al. (Biosci. Biotechnol. Biochem. 58:1500 (1994)) and by Ghosh et al. (J. Biotechnol. 32:1 (1994)). The nucleic acids of the invention are used to confer desired traits on essentially any plant.

Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and, Zea.

One of skill recognizes that after the expression cassette is stably incorporated in transgenic plants and confirmed to be operable, it can be introduced into other plants by sexual crossing. Any of a number of standard breeding techniques are used, depending upon the species to be crossed.

The particular sequences of SDFs identified are provided in the attached Reference and Sequence tables.

### IX. DEFINITIONS

The following terms are utilized throughout this application:

Allelic variant: An “allelic variant” is an alternative form of the same SDF, which resides at the same chromosomal locus in the organism. Allelic variations can occur in any portion of the gene sequence, including regulatory regions. Allelic variants can arise by normal genetic variation in a population. Allelic variants can also be produced by genetic engineering methods. An allelic variant can be one that is found in a naturally occurring plant, including a cultivar or ecotype. An allelic variant may or may not give rise to a phenotypic change, and may or may not be expressed. An allele can result in a detectable change in the phenotype of the trait represented by the locus. A phenotypically silent allele can give rise to a product. Alternatively spliced messages: Within the context of the current invention, “alternatively spliced messages” refers to mature mRNAs originating from a single gene with variations in the number and/or identity of exons, introns and/or intron-exon junctions.

Chimeric: The term “chimeric” is used to describe genes, as defined supra, or contructs wherein at least two of the elements of the gene or construct, such as the promoter and the coding sequence and/or other regulatory sequences and/or filler sequences and/or complements thereof, are heterologous to each other.

Constitutive Promoter: Promoters referred to herein as “constitutive promoters” actively promote transcription under most, but not necessarily all, environmental conditions and states of development or cell differentiation. Examples of constitutive promoters include the cauliflower mosaic virus (CaMV) 35S transcript initiation region and the 1′ or 2′ promoter derived from T-DNA of Agrobacterium tumefaciens, and other transcription initiation regions from various plant genes, such as the maize ubiquitin-1 promoter, known to those of skill.

Coordinately Expressed: The term “coordinately expressed,” as used in the current invention, refers to genes that are expressed at the same or a similar time and/or stage and/or under the same or similar environmental conditions.

Domain: Domains are fingerprints or signatures that can be used to characterize protein families and/or parts of proteins. Such fingerprints or signatures can comprise conserved (1) primary sequence, (2) secondary structure, and/or (3) three-dimensional conformation. Generally, each domain has been associated with either a family of proteins or motifs. Typically, these families and/or motifs have been correlated with specific in-vitro and/or in-vivo activities. A domain can be any length, including the entirety of the sequence of a protein. Detailed descriptions of the domains, associated families and motifs, and correlated activities of the polypeptides of the instant invention are described below. Usually, the polypeptides with designated domain(s) can exhibit at least one activity that is exhibited by any polypeptide that comprises the same domain(s).

Endogenous: The term “endogenous,” within the context of the current invention refers to any polynucleotide, polypeptide or protein sequence which is a natural part of a cell or organisms regenerated from said cell.

Exogenous: “Exogenous,” as referred to within, is any polynucleotide, polypeptide or protein sequence, whether chimeric or not, that is initially or subsequently introduced into the genome of an individual host cell or the organism regenerated from said host cell by any means other than by a sexual cross. Examples of means by which this can be accomplished are described below, and include Agrobacterium-mediated transformation (of dicots—e.g. Salomon et al. EMBO J. 3:141 (1984); Herrera-Estrella et al. EMBO J. 2:987 (1983); of monocots, representative papers are those by Escudero et al., Plant J. 10:355 (1996), Ishida et al., Nature Biotechnology 14:745 (1996), May et al., Bio/Technology 13:486 (1995)), biolistic methods (Armaleo et al., Current Genetics 17:97 1990)), electroporation, in planta techniques, and the like. Such a plant containing the exogenous nucleic acid is referred to here as a T₀ for the primary transgenic plant and T₁ for the first generation. The term “exogenous” as used herein is also intended to encompass inserting a naturally found element into a non-naturally found location.

Filler sequence: As used herein, “filler sequence” refers to any nucleotide sequence that is inserted into DNA construct to evoke a particular spacing between particular components such as a promoter and a coding region and may provide an additional attribute such as a restriction enzyme site.

Gene: The term “gene,” as used in the context of the current invention, encompasses all regulatory and coding sequence contiguously associated with a single hereditary unit with a genetic function (see SCHEMATIC 1). Genes can include non-coding sequences that modulate the genetic function that include, but are not limited to, those that specify polyadenylation, transcriptional regulation, DNA conformation, chromatin conformation, extent and position of base methylation and binding sites of proteins that control all of these. Genes comprised of “exons” (coding sequences), which may be interrupted by “introns” (non-coding sequences), encode proteins. A gene's genetic function may require only RNA expression or protein production, or may only require binding of proteins and/or nucleic acids without associated expression. In certain cases, genes adjacent to one another may share sequence in such a way that one gene will overlap the other. A gene can be found within the genome of an organism, artificial chromosome, plasmid, vector, etc., or as a separate isolated entity.

Gene Family: “Gene family” is used in the current invention to describe a group of functionally related genes, each of which encodes a separate protein.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence originates from, are considered heterologous to said coding sequence. Elements operatively linked in nature and—contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

Homologous gene: In the current invention, “homologous gene” refers to a gene that shares sequence similarity with the gene of interest. This similarity may be in only a fragment of the sequence and often represents a functional domain such as, examples including without limitation a DNA binding domain, a domain with tyrosine kinase activity, or the like. The functional activities of homologous genes are not necessarily the same.

Inducible Promoter: An “inducible promoter” in the context of the current invention refers to a promoter which is regulated under certain conditions, such as light, chemical concentration, protein concentration, conditions in an organism, cell, or organelle, etc. A typical example of an inducible promoter, which can be utilized with the polynucleotides of the present invention, is PARSK1, the promoter from the Arabidopsis gene encoding a serine-threonine kinase enzyme, and which promoter is induced by dehydration, abscissic acid and sodium chloride (Wang and Goodman, Plant J. 8:37 (1995)) Examples of environmental conditions that may affect transcription by inducible promoters include anaerobic conditions, elevated temperature, or the presence of light.

Intergenic region: “Intergenic region,” as used in the current invention, refers to nucleotide sequence occurring in the genome that separates adjacent genes.

Mutant gene: In the current invention, “mutant” refers to a heritable change in DNA sequence at a specific location. Mutants of the current invention may or may not have an associated identifiable function when the mutant gene is transcribed.

Orthologous Gene: In the current invention “orthologous gene” refers to a second gene that encodes a gene product that performs a similar function as the product of a first gene. The orthologous gene may also have a degree of sequence similarity to the first gene. The orthologous gene may encode a polypeptide that exhibits a degree of sequence similarity to a polypeptide corresponding to a first gene. The sequence similarity can be found within a functional domain or along the entire length of the coding sequence of the genes and/or their corresponding polypeptides.

Percentage of sequence identity: “Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity compared to a reference sequence using the programs.

Plant Promoter: A “plant promoter” is a promoter capable of initiating transcription in plant cells and can drive or facilitate transcription of a fragment of the SDF of the instant invention or a coding sequence of the SDF of the instant invention. Such promoters need not be of plant origin. For example, promoters derived from plant viruses, such as the CaMV35S promoter or from Agrobacterium tumefaciens such as the T-DNA promoters, can be plant promoters. A typical example of a plant promoter of plant origin is the maize ubiquitin-l (ubi-l)promoter known to those of skill.

Promoter: The term “promoter,” as used herein, refers to a region of sequence determinants located upstream from the start of transcription of a gene and which are involved in recognition and binding of RNA polymerase and other proteins to initiate and modulate transcription. A basal promoter is the minimal sequence necessary for assembly of a transcription complex required for transcription initiation. Basal promoters frequently include a “TATA box” element usually located between 15 and 35 nucleotides upstream from the site of initiation of transcription. Basal promoters also sometimes include a “CCAAT box” element (typically a sequence CCAAT) and/or a GGGCG sequence, usually located between 40 and 200 nucleotides, preferably 60 to 120 nucleotides, upstream from the start site of transcription.

Public sequence: The term “public sequence,” as used in the context of the instant application, refers to any sequence that has been deposited in a publicly accessible database. This term encompasses both amino acid and nucleotide sequences. Such sequences are publicly accessible, for example, on the BLAST databases on the NCBI FTP web site (accessible at ncbi.nlm.gov/blast). The database at the NCBI GTP site utilizes “gi” numbers assigned by NCBI as a unique identifier for each sequence in the databases, thereby providing a non-redundant database for sequence from various databases, including GenBank, EMBL, DBBJ, (DNA Database of Japan) and PDB (Brookhaven Protein Data Bank).

Regulatory Sequence: The term “regulatory sequence,” as used in the current invention, refers to any nucleotide sequence that influences transcription or translation initiation and rate, and stability and/or mobility of the transcript or polypeptide product. Regulatory sequences include, but are not limited to, promoters, promoter control elements, protein binding sequences, 5′ and 3′ UTRs, transcriptional start site, termination sequence, polyadenylation sequence, introns, certain sequences within a coding sequence, etc.

Related Sequences: “Related sequences” refer to either a polypeptide or a nucleotide sequence that exhibits some degree of sequence similarity with a sequence described by The Reference tables and The Sequence tables.

Scaffold Attachment Region (SAR): As used herein, “scaffold attachment region” is a DNA sequence that anchors chromatin to the nuclear matrix or scaffold to generate loop domains that can have either a transcriptionally active or inactive structure (Spiker and Thompson (1996) Plant Physiol. 110: 15-21).

Sequence-determined DNA fragments (SDFs): “Sequence-determined DNA fragments” as used in the current invention are isolated sequences of genes, fragments of genes, intergenic regions or contiguous DNA from plant genomic DNA or cDNA or RNA the sequence of which has been determined.

Signal Peptide: A “signal peptide” as used in the current invention is an amino acid sequence that targets the protein for secretion, for transport to an intracellular compartment or organelle or for incorporation into a membrane. Signal peptides are indicated in the tables and a more detailed description located below.

Specific Promoter: In the context of the current invention, “specific promoters” refers to a subset of inducible promoters that have a high preference for being induced in a specific tissue or cell and/or at a specific time during development of an organism. By “high preference” is meant at least 3-fold, preferably 5-fold, more preferably at least 10-fold still more preferably at least 20-fold, 50-fold or 100-fold increase in transcription in the desired tissue over the transcription in any other tissue. Typical examples of temporal and/or tissue specific promoters of plant origin that can be used with the polynucleotides of the present invention, are: PTA29, a promoter which is capable of driving gene transcription specifically in tapetum and only during anther development (Koltonow et al., Plant Cell 2:1201 (1990); RCc2 and RCc3, promoters that direct root-specific gene transcription in rice (Xu et al., Plant Mol. Biol. 27:237 (1995); TobRB27, a root-specific promoter from tobacco (Yamamoto et al., Plant Cell 3:371 (1991)). Examples of tissue-specific promoters under developmental control include promoters that initiate transcription only in certain tissues or organs, such as root, ovule, fruit, seeds, or flowers. Other suitable promoters include those from genes encoding storage proteins or the lipid body membrane protein, oleosin. A few root-specific promoters are noted above.

Stringency: “Stringency” as used herein is a function of probe length, probe composition (G+C content), and salt concentration, organic solvent concentration, and temperature of hybridization or wash conditions. Stringency is typically compared by the parameter Tₘ, which is the temperature at which 50% of the complementary molecules in the hybridization are hybridized, in terms of a temperature differential from Tm. High stringency conditions are those providing a condition of Tₘ−5° C. to Tₘ−10° C. Medium or moderate stringency conditions are those providing Tₘ−20° C. to Tₘ−29° C. Low stringency conditions are those providing a condition of Tₘ−40° C. to Tₘ−48° C. The relationship of hybridization conditions to Tₘ (in ° C.) is expressed in the mathematical equation
Tₘ=81.5−16.6(log₁₀[Na⁺])+0.41(% G+C)−(600/N) (1)
where N is the length of the probe. This equation works well for probes 14 to 70 nucleotides in length that are identical to the target sequence. The equation below for Tₘ of DNA-DNA hybrids is useful for probes in the range of 50 to greater than 500 nucleotides, and for conditions that include an organic solvent (formamide).
Tₘ=81.5+16.6 log {[Na⁺]/(1+0.7[Na⁺])}+0.41(% G+C)−500/L 0.63(% fornanude) (2)
where L is the length of the probe in the hybrid. (P. Tijessen, “Hybridization with Nucleic Acid Probes” in Laboratory Techniques in Biochemistry and Molecular Biology, P. C. vand der Vliet, ed., c. 1993 by Elsevier, Amsterdam.) The Tₘ of equation (2) is affected by the nature of the hybrid; for DNA-RNA hybrids Tₘ is 10-15° C. higher than calculated, for RNA-RNA hybrids Tₘ is 20-25° C. higher. Because the Tm decreases about 1° C. for each 1% decrease in homology when a long probe is used (Bonner et al., J. Mol. Biol. 81:123 (1973)), stringency conditions can be adjusted to favor detection of identical genes or related family members.

Equation (2) is derived assuming equilibrium and therefore, hybridizations according to the present invention are most preferably performed under conditions of probe excess and for sufficient time to achieve equilibrium. The time required to reach equilibrium can be shortened by inclusion of a hybridization accelerator such as dextran sulfate or another high volume polymer in the hybridization buffer.

Stringency can be controlled during the hybridization reaction or after hybridization has occurred by altering the salt and temperature conditions of the wash solutions used. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

Substantially free of: A composition containing A is “substantially free of” B when at least 85% by weight of the total A+B in the composition is A. Preferably, A comprises at least about 90% by weight of the total of A+B in the composition, more preferably at least about 95% or even 99% by weight. For example, a plant gene or DNA sequence can be considered substantially free of other plant genes or DNA sequences.

Translational start site: In the context of the current invention, a “translational start site” is usually an ATG in the cDNA transcript, more usually the first ATG. A single cDNA, however, may have multiple translational start sites.

Transcription start site: “Transcription start site” is used in the current invention to describe the point at which transcription is initiated. This point is typically located about 25 nucleotides downstream from a TFIID binding site, such as a TATA box. Transcription can initiate at one or more sites within the gene, and a single gene may have multiple transcriptional start sites, some of which may be specific for transcription in a particular cell-type or tissue.

Untranslated region (UTR): A “UTR” is any contiguous series of nucleotide bases that is transcribed, but is not translated. These untranslated regions may be associated with particular functions such as increasing mRNA message stability. Examples of UTRs include, but are not limited to polyadenylation signals, terminations sequences, sequences located between the transcriptional start site and the first exon (5′ UTR) and sequences located between the last exon and the end of the mRNA (3′ UTR).

Variant: The term “variant” is used herein to denote a polypeptide or protein or polynucleotide molecule that differs from others of its kind in some way. For example, polypeptide and protein variants can consist of changes in amino acid sequence and/or charge and/or post-translational modifications (such as glycosylation, etc).

### X. EXAMPLES

The invention is illustrated by way of the following examples. The invention is not limited by these examples as the scope of the invention is defined solely by the claims following.

### Example 1

### cDNA Preparation

A number of the nucleotide sequences disclosed in the Reference and Sequence tables herein as representative of the SDFs of the invention are obtained by sequencing genomic DNA (gDNA) and/or cDNA from corn plants grown from HYBRID SEED # 35A19, purchased from Pioneer Hi-Bred International, Inc., Supply Management, P.O. Box 256, Johnston, Iowa 50131-0256.

A number of the nucleotide sequences disclosed in the Reference and Sequence tables herein as representative of the SDFs of the invention are also obtained by sequencing genomic DNA from Arabidopsis thaliana, Wassilewskija ecotype or by sequencing cDNA obtained from mRNA from such plants as described below. A. thaliana Wassilewskija is a true breeding strain. Seeds of the plant are available from the Arabidopsis Biological Resource Center at the Ohio State University, under the accession number CS2360. Seeds of this plant were deposited under the terms and conditions of the Budapest Treaty at the American Type Culture Collection, Manassas, Va. on Aug. 31, 1999, and were assigned ATCC No. PTA-595.

Other methods for cloning full-length cDNA are described, for example, by Seki et al., Plant Journal 15:707-720 (1998) “High-efficiency cloning of Arabidopsis full-length cDNA by biotinylated Cap trapper”; Maruyama et al., Gene 138:171 (1994) “Oligo-capping a simple method to replace the cap structure of eukaryotic mRNAs with oligoribonucleotides”; and WO 96/34981.

Tissues are, or each organ is, individually pulverized and frozen in liquid nitrogen. Next, the samples are homogenized in the presence of detergents and then centrifuged. The debris and nuclei are removed from the sample and more detergents were added to the sample. The sample is centrifuged and the debris is removed. Then the sample is applied to a 2M sucrose cushion to isolate polysomes. The RNA is isolated by treatment with detergents and proteinase K followed by ethanol precipitation and centrifugation. The polysomal RNA from the different tissues are pooled according to the following mass ratios: 15/15/1 for male inflorescences, female inflorescences and root, respectively. The pooled material is then used for cDNA synthesis by the methods described below.

Starting material for cDNA synthesis for the exemplary corn cDNA clones with sequences presented in the Reference and Sequence tables is poly(A)-containing polysomal mRNAs from inflorescences and root tissues of corn plants grown from HYBRID SEED # 35A19. Male inflorescences and female (pre-and post-fertilization) inflorescences are isolated at various stages of development. Selection for poly(A) containing polysomal RNA is done using oligo d(T) cellulose columns, as described by Cox and Goldberg, “Plant Molecular Biology: A Practical Approach”, pp. 1-35, Shaw ed., c. 1988 by IRL, Oxford. The quality and the integrity of the polyA+RNAs are evaluated.

Starting material for cDNA synthesis for the exemplary Arabidopsis cDNA clones with sequences presented in the Reference and Sequence tables is polysomal RNA isolated from the top-most inflorescence tissues of Arabidopsis thaliana Wassilewskija (Ws.) and from roots of Arabidopsis thaliana Landsberg erecta (L. er.), also obtained from the Arabidopsis Biological Resource Center. Nine parts inflorescence to every part root is used, as measured by wet mass. Tissue is pulverized and exposed to liquid nitrogen. Next, the sample is homogenized in the presence of detergents and then centrifuged. The debris and nuclei are removed from the sample and more detergents are added to the sample. The sample is centrifuged and the debris removed and the sample applied to a 2M sucrose cushion to isolate polysomal RNA. Cox et al., “Plant Molecular Biology: A Practical Approach”, pp. 1-35, Shaw ed., c. 1988 by IRL, Oxford. The polysomal RNA is used for cDNA synthesis by the methods described below. Polysomal mRNA is then isolated as described above for corn cDNA. The quality of the RNA is assessed electrophoretically.

Following preparation of the mRNAs from various tissues as described above, selection of mRNA with intact 5′ ends and specific attachment of an oligonucleotide tag to the 5′ end of such mRNA is performed using either a chemical or enzymatic approach. Both techniques take advantage of the presence of the “cap” structure, which characterizes the 5′ end of most intact mRNAs and which comprises a guanosine generally methylated once, at the 7 position.

The chemical modification approach involves the optional elimination of the 2′, 3′-cis diol of the 3′ terminal ribose, the oxidation of the 2′, 3′-cis diol of the ribose linked to the cap of the 5′ ends of the mRNAs into a dialdehyde, and the coupling of the such obtained dialdehyde to a derivatized oligonucleotide tag. Further detail regarding the chemical approaches for obtaining mRNAs having intact 5′ ends is disclosed in International Application No. WO96/34981 published Nov. 7, 1996.

The enzymatic approach for ligating the oligonucleotide tag to the intact 5′ ends of mRNAs involves the removal of the phosphate groups present on the 5′ ends of uncapped incomplete mRNAs, the subsequent decapping of mRNAs having intact 5′ ends and the ligation of the phosphate present at the 5′ end of the decapped mRNA to an oligonucleotide tag. Further detail regarding the enzymatic approaches for obtaining mRNAs having intact 5′ ends is disclosed in Dumas Milne Edwards J. B. (Doctoral Thesis of Paris VI University, Le clonage des ADNc complets: difficultes et perspectives nouvelles. Apports pour le'tude de la regulation de l'expression de la tryptophane hydroxylase de rat, 20 Dec. 1993), EP 0625572 and Kato et al., Gene 150:243-250 (1994).

In both the chemical and the enzymatic approach, the oligonucleotide tag has a restriction enzyme site (e.g. an EcoRI site) therein to facilitate later cloning prqedures. Following attachment of the oligonucleotide tag to the mRNA, the integrity of the mRNA is examined by performing a Northern blot using a probe complementary to the oligonucleotide tag.

For the mRNAs joined to oligonucleotide tags using either the chemical or the enzymatic method, first strand cDNA synthesis is performed using an oligo-dT primer with reverse transcriptase. This oligo-dT primer contains an internal tag of at least 4 nucleotides, which can be different from one mRNA preparation to another. Methylated dCTP is used for cDNA first strand synthesis to protect the internal EcoRi sites from digestion during subsequent steps. The first strand cDNA is precipitated using isopropanol after removal of RNA by alkaline hydrolysis to eliminate residual primers.

Second strand cDNA synthesis is conducted using a DNA polymerase, such as Klenow fragment and a primer corresponding to the 5′ end of the ligated oligonucleotide. The primer is typically 20-25 bases in length. Methylated dCTP is used for second strand synthesis in order to protect internal EcoRI sites in the cDNA from digestion during the cloning process.

Following second strand synthesis, the full-length cDNAs are cloned into a phagemid vector, such as pBlueScript™ (Stratagene). The ends of the full-length cDNAs are blunted with T4 DNA polymerase (Biolabs) and the cDNA is digested with EcoRI. Since methylated dCTP is used during cDNA synthesis, the EcoRI site present in the tag is the only hemi-methylated site; hence the only site susceptible to EcoRI digestion. In some instances, to facilitate subcloning, an Hind III adapter is added to the 3′ end of full-length cDNAs.

The full-length cDNAs are then size fractionated using either exclusion chromatography (AcA, Biosepra) or electrophoretic separation which yields 3 to 6 different fractions. The full-length cDNAs are then directionally cloned either into pBlueScript™ using either the EcoRI and SmaI restriction sites or, when the Hind III adapter is present in the full-length cDNAs, the EcoRI and Hind III restriction sites. The ligation mixture is transformed, preferably by electroporation, into bacteria, which are then propagated under appropriate antibiotic selection.

Clones containing the oligonucleotide tag attached to full-length cDNAs are selected as follows.

The plasmid cDNA libraries made as described above are purified (e.g. by a column available from Qiagen). A positive selection of the tagged clones is performed as follows. Briefly, in this selection procedure, the plasmid DNA is converted to single stranded DNA using phage F1 gene II endonuclease in combination with an exonuclease (Chang et al., Gene 127:95 (1993)) such as exonuclease III or T7 gene 6 exonuclease. The resulting single stranded DNA is then purified using paramagnetic beads as described by Fry et al., Biotechniques 13: 124 (1992). Here the single stranded DNA is hybridized with a biotinylated oligonucleotide having a sequence corresponding to the 3′ end of the oligonucleotide tag. Preferably, the primer has a length of 20-25 bases. Clones including a sequence complementary to the biotinylated oligonucleotide are selected by incubation with streptavidin coated magnetic beads followed by magnetic capture. After capture of the positive clones, the plasmid DNA is released from the magnetic beads and converted into double stranded DNA using a DNA polymerase such as ThermoSequenase™ (obtained from Amersham Pharmacia Biotech). Alternatively, protocols such as the Gene Trapper™ kit (Gibco BRL) can be used. The double stranded DNA is then transformed, preferably by electroporation, into bacteria. The percentage of positive clones having the 5′ tag oligonucleotide is typically estimated to be between 90 and 98% from dot blot analysis.

Following transformation, the libraries are ordered in microtiter plates and sequenced. The Arabidopsis library was deposited at the American Type Culture Collection on Jan. 7, 2000 as “E-coli liba 010600” under the accession number PTA-1161.

### A. Example 2

### Southern Hybridizations

The SDFs of the invention are used in Southern hybridizations as described above. The following describes extraction of DNA from nuclei of plant cells, digestion of the nuclear DNA and separation by length, transfer of the separated fragments to membranes, preparation of probes for hybridization, hybridization and detection of the hybridized probe.

The procedures described herein are used to isolate related polynucleotides or for diagnostic purposes. Moderate stringency hybridization conditions, as defined above, are described in the present example. These conditions result in detection of hybridization between sequences having at least 70% sequence identity. As described above, the hybridization and wash conditions can be changed to reflect the desired percenatge of sequence identity between probe and target sequences that can be detected.

In the following procedure, a probe for hybridization is produced from two PCR reactions using two primers from genomic sequence of Arabidopsis thaliana. As described above, the particular template for generating the probe can be any desired template.

The first PCR product is assessed to validate the size of the primer to assure it is of the expected size. Then the product of the first PCR is used as a template, with the same pair of primers used in the first PCR, in a second PCR that produces a labeled product used as the probe.

Fragments detected by hybridization, or other bands of interest, are isolated from gels used to separate genomic DNA fragments by known methods for further purification and/or characterization.

Buffers for nuclear DNA extraction
1. 10X HB

| | 1000 ml | |
|---|---|---|
| 40 mM | 10.2 g | Spermine (Sigma S-2876) and spermidine |
| spermidine | | (Sigma S-2501) |
| 10 mM spermine | 3.5 g | Stabilize chromatin and the nuclear |
| | | membrane |
| 0.1 M EDTA | 37.2 g | EDTA inhibits nuclease |
| (disodium) | | |
| 0.1 M Tris | 12.1 g | Buffer |
| 0.8 M KCl | 59.6 g | Adjusts ionic strength for stability of nuclei |

Adjust pH to 9.5 with 10 N NaOH. It appears that there is a nuclease present in leaves.

Use of pH 9.5 appears to inactivate this nuclease.
2. 2 M sucrose (684 g per 1000 ml)
   Heat about half the final volume of water to about 50° C. Add the sucrose slowly then bring the mixture to close to final volume; stir constantly until it has dissolved. Bring the solution to volume.

3. Sarkosyl solution (lyses nuclear membranes)

| | 1000 ml |
|---|---|
| N-lauroyl sarcosine (Sarkosyl) | 20.0 g |
| 0.1 M Tris | 12.1 g |
| 0.04 M EDTA (Disodium) | 14.9 g |

Adjust the pH to 9.5 after all the components are dissolved and bring up to the proper volume.
4. 20% Triton X-100
   80 ml Triton X-100
   320 ml 1×HB (w/o β-ME and PMSF)
   Prepare in advance; Triton takes some time to dissolve
      A. Procedure

1. Prepare 1×“H” buffer (keep ice-cold during use)

| | 1000 ml |
|---|---|
| 10X HB | 100 ml |
| 2 M sucrose | 250 ml a non-ionic osmoticum |
| Water | 634 ml |

Added just before use:

| | |
|---|---|
| 100 mM PMSF* | 10 ml a protease inhibitor; protects nuclear |
| | membrane proteins |
| β-mercaptoethanol | 1 ml inactivates nuclease by reducing |
| | disulfide bonds |

*100 mM PMSF (phenyl methyl sulfonyl fluoride, Sigma P-7626) (add 0.0875 g to 5 ml 100% ethanol)

2. Homogenize the tissue in a blender (use 300-400 ml of 1×HB per blender). Be sure that you use 5-10 ml of HB buffer per gram of tissue. Blenders generate heat so be sure to keep the homogenate cold. It is necessary to put the blenders in ice periodically.
3. Add the 20% Triton X-100 (25 ml per liter of homogenate) and gently stir on ice for 20 min. This lyses plastid, but not nuclear, membranes.
4. Filter the tissue suspension through several nylon filters into an ice-cold beaker. The first filtration is through a 250-micron membrane; the second is through an 85-micron membrane; the third is through a 50-micron membrane; and the fourth is through a 20-micron membrane. Use a large funnel to hold the filters. Filtration can be sped up by gently squeezing the liquid through the filters.
5. Centrifuge the filtrate at 1200×g for 20 min. at 4° C. to pellet the nuclei.
6. Discard the dark green supernatant. The pellet will have several layers to it. One is starch; it is white and gritty. The nuclei are gray and soft. In the early steps, there may be a dark green and somewhat viscous layer of chloroplasts.
   Wash the pellets in about 25 ml cold H buffer (with Triton X-100) and resuspend by swirling gently and pipetting. After the pellets are resuspended pellet the nuclei again at 1200-1300×g. Discard the supernatant.
   Repeat the wash 3-4 times until the supernatant has changed from a dark green to a pale green. This usually happens after 3 or 4 resuspensions. At this point, the pellet is typically grayish white and very slippery. The Triton X-100 in these repeated steps helps to destroy the chloroplasts and mitochondria that contaminate the prep.
   Resuspend the nuclei for a final time in a total of 15 ml of H buffer and transfer the suspension to a sterile 125 ml Erlenmeyer flask.
7. Add 15 ml, dropwise, cold 2% Sarkosyl, 0.1 M Tris, 0.04 M EDTA solution (pH 9.5) while swirling gently. This lyses the nuclei. The solution will become very viscous.
8. Add 30 grams of CsCl and gently swirl at room temperature until the CsCl is in solution. The mixture will be gray, white and viscous.
9. Centrifuge the solution at 11,400×g at 4° C. for at least 30 min. The longer this spin is, the firmer the protein pellicle.
10. The result is typically a clear green supernatant over a white pellet, and (perhaps) under a protein pellicle. Carefully remove the solution under the protein pellicle and above the pellet. Determine the density of the solution by weighing 1 ml of solution and add CsCl if necessary to bring to 1.57 g/ml. The solution contains dissolved solids (sucrose etc) and the refractive index alone will not be an accurate guide to CsCl concentration.
11. Add 20 μl of 10 mg/ml EtBr per ml of solution.
12. Centrifuge at 184,000×g for 16 to 20 hours in a fixed-angle rotor.
13. Remove the dark red supernatant that is at the top of the tube with a plastic transfer pipette and discard. Carefully remove the DNA band with another transfer pipette. The DNA band is usually visible in room light; otherwise, use a long wave UV light to locate the band.
14. Extract the ethidium bromide (EtBr) with isopropanol saturated with water and salt.
   Once the solution is clear, extract at least two more times to ensure that all of the EtBr is gone. Be very gentle, as it is very easy to shear the DNA at this step. This extraction may take a while because the DNA solution tends to be very viscous. If the solution is too viscous, dilute it with TE.
15. Dialyze the DNA for at least two days against several changes (at least three times) of TE (10 mM Tris, 1 mM EDTA, pH 8) to remove the cesium chloride.
16. Remove the dialyzed DNA from the tubing. If the dialyzed DNA solution contains a lot of debris, centrifuge the DNA solution at least at 2500×g for 10 min. and carefully transfer the clear supernatant to a new tube. Read the A260 concentration of the DNA.
17. Assess the quality of the DNA by agarose gel electrophoresis (1% agarose gel) of the DNA. Load 50 ng and 100 ng (based on the OD reading) and compare it with known and good quality DNA. Undigested lambda DNA and a lambda-HindIII-digested DNA are good molecular weight makers.
   Protocol for Digestion of Genomic DNA
   Protocol:
1. The relative amounts of DNA for different crop plants that provide approximately a balanced number of genome equivalent is given in Table 3 below. Note that due to the size of the wheat genome, wheat DNA will be underrepresented. Lambda DNA provides a useful control for complete digestion.
2. Precipitate the DNA by adding 3 volumes of 100% ethanol. Incubate at −20° C. for at least two hours. Yeast DNA can be purchased and made up at the necessary concentration, therefore no precipitation is necessary for yeast DNA.
3. Centrifuge the solution at 11,400×g for 20 min. Decant the ethanol carefully (be careful not to disturb the pellet). Be sure that the residual ethanol is completely removed either by vacuum desiccation or by carefully wiping the sides of the tubes with a clean tissue.
4. Resuspend the pellet in an appropriate volume of water. Be sure the pellet is fully resuspended before proceeding to the next step. This may take about 30 min.
5. Add the appropriate volume of 10×reaction buffer provided by the manufacturer of the restriction enzyme to the resuspended DNA followed by the appropriate volume of enzymes. Be sure to mix it properly by slowly swirling the tubes.
6. Set-up the lambda digestion-control for each DNA that you are digesting.
7. Incubate both the experimental and lambda digests overnight at 37° C. Spin down condensation in a microfuge before proceeding.
8. After digestion, add 2 μl of loading dye (typically 0.25% bromophenol blue, 0.25% xylene cyanol in 15% Ficoll or 30% glycerol) to the lambda-control digests and load in 1% TPE-agarose gel (TPE is 90 mM Tris-phosphate, 2 mM EDTA, pH 8). If the lambda DNA in the lambda control digests are completely digested, proceed with the precipitation of the genomic DNA in the digests.
9. Precipitate the digested DNA by adding 3 volumes of 100% ethanol and incubating in −20° C. for at least 2 hours (preferably overnight).
   EXCEPTION: Arabidopsis and yeast DNA are digested in an appropriate volume; they don't have to be precipitated.
10. Resuspend the DNA in an appropriate volume of TE (e.g., 22 μl×50 blots=1100 μl) and an appropriate volume of 10×loading dye (e.g., 2.4 μl×50 blots=120 μl). Be careful in pipetting the loading dye—it is viscous. Be sure you are pipetting the correct volume.
   Table 3

Some guide points in digesting genomic DNA.

| | | | Genome | |
|---|---|---|---|---|
| | | Size | Equivalent | Amount |
| | | Relative to | to 2 μg | of DNA |
| Species | Genome Size | Arabidopsis | Arabidopsis DNA | per blot |
| Arabidopsis | 120 Mb | 1X | 1X | 2 μg |
| Brassica | 1,100 Mb | 9.2X | 0.54X | 10 μg |
| Corn | 2,800 Mb | 23.3X | 0.43X | 20 μg |
| Cotton | 2,300 Mb | 19.2X | 0.52X | 20 μg |
| Oat | 11,300 Mb | 94X | 0.11X | 20 μg |
| Rice | 400 Mb | 3.3X | 0.75X | 5 μg |
| Soybean | 1,100 Mb | 9.2X | 0.54X | 10 μg |
| Sugarbeet | 758 Mb | 6.3X | 0.8X | 10 μg |
| Sweetclover | 1,100 Mb | 9.2X | 0.54X | 10 μg |
| Wheat | 16,000 Mb | 133X | 0.08X | 20 μg |
| Yeast | 15 Mb | 0.12X | 1X | 0.25 μg |

Protocol for Southern Blot Analysis

The digested DNA samples are electrophoresed in 1% agarose gels in 1× TPE buffer. Low voltage, overnight separations are preferred. The gels are stained with EtBr and photographed.
1. For blotting the gels, first incubate the gel in 0.25 N HCl (with gentle shaking) for about 15 min.
2. Then briefly rinse with water. The DNA is denatured by 2 incubations. Incubate (with shaking) in 0.5 M NaOH in 1.5 M NaCl for 15 min.
3. The gel is then briefly rinsed in water and neutralized by incubating twice (with shaking) in 1.5 M Tris pH 7.5 in 1.5 M NaCl for 15 min.
4. A nylon membrane is prepared by soaking it in water for at least 5 min, then in 6×SSC for at least 15 min. before use. (20×SSC is 175.3 g NaCl, 88.2 g sodium citrate per liter, adjusted to pH 7.0.)
5. The nylon membrane is placed on top of the gel and all bubbles in between are removed. The DNA is blotted from the gel to the membrane using an absorbent medium, such as paper toweling and 6×SCC buffer. After the transfer, the membrane may be lightly brushed with a gloved hand to remove any agarose sticking to the surface.
6. The DNA is then fixed to the membrane by UV crosslinking and baking at 80° C. The membrane is stored at 4° C. until use.

B. Protocol for PCR Amplification of Genomic Fragments in Arabidopsis

Amplification procedures:
1. Mix the following in a 0.20 ml PCR tube or 96-well PCR plate:

| Volume | Stock | Final Amount or Conc. |
|---|---|---|
| 0.5 μl | ˜10 ng/μl genomic DNA¹ | 5 ng |
| 2.5 μl | 10X PCR buffer | 20 mM Tris, 50 mM KCl |
| 0.75 μl | 50 mM MgCl₂ | 1.5 mM |
| 1 μl | 10 pmol/μl Primer 1 (Forward) | 10 pmol |
| 1 μl | 10 pmol/μl Primer 2 (Reverse) | 10 pmol |
| 0.5 μl | 5 mM dNTPs | 0.1 mM |
| 0.1 μl | 5 units/μl Platinum Taq ™ (Life | 1 units |
| | Technologies, Gaithersburg, MD) | |
| | DNA Polymerase | |
| (to 25 μl) | Water | |

¹Arabidopsis DNA is used in the present experiment, but the procedure is a general one.

2. The template DNA is amplified using a Perkin Elmer 9700 PCR machine:

1) 94° C. for 10 min. followed by

| 2) | 3) | 4) |
|---|---|---|
| 5 cycles: | 5 cycles: | 25 cycles: |
| 94° C. - 30 sec | 94° C. - 30 sec | 94° C. - 30 sec |
| 62° C. - 30 sec | 58° C. - 30 sec | 53° C. - 30 sec |
| 72° C. - 3 min | 72° C. - 3 min | 72° C. - 3 min |

5) 72° C. for 7 min. Then the reactions are stopped by chilling to 4° C.

The procedure cna be adapted to a multi-well format if necessary.

Quanification and Dilution of PCR Products:

1. The product of the PCR is analyzed by electrophoresis in a 1% agarose gel. A linearized plasmide DNA can be used as a quantification standard (usually at 50, 100, 200, and 400 HindIII-digested Lambda DNA is useful as a molecular weight marker. The gel can be run fairly quickly; e.g., at 100 volts. The standard gel is examined to determine that the size of the PCR products is consistent with the expected size and if there are significant extra bands or smeary products in the PCR reactions.
2. The amounts of PCR products are estimated on the basis of the plasmid standard.
3. For the small number of reactions that produce extraneous bands, a small amount of DNA from bands with the correct size can be isolated by dipping a sterile 10-μl tip into the band while viewing though a UV Transilluminator. The small amount of agarose gel (with the DNA fragment) is used in the labeling reaction.
   C. Protocol for PCR-DIG-Labeling of DNA
   Solutions:
Reagents in PCR reactions (diluted PCR products, 10 × PCR Buffer, 50 mM MgCl₂, 5 U/μl Platinum Taq Polymerase, and the primers)
10 ×dNTP+DIG-11-dUTP [1:5]: (2 mM dATP, 2 mM dCTP, 2 mM dGTP, 1.65 mM dTTP, 0.35 mM DIG-11-dUTP)
10 ×dNTP+DIG-11-dUTP [1:10]: (2 mM dATP, 2 mM dCTP, 2 mM dGTP, 1.81 mM dTTP, 0.19 mM DIG-11-dUTP)
10×dNTP+DIG-11-dUTP [1:15]: (2 mM dATP, 2 mM dCTP, 2 mM dGTP, 1.875 mM dTTP, 0.125 mM DIG-11-dUTP)
TE buffer (10 mM Tris, 1 mM EDTA, pH 8)
Maleate buffer: In 700 ml of deionized distilled water, dissolve 11.61 g maleic acid and 8.77 g NaCl. Add NaOH to adjust the pH to 7.5. Bring the volume to 1 L. Stir for 15 min. and sterilize.
10% blocking solution: In 80 ml deionized distilled water, dissolve 1.16g maleic acid. Next, add NaOH to adjust the pH to 7.5. Add 10 g of the blocking reagent powder (Boehringer Mannheim, Indianapolis, Ind., Cat. no. 1096176). Heat to 60° C. while stirring to dissolve the powder. Adjust the volume to 100 ml with water. Stir and sterilize.
1% blocking solution: Dilute the 10% stock to 1 % using the maleate buffer.
Buffer 3 (100 mM Tris, 100 mM NaCl, 50 mM MgCl₂, pH9.5). Prepared from autoclaved solutions of IM Tris pH 9.5, 5 M NaCl, and 1 M MgCl₂ in autoclaved distilled water.
   Procedure:

1. PCR reactions are performed in 25 μl volumes containing:

| | |
|---|---|
| PCR buffer | 1X |
| MgCl₂ | 1.5 mM |
| 10X dNTP + DIG-11-dUTP | 1X (please see the note below) |
| Platinum Taq ™ Polymerase | 1 unit |
| 10 pg probe DNA | |
| 10 pmol primer 1 | |

| | |
|---|---|
| Note: | |
| | Use for: |
| 10X dNTP + DIG-11-dUTP (1:5) | <1 kb |
| 10X dNTP + DIG-11-dUTP (1:10) | 1 kb to 1.8 kb |
| 10X dNTP + DIG-11-dUTP (1:15) | >1.8 kb |

2. The PCR reaction uses the following amplification cycles:

1) 94° C. for 10 min. 2) 3) 4)

| 2) | 3) | 4) |
|---|---|---|
| 5 cycles: | 5 cycles: | 25 cycles: |
| 95° C. - 30 sec | 95° C. - 30 sec | 95° C. - 30 sec |
| 61° C. - 1 min | 59° C. - 1 min | 51° C. - 1 min |
| 73° C. - 5 min | 75° C. - 5 min | 73° C. - 5 min |

5) 72° C. for 8 min. The reactions are terminated by chilling to 4° C. (hold).
3. The products are analyzed by electrophoresis-in a 1% agarose gel, comparing to an aliquot of the unlabelled probe starting material.
4. The amount of DIG-labeled probe is determined as follows:

Make serial dilutions of the diluted control DNA in dilution buffer (TE: 10 mM Tris and 1 mM EDTA, pH 8) as shown in the following table:

| DIG-labeled control | | Final Conc. (Dilution |
|---|---|---|
| DNA starting conc. | Stepwise Dilution | Name) |
| 5 ng/μl | 1 μl in 49 μl TE | 100 pg/μl (A) |
| 100 pg/μl (A) | 25 μl in 25 μl TE | 50 pg/μl (B) |
| 50 pg/μl (B) | 25 μl in 25 μl TE | 25 pg/μl (C) |
| 25 pg/μl (C) | 20 μl in 30 μl TE | 10 pg/μl (D) |

a. Serial deletions of a DIG-labeled standard DNA ranging from 100 pg to 10 pg are spotted onto a positively charged nylon membrane, marking the membrane lightly with a pencil to identify each dilution.
b. Serial dilutions (e.g., 1:50, 1:2500, 1:10,000) of the newly labeled DNA probe are spotted.
c. The membrane is fixed by UV crosslinking.
d. The membrane is wetted with a small amount of maleate buffer and then incubated in 1% blocking solution for 15 min at room temp.
e. The labeled DNA is then detected using alkaline phosphatase conjugated anti-DIG antibody (Boehringer Mannheim, Indianapolis, IN, cat. no. 1093274) and an NBT substrate according to the manufacture's instruction.
f. Spot intensities of the control and experimental dilutions are then compared to estimate the concentration of the PCR-DIG-labeled probe.
   D. Prehydrization and Hybridization of Southern Blots

Solutions:

| | |
|---|---|
| 100% Formamide | purchased from Gibco |
| 20X SSC | (1X = 0.15 M NaCl, 0.015 M Na₃citrate) |
| per L: | 175 g NaCl |
| | 87.5 g Na₃citrate.2H₂0 |

20% Sarkosyl (N-lauroyl-sarcosine)
20% SDS (sodium dodecyl sulphate)
10% Blocking Reagent: In 80 ml deionized distilled water, dissolve 1.16 g maleic acid.
   Next, add NaOH to adjust the pH to 7.5. Add 10 g of the blocking reagent powder.

Heat to 60° C. while stirring to dissolve the powder. Adjust the volume to 100 ml with water. Stir and sterilize.

Prehybridization Mix:

| Final | | Volume | |
|---|---|---|---|
| Concentration | Components | (per 100 ml) | Stock |
| 50% | Formamide | 50 ml | 100% |
| 5X | SSC | 25 ml | 20X |
| 0.1% | Sarkosyl | 0.5 ml | 20% |
| 0.02% | SDS | 0.1 ml | 20% |
| 2% | Blocking Reagent | 20 ml | 10% |
| | Water | 4.4 ml | |

General Procedures:
1. Place the blot in a heat-sealable plastic bag and add an appropriate volume of prehybridization solution (30 ml/100 cm²) at room temperature. Seal the bag with a heat sealer, avoiding bubbles as much as possible. Lay down the bags in a large plastic tray (one tray can accommodate at least 4-5 bags). Ensure that the bags are lying flat in the tray so that the prehybridization solution is evenly distributed throughout the bag. Incubate the blot for at least 2 hours with gentle agitation using a waver shaker.
2. Denature DIG-labeled DNA probe by incubating for 10 min. at 98° C. using the PCR machine and immediately cool it to 4° C.
3. Add probe to prehybridization solution (25 ng/ml; 30 ml=750 ng total probe) and mix well but avoid foaming. Bubbles may lead to background.
4. Pour off the prehybridization solution from the hybridization bags and add new prehybridization and probe solution mixture to the bags containing the membrane.
5. Incubate with gentle agitation for at least 16 hours.
6. Proceed to medium stringency post-hybridization wash:
   Three times for 20 min. each with gentle agitation using 1×SSC, 1% SDS at 60° C.
   All wash solutions must be prewarmed to 60 C. Use about 100 ml of wash solution per membrane.
   To avoid background keep the membranes fully submerged to avoid drying in spots; agitate sufficiently to avoid having membranes stick to one another.
7. After the wash, proceed to immunological detection and CSPD development.
   E. Procedure for Immunological Detection with CSPD
   Solutions:
Buffer 1: Maleic acid buffer (0.1 M maleic acid, 0.15 M NaCl; adjusted to pH 7.5 with NaoH)
Washing buffer: Maleic acid buffer with 0.3% (v/v) Tween 20.
Blocking stock solution 10% blocking reagent in buffer 1. Dissolve (10×concentration): blocking reagent powder (Boehringer Mannheim, Indianapolis, Ind., cat. no. 1096176) by constantly stirring on a 65° C. heating block or heat in a microwave, autoclave and store at 4° C.
Buffer 2
(1×blocking solution): Dilute the stock solution 1:10 in Buffer 1.
Detection buffer: 0.1 M Tris, 0.1 M NaCl, pH 9.5
   Procedure:
1. After the post-hybridization wash the blots are briefly rinsed (1-5 min.) in the maleate washing buffer with gentle shaking.
2. Then the membranes are incubated for 30 min. in Buffer 2 with gentle shaking.
3. Anti-DIG-AP conjugate (Boehringer Mannheim, Indianapolis, Ind., cat. no. 1093274) at 75 mU/ml (1 :10,000) in Buffer 2 is used for detection. 75 ml of solution can be used for 3 blots.
4. The membrane is incubated for 30 min. in the antibody solution with gentle shaking.
5. The membrane are washed twice in washing buffer with gentle shaking. About 250 mls is used per wash for 3 blots.
6. The blots are equilibrated for 2-5 min in 60 ml detection buffer.
7. Dilute CSPD (1:200) in detection buffer. (This can be prepared ahead of time and stored in the dark at 4° C.).
   The following steps must be done individually. Bags (one for detection and one for exposure) are generally cut and ready before doing the following steps.
8. The blot is carefully removed from the detection buffer and excess liquid removed without drying the membrane. The blot is immediately placed in a bag and 1.5 ml of CSPD solution is added. The CSPD solution can be spread over the membrane. Bubbles present at the edge and on the surface of the blot are typically removed by gentle rubbing. The membrane is incubated for 5 min. in CSPD solution.
9. Excess liquid is removed and the membrane is blotted briefly (DNA side up) on Whatman 3MM paper. Do not let the membrane dry completely.
10. Seal the damp membrane in a hybridization bag and incubate for 10 min at 37° C. to enhance the luminescent reaction.
11. Expose for 2 hours at room temperature to X-ray film. Multiple exposures can be taken. Luminescence continues for at least 24 hours and signal intensity increases during the first hours.

### Example 3

### Microarray Experiments and Results

1. Sample Tissue Preparation

(a) Abscissic Acid (ABA)

Seeds of Arabidopsis thaliana (ecotype Wassilewskija) are sown in trays and left at 4° C. for 4 days to vernalize. They are then transferred to a growth chamber having grown 16 hr light/8 hr dark, 13,000 LUX, 70% humidity, and 20° C. and watered twice a week with 1 L of 1×Hoagland's solution. Approximately 1,000 14 day old plants are sprayed with 200-250 mls of 100 μM ABA in a 0.02% solution of the detergent Silwet L-77. Whole seedlings, including roots, are harvested within a 15 to 20 minute time period at 1 hr and 6 hr after treatment, flash-frozen in liquid nitrogen and stored at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1 -liter beakers with 100 μM ABA for treatment. Control plants are treated with water. After 6 hr and 24 hr, aerial and root tissues are separated and flash frozen in liquid nitrogen prior to storage at ⁻80° C.

(b) Ap2

Seeds of Arabidopsis thaliana (ecotype Landesberg erecta) and floral mutant apetala2 (Jofuku et al., 1994, Plant Cell 6:1211-1225) are sown in pots and left at 4° C. for two to three days to vernalize. They are then transferred to a growth chamber. Plants are grown under long-day (16 hr light, 8 hr dark) conditions 7000-8000 LUX light intensity, 70% humidity and 22° C. temperature. Inflorescences containing immature floral buds (stages 1-7; Bowman, 1994) as wel as the inflorescence meristem are harvested and flashfrozen. Polysomal polyA+RNA is isolated from tissue according to Cox and Goldberg, 1988).

(c) Arabidopsis Endosperm

mea/mea Fruits 0-10 mm

Seeds of Arabidopsis thaliana heterozygous for thefertilization—independent endosperml (fie1) [Ohad et al., 1996; ecotype Landsberg erecta (Ler)] are sown in pots and left at 4° C. for two to three days to vernalize. Kiyosue et al. (1999) subsequently determined that fie1 was allelic to the gametophytic maternal effect mutant medea (Grossniklaus et al., 1998). Imbibed seeds are then transferred to a growth chamber. Plants are grown under long-day (16 hr light: 8 hr dark) conditions, 7000-8000 LUX light intensity, 70% humidity, and 22° C. temperature. 1-2 siliques (fruits) bearing developing seeds just prior to dessication [9 days after flowering (DAF)] are selected from each plant and are hand-dissected to identify wild-type, mea/+heterozygotes, and mea/mea homozygous mutant plants. At this stage, homozygous mea/mea plants produce short siliques that contain >70% aborted seed and can be distinguished from those produced by wild-type (100% viable seed) and mea/+heterozygous (50% viable seed) plants (Ohad et al., 1996; Grossniklaus et al., 1998; Kiyosue et al., 1999). Siliques 0-10 mm in length containing developing seeds 0-9 DAF produced by homozygous mea/mea plants are harvested and flash frozen in liquid nitrogen.

Pods 0-10 mm (Control Tissue for Sample 70)

Seeds of Arabidopsis thaliana heterozygous for thefertilization—independent endosperml (fie1) [Ohad et al., 1996; ecotype Landsberg erecta (Ler)] are sown in pots and left at 4° C. for two to three days to vernalize. Kiyosue et al. (1999) subsequently determined that fie1 was allelic to the gametophytic maternal effect mutant medea (Grossniklaus et al., 1998). Imbibed seeds are then transferred to a growth chamber. Plants are grown under long-day (16 hr light: 8 hr dark) conditions, 7000-8000 LUX light intensity, 70% humidity, and 22° C. temperature. 1-2 siliques (fruits) bearing developing seeds just prior to dessication [9 days after flowering (DAF)] are selected from each plant and are hand-dissected to identify wild-type, mea/+heterozygotes, and mea/mea homozygous mutant plants. At this stage, homozygous mealmea plants produce short siliques that contain >70% aborted seed and can be distinguished from those produced by wild-type (100% viable seed) and mea/+heterozygous (50% viable seed) plants (Ohad et al., 1996; Grossniklaus et al., 1998; Kiyosue et al., 1999). Siliques 0-10 mm in length containing developing seeds 0-9 DAF produced by segregating wild-type plants are opened and the seeds removed. The remaining tissues (pods minus seed) are harvested and flash frozen in liquid nitrogen.

(d) Arabidopsis Seeds

Fruits (Pod+Seed) 0-5 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) is represented by the enclosed embryos. Description of the stages of Arabidopsis embryogenesis used in this determination were summarized by Bowman (1994). Silique lengths are then determined and used as an approximate determinant for embryonic stage. Siliques 0-5 mm in length containing post fertilization through pre-heart stage [0-72 hours after fertilization (HAF)] embryos are harvested and flash frozen in liquid nitrogen.

Fruits(Pod+Seed) 5-10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds were selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Siliques 5-10 mm in length containing heart- through early upturned-U-stage [72-120 hours after fertilization (HAF)] embryos are harvested and flash frozen in liquid nitrogen.

Fruits(Pod+Seed) >10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and were hand-dissected to determine what developmental stage(s) are represented by the enclosed embryos. Description of the stages of Arabidopsis embryogenesis used in this determination were summarized by Bowman (1994). Silique lengths are then determined and used as an approximate determinant for embryonic stage. Siliques >10 mm in length containing green, late upturned-U- stage [>120 hours after fertilization (HAF)-9 days after flowering (DAF)] embryos are harvested and flash frozen in liquid nitrogen.

Green Pods 5-10 mm (Control Tissue for Samples 72-74)

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination are summarized by Bowman (1994). Silique lengths are then determined and used as an approximate determinant for embryonic stage. Green siliques 5-10 mm in length containing developing seeds 72-120 hours after fertilization (HAF)] are opened and the seeds removed. The remaining tissues (green pods minus seed) were harvested and flash frozen in liquid nitrogen.

Green Seeds from Fruits >10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination were summarized by Bowman (1994). Silique lengths are then determined and used as an approximate determinant for embryonic stage. Green siliques >10 mm in length containing developing seeds up to 9 days after flowering (DAF)] are opened and the seeds removed and harvested and flash frozen in liquid nitrogen.

Brown Seeds from Fruits >10 mm

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination were summarized by Bowman (1994). Silique lengths are then determined and used as an approximate determinant for embryonic stage. Yellowing siliques >10 mm in length containing brown, dessicating seeds >11 days after flowering (DAF)] are opened and the seeds removed and harvested and flash frozen in liquid nitrogen.

Green/Brown Seeds from Fruits >10 mm

Seeds of Arabidopsis thaliana (ecotype Wassilewskija) are sown in pots and left at 4° C. for two to three days to vernalize. They were then transferred to a growth chamber. Plants are grown under long-day (16 hr light: 8 hr dark) conditions, 7000-8000 LUX light intensity, 70% humidity, and 22° C. temperature. 3-4 siliques (fruits) bearing developing seeds are selected from at least 3 plants and are hand-dissected to determine what developmental stage(s) are represented by the enclosed embryos. Description of the stages of Arabidopsis embryogenesis used in this determination were summarized by Bowman (1994). Silique lengths are then determined and used as an approximate determinant for embryonic stage. Green siliques >10 mm in length containing both green and brown seeds >9 days after flowering (DAF)] are opened and the seeds removed and harvested and flash frozen in liquid nitrogen.

Mature Seeds (24 Hours After Imbibition)

Mature dry seeds of Arabidopsis thaliana (ecotype Wassilewskija) are sown onto moistened filter paper and left at 4° C. for two to three days to vernalize. Imbibed seeds are then transferred to a growth chamber [16 hr light: 8 hr dark conditions, 7000-8000 LUX light intensity, 70% humidity, and 22° C. temperature], the emerging seedlings harvested after 48 hours and flash frozen in liquid nitrogen.

Mature Seeds (Dry)

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature and taken to maturity. Mature dry seeds are collected, dried for one week at 28° C., and vernalized for one week at 4° C. before use as a source of RNA.

Ovules(Ler-pi)

Seeds of Arabidopsis thaliana heterozygous for pistillata (pi) (ecotype Landsberg erecta (Ler)) are sown in pots and left at 4° C. for two to three days to vernalize. They are then transferred to a growth chamber. Plants are grown under long-day (16 hr light: 8 hr dark) conditions, 7000-8000 LUX light intensity, 76% humidity, and 24° C. temperature. Inflorescences are harvested from seedlings about 40 days old. The inflorescences are cut into small pieces and incubated in the following enzyme solution (pH 5) at room temperature for 0.5-1 hr.: 0.2% pectolyase Y-23, 0.04% pectinase, 5 mM MES, 3% Sucrose and MS salts (1900 mg/l KNO₃, 1650 mg/l NH₄NO₃, 370 mg/l MgSO₄.7 H₂O, 170 mg/l KH₂PO₄,440 mg/l CaCl₂.2 H₂O, 6.2 mg/l H₂BO₃, 15.6 mg/l MnSO₄.4 H₂O, 8.6 mg/l ZnSO₄.7 H₂O, 0.25 mg/l NaMoO₄. 2 H₂O, 0.025 mg/l CuCO₄.5 H₂O, 0.025 mg/l CoCl₂.6 H₂O, 0.83 mg/l KI, 27.8 mg/l FeSO₄. 7 H₂O, 37.3 mg/l Disodium EDTA, pH 5.8). At the end of the incubation the mixture of inflorescence material and enzyme solution is passed through a size 60 sieve and then through a sieve with a pore size of 125 μm. Ovules greater than 125 μm in diameter are collected, rinsed twice in B5 liquid medium (2500 mg/l KNO₃, 250 mg/l MgSO₄.7 H₂O, 150 mg/l NaH2PO4. H₂O, 150 mg/l CaCl₂.2 H₂O, 134 mg/l (NH4)2 CaCl₂.SO₄, 3 mg/i H₂BO₃, 10 mg/l MnSO₄. 4 H₂O, 2 ZnSO₄.7 H₂O, 0.25 mg/l NaMoO₄.2 H₂O, 0.025 mg/l CuCO₄.5 H₂O, 0.025 mg/l CoCl₂.6 H₂O, 0.75 mg/l KI, 40 mg/l EDTA sodium ferric salt, 20 g/l sucrose, 10 mg/l Thiamine hydrochloride, 1 mg/l Pyridoxine hydrochloride, 1 mg/l Nicotinic acid, 100 mg/l myo-inositol, pH 5.5)), rinsed once in deionized water and flash frozen in liquid nitrogen. The supernatant from the 125 μm sieving is passed through subsequent sieves of 50 μm and 32 μm. The tissue retained in the 32 μm sieve is collected and mRNA prepared for use as a control.

(e) Auxin Responsive (NAA)

Seeds of Arabidopsis thaliana (ecotype Wassilewskija) are sown in trays and left at 4° C. for 4 days to vernalize. They are then transferred to a growth chamber having 16 hr light/8 hr dark, 13,000 LUX, 70% humidity, 20° C. and watered twice a week with 1 L of 1×Hoagland's solution (recipe recited in Feldmann et al., (1987) Mol. Gen. Genet. 208: 1-9 and described as complete nutrient solution). Approximately 1,000 14 day old plants are spayed with 200-250 mls of 100 μM NAA in a 0.02% solution of the detergent Silwet L-77. Aerial tissues (everything above the soil line) are harvested within a 15 to 20 minute time period 1 hr and 6 hrs after treatment, flash-frozen in liquid nitrogen and stored at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1 -liter beakers with 100 μM NAA for treatment. Control plants are treated with water. After 6 hr and 24 hr, aerial and root tissues are separated and flash frozen in liquid nitrogen prior to storage at ⁻80° C.

(f) Brassinosteroid Responsive (Br, Bz)

Two separate experiments are performed, one with epi-brassinolide and one with the brassinosteroid biosynthetic inhibitor brassinazole. In the epi-brassinolide experiments, seeds of wild-type Arabidopsis thaliana (ecotype Wassilewskija) and the brassinosteroid biosynthetic mutant dwf4-1 are sown in trays and left at 4° C. for 4 days to vernalize. They are then transferred to a growth chamber having 16 hr light/8 hr dark, 11,000 LUX, 70% humidity and 22° C. temperature. Four week old plants are spayed with a 1 μM solution of epi-brassinolide and shoot parts (unopened floral primordia and shoot apical meristems) harvested three hours later. Tissue is flash-frozen in liquid nitrogen and stored at ⁻80° C.

In the brassinazole experiments, seeds of wild-type Arabidopsis thaliana (ecotype Wassilewskija) are grown as described above. Four week old plants are sprayed with a 1 μM solution of brassinazole and shoot parts (unopened floral primordia and shoot apical meristems) harvested three hours later. Tissue is flash-frozen in liquid nitrogen and stored at ⁻80° C.

In addition to the spray experiments, tissue is prepared from two different mutants; (1) a dwf4-1 knock out mutant and (2) a mutant overexpressing the dwf4-1 gene Seeds of wild-type Arabidopsis thaliana (ecotype Wassilewskija) and of the dwf4-1 knock out and overexpressor mutants are sown in trays and left at 4° C. for 4 days to vernalize. They are then transferred to a growth chamber having 16 hr light/8 hr dark, 11,000 LUX, 70% humidity and 22° C. temperature. Tissue from shoot parts (unopened floral primordia and shoot apical meristems) is flash-frozen in liquid nitrogen and stored at ⁻80° C.

Another experiment is completed with seeds of Arabidopsis thaliana (ecotype Wassilewskija) that are sown in trays and left at 4° C. for 4 days to vernalize. They are then transferred to a growth chamber. Plants are grown under long-day (16 hr light: 8 hr. dark) conditions, 13,000 LUX light intensity, 70% humidity, 20° C. temperature and watered twice a week with 1 L 1×Hoagland's solution (recipe recited in Feldmann et al., (1987) Mol. Gen. Genet. 208: 1-9 and described as complete nutrient solution). Approximately 1,000 14 day old plants are spayed with 200-250 mls of 0.1 μM Epi-Brassinolite in 0.02% solution of the detergent Silwet L-77. At 1 hr. and 6 hrs. after treatment aerial tissues are harvested within a 15 to 20 minute time period and flash-frozen in liquid nitrogen.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1 -liter beakers with 0.1 μM epi-brassinolide for treatment. Control plants are treated with distilled deionized water. After 24 hr, aerial and root tissues are separated and flash frozen in liquid nitrogen prior to storage at ⁻80° C.

(g) Cold Shock Treatment

Seeds of Arabidopsis thaliana (ecotype Wassilewskija) are sown in trays and left at 4° C. for three days to vernalize before being transferred to a growth chamber having 16 hr light/8 hr dark, 12,000-14,000 LUX, 20° C. and 70% humidity. Fourteen day old plants are transferred to a 4° C. dark growth chamber and aerial tissues are harvested 1 hour and 6 hours later. Control plants are maintained at 20° C. and covered with foil to avoid exposure to light. Tissues are flash-frozen in liquid nitrogen and stored at 80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers containing 4° C. water for treatment. Control plants are treated with water at 25° C. After 1 hr and 6 hr aerial and root tissues are separated and flash frozen in liquid nitrogen prior to storage at −80° C.

(h) Cytokinin (BA)

Seeds of Arabidopsis thaliana (ecotype Wassilewskija) are sown in trays and left at 4° C. for 4 days to vernalize. They are then transferred to a growth chamber having 16 hr light/8 hr dark, 13,000 LUX, 70% humidity, 20° C. temperature and watered twice a week with 1 L of 1X Hoagland's solution. Approximately 1,000 14 day old plants are spayed with 200-250 mls of 100 μM BA in a 0.02% solution of the detergent Silwet L-77. Aerial tissues (everything above the soil line) are harvested within a 15 to 20 minute time period 1 hr and 6 hrs after treatment, flash-frozen in liquid nitrogen and stored at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats were watered every three days for 7 days. Seedlings are carefully removed from the sand and placed in 1-liter beakers with 100 μM BA for treatment. Control plants are treated with water. After 6 hr, aerial and root tissues are separated and flash frozen in liquid nitrogen prior to storage at ⁻80° C.

(i) Drought Stress

Seeds of Arabidopsis thaliana (ecotype Wassilewskija) are sown in pots and left at 4° C. for three days to vernalize before being transferred to a growth chamber having 16 hr light/8 hr dark, 150,000-160,000 LUX, 20° C. and 70% humidity. After 14 days, aerial tissues are cut and left to dry on 3MM Whatman paper in a petri-plate for 1 hour and 6 hours. Aerial tissues exposed for 1 hour and 6 hours to 3 MM Whatman paper wetted with IX Hoagland's solution serve as controls. Tissues are harvested, flash-frozen in liquid nitrogen and stored at −80° C.

Alternatively, Arabidopsis thaliana (ecotype Wassilewskija) seed is vernalized at 4° C. for 3 days before sowing in Metromix soil type 350. Flats are placed in a growth chamber with 23° C., 16 hr light/8 hr. dark, 80% relative humidity, ˜13,000 LUX for germination and growth. Plants are watered with 1 -1.5 L of water every four days. Watering is stopped 16 days after germination for the treated samples, but continues for the control samples. Rosette leaves and stems, flowers and siliques are harvested 2d, 3d, 4d, 5d, 6d and 7d after watering is stopped. Tissue is flash frozen in liquid nitrogen and kept at ⁻80° C. until RNA is isolated. Flowers and siliques are also harvested on day 8 from plants that had undergone a 7 d drought treatment followed by 1 day of watering. Control plants (whole plants) are harvested after 5 weeks, flash frozen in liquid nitrogen and stored as above.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in empty 1-liter beakers at room temperature for treatment. Control plants are placed in water. After 1 hr, 6 hr, 12 hr and 24 hr aerial and root tissues are separated and flash frozen in liquid nitrogen prior to storage at -80° C.

(i) Flowers (Green, White or Buds)

Approximately 10 μl of Arabidopsis thaliana seeds (ecotype Wassilewskija) are sown on 350 soil (containing 0.03% marathon) and vernalized at 4° C. for 3 days. Plants are then grown at room temperature under fluorescent lighting until flowering. Flowers are harvested after 28 days in three different categories. Buds that had not opened at all and are completely green are categorized as “flower buds” (also referred to as green buds by the investigator). Buds that had started to open, with white petals emerging slightly are categorized as “green flowers” (also referred to as white buds by the investigator). Flowers that are mostly opened (with no silique elongation) with white petals completely visible are categorized as “white flowers” (also referred to as open flowers by the investigator). Buds and flowers are harvested with forceps, flash frozen in liquid nitrogen and stored at ⁻80° C. until RNA is isolated.

(k) Germination

Arabidopsis thaliana seeds (ecotype Wassilewskija) is sterilized in bleach and rinsed with sterile water. The seeds are placed in 100 mm petri plates containing soaked autoclaved filter paper. Plates are foil-wrapped and left at 4° C. for 3 nights to vernalize. After cold treatment, the foil is removed and plates are placed into a growth chamber having 16 hr light/8 hr dark cycles, 23° C., 70% relative humidity and ˜1,000 lux. Seeds are collected 1 d, 2 d, 3 d and 4 d later, flash frozen in liquid nitrogen and stored at −80° C. until RNA is isolated.

(1) Gibberillic Acid (GA)

Seeds of Arabidopsis thaliana (ecotype Wassilewskija) are sown in trays and left at 4° C. for 4 days to vernalize. They are then transferred to a growth chamber having 16 hr light/8 hr. dark, 13,000 LUX, 70% humidity, 20° C. and watered twice a week with 1 L of IX Hoagland's solution. Approximately 1,000 14 day old plants are sprayed with 200-250 mls of 100 μM gibberillic acid in a 0.02% solution of the detergent Silwet L-77. At 1 hr. and 6 hrs. after treatment, aerial tissues (everything above the soil line) are harvested within a 15 to 20 minute time period, flash-frozen in liquid nitrogen and stored at -80° C.

Alternatively, seeds of Arabidopsis thaliana (ecotype Ws) are sown in Metro-mix soil type 350 and left at 4° C. for 3 days to vernalize. They are then transferred to a growth chamber having 16 hr light/8 hr dark, 13,000 LUX, 80% humidity, 20° C. temperature and watered every four days with 1.5 L water. Fourteen (14) days after germination, plants are sprayed with 100 μM gibberillic acid or with water. Aerial tissues are harvested 1 hr 6 hrs 12 hrs and 24 hrs post-treatment, flash frozen and stored at -80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1 -liter beakers with 100 tM gibberillic acid for treatment. Control plants are treated with water. After 1 hr, 6 hr and 12 hr, aerial and root tissues were separated and flash frozen in liquid nitrogen prior to storage at -80° C.

(m) Heat Shock Treatment

Seeds of Arabidopsis thaliana (ecotype Wassilewskija) are sown in trays and left at 4° C. for three days to vernalize before being transferred to a growth chamber with 16 hr light/8 hr dark, 12,000-14,000 LUX, 70% humidity and 20° C., fourteen day old plants are transferred to a 42° C. growth chamber and aerial tissues are harvested 1 hr and 6 hr after transfer. Control plants are left at 20° c and aerial tissues are harvested. Tissues are flash-frozen in liquid nitrogen and stored at ⁻80° c.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers containing 42° C. water for treatment. Control plants are treated with water at 25° C. After 1 hr and 6 hr aerial and root tissues are separated and flash frozen in liquid nitrogen prior to storage at -80° C.

(n) Herbicide Treatment

Arabidopsis thaliana (ecotype Wassilewskija) seeds are sterilized for 5 min. with 30% bleach, 50 μl Triton in a total volume of 50 ml. Seeds are vernalized at 4° C. for 3 days before being plated onto GM agar plates at a density of about 144 seeds per plate. Plates are incubated in a Percival growth chamber having 16 hr light/8 hr dark, 80% relative humidity, 22 ° C. and 11,000 LUX for 14 days.

Plates are sprayed (˜0.5 mls/plate) with water, Finale (1.128 g/L), Glean (1.88 g/L), RoundUp (0.01 g/L) or Trimec (0.08 g/L). Tissue is collected and flash frozen in liquid nitrogen at the following time points: 0, 1, 2, 4, 8, 12, and 24 hours. Frozen tissue is stored at ⁻80° C. prior to RNA isolation.

(o) Imbibed Seed

Seeds of maize hybrid 35A (Pioneer) are sown in water-moistened sand in covered flats (10 rows, 5-6 seed/row) and covered with clear, plastic lids before being placed in a growth chamber having 16 hr light (25° C.)/8 hr dark (20° C.), 75% relative humidity and 13,000-14,00 LUX. One day after sowing, whole seeds are flash frozen in liquid nitrogen prior to storage at −80° C. Two days after sowing, embryos and endosperm are isolated and flash frozen in liquid nitrogen prior to storage at −80° C. On days 3-6, aerial tissues, roots and endosperm are isolated and flash frozen in liquid nitrogen prior to storage at ⁻80° C.

(p) Leaf Mutant 3642:

Mutant 3642 is a recessive mutation that causes abnormal leaf development. The leaves of mutant 3642 plants are characterized by leaf twisting and irregular leaf shape. Mutant 3642 plants also exhibit abnormally shaped floral organs which results in reduced fertility.

Seed segregating for the mutant phenotype are sown in Metro-mix 350 soil and grown in a Conviron growth chamber with watering by sub-irrigation twice a week. Environmental conditions are set at 20 degrees Celsius, 70% humidity with an 8 hour day, 16 hour night light regime. Plants are harvested after 4 weeks of growth and the entire aerial portion of the plant is harvested and immediately frozen in liquid nitrogen and stored at ⁻80° C. Mutant phenotype plants are harvested separately from normal phenotype plants, which serve as the control tissue.

(g) Methyl Jasmonate (MeJ)

Seeds of Arabidopsis thaliana (ecotype Wassilewskija) are sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1X Hoagland's solution. Approximately 1,000 14 day old plants are sprayed with 200-250 mls of 0.001% methyl jasmonate in a 0.02% solution of the detergent Silwet L-77. At 1 hr and 6 hrs after treatment, whole seedlings, including roots, are harvested within a 15 to 20 minute time period, flash-frozen in liquid nitrogen and stored at -80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 0.001% methyl jasmonate for treatment. Control plants are treated with water. After 24 hr, aerial and root tissues are separated and flash frozen in liquid nitrogen prior to storage at ⁻80° C.

(r) Nitric Oxide Treatment (Nanp)

Seeds of Arabidopsis thaliana (ecotype Wassilewskija) are sown in trays and left at 4° C. for three days to vernalize before being transferred to a growth chamber having 16 hr light/8 hr dark, 12,000-14,000 LUX, 20° C. and 70% humidity. Fourteen day old plants are sprayed with 5 mM sodium nitroprusside in a 0.02% Silwett L-77 solution. Control plants are sprayed with a 0.02% Silwett L-77 solution. Aerial tissues are harvested 1 hour and 6 hours after spraying, flash-frozen in liquid nitrogen and stored at ⁻80° C.

Seeds of maize hybrid 3 5A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 5 mM nitroprusside for treatment. Control plants are treated with water. After 1 hr, 6 hr and 12 hr, aerial and root tissues are separated and flash frozen in liquid nitrogen prior to storage at ⁻80° C.

(s) Nitrogen: Low to High

Arabidopsis thaliana (ecotype Wassilewskija) seeds are sown on flats containing 4 L of a 1:2 mixture of Grace Zonolite vermiculite and soil. Flats are watered with 3 L of water and vernalized at 4° C. for five days. Flats are placed in a Conviron growth chamber having 16 hr light/8 hr dark at 20° C., 80% humidity and 17,450 LUX. Flats are watered with approximately 1.5 L of water every four days. Mature, bolting plants (24 days after germination) are bottom treated with 2 L of either a control (100 mM mannitol pH 5.5) or an experimental (50 mM ammonium nitrate, pH 5.5) solution. Roots, leaves and siliques are harvested separately 30, 120 and 240 minutes after treatment, flash frozen in liquid nitrogen and stored at ⁻80° C.

Hybrid maize seed (Pioneer hybrid 35A19) are aerated overnight in deionized water. Thirty seeds are plated in each flat, which contained 4 liters of Grace zonolite vermiculite. Two liters of water are bottom fed and flats were kept in a Conviron growth chamber with 16 hr light/8 hr dark at 20° C. and 80% humidity. Flats are watered with 1 L of tap water every three days. Five day old seedlings are treated as described above with 2 L of either a control (100 mM mannitol pH 6.5) solution or 1 L of an experimental (50 mM ammonium nitrate, pH 6.8) solution. Fifteen shoots per time point per treatment are harvested 10, 90 and 180 minutes after treatment, flash frozen in liquid nitrogen and stored at ⁻80° C.

Alternatively, seeds of Arabidopsis thaliana (ecotype Wassilewskija) are left at 4° C. for 3 days to vernalize. They are then sown on vermiculite in a growth chamber having 16 hours light/8 hours dark, 12,000-14,000 LUX, 70% humidity, and 20° C. They are bottom-watered with tap water, twice weekly. Twenty-four days old plants are sprayed with either water (control) or 0.6% ammonium nitrate at 4 EL/cm of tray surface. Total shoots and some primary roots are cleaned of vermiculite, flash-frozen in liquid nitrogen and stored at ⁻80° C.

(t) Nitrogen High to Low

Wild type Arabidopsis thaliana seeds (ecotype Wassilewskija) are surface sterilized with 30% Clorox, 0.1% Triton X-100 for 5 minutes. Seeds are then rinsed with 4-5 exchanges of sterile double distilled deionized water. Seeds are vernalized at 4° C. for 2-4 days in darkness. After cold treatment, seeds are plated on modified 1×MS media (without NH4NO₃ or KNO₃), 0.5% sucrose, 0.5g/L MES pH5.7, 1% phytagar and supplemented with KNO₃ to a final concentration of 60 mM (high nitrate modified 1X MS media). Plates are then grown for 7 days in a Percival growth chamber at 22° C. with 16 hr. light/8 hr dark.

Germinated seedlings are then transferred to a sterile flask containing 50 mL of high nitrate modified 1×MS liquid media. Seedlings are grown with mild shaking for 3 additional days at 22° C. in 16 hr. light/8 hr dark (in a Percival growth chamber) on the high nitrate modified 1×MS liquid media.

After three days of growth on high nitrate modified 1×MS liquid media, seedlings are transferred either to a new sterile flask containing 50 mL of high nitrate modified 1×MS liquid media or to low nitrate modified 1×MS liquid media (containing 20 μM KNO₃). Seedlings are grown in these media conditions with mild shaking at 22° C. in 16 hr light/ 8 hr dark for the appropriate time points and whole seedlings harvested for total RNA isolation via the Trizol method (LifeTech.). The time points used for the microarray experiments are 10 min. and 1 hour time points for both the high and low nitrate modified 1×MS media.

Alternatively, seeds that are surface sterilized in 30% bleach containing 0.1 % Triton X-00 and further rinsed in sterile water, are planted on MS agar, (0.5% sucrose) plates containing 50 mM KNO₃ (potassium nitrate). The seedlings are grown under constant light (3500 LUX) at 22° C. After 12 days, seedlings are transferred to MS agar plates containing either 1 mM KNO₃ or 50 mM KNO3. Seedlings transferred to agar plates containing 50 mM KNO₃ are treated as controls in the experiment. Seedlings transferred to plates with 1mM KNO₃ are rinsed thoroughly with sterile MS solution containing 1 mM KNO₃. There are ten plates per transfer. Root tissue was collected and frozen in 15 mL Falcon tubes at various time points which included 1 hour, 2 hours, 3 hours, 4 hours, 6 hours, 9 hours, 12 hours, 16 hours, and 24 hours.

Maize 35A19 Pioneer hybrid seeds are sown on flats containing sand and grown in a Conviron growth chamber at 25° C., 16 hr light/8 hr dark, -13,000 LUX and 80% relative humidity. Plants are watered every three days with double distilled deionized water. Germinated seedlings are allowed to grow for 10 days and are watered with high nitrate modified 1×MS liquid media (see above). On day 11, young corn seedlings are removed from the sand (with their roots intact) and rinsed briefly in high nitrate modified I X MS liquid media. The equivalent of half a flat of seedlings is then submerged (up to their roots) in a beaker containing either 500 mL of high or low nitrate modified 1×MS liquid media (see above for details).

At appropriate time points, seedlings are removed from their respective liquid media, the roots separated from the shoots and each tissue type flash frozen in liquid nitrogen and stored at ⁻80° C. This is repeated for each time point. Total RNA is isolated using the Trizol method (see above) with root tissues only.

Corn root tissues isolated at the 4 hr and 16 hr time points are used for the microarray experiments. Both the high and low nitrate modified 1×MS media are used.

(u) Osmotic Stress (PEG)

Seeds of Arabidopsis thaliana (ecotype Wassilewskija) are sown in trays and left at 4° C. for three days to vernalize before being transferred to a growth chamber having 16 hr light/8 hr dark, 12,000-14,000 LUX, 20° C., and 70% humidity. After 14 days, the aerial tissues are cut and placed on 3 MM Whatman paper in a petri-plate wetted with 20% PEG (polyethylene glycol-Mr 8,000) in 1×Hoagland's solution. Aerial tissues on 3 MM Whatman paper containing 1×Hoagland's solution alone serve as the control. Aerial tissues are harvested at 1 hour and 6 hours after treatment, flash-frozen in liquid nitrogen and stored at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 20% PEG (polyethylene glycol-Mr 8,000) for treatment. Control plants are treated with water. After 1 hr and 6 hr aerial and root tissues are separated and flash frozen in liquid nitrogen prior to storage at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1 -liter beakers with 15OmM NaCl for treatment. Control plants were treated with water. After 1 hr, 6hr, and 24 hr aerial and root tissues are separated and flash frozen in liquid nitrogen prior to storage at ⁻80° C.

(v) Oxidative Stress-Hydrogen Peroxide Treatment (H₂O₂)

Seeds of Arabidopsis thaliana (ecotype Wassilewskija) are sown in trays and left at 4° C. for three days to vernalize before being transferred to a growth chamber having 16 hr light/8 hr dark, 12,000-14,000 LUX, 20° C. and 70% humidity. Fourteen day old plants are sprayed with 5 mM H₂O₂ (hydrogen peroxide) in a 0.02% Silwett L-77 solution. Control plants are sprayed with a 0.02% Silwett L-77 solution. Aerial tissues are harvested 1 hour and 6 hours after spraying, flash-frozen in liquid nitrogen and stored at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 5 mM H₂O₂ for treatment. Control plants are treated with water. After 1 hr, 6 hr and 24 hr, aerial and root tissues are separated and flash frozen in liquid nitrogen prior to storage at ⁻80° C.

(w) Protein Degradation

Arabidopsis thaliana (ecotype Wassilewskija) wild-type and 13B12-1 (homozygous) mutant seed are sown in pots containing Metro-mix 350 soil and incubated at 4° C. for four days. Vernalized seeds are germinated in the greenhouse (16 hr light/8 hr dark) over a 7 day period. Mutant seedlings are sprayed with 0.02% (active ingredient) Finale to confirm their transgenic standing. Plants were grown until the mutant phenotype (either multiple pistils in a single flower and/or multiple branching per node) is apparent. Young inflorescences immediately forming from the multiple-branched stems are cut and flash frozen in liquid nitrogen. Young inflorescences from wild-type plants grown in parallel and under identical conditions are collected as controls. All collected tissue is stored at ⁻80° C. until RNA isolation.

(x) Roots

Seeds of Arabidopsis thaliana (ecotype Wassilewskija) are sterilized in full strength bleach for less than 5 min., washed more than 3 times in sterile distilled deionized water and plated on MS agar plates. The plates are placed at 4° C. for 3 nights and then placed vertically into a growth chamber having 16 hr light/8 hr dark cycles, 23 ° C., 70% relative humidity and ˜11,000 LUX. After 2 weeks, the roots are cut from the agar, flash frozen in liquid nitrogen and stored at ⁻80° C.

(v) Root Hairless Mutants

Plants mutant at the rhl gene locus lack root hairs. This mutation is maintained as a heterozygote.

Seeds of Arabidopsis thaliana (ecotype Landsberg erecta) mutated at the rhl gene locus are sterilized using 30% bleach with 1 ul/ml 20% Triton −X 100 and then vernalized at 4° C. for 3 days before being plated onto GM agar plates. Plates are placed in growth chamber with 16 hr light/8 hr. dark, 23° C., 14,500-15,900 LUX, and 70% relative humidity for germination and growth.

After 7 days, seedlings are inspected for root hairs using a dissecting microscope. Mutants are harvested and the cotyledons removed so that only root tissue remained. Tissue is then flash frozen in liquid nitrogen and stored at ⁻80° C.

Arabidopsis thaliana (Landsberg erecta) seedlings grown and prepared as above are used as controls.

Alternatively, seeds of Arabidopsis thaliana (ecotype Landsberg erecta), heterozygous for the rhll (root hairless) mutation, are surface-sterilized in 30% bleach containing 0.1% Triton X-100 and further rinsed in sterile water. They are then vernalized at 4° C. for 4 days before being plated onto MS agar plates. The plates are maintained in a growth chamber at 24° C. with 16 hr light/8 hr dark for germination and growth. After 10 days, seedling roots that expressed the phenotype (i.e. lacking root hairs) are cut below the hypocotyl junction, frozen in liquid nitrogen and stored at −80° C. Those seedlings with the normal root phenotype (heterozygous or wt) are collected as described for the mutant and used as controls.

(z) Root Tips

Seeds of Arabidopsis thaliana (ecotype Wassilewskija) are placed on MS plates and vernalized at 4° C. for 3 days before being placed in a 25° C. growth chamber having 16 hr light/8 hr dark, 70% relative humidty and about 3 W/m². After 6 days, young seedlings are transferred to flasks containing B5 liquid medium, 1% sucrose and 0.05 mg/l indole-3-butyric acid. Flasks are incubated at room temperature with 100 rpm agitation. Media is replaced weekly. After three weeks, roots are harvested and incubated for 1 hr with 2% pectinase, 0.2% cellulase, pH 7 before straining through a #80 (Sigma) sieve. The root body material remaining on the sieve (used as the control) is flash frozen and stored at −80° C. until use. The material that passes through the #80 sieve is strained through a #200 (Sigma) sieve and the material remaining on the sieve (root tips) is flash frozen and stored at ⁻80° C. until use. Approximately 10 mg of root tips are collected from one flask of root culture.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C..), 75% relative humidity and 13,000-14,000 LUX. Covered flats are watered every three days for 8 days. Seedlings are carefully removed from the sand and the root tips (˜2 mm long) are removed and flash frozen in liquid nitrogen prior to storage at -80° C. The tissues above the root tips (˜1 cm long) are cut, treated as above and used as control tissue.

(aa) Rosette Leaves, Stems, and Siliques

Arabidopsis thaliana (ecotype Wassilewskija) seed was vernalized at 4° C. for 3 days before sowing in Metro-mix soil type 350. Flats are placed in a growth chamber having 16 hr light/8 hr dark, 80% relative humidity, 23° C. and 13,000 LUX for germination and growth. After 3 weeks, rosette leaves, stems, and siliques are harvested, flash frozen in liquid nitrogen and stored at −80° C. until use. After 4 weeks, siliques (<5 mm, 5-10 mm and >10 mm) are harvested, flash frozen in liquid nitrogen and stored at −80° C. until use. Five week old whole plants (used as controls) are harvested, flash frozen in liquid nitrogen and kept at ⁻80° C. until RNA is isolated.

(bb) Rough Sheath2-R (rs2-R) Mutants (1400-6/S-1 7)

This experiment is conducted to identify abnormally expressed genes in the shoot apex of rough sheath2-R (rs2-R) mutant plants. rs2 encodes a myb domain DNA binding protein that functions in repression of several shoot apical meristem expressed homeobox genes. Two homeobox gene targets are known for rs2 repression, rough sheath], liguleless 3. The recessive loss of function phenotype of rs2-R homozygous plants is described in Schneeberger et al. 1998, Development 125: 2857-2865.

The seed stock genetically segregates 1:1 for rs2-R/rs2-R : rs2-R/+

Preparation of tissue samples: 160 seedlings pooled from 2 and 3 week old plants grown in sand. Growth conditions; Conviron #107 at 12 hr days/12 hr night, 25° C., 75% humidity. Shoot apex was dissected to include leaf three and older.

1) rough sheath2-R homozygous (mutant) shoot apex

2) rough sheath2-R heterozygous (wild-type, control) shoot apex.

(cc) rt1

The rt1 allele is a variation of rt1 rootless1 and is recessive. Plants displaying the rt1 phenotype have few or no secondary roots.

Seed from plants segregating for rt1 are sown on sand and placed in a growth chamber having 16 hr light/8 hr dark, 13,000 LUX, 70% humidity and 20° C. temperature. Plants are watered every three days with tap water. Eleven (11) day old seedlings are carefully removed from the sand, keeping the roots intact. rt1-type seedlings are separated from their wild-type counterparts and the root tissue isolated. Root tissue from normal seedlings (control) and rt1 mutants is flash frozen in liquid nitrogen and stored at ⁻80° C. until use.

(dd) S4 Immature Buds, Inflorescence Meristem

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. Inflorescences containing immature floral buds [stages 1-12; Smyth et al., 1990] as well as the inflorescence meristem are harvested and flash frozen in liquid nitrogen

(ee) S5 Flowers (Opened)

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. Mature, unpollinated flowers [stages 12-14; Smyth et al. 1990] are harvested and flash frozen in liquid nitrogen.

(ff) S6 Siliques (All Stages)

Seeds of Arabidopsis thaliana (ecotype Wassilewskija) are sown in pots and left at 4° C. for two to three days to vernalize. They are then transferred to a growth chamber. Plants are grown under long-day (16 hr light: 8 hr dark) conditions, 7000-8000 LUX light intensity, 70% humidity, and 22° C. temperature. Siliques bearing developing seeds containing post fertilization through pre-heart stage [0-72 hours after fertilization (HAF)], heart- through early curled cotyledon stage [72-120 HAF] and late-curled cotyledon stage [>120 HAF] embryos are harvested separately and pooled prior to RNA isolation in a mass ratio of 1:1:1. The tissues are then flash frozen in liquid nitrogen. Bowman (1994) reviews and provides a description of the stages of Arabidopsis embryogenesis used.

(gi) Salicylic Acid (Sa)

Seeds of Arabidopsis thaliana (ecotype Wassilewskija) are sown in trays and left at 4° C. for 4 days to vernalize before being transferred to a growth chamber having 16 hr light/8 hr. dark, 13,000 LUX, 70% humidity, 20° C. temperature and watered twice a week with 1 L of a 1X Hoagland's solution. Approximately 1,000 14 day old plants are sprayed with 200-250 mls of 5 mM salicylic acid (solubilized in 70% ethanol) in a 0.02% solution of the detergent Silwet L-77. At 1 hr and 6 hrs after treatment, whole seedlings, including roots, are harvested within a 15 to 20 minute time period flash-frozen in liquid nitrogen and stored at ⁻80° C.

Alternatively, seeds of wild-type Arabidopsis thaliana (ecotype Columbia) and mutant CS3726 are sown in soil type 200 mixed with osmocote fertilizer and Marathon insecticide and left at 4° C. for 3 days to vernalize. Flats are incubated at room temperature with continuous light. Sixteen days post germination plants are sprayed with 2 mM SA, 0.02% SilwettL-77 or control solution (0.02% SilwettL-77. Aerial parts or flowers were harvested 1 hr, 4 hr, 6 hr, 24 hr and 3 weeks post-treatment flash frozen and stored at −80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are carefully removed from the sand and placed in 1-liter beakers with 2 mM SA for treatment. Control plants are treated with water. After 12 hr and 24 hr, aerial and root tissues are separated and flash frozen in liquid nitrogen prior to storage at ⁻80° C.

(hh) Shoot Apical Meristem (stm)

Arabidopsis thaliana (ecotype Landsberg erecta) plants mutant at the stm gene locus lack shoot meristems, produce aerial rosettes, have a reduced number of flowers per inflorescence, as well as a reduced number of petals, stamens and carpels, and is female sterile. This mutation is maintained as a heterozygote.

Seeds of Arabidopsis thaliana (ecotype Landsberg erecta) mutated at the stm locus are sterilized using 30% bleach with 1 ul/ml 20% Triton -XI 00. The seeds are vernalized at 4° C. for 3 days before being plated onto GM agar plates. Half are then put into a 22° C., 24 hr light growth chamber and half in a 24° C. 16 hr light/8 hr dark growth chamber having 14,500-15,900 LUX, and 70% relative humidity for germination and growth.

After 7 days, seedlings are examined for leaf primordia using a dissecting microscope. Presence of leaf primordia indicated a wild type phenotype. Mutants are selected based on lack of leaf primordia. Mutants are then harvested and hypocotyls removed leaving only tissue in the shoot region. Tissue is then flash frozen in liquid nitrogen and stored at -80° C.

Control tissue is isolated from 5 day old Landsberg erecta seedlings grown in the same manner as above. Tissue from the shoot region is harvested in the same manner as the stm tissue, but only contains material from the 24° C., 16 hr light/8 hr dark long day cycle growth chamber.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 8 days. Seedlings are carefully removed from the sand and the outer layers of leaf shealth removed. About 2 mm sections are cut and flash frozen in liquid nitrogen prior to storage at ⁻80° C. The tissues above the shoot apices (˜1 cm long) are cut, treated as above and used as control tissue.

(ii) Trichomes

Arabidopsis thaliana (Colombia glabrous) inflorescences are used as a control and CS8143 (hairy inflorescence ecotype) inflorescences, having increased trichomes, are used as the experimental sample.

Approximately 10 μl of each type of seed is sown on a flat of 350 soil (containing 0.03% marathon) and vernalized at 40° C. for 3 days. Plants are then grown at room temperature under florescent lighting. Young inflorescences are collected at 30 days for the control plants and 37 days for the experimental plants. Each inflorescence is cut into one-half inch (½″) pieces, flash frozen in liquid nitrogen and stored at −80° C. until RNA is isolated.

(ii) Wounding

Seeds of Arabidopsis thaliana (Wassilewskija) are sown in trays and left at 4° C. for three days to vernalize before being transferred to a growth chamber having 16 hr light/8 hr dark, 12,000-14,000 LUX, 70% humidity and 20° C. After 14 days, the leaves are wounded with forceps. Aerial tissues are harvested 1 hour and 6 hours after wounding. Aerial tissues from unwounded plants serve as controls. Tissues are flash-frozen in liquid nitrogen and stored at ⁻80° C.

Seeds of maize hybrid 35A (Pioneer) are sown in water-moistened sand in flats (10 rows, 5-6 seed/row) and covered with clear, plastic lids before being placed in a growth chamber having 16 hr light (25° C.)/8 hr dark (20° C.), 75% relative humidity and 13,000-14,000 LUX. Covered flats are watered every three days for 7 days. Seedlings are wounded (one leaf nicked by scissors) and placed in 1-liter beakers of water for treatment. Control plants are treated not wounded. After 1 hr and 6 hr aerial and root tissues are separated and flash frozen in liquid nitrogen prior to storage at ⁻80° C.

2. Microarray Hybridization Procedures

Microarray technology provides the ability to monitor mRNA transcript levels of thousands of genes in a single experiment. These experiments simultaneously hybridize two differentially labeled fluorescent cDNA pools to glass slides that have been previously spotted with cDNA clones of the same species. Each arrayed cDNA spot will have a corresponding ratio of fluorescence that represents the level of disparity between the respective mRNA species in the two sample pools. Thousands of polynucleotides can be spotted on one slide, and each experiment generates a global expression pattern.

The microarray consists of a chemically coated microscope slide, referred herein as a “chip” with numerous polynucleotide samples arrayed at a high density. The poly-L-lysine coating allows for this spotting at high density by providing a hydrophobic surface, reducing the spreading of spots of DNA solution arrayed on the slides. Glass microscope slides (Gold Seal #3010 manufactured by Gold Seal Products, Portsmouth, N.H., USA) are coated with a 0.1 %WNV solution of Poly-L-lysine (Sigma, St. Louis, Mo.).

Polynucleotides are amplified from Arabidopsis cDNA clones using insert specific probes. The resulting 100 uL PCR reactions are purified and PCR products from cDNA clones are spotted onto the poly-L-Lysine coated glass slides using an arrangement of quill-tip pins (ChipMaker 3 spotting pins; Telechem, International, Inc., Sunnyvale, Calif., USA) and a robotic arrayer (PixSys 3500, Cartesian Technologies, Irvine, Calif., USA). Slides containing maize sequences are purchased from Agilent Technology (Palo Alto, Calif. 94304).

After arraying, slides are processed through a series of steps—rehydration, UV cross-linking, blocking and denaturation—required prior to hybridization. Slides are rehydrated by placing them over a beaker of warm water (DNA face down), for 2-3 sec, to distribute the DNA more evenly within the spots, and then snap dried on a hot plate (DNA side, face up). The DNA is then cross-linked to the slides by UV irradiation (60-65mJ; 2400 Stratalinker, Stratagene, La Jolla, Calif., USA).

The Hybridization process begins with the isolation of mRNA from the two tissues (see “Isolation of total RNA” and “Isolation of mRNA”, below) in question followed by their conversion to single stranded cDNA (see “Generation of probes for hybridization”, below). The cDNA from each tissue is independently labeled with a different fluorescent dye and then both samples are pooled together. This final differentially labeled cDNA pool is then placed on a processed microarray and allowed to hybridize (see “Hybridization and ish conditions”, below).

mRNA is isolated using the Qiagen Oligotex mRNA Spin-Column protocol (Qiagen, Valencia, Calif.) or using the Stratagene Poly(A) Quik mRNA Isolation Kit (Startagene, La Jolla, Calif.).

Plasmid DNA is isolated from the following yeast clones using Qiagen filtered maxiprep kits (Qiagen, Valencia, Calif.): YAL022c(Fun26), YAL031c(Fun21), YBR032w, YDL131w, YDL182w, YDL194w, YDL196w, YDR050c and YDR116c. Plasmid DNA is linearized with either BsrBI (YAL022c(Fun26), YAL031c(Fun21), YDL131w, YDL182w, YDL194w, YDL196w, YDR050c) or AflIII (YBR032w, YDR116c) and isolated.

Generation of Probes for Hybridization

Generation of Labeled Probes for Hybridization from First-Strand cDNA

Hybridization probes are generated from isolated mRNA using an AtlasTM Glass Fluorescent Labeling Kit (Clontech Laboratories, Inc., Palo Alto, Calif., USA). This entails a two step labeling procedure that first incorporates primary aliphatic amino groups during cDNA synthesis and then couples fluorescent dye to the cDNA by reaction with the amino functional groups.

The probe is purified using a Qiagen PCR cleanup kit (Qiagen, Valencia, Calif., USA), and eluted with 100 ul EB (kit provided). The sample is loaded on a Microcon YM-30 (Millipore, Bedford, Mass., USA) spin column and concentrated to 4-5 ul in volume.

Probes for the maize microarrays are generated using the Fluorescent Linear Amplification Kit (cat. No. G2556A) from Agilent Technologies (Palo Alto, Calif.).

Maize microarrays are hybridized according to the instructions included Fluorescent Linear Amplification Kit (cat. No. G2556A) from Agilent Technologies (Palo Alto, Calif.).

The chips are scanned using a ScanArray 3000 or 5000 (General Scanning, Watertown, Mass., USA). The chips are scanned at 543 and 633nm, at 10 um resolution to measure the intensity of the two fluorescent dyes incorporated into the samples hybridized to the chips.

The images generated by scanning slides consisted of two 16-bit TIFF images representing the fluorescent emissions of the two samples at each arrayed spot. These images are then quantified and processed for expression analysis using the data extraction software Imagene ™ (Biodiscovery, Los Angeles, Calif., USA). Imagene output is subsequently analyzed using the analysis program Genespring™ (Silicon Genetics, San Carlos, Calif., USA). In Genespring, the data is imported using median pixel intensity measurements derived from Imagene output. Background subtraction, ratio calculation and normalization are all conducted in Genespring. Normalization is achieved by breaking the data in to 32 groups, each of which represented one of the 32 pin printing regions on the microarray. Groups consist of 360 to 550 spots. Each group is independently normalized by setting the median of ratios to one and multiplying ratios by the appropriate factor.

### Example 4

### AFLP Experiments and Results

Production of Samples

mRNA is prepared from 27 plant tissues. Based on preliminary cDNA-AFLP analysis with a few primer combinations, 11 plant tissues and/or pooled samples are selected. Samples are selected to give the greatest representation of unique band upon electrophoresis. The final 11 samples or pooled samples are used in the cDNA-AFLP analysis were:

| | |
|---|---|
| S1 | Dark adapted seedlings |
| S2 | Roots/Etiolated Seedlings |
| S3 | Mature leaves, soil grown |
| S4 | Immature buds, inflorescence meristem |
| S5 | Flowers opened |
| S6 | Siliques, all stages |
| S7 | Senescing leaves (just beginning to yellow) |
| S8 | Callus Inducing medium |
| | Callus shoot induction |
| | Callus root induction |
| S9 | Wounding |
| | Methyl-jasmonate-treated |
| S10 | Oxidative stress |
| | Drought stress |
| | Oxygen Stress-flooding |
| S11 | Heat treated light grown seedling |
| | Cold treated light grown seedlings |

cDNA from each of the 11 samples is digested with two restriction endonucleases, namely TaqI and MseI. TaqI and MseI adapters are then ligated to the restriction enzyme fragments. Using primers to these adapters that are specific in sequence (i.e. without extensions), the restriction fragments are subjected to cycles of non-radioactive pre-amplification.

Selective PCR

In order to limit the number of fragments or bands on each lane of the AFLP gel, fragments are subjected to another round of selective radioactive polymerase chain amplification. The TaqI primers used in this amplification are 5′-labelled with P³³. For these amplifications, the TaqI primers have two extra nucleotides at their 3′ end and the MseI primers have three extra nucleotides at their 3′ end. This results in 16 primer designs for the TaqI primer and 64 primer designs for the MseI primer. Altogether, this gives rise to a total of 1024 primer designs. Fragments generated in this selective amplification protocol are run with labeled molecular weight markers on polyacrylamide gels to separate fragments in the size range of 100-600 nucleotides.

Following gel electrophoresis, profiles are analyzed with a phosphoimager. From these images, electronic files, giving the mobilities of all bands on the gels and their intensities in each of the samples, are compiled.

All unique bands are cut out of the gels. The gel pieces are placed in 96 well plates for elution and their plate designation linked to their electrophoretic mobilities recorded in the electronic files. The eluted fragments are then subjected to another round of amplification, this time using reamplification primers (see below). After amplification, DNA fragments are sequenced.

A computer database is established linking the mobilities of all the bands observed on the cDNA-AFLP gels with the sequence of the correspondingly isolated fragment. The sequence allows for identification of the gene from which the cDNA-AFLP fragment is derived, allowing for a linkage of band mobility with the transcript of a specific gene. Also linked to the band mobilities are their intensities recorded for each of the eleven samples used in constructing the database.

This cDNA-AFLP analysis with TaqllMseI and 1024 primer combinations is repeated using the enzymes NlalIl in place of TaqI, and Csp6I in place of MseI.

Using the Database for the Transcript Profiling of Experimental Samples

Experimental Samples are subjected to cDNA-AFLP as described above, resulting in electronic files recording band mobilities and intensities. Through use of the database established above, band mobilities are linked to specific cDNAs, and therefore genes. Furthermore, the linkage with the intensities in the respective samples allows for the quantification of specific cDNAs in these samples, and thus the relative concentration of specific transcripts in the samples, indicating the level to which specific genes are expressed.

Reamplification primers 99G24

CGCCAGGGTTTTCCCAGTCACGAC|ACGACTCACT|
gatgagtcctgagtaa|

M13 forward+10 MseI+0

99G20

AGCGGATAACAATTTCACACAGGA|CACACTGGTA|
tagactgcgtaccga|

M13 reverse+10 TaqI+0

Purification of the Reamplifiction reaction before sequencing

5 μl reamplification reaction

0,25 μl 10 × PCR buffer

0,33 μl Shrimp Alkaline Phosphatase (Amersham Life Science)

0,033 μl Exonuclease I (USB)

0,297 μl SAP dilution buffer

1,59 μl MQ

7.5 μl total

30′ 37° C.

10′ 80° C.

4° C.

Sample Preparation

S1: Dark adapted seedlings: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 8 days, the seedlings are foil-wrapped and harvested after two days.

S2: Roots/Etiolated seedlings: Seeds of Arabidopsis thaliana (wassilewskija) are germinated on solid germination media (1×MS salts, 1×MS vitamins, 20g/L sucrose, 50 mg/L MES pH 5.8) in the dark. Tissues are harvested 14 days later.

S3: Mature leaves, soil grown: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. Leaves are harvested 17 days later from plants that have not yet bolted.

S4:l Immature buds, inflorescence meristem: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark.

S5: Flowers opened: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark.

S6: Siliques, all stages: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark.

S7: Senescing leaves (just beginning to yellow): Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. When the plant has leaves that are less than 50% yellow, the leaves that are just beginning to yellow are harvested.

S8:

Callus Inducing Medium: Seeds of Arabidopsis thaliana (wassilewskija) are surface sterilized (1 min-75% Ethanol, 6 min-bleach 100%+Tween 20, rinse) and incubated on MS medium containing 2,4-Dichlorophenoxyacetic acid (2,4-D) 1 mg/l and Kinetin 1 mg/l in the dark for 3 weeks to generate primary callus.

Hypocotyls and roots of the seedling are swollen after a week after incubation in this callus induction medium and subsequently callus is initiated from these swollen areas.

Callus shoot induction: Primary calluses are transferred to the fresh callus induction medium for another 2 weeks growth to generate secondary callus. Secondary callus is transferred to shoot induction medium containing MS basal medium and Benzyladenine (BA) 2 mg/l and Naphthaleneacetic acid (NAA) ).1 mg/l for 2 weeks growth in the light before it is harvested and frozen and sent to Keygene. Many shoot meristems are observed under the microscope.

Callus root induction: Secondary calluses is transferred to root induction medium containing MS basal medium, sucrose 1% and Indolebutyric acid (IBA) 0.05 mg/l in the dark. Many root primordia are observed under microscope after 10 days in the root induction medium. Those callus tissue are harvested and frozen and sent to Keygene.

S9:

Wounding: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 20 days, leaves of plants are wounded with pliers. Wounded leaves are harvested 1 hour and 4 hours after wounding.

Methyl jasmonate treatment: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 13 days, plants are sprayed with 0.001% methyl jasmonate. Leaves are harvested 1.5 hours and 6 hours after spraying

S10:

Oxidative stress: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 24 days, a few leaves are inoculated with a mixture of 2.5 mM D-glucose, 2.5 U/mL glucose oxidase in 20 mM sodium phosphate buffer pH 6.5. After an hour, 3 hours, or 5 hours after inoculation, whole plant, except for the inoculated leaves, is harvested. This sample is mixed with sample from plants that are sitting in full sun (152,000 LUX) for 2 hours or four hours.

Drought stress: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 20 days, aerial tissues are harvested and left to dry in 3MM Whatman paper for I hour or 4 hours.

Oxygen stress: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 21 days, the plant is flooded by immersing its pot in a beaker of tap water. After 6 days, the upper tissues are harvested.

S11: Heat-treated light grown seedlings: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. Over a 5 hour period, the temperature is raised to 42° C. at the rate of approximately 4° C. per hour. After 1 hour at 42° C., the aerial tissues re collected. This sample is mixed with an equal volume of sample that has gone through a heat-recovery treatment namely bringing down the temperature to 22° C. from 42° C. over a 5 hour period at the rate of 4° C. per hour.

Cold-treated light grown seedlings: Seeds of Arabidopsis thaliana (wassilewskija) are sown in pots and left at 4° C. for two to three days to vernalize. They are transferred to a growth chamber after three days. The intensity of light in the growth chamber is 7000-8000 LUX, temperature is 22° C., with 16 h light and 8 h dark. After 18 days, the plant is transferred to 4° C. for an hour before the aerial tissues are harvested. This sample is mixed with aerial tissues from another plant that is transferred to 4° C. for 27 hours before being harvested.

Analysis of Data:

Intensity: The intensity of the band corresponds to the value in each lane marked S1, S2 etc. P-values: The data shows P- values of each of the samples 1-11. P-values are calculated using the following formula 2* (1 -NORMDIST(ABS(Sx-AVERAGE(of S1 to S11, not including Sx))/STDEV(of S1 to S11 not including Sx),0, 1, TRUE)) using Excel functions.

The equivalent mathematical formula of P-value is as follows:

$$\int φ ( x ) ⅆ x , intergrated from a to ∞ , \\ where φ ( x ) is a normal distribution \text{:}$$

$$where a = \vert \underline{Sx - μ} \vert σ ( S 1 … S 11 , not including Sx ) ;$$

$$where$$

$$\begin{matrix}μ = is the average of the intensities of all samples except Sx , \\ = \frac{( \sum S 1 … Sn ) - Sx}{n - 1}\end{matrix}$$

where σ(S1 . . . S11, not including Sx)=the standard deviation of all sample intensities except Sx.

Results:

The results are shown in the MA tables.

### Example 5

### Transformation of Carrot Cells

Transformation of plant cells can be accomplished by a number of methods, as described above. Similarly, a number of plant genera can be regenerated from tissue culture following transformation. Transformation and regeneration of carrot cells as described herein is illustrative.

Single cell suspension cultures of carrot (Daucus carota) cells are established from hypocotyls of cultivar Early Nantes in B₅ growth medium (O. L. Gamborg et al., Plant Physiol. 45:372 (1970)) plus 2,4-D and 15 mM CaCl₂ (B₅-44 medium) by methods known in the art. The suspension cultures are subcultured by adding 10 ml of the suspension culture to 40 ml of B₅-44 medium in 250 ml flasks every 7 days and are maintained in a shaker at 150 rpm at 27 ° C. in the dark.

The suspension culture cells are transformed with exogenous DNA as described by Z. Chen et al. Plant Mol. Bio. 36:163 (1998). Briefly, 4-days post-subculture cells are incubated with cell wall digestion solution containing 0.4 M sorbitol, 2% driselase, 5mM MES (2-[N-Morpholino] ethanesulfonic acid) pH 5.0 for 5 hours. The digested cells are pelleted gently at 60 xg for 5 min. and washed twice in W5 solution containing 154 mM NaCl, 5 mM KCl, 125 mM CaCl₂ and 5 mM glucose, pH 6.0. The protoplasts are suspended in MC solution containing 5 mM MES, 20 mM CaCl₂, 0.5 M mannitol, pH 5.7 and the protoplast density is adjusted to about 4×10⁶ protoplasts per ml.

15-60 μg of plasmid DNA is mixed with 0.9 ml of protoplasts. The resulting suspension is mixed with 40% polyethylene glycol (MW 8000, PEG 8000), by gentle inversion a few times at room temperature for 5 to 25 min. Protoplast culture medium known in the art is added into the PEG-DNA-protoplast mixture. Protoplasts are incubated in the culture medium for 24 hour to 5 days and cell extracts can be used for assay of transient expression of the introduced gene. Alternatively, transformed cells can be used to produce transgenic callus, which in turn can be used to produce transgenic plants, by methods known in the art. See, for example, Nomura and Komamine, Plt. Phys. 79:988-991 (1985), Identification and Isolation of Single Cells that Produce Somatic Embryos in Carrot Suspension Cultures.

### Example 6

### Phenotype Screens and Results

A: Triparental Mating and Vacuum Infiltration Transformation of Plants

Standard laboratory techniques are as described in Sambrook et al. (1989) unless otherwise stated. Single colonies of Agrobacterium C58C1Rif, E. coli helper strain HB101 and the E. coli strain containing the transformation construct to be mobilized into Agrobacterium areseparately inoculated into appropriate growth media and stationary cultures produced. 100 μl of each of the three cultures are mixed gently, plated on YEB (5 g Gibco beef extract, 1 g Bacto yeast extract, 1 g Bacto peptone, 5 g sucrose, pH 7.4) solid growth media and incubated overnight at 28° C. The bacteria from the triparental mating are collected in 2 ml of lambda buffer (20 mM Tris (pH 7.5), 100 mM NaCl, 10 mM MgCl₂) and serial dilutions made. An aliquot of the each dilution is then plated and incubated for 2 days at 28° C. on YEB plates supplemented with 100 μg/ml rifampicin and 100 μg/ml carbenicillin for calculation of the number of acceptor cells and on YEB plates supplemented with 100 μg/ml rifampicin, 100 μg/ml carbenicillin and 100 jig/ml spectinomycin for selection of transconjugant cells. The cointegrate structure of purified transconjugants is verified via Southern blot hybridization.

A transconjugant culture is prepared for vacuum infiltration by innoculating 1 ml of a stationary culture arising from a single colony into liquid YEB media and incubating at 28° C. for approximately 20 hours with shaking (220 rpm) until the OD taken at 600 nm was 0.8-1.0. The culture is then pelleted (8000 rpm, 10 min, 4° C. in a Sorvall SLA 3000 rotor) and the bacteria resuspended in infiltration medium (0.5×MS salts, 5% w/v sucrose, 10 gg/l BAP, 200 μl/l Silwet L-77, pH 5.8) to a final OD₆₀₀ of 1.0. This prepared transconjugant culture is used within 20 minutes of preparation.

Wild-type plants for vacuum infiltration are grown in 4-inch pots containing Metromix 200 and Osmocote. Briefly, seeds of Arabidopsis thaliana (ecotype Wassilewskija) are sown in pots and left at 4° C. for two to four days to vernalize. They are then transferred to 22-25° C. and grown under long-day (16 hr light: 8 hr dark) conditions, sub-irrigated with water. After bolting, the primary inflorescence is removed and, after four to eight days, the pots containing the plants are inverted in the vacuum chamber to submerge all of the plants in the prepared transconjugant culture. Vacuum is drawn for two minutes before pots are removed, covered with plastic wrap and incubated in a cool room under darkness or very low light for one to two days. The plastic wrap is then removed, the plants returned to their previous growing conditions and subsequently produced (Tl) seed collected.

B: Selection of T-DNA Insertion Lines

Approximately 10,750 seeds from the initial vacuum infiltrated plants are sown per flat of Metromix 350 soil. Flats are vernalized for four to five days at 4° C. before being transferred to 22-25° C. and grown under long-day (16 hr light: 8 hr dark) conditions, sub-irrigated with water. Approximately seven to ten days after germination, the (Tl) seedlings are sprayed with 0.02% Finale herbicide (AgrEvo). After another five to seven days, herbicide treatment is repeated. Herbicide resistant Ti plants are allowed to self-pollinate and T2 seed are collected from each individual. In the few cases where the T1 plant produced few seed, the T2 seed is planted in bulk, the T2 plants allowed to self-pollinate and T3 seed collected.

C: Phenotype Screening

Approximately 40 seed from each T2 (or T3) line are planted in a 4-inch pot containing either Sunshine mix or Metromix 350 soil. Pots are vernalized for four to five days at 4° C. before being transferred to 22-25° C. and grown under long-day (16 hr light: 8 hr dark) conditions, sub-irrigated with water. A first phenotype screen is conducted by visually inspecting the seedlings five to seven days after germination and aberrant phenotypes noted. Plants are then sprayed with Finale herbicide within four days (i.e. about seven to nine days after germination). The second visual screen is conducted on surviving T2 (or T3) plants about sixteen to seventeen days after germination and the final screen was conducted after the plants have bolted and formed siliques. Here, the third and fourth green siliques are collected and aberrant phenotypes noted. The Knock-in Table contains descriptions of identified phenotypes.

Alternatively, seed are surface sterilized and transferred to agar solidified medium containing Murashige and Skoog salts (1×), 1% sucrose (wt/v) pH 5.7 before autoclaving. Seed re cold treated for 48 hours and transferred to long days [16 hours light and 8 hours dark], 25° C. Plants are screened at 5 and 10 days.

In another screen, seed are surface sterilized and transferred to agar solidified medium containing Murashige and Skoog salts (1×), and combinations of various nitrogen and sucrose amounts as specified below::

Medium 1: no sucrose, 20.6 mM NH₄NO₃, 18.8 mM KNO₃;

Medium 2: 0.5% sucrose, 20.6 mM NH₄NO3, 18.8 mM KNO₃;

Medium 3: 3% sucrose, 20.6 mM NH₄NO₃, 18.8 mM KNO₃;

Medium 4: no sucrose, 20.6 μM NH₄NO₃, 18.8 μM KNO₃;

Medium 5: 0.5% sucrose, 20.6 μM NH₄NO₃, 18.8 μM KNO₃; and

Medium 6: 3% sucrose, 20.6 μM NH₄NO₃, 18.8 μM KNO₃.

The 0.5% sucrose is the control concentration for the sucrose. The low nitrogene, 20.6 μM NH₄NO₃, 18.8 tM KNO₃, is the control for the nitrogen. Seed are cold treated for 48 hours and transferred to long days [16 hours light and 8 hours dark], 25° C. Plants are screened at 2, 5, and 10 days.

D: Tail-PCR and Fragment Sequencing

Rosette leaves are collected from each putative mutant and crushed between parafilm and FTA paper (Life Technologies). Two 2 mm² hole punches are isolated from each FTA sample and washed according to the manufacturer's instructions by vortexing with 200 ul of the provided FTA purification reagent. The FTA reagent is removed and the washing procedure repeated two more times. The sample is then washed twice with 200 ul of FTA TE (10 mM Tris, 0.1 mM EDTA, pH 8.0) and vortexing prior to PCR.

Primers used for TAIL-PCR are as follows:

AD2: 5′ NGTCGASWGANAWGAA 3′

(128-fold degeneracy) S=G or C, W=A or T, and N=A, G, C, or T

LB1: 5′ GTTTAACTGCGGCTCAACTGTCT 3′
LB2: 5′ CCCATAGACCCTTACCGCTTTAGTT 3′
LB3: 5′ GAAAGAAAAAGAGGTATAACTGGTA 3′

The extent to which the left and right borders of the T-DNA insert are intact is measured for each line by PCR. The following components are mixed for PCR: 12 mm² FTA sample, 38.75 μl distilled water, 5 μl 10× Platinum PCR buffer (Life Technologies), 2 μl 50 mM MgCl₂, 1 μl 10 mM dNTPs, 1 μl 10 μM primer LB1 (or RB1 for analysis of the right border), 1 μl 10 μM primer LB3R (or RB3R for analysis of the right border) and 1.25 U Platinum Taq (Life Technologies). Cycling conditions are: 94° C., 10 sec.; thirty cycles of 94° C., 1 sec. −54° C., 72° C., 1 sec.; 72° C., 4 sec. The expected band size for an intact left border is bp, while an intact right border generates a bp band.

Fragments containing left or right border T-DNA sequence and adjacent genomic DNA sequence are obtained via PCR. First product PCR reactions use the following reaction mixture: 1 2 mm² FTA sample, 12.44 μl distilled water, 2 μl 10×Platinum PCR buffer (Life Technologies), 0.6 μl 50 mM MgCl₂, 0.4 μl 10 mM dNTPs, 0.4 μl 10 [M primer LB1 (or RB1 for analysis of the right border), 3 μl 20 μM primer AD2 and 0.8 U Platinum Taq (Life Technologies). Cycling conditions for these reactions are: 93° C., 1 min.; 95° C., 1 min.; three cycles of 94° C., 45 sec. −62° C., 1 min. −72° C., 2.5 min.; 94° C., 45 sec.; 25° C., 3 min.; ramp to 72° C. in 3 min.; 72° C., 2.5 min.; fourteen cycles of 94° C., 20 sec. −68° C., 1 min. −72° C., 2.5 min. −94° C., 20 sec.; −68° C., 1 min. −72° C., 2.5 min. −94° C., 20 sec. −44° C., 1 min. −72° C., 2.5 min,; 72° C., 5 min.; end; ˜4.5 hrs. For second product PCR reactions 1 μl of a 1:50 dilution of the first PCR product reaction is mixed with 13.44 μl distilled water, 2 μl 10×Platinum PCR buffer (Life Technologies), 0.6 μl 50 mM MgCl₂, 0.4 μl 10 mM dNTPs, 0.4 μl 10 μM primer LB2 (or RB2 for analysis of the right border), 2 μl 20 μM primer AD2 and 0.8 U Platinum Taq (Life Technologies). Second product cycling conditions are: eleven cycles of 94° C., 20 sec. −64° C., 1 min. −72° C., 2.5 min. −94° C., 20 sec. −64° C., 1 min. −72° C., 2.5 min. −94° C., 20 sec. −44° c., 1 min.; 72° C., 5 min.; end; 3 hrs. Third product PCR reactions were prepared by first diluting 2 μl of the second PCR product with 98 μl of distilled water and then adding 1 μl of the dilution to 13.44 μl distilled water, 2 μl 10×Platinum PCR buffer (Life Technologies), 0.6 μl 50 mM MgCl₂, 0.4 μl 10 mM dNTPs, 0.4 μl 10 μM primer LB3 (or RB3 for analysis of the right border), 2 μl 20 μM primer AD2 and 0.8 U Platinum Taq (Life Technologies). Third product cycling conditions are: twenty cycles of 94° C., 38 sec. −44° C., 1 min. −72° C., 2.5 min.; 72° C., 5 min.; end; ˜2 hrs. Aliquots of the first, second and third PCR products are electrophoresed on 1% TAE (40 mM Tris-acetate, 1 mM EDTA) to determine their size.

Reactions are purified prior to sequencing by conducting a final PCR reaction. Here, 0.25 μI Platinum PCR Buffer (Life Technologies), 0.1 μl 50 mM MgCl₂, 3.3 U SAP shrimp alkaline phosphatase, 0.33 U Exonuclease and 1.781 μl distilled water are added to a 5 μl third product and the reaction cycled at 37° C., 30 min.; 80° C., 10 min.; 4° C. indefinitely.

Di-deoxy “Big Dye” sequencing is conducted on Perkin-Elmer 3700 or 377 machines.

Knock-in Experiments

For the following examples, a two-component system is constructed in a plant to ectopically express the desired cDNA.

First, a plant is generated by inserting a sequence encoding a transcriptional activator downstream of a desired promoter, thereby creating a first component where the desired promoter facilitates expression of the activator generated a plant. The first component is also referred to as the activator line.

Next, the second component is constructed by linking a desired cDNA to a sequence that the transcriptional activator binds to and facilitate expression of the desired cDNA. The second component is inserted into the activator line by transformation. Alternatively, the second component is inserted into a separate plant, also referred to as the target line. Then, the target and activator lines are crossed to generate progeny that have both components.

Two component lines are generated by both means.

Part I—From Crosses

Target lines containing cDNA constructs are generated using the Agrobacterium-mediated transformation. Selected target lines are genetically crossed to activation lines (or promoter lines). Generally, the promoter lines used are as described above. Evaluation of phenotypes is done on the resulting F1 progenies.

Part II—From Type I Supertransformation

Promoter activation lines (generally Vascular/Ovule/Young Seed/Embryo line, Seed/Epidermis/Ovary/Fruit line, Roots/Shoots/Ovule line, and Vasculature/Meristem are transformed with cDNA constructs using the Agrobacterium mediated transformation. Selected transformants (and their progenies) are evaluated for changes in phenotypes. The table for the knock-in of the Type I supertransformation comprises the following information

Clone ID,

Pfam,

Gemini ID

Trans. Unique ID (which indicates what promoter activation line was transformed

S Ratio: segregation ratio after the transformed plants are selected for the marker.

Assay

Stage: phenotype was observed

Feature: Where the phenotype was observed

Phenotype

P Ratio: phenotype ratio

Comments

Part III—From Type II Supertransformation

Target lines generated using the procedure mentioned in Part I are transformed with T-DNA construct containing constitutive promoter. Selected transformants (and their progenies) are evaluated for changes in phenotypes.

An additional deposit of an E. coli Library, E. coli LibA021800, was made at the American Type Culture Collection in Manassas, Va., USA on Feb. 22, 2000 to meet the requirements of Budapest Treaty for the international recognition of the deposit of microorganisms. This deposit was assigned ATCC accession no. PTA-1411. Additionaly, ATCC Library deposits; PTA-1161, PTA-1411 and PTA-2007 were made at the American Type Culture Collection in Manassas, Va., USA on; Jan. 7, 2000, Feb. 23, 2000 and Jun. 8, 2000 respectively, to meet the requirements of Budapest Treaty for the international recognition of the deposit of microorganisms.

The invention being thus described, it will be apparent to one of ordinary skill in the art that various modifications of the materials and methods for practicing the invention can be made. Such modifications are to be considered within the scope of the invention as defined by the following claims.

The scientific periodical and patent publications that follow are discussed in the Specification and are hereby incorporated by reference in their entirety:
Ahn et al. (1993) Molecular and General Genetics 241: 483-90
Albert et al. (1995) Plant J. 7:649
Alonso-Blanco et al. (c. 1998 ) Methods in Molecular Biology, vol.82, “Arabidopsis Protocols”, pp. 137-146, J. M. Martinez-Zapater and J. Salinas, eds., Humana Press, Totowa, N.J.
Armaleo et al. (1990) Current Genetics 17:97
Ausubel et al. (1992) Current Protocols in Molecular Biology, Greene Publishing, New York). Azpiroz-Leehan et al. (1997) Trends in Genetics 13:152
Binding (1988) Regeneration of Plants, Plant Protoplasts, pp. 21-73, CRC Press, Boca Raton,
Bonneretal. (1973) J. Mol. Biol. 81:123
Bradshaw et al. (1995) Nucl Acids Res 23: 4850-4856
Buchanan, Gruissem, and Russel (eds) 2000, Biochemistry and Molecular Biology of Plants,, Amer. Soc. of Plant Physiologists, Rockville, Md.
Burke et al. (1987) Science 236:806-812
Burr (c. 1994 ) “Mapping Genes with Recombinant Inbreds”, pp. 249-254. In Freeling, M. and V. Walbot (Ed.), The Maize Handbook Springer-Verlag New York, Inc.: New York, N.Y., USA; Berlin Germany
Burr et al. (1998) Genetics 118: 519
Carels et al. (1998) J. Mol. Evol. 46: 45
Chang et al. (1993) Gene 127:95
Christou, (1995) Euphytica 85 (1-3):13-27
Chusacultanachai et al. (1999) J. Biol. Chem. 274:23591
Cox and Goldberg (c. 1988 ) “Plant Molecular Biology: A Practical Approach”, pp. 1-35, Shaw ed., IRL, Oxford
De Martinis et al. (1999) Plant Cell 11:1061
de Vicente and Tanksley (1993) Genetics 134:585
Dumas Milne Edwards J. B. (20 Dec. 1993) Doctoral Thesis of Paris VI University, Le clonage des ADNc complets: difficultés et perspectives nouvelles. Apports pour l'étude de la régulation de l'expression de la tryptophane hydroxylase de rat.
Escudero et al. (1996) Plant J. 10:355
Evans et al., (1983)Protoplasts Isolation and Culture in “Handbook of Plant Cell Culture,” pp. 124-176, MacMillan Publishing Company, New York
Fennoy et al. (1993) Nuc. Acids Res. 21(23): 5294 (scientific periodical and patent publications incorporated by reference continued)
Frischaufet al. (1983) J. Mol Biol 170: 827-842
Fromm et al. (1985) Proc. Natl Acad. Sci. USA 82:5824
Fry et al. (1992) Biotechniques 13: 124
Gardiner, J. et al. (1993) Genetics 134: 917
Ghosh et al. (1994) J. Biotechnol. 32:1
Gleave (1992) Plant Mol. Biol. 20:1203
Golovkin et al. (1999) J. Biol. Chem. 274:36428
Gould et al. (1991) Plant Physiology 95:426
Graves and Goldman (1986) Plant Mol. Biol. 7:34
Hamilton et al (1990) Nature 346:284-287
Hamilton et al. (1996) Proc. Natl. Acad. Sci. USA 93: 9975-9979
Hamilton (1997) Gene 200:107
Haseloff et al. (1988) Nature, 334:585
Herrera-Estrella et al. (1983) EMBO J. 2:987
Hochstrasser (2000) Science 289: 563
Hosoyama et al. (1994) Biosci. Biotechnol. Biochem. 58:1500
Hu et al. (2000) Methods 20:80
Huynh et al., In: Glover N. Mex. (ed) DNA Cloning: A practical Approach, Vol.1 Oxford: IRL Press (1985
Ichimura et al. (1998) Biochem. Biophys. Res. Comm. 253:532
Ishida et al. (1996) Nature Biotechnology 14:745
Joazeiro and Hunter (2001) Science 289: 2061;
Kaczor et al. (1994) Plant Physiol. 1-4: 1411-7
Kato et al. (1994) Gene 150:243-250
Keegstra and Cline (1999) The Plant Cell 11: 557-570
Keller and M. M. Manak (c. 1993 )“DNA Probes”, 2^{nd} Ed. pp. 1-25, by Stockton Press, New York, N.Y.
Klee et al. (1987) Ann. Rev. ofPlant Phys. 38:467
Klein et al. (1987) Nature 327:773
Kohler and Milstein (1975) Nature 256: 495
Koltonow et al. (1990) Plant Cell 2:1201
Komari et al. (1996) Plant J. 10:165
Liljegren (1999) Plant Cell 11:1007
Lin et al. (1997) J. Biol. Chem. 272:27274
Luo et al. (1997) J. Mol. Biol. 266:479
Marra et al. (1997) Genomic Research 7:1072-1084
Mariani et al. (1992) Nature 357: 384-387
Martinez et al. (1999) Mol. Gen. Genet. 261:546
Maruyama et al. (1994) Gene 138:171
Marty (1999) The Plant Cell 11: 587-599
Matteucci et al. ( 1981) J. Am. Chem. Soc. 103:3185
May et al. (1995) Bio/Technology 13:486
McAlister-Henn et al. (1999) Methods 19:330
McCormac et al. (1997) Mol. Biotechnol. 8:199 (scientific periodical and patent publications incorporated by reference continued)
Michaels et al. (1999) Plant Cell 11:949
Morgante et al. (1993) The Plant Journal 3: 165
Müiller et al. (1987) Mol. Gen. Genet. 207:171
Napoli et al. (1990) The Plant Cell 2:279
Oeller, et al (1991) Science 254:437-439
Panaud et al. (1995) Genome 38: 1170
Paszkowski et al. (1984)EMBO J. 3:2717
Pearson and Lipman (1988) Proc. Natl. Acad Sci. (USA) 85: 2444
Pierrou et al., (1995) Anal. Biochem. 229:99
Refseth et al. (1997) Electrophoresis 18: 1519
Sakamoto et al. (2001) PNAS Online 141230798
Salomon et al. (1984) EMBO J. 3:141
Sambrook et al. (1989) Molecular Cloning: A Laboratory Manual, 2^{nd} ed. Cold Spring Harbor Laboratory Press, New York)
Schneider (1976) Z. naturforsch. [C] 31: 55-63
Schwarz et al. (1992) Mol. Cell. Biol. 12:266
Seki et al. (1998) Plant Journal 15:707-720
Senior et al. (1996) Crop Science 36: 1676
Sheehy et al. (1988) Proc. Nat. Acad. Sci. USA, 85:8805
Shizuya et al. (1992) Proc. Natl. Acad. Sci. USA 89: 8794-8797
Smith (1988) Biochem. J. 249: 423-8
Smith and Waterman (1981) Add. APL. Math. 2:482
Spiker and Thompson (1996) Plant Physiol. 110: 15-21
Stemberg N. et al. (1990) Proc Natl Acad Sci US A. 87(1):103-7
Tanksley et al. (1997) Science 277:1063
Tanksley and McCouch (1997) Science 277:1063
Taramino et al. (1996) Genome 39: 277
Tijessen (c. 1993) “Hybridization with Nucleic Acid Probes” in Laboratory Techniques in Biochemistry and Molecular Biology, P. C. vand der Vliet, ed., Elsevier, Amsterdam.
Urdea et al. (1981) Proc. Natl. Acad. 80:7461
van der Krol et al. (1990) Plant Cell 2:291
Venkateswarlu et al. (1991) Biotechnology 9:1103
Vergunst et al. (1998) Nucleic Acids Res. 26:2729
Vergunst et al. (1998) Plant Mol. Biol. 38:393
Vitale and Denecke (1999) The Plant Cell 11: 615-628
Walden et al. (1990) Mol Cell Biol 1: 175-194
Weigel and Meyerowitz, (1993) In Molecular Basis of Morphogenesis (ed. M. Bemfield). 51st Annual Symposium of the Society for Developmental Biology, pp. 93-107, New York
Weising et al. (1988) Ann. Rev. Genet. 22:421
Wunsch (1970) J. Mol. Biol. 48:443
Xu et al. (1995) Plant Mol. Biol. 27:237
Yamamoto et al. (1991) Plant Cell 3:371 (scientific periodical and patent publications incorporated by reference continued)
Bouckaert et al., U.S. Ser. No. 60/121,700 Atty. Dkt. No. 2750-117P, Client Dkt. No. 00010.001
EPO 625572
U.S. Pat. No. 4,801,340
U.S. Pat. No. 5,034,323
U.S. Pat. No. 5,231,020
U.S. Pat. No. 5,283,184
U.S. Pat. No. 5,410,270
U.S. Pat. No. 5,445,943
U.S. Pat. No. 5,766,847
U.S. Pat. No. 5,945,306
U.S. Pat. No. 5,945,287

[image]

1. An isolated nucleic acid molecule comprising:
a) a full length cDNA nucleic acid having a nucleotide sequence which encodes an amino acid sequence exhibiting at least 40% sequence identity to an amino acid sequence encoded by
(1) a full length cDNA nucleotide sequence described in the Seqeuence Listing or the Sequence Listing-Miscellaneous Feature documents, or a fragment thereof; or
(2) a complement of a full-length cDNA nucleotide sequence shown in the Seqeuence Listing or the Sequence Listing-Miscellaneous Feature documents, or a fragment thereof;
b) a nucleic acid which is the reverse of the nucleotide sequence according to subparagraph (a), such that the reverse nucleotide sequence has a sequence order which is the reverse of the sequence order of the nucleotide sequence according to subparagraph (a);
c) a nucleic acid capable of hybridizing to a nucleic acid having a sequence selected from the group consisting of: a full-length cDNA nucleotide sequence which is shown in the Seqeuence Listing or the Sequence Listing-Miscellaneous Feature documents; and a nucleotide sequence which is complementary to a full-length cDNA nucleotide sequence shown in the Seqeuence Listing or the Sequence Listing-Miscellaneous Feature documents, under conditions that permit formation of a nucleic acid duplex at a temperature from about 40° C. and 48° C. below the melting temperature of the nucleic acid duplex, with the proviso that said nucleotide sequence is not any of the sequences described in the Tables of any of Patent Publication Nos. WO 200040695, CA 2300692 A1, EP 1033405 A2, CA 2302828 A1 and EP 1059354 A2 and any proteins listed in the application that are identified by gi number or otherwise as being from the non-redundant GenBank CDS translations or Protein Database (PDB), available via the internet, or (PIR-International) Database (PIR), available via the internet.

2. An isolated nucleic acid molecule comprising a nucleic acid having a nucleotide sequence which exhibits at least 65% sequence identity to
a) a full-length cDNA nucleotide sequence shown in the Seqeuence Listing or the Sequence Listing-Miscellaneous Feature documents, or a fragment thereof; or
b) a complement of a full-length cDNA nucleotide sequence described in the Seqeuence Listing or the Sequence Listing-Miscellaneous Feature documents, or a fragment thereof, with the proviso that said nucleotide sequence is not any of the sequences described in the Tables of any of Patent Publication Nos. WO 200040695, CA 2300692 A1, EP 1033405 A2, CA 2302828 A1 and EP 1059354 A2 and any proteins listed in the application that are identified by gi number or otherwise as being from the non-redundant GenBank CDS translations or Protein Database (PDB), or (PIR-International) Database (PIR).

3. The nucleic acid molecule according to claim 1, wherein said nucleic acid comprises an open reading frame.

4. (canceled)

5. (canceled)

6. A vector construct comprising:
a) a first nucleic acid having a regulatory sequence capable of causing transcription and/or translation; and
b) a second nucleic acid having the sequence of the isolated nucleic acid molecule according to claim 1;
wherein said first and second nucleic acids are operably linked and wherein said second nucleic acid is heterologous to any element in said vector construct.

7. The vector construct according to claim 6, wherein said first nucleic acid is native to said second nucleic acid.

8. The vector construct according to claim 6, wherein said first nucleic acid is heterologous to said second nucleic acid.

9. A host cell comprising an isolated nucleic acid molecule according to claim 1, wherein said nucleic acid molecule is flanked by exogenous sequence

10. A host cell comprising a vector construct of claim 6.

11. An isolated polypeptide comprising an amino acid sequence
a) exhibiting at least 40%, or 75%, or 85%, or 90% sequence identity of an amino acid sequence encoded by a sequence shown in the Seqeuence Listing or the Sequence Listing-Miscellaneous Feature documents, or a fragment thereof; and
b) capable of exhibiting at least one of the biological activities of the polypeptide encoded by said nucleotide sequence shown in the Seqeuence Listing or the Sequence Listing-Miscellaneous Feature documents, or a fragment thereof, with the proviso that said nucleotide sequence is not any of the sequences described in the Tables of any of Patent Publication Nos. WO 200040695, CA 2300692 A1, EP 1033405 A2, CA 2302828 A1 and EP 1059354 A2 and any proteins listed in the application that are identified by gi number or otherwise as being from the non-redundant GenBank CDS translations or Protein Database (PDB), or (PIR-International) Database (PIR),.

12. An antibody capable of binding the isolated polypeptide of claim 11.

13. A method of introducing an isolated nucleic acid into a host cell comprising:
a) providing an isolated nucleic acid molecule according to claim 1; and
b) contacting said isolated nucleic with said host cell under conditions that permit insertion of said nucleic acid into said host cell.
c) A method of transforming a host cell which comprises contacting a host cell with a vector construct according to claim 6.
d) A method of modulating transcription and/or translation of a nucleic acid in a host cell comprising:
e) providing the host cell of claim 9; and
f) culturing said host cell under conditions that permit transcription or translation.

14. A method for detecting a nucleic acid in a sample which comprises:
a) providing an isolated nucleic acid molecule according to claim 1;
b) contacting said isolated nucleic acid molecule with a sample under conditions which permit a comparison of the sequence of said isolated nucleic acid molecule with the sequence of DNA in said sample; and
c) analyzing the result of said comparison.

15. A plant or cell of a plant which comprises a nucleic acid molecule according to claim 1 which is exogenous or heterologous to said plant or plant cell.

16. A plant or cell of a plant which comprises a vector construct according to claim 6.

17. A plant which has been regenerated from a plant cell according to claim 17.

18. A plant which has been regenerated from a plant cell according to claim
